SEQUENCE LISTING (110 sequences)

SEQ ID NO: 1 - DNA, 1152 bp, Novosphingobium aromaticivorans
  source 1..1152: mol_type = genomic DNA
atgctatcggaccgccacgtcaaagggagaccgcacgaaatgaagacccgcgccgcagttgcgttcgcgcccaagcagccgctcgagatcgtcgaactggacctcgaaggccccaaggctggcgaagtgctggtcgagatcatggcgaccggcgtgtgccacaccgatgcctacacgctcgacgggttcgacagcgaaggcatcttccccagcgtgctgggccacgaaggcgccggtatcgtgcgcgaggtgggccctggggtcacttcggtgaagcccggcgatcacgtgatcccgctctacacgccggaatgccgccagtgcaaatcgtgcctctcgggcaagaccaacctgtgcaccgcgatccgcgccacgcaagggcagggcctgatgcccgacggcaccagccgcttttcgtacaagggccagaccgtgttccactacatgggctgctcgaccttctctaacttcaccgtcctgcccgagatcgcggttgccaagatccgcgaggacgcgccgttcaagacctcgtgctatatcggctgcggcgtgacgacgggcgtcggcgcggtgatcaacaccgccaaggtccaggtcggtgacaacgtcgtggtcttcggcctcggcggcatcggcctcaacgtgatccagggcgcgcggcttgccggtgccggcaagatcatcggcgtcgacatcaaccccgaccgcgaggaatggggccgcaagttcggcatgaccgacttcctcaacagcaagggcatgagccgcgaggacgtcgtcgccaaggtcgtcgccatgaccgacggcggcgcggactacaccttcgacgccaccggcaacaccgaagtgatgcgcacggcgcttgaagcctgccatcgcggctggggcacctccatcatcatcggcgtggccgaggcgggcaaggaaatcagcacgcgtccgttccagctcgtcaccggccgcaactggcgcggcacggccttcggcggcgccaagggccgcaccgacgtgcccaagatcgtcgacatgtacatgaccggcaagatcgagatcgacccgatgatcacccatgtcatgggcctggaagagatcaacaccgccttcgacctgatgcacgccggcaagtcgatccgttcagtcgtggtgttctga

SEQ ID NO: 2 - protein, 383 aa, Novosphingobium aromaticivorans
MLSDRHVKGRPHEMKTRAAVAFAPKQPLEIVELDLEGPKAGEVLVEIMATGVCHTDAYTLDGFDSEGIFPSVLGHEGAGIVREVGPGVTSVKPGDHVIPLYTPECRQCKSCLSGKTNLCTAIRATQGQGLMPDGTSRFSYKGQTVFHYMGCSTFSNFTVLPEIAVAKIREDAPFKTSCYIGCGVTTGVGAVINTAKVQVGDNVVVFGLGGIGLNVIQGARLAGAGKIIGVDINPDREEWGRKFGMTDFLNSKGMSREDVVAKVVAMTDGGADYTFDATGNTEVMRTALEACHRGWGTSIIIGVAEAGKEISTRPFQLVTGRNWRGTAFGGAKGRTDVPKIVDMYMTGKIEIDPMITHVMGLEEINTAFDLMHAGKSIRSVVVF

SEQ ID NO: 3 - DNA, 1089 bp, Novosphingobium aromaticivorans
  source 1..1089: mol_type = genomic DNA
atgaaagccgccgtactcgtcgaaccgggcaagccgctggatattcagcatctcagcgtgtccaagcccggcccgcatgaagtccttatccgcaccgcagcctgcgggctgtgccattcggacttgcacttcatcgaaggtgcctatccccatccgctgcccgcggtgccggggcacgaggcggcggggatcgtcgaggcggtcggctcggaagtgcgcacggtcaaggtgggtgacgcggtcgtcacctgcctgtccgcgttctgcggtcattgcgagttctgcgtgaccggccggatgtcgctgtgccttggcggcgacacccggcgcggcgcgggcgaggcacctcgccttacccgcaccgacgacggcagcgccgtgaaccagatgctcaacctctcggcctttgccgaacagatgctggtgcacgaacatgcctgcgtggcgatcaatcccgagatgccgctcgaccgcgcggcggtgatcggctgcgcggtcaccactggcgcgggtgcggtgttcaacgcggcgaagctgaccccgggcgagacggtctgcgtggtcggctgtggcggcgtcggccttgccacggtcaacgccgcgaagatcgccggcgcaggccggatcatcgcggtggacccgatgccggaaaagcgcgaactggccatgaagctgggcgcgaccgatgtgatggacgcgggacccgatgcggcggcacagatcgtcgagatgacgaaaggcggcgtccaccatgcgatcgaggccgtggggcgtccggcatcgggcgaccttgcggtcgcgacgctgcgccgcggcggcaccgccacgatccttggcatgatgccgctggcacacaaggtcggactttccgcgatggacctgctgtcggacaagaagctgcagggcgccatcatgggccgcaaccacttcccggtggacctgccgcgcctggtcgacttctacatgcgcggcttgctcgatctcgacacgatcattgccgaacgcatcccgctcgaagggatcaacgatggcttcgagaagatgaagcagggccattccgcccgctctgtcatcgtgttcgaccaatga

SEQ ID NO: 4 - protein, 362 aa, Novosphingobium aromaticivorans
MKAAVLVEPGKPLDIQHLSVSKPGPHEVLIRTAACGLCHSDLHFIEGAYPHPLPAVPGHEAAGIVEAVGSEVRTVKVGDAVVTCLSAFCGHCEFCVTGRMSLCLGGDTRRGAGEAPRLTRTDDGSAVNQMLNLSAFAEQMLVHEHACVAINPEMPLDRAAVIGCAVTTGAGAVFNAAKLTPGETVCVVGCGGVGLATVNAAKIAGAGRIIAVDPMPEKRELAMKLGATDVMDAGPDAAAQIVEMTKGGVHHAIEAVGRPASGDLAVATLRRGGTATILGMMPLAHKVGLSAMDLLSDKKLQGAIMGRNHFPVDLPRLVDFYMRGLLDLDTIIAERIPLEGINDGFEKMKQGHSARSVIVFDQ

SEQ ID NO: 5 - DNA, 1098 bp, Novosphingobium aromaticivorans
  source 1..1098: mol_type = genomic DNA
atggacgcatacgcggcaattatcgagcgtcaaggcggcgaattcgttctggataacgtctctatcgaggatccgcgcgacggcgaagtgctggtcaaggttgccgcagctggcatgtgtcataccgacctgacggttcgcgatcaatattacccgacgccgctgccggcggtgctgggccatgaaggttcgggcgttgtcgaaaaggtcggacgtggcgtcaccactgtcaagccaggcgacaaggtcgtgctctccttcagctattgcggcacctgtccatcgtgcctcaaggggcatcaggcctattgtccgagcctgttcccgctcaatttcatgggccgccgcctggatggttcgacgccgattacccgcaacggccaagaggtcaacgcctgcttcttcgggcaatcctcgttcgcgacctattcgatcgcgtcggaaaacaactgcgtcaaggttgccgacgacgcacagatcgaacttttgggcccactgggctgcggcatccagaccggggcgggcagcatcctcaatgcgctttgtcccgaacctggctcctcgatcgcgatcttcggggtcgggtcggtcggcctcagcgccgtgatggccgccaaggcctcgggctgcctcaagatcatcgcggttgaccgcaacgcaggccgcttggaactggcgcgtgaactgggcgccaccgatgtgatcgacgccaacacggtcaacgctcaggaagcgatcgtcgcgatgaccggtggcggcgccgactatgccatggataccaccgccattccagcggtgctgcgctcggcggtggacagcacgcacaacatgggtgaaaccgcagtggtcggcggggcgaagctgggcaccgagttttcgctagacatgaacaacatgctgtttggccgcaagttgcgcggcgtagtcgaaggatcgagcaccccgcaggtcttcatcccgcaactgattgcgatgcagaaggccgggctgttcccgttcgagaagctctgcaccttctatgatctcgaccagatcaaccaggccgtcgaggataccgaaaagaccggcaaggcgatcaaggccattctcaaaatgtag

SEQ ID NO: 6 - protein, 365 aa, Novosphingobium aromaticivorans
MDAYAAIIERQGGEFVLDNVSIEDPRDGEVLVKVAAAGMCHTDLTVRDQYYPTPLPAVLGHEGSGVVEKVGRGVTTVKPGDKVVLSFSYCGTCPSCLKGHQAYCPSLFPLNFMGRRLDGSTPITRNGQEVNACFFGQSSFATYSIASENNCVKVADDAQIELLGPLGCGIQTGAGSILNALCPEPGSSIAIFGVGSVGLSAVMAAKASGCLKIIAVDRNAGRLELARELGATDVIDANTVNAQEAIVAMTGGGADYAMDTTAIPAVLRSAVDSTHNMGETAVVGGAKLGTEFSLDMNNMLFGRKLRGVVEGSSTPQVFIPQLIAMQKAGLFPFEKLCTFYDLDQINQAVEDTEKTGKAIKAILKM

SEQ ID NO: 7 - DNA, 1431 bp, Novosphingobium aromaticivorans
  source 1..1431: mol_type = genomic DNA
gtgactgcgtacccttcgctccacatgatcatcgacggcgcccgcgtcagcggcggcggacgtcgcacccacgcggtcgtcaatcccgctaccggagagaccatcggcgaactgccgctggccgaagtcgccgatctcgaccgcgcgctcgaagtcgcggcgaagggcttccgcatctggcgcgacagcacgccgcagcagcgcgcagccgtgctccagggcgcggcccgcctgatgctggaacggcaggaggacctcgcccgcatcgccacgatggaagaaggcaagaccctgcccgaggcgcgcatcgaagtcctgatgaacgtgggcctgttcaacttctacgccggcgaggtattccggctctatggccgcaccctcgtgcgccctgcgggtcagcgcagcacgatcacgcatgaaccggtcgggcccgtggccgcctttgcgccgtggaactttccgctcggcaaccccggccgcaagctcggcgcgcccattgccgccggttgctcggtgatcctcaaggcggcggaagaaacgccggcctccgcgctcggggtgctgcaatgcctgctcgatgcgggcctgcccaaggaagtggcccaggccgtgttcggtgtgcctgacgaggtgagtcgccacctgctcggctcgtccgtcatccgcaagctctcgttcaccggctcgaccgtcatcggcaagcacctcatgcgccttgccgccgacaacatgttgcgcacaacgatggagcttggcggccacggccctgtcctcgtcttcggcgatgccgatatcgacaaggcgctcgataccatggccgcgtccaagtatcgcaacgcgggccaggtctgcgtctcgccaacccgcttcatcgtggaagagagcgtgttcgaacgcttccgcgacggttttgccgagcgcgtcggccggatcaaggtcggcaacggcctcgatcaggatgcgcagatgggccccatggccaacgcccgccgccccgaggcgatggatcgcctgatcggggacgccgtgacccgcggcgcaaggctccacaccgggggcgagcgcgtcggcaacgccggctatttctacgcccccacggtcctgtccgaagtcccgctcgacgcggcgatcatgaacgaggagccgttcggcccggtcgcgctgatcaatcccttcggcggcgaggaagcgatgatcgccgaggccaaccgcctgccctacggcctcgccgcctacgcctggaccgacagcgcggcgcgggccaagcgcctcgcccgcgagatcgagacggggatgctcgggcttaactcgaccatgatcggcggcgcggattcgcccttcggcggggtcaagtggtccggccacggctccgaggacggtcccgaaggcgtcatggcctgccttgtcaccaaggcggtccacgaagggtaa

SEQ ID NO: 8 - protein, 476 aa, Novosphingobium aromaticivorans
VTAYPSLHMIIDGARVSGGGRRTHAVVNPATGETIGELPLAEVADLDRALEVAAKGFRIWRDSTPQQRAAVLQGAARLMLERQEDLARIATMEEGKTLPEARIEVLMNVGLFNFYAGEVFRLYGRTLVRPAGQRSTITHEPVGPVAAFAPWNFPLGNPGRKLGAPIAAGCSVILKAAEETPASALGVLQCLLDAGLPKEVAQAVFGVPDEVSRHLLGSSVIRKLSFTGSTVIGKHLMRLAADNMLRTTMELGGHGPVLVFGDADIDKALDTMAASKYRNAGQVCVSPTRFIVEESVFERFRDGFAERVGRIKVGNGLDQDAQMGPMANARRPEAMDRLIGDAVTRGARLHTGGERVGNAGYFYAPTVLSEVPLDAAIMNEEPFGPVALINPFGGEEAMIAEANRLPYGLAAYAWTDSAARAKRLAREIETGMLGLNSTMIGGADSPFGGVKWSGHGSEDGPEGVMACLVTKAVHEG

SEQ ID NO: 9 - DNA, 1425 bp, Novosphingobium aromaticivorans
  source 1..1425: mol_type = genomic DNA
atgcgcgaacggctacagcaatacattgatggcaagtgggtagacagcgagggtggcaagcgccacgaggtcatcaatccgacgaccgaggaaccctgctgcgtcatcacgctgggcacgcaggccgatgtcgacaaggcagtggccgcggcccagcgcgccttcaagaccttcagcaagacgacgcgcgaggagcgactcgcgctgcttgaacgcatcgtcgaggaatacaagaagcgcgtccccgatctcgccgccgcgatggccgaggaaatgggcgctccggtaagcttcgccagcaccgcgcaggtcggcgccggcatcggcgccttcctcggcaccatggccgcgctccgcaacttctccttcgtcgaggacaacggtgcgttcaaggtcgcctacgaaccgatcggcgtcgtcggcatgatcacgccatggaactggcccctcaaccagatcgcgctcaaggtcgcaccggcgctggccgcgggcaacaccatgatcctcaagccgtccgaggaatgccccaccaacgccgcgatctttaccgagatcctcgatgccgccggcgtcccgccaggcgtcttcaacctcatccagggcgatggtcccggcgtcggcactgcgatcagctcgcacccgggcatcgacatggtcagcttcaccggctcgacccgcgcgggcatcctcgtggcgaaggctgcggccgataccgtcaagcgcgtccatcaggagcttggcggcaagtcgcccaacgtcgtcctgcccgatgcagacttcgccaagtacctgccgtcgaccgcgtccggcccgttggtcaacagcggccagagctgcatttcgcccacccgcattctcgtaccccgcgaacgcgaagccgaagccgcggcgttcgtttcggcgatgtactcggcaaccccggtcggcgatccgatgcaggaaggtgcgcacatcggcccggtggtcaacaaggcgcagttcgacaagatccgcggcctgatccagtcggcgatcgacgaaggcgcgaagctcgagaccggcggccccgacctcccggccaacgtcaaccgcggctactacatcaagcccacggtcttctccggcgtcacgcccgacatgcgcattgcgcaggaggaaatcttcggcccggtcgcgacgatcatggcgtacgacagcctcgaggaggccatcgagatcgccaacgacaccgcctatggcctgtcggcctgcatcaccggcgatccggcgaaggcggctgaagtcgcgcccgagcttcgcgccggcatggtcgcgatcaacaactggggccccaccccgggcgcgccgttcggcggctacaagcagtccggcaacggccgcgagggcgggctctatggcctcaaggacttcatggaaatgaaggcgatcagcggcctgcctgcctga

SEQ ID NO: 10 - protein, 474 aa, Novosphingobium aromaticivorans
MRERLQQYIDGKWVDSEGGKRHEVINPTTEEPCCVITLGTQADVDKAVAAAQRAFKTFSKTTREERLALLERIVEEYKKRVPDLAAAMAEEMGAPVSFASTAQVGAGIGAFLGTMAALRNFSFVEDNGAFKVAYEPIGVVGMITPWNWPLNQIALKVAPALAAGNTMILKPSEECPTNAAIFTEILDAAGVPPGVFNLIQGDGPGVGTAISSHPGIDMVSFTGSTRAGILVAKAAADTVKRVHQELGGKSPNVVLPDADFAKYLPSTASGPLVNSGQSCISPTRILVPREREAEAAAFVSAMYSATPVGDPMQEGAHIGPVVNKAQFDKIRGLIQSAIDEGAKLETGGPDLPANVNRGYYIKPTVFSGVTPDMRIAQEEIFGPVATIMAYDSLEEAIEIANDTAYGLSACITGDPAKAAEVAPELRAGMVAINNWGPTPGAPFGGYKQSGNGREGGLYGLKDFMEMKAISGLPA

SEQ ID NO: 11 - DNA, 1347 bp, Novosphingobium aromaticivorans
  source 1..1347: mol_type = genomic DNA
atgactgccccgaccgccgccgacctttccgccgacatcgcacgcgtcttcgcactccagcaggcgcacatgtgggaggccaaggcctccaccgcggccgagcgcaaggaaaagctcgcgcgcctcaaggccgccgtcgaagcccacgccgacgacatcgtcgccgccgtcctcgaagacacgcgcaagccggttggcgaaatccgcgtgaccgaagtcctcaacgtcaccgccaacatccagcgcaacatcgacaatctcgatgaatggatgaagccggtcgaggtcgccacctcgctcaatcccgccgaccgcgcgcagatcatccacgaagcgcgcggcgtctgcctgatccttggcccctggaacttccccctcggcctcgcgctcggtccggtcgccgctgccatcgccgcaggcaacacctgcatcgtgaagctcaccgacctctgccccgccaccgcaagggtggcctcggtgatcgtcagggaagcgttcgacgaaaaggatgtggctctgttcgaaggcgacgtctcggtcgccaccgcgctcctcgatctgccgttcaaccacgtcttcttcaccggctcgccccgcgtcggcaagatcgtgatggccgctgccgcaaagcacctcaccagcgtcacgctcgaacttggcgggaagtcgcccgtcatcgtcgacgatagcgccgacatcgatcaggtcgccgcccagctcgccgcggccaagcagttcaacggcgggcaggcctgcatcagcccggactacgtcttcgtgaaggaagacaagaaggccgcgctggtcgaaggcttccgggccaacgtgcagaagaacctctatgacgatgccggcaacctgaagaaggacagcatcgcccaggtggtcaacaaggcgaacttcgaccgcgtgaaggccatgttcgacgatgccgtcgccaagggcgcgaccgtcgccgccggcggaacgttcgaagccgatgacctcaccatccatccgaccatgctgaccggcgtcaccccgcagatgaccatcctccaggacgaaatcttcgcccccgtcatcccggtgatgacctacgacacgctcgaccaggcgatcggctacatcgaagcccgcgacaagccgctcgcactctatgtctacagcaaggacgaagcgaacgtcgaaaaggtcctcgcccgcacctcgtcgggcggtgtcacggtgaatggcgtgttctcgcactacctggaaaacaacctgccgttcggcggcgtcaacaccagcggcatgggcagctaccacggcgtgttcggcttcaagtgcttcagccacgaacgggctgtctaccgccaccagcagtaa

SEQ ID NO: 12 - protein, 448 aa, Novosphingobium aromaticivorans
MTAPTAADLSADIARVFALQQAHMWEAKASTAAERKEKLARLKAAVEAHADDIVAAVLEDTRKPVGEIRVTEVLNVTANIQRNIDNLDEWMKPVEVATSLNPADRAQIIHEARGVCLILGPWNFPLGLALGPVAAAIAAGNTCIVKLTDLCPATARVASVIVREAFDEKDVALFEGDVSVATALLDLPFNHVFFTGSPRVGKIVMAAAAKHLTSVTLELGGKSPVIVDDSADIDQVAAQLAAAKQFNGGQACISPDYVFVKEDKKAALVEGFRANVQKNLYDDAGNLKKDSIAQVVNKANFDRVKAMFDDAVAKGATVAAGGTFEADDLTIHPTMLTGVTPQMTILQDEIFAPVIPVMTYDTLDQAIGYIEARDKPLALYVYSKDEANVEKVLARTSSGGVTVNGVFSHYLENNLPFGGVNTSGMGSYHGVFGFKCFSHERAVYRHQQ

SEQ ID NO: 13 - DNA, 1515 bp, Novosphingobium aromaticivorans
  source 1..1515: mol_type = genomic DNA
atgaacgacatgaccaccatctcgcgcacgcagcgcgaatactcggaggccgccaaggccttcctcgcgcgcaagccgcagttgttcatcaacaacgagtgggtcgacagcagccacgacgccgtgatcgaggtggaagacccctcgaacggcaggatcgtcggtcatgtcgtcgatgcctcggacaaggacgtcgaccgggcggttgccgcggcgcgcgccgcgttcgacgatggccgctggtccaacctgccgccaatggtccgcgatcgcaccatgaatcgcctggccgacctgcttgaagccaacgccgatctctttgccgagctcgaagcgatcgacaacggcaagcccaagggcatggccggcgccgtcgacatccccggcgcgatcagccagctccgcttcatggccggctgggccagcaaggtcgcgggcgagacgacgcagccctacacgatgcccaacggcaccgtgttcagctacaccgtcaaggaacccgtcggcgtctgcgcgcagatcgtgccgtggaacttcccgctgctgatggcctcgctcaagatcgccccggcgctggcggctggctgcaccctggtgctgaagcccgccgaacagacctcgcttaccgcgctcaagcttgccgatctcgtggtcgaggccggcttccctgcgggcgtgatcaacatcatcaccggcaacggccacaccgccggtgaccgcatggtcaagcatcccgacgtcgacaaggtcgccttcaccggctcgaccgagatcggcaagctgatcaatcgcaacgccaccaccacgctcaagcgggtcacgctcgaactgggcgggaagagccccgtcgtggtcatgcccgacgtcgacgtggcgcagaccgcgcctggcgttgccggcgcgatcttcttcaacgcgggccaggtctgcgttgccggttcgcgtctctatgcgcaccgttcggtgttcgattccgtgctcgaaggcatgacccagaccgcgccgttctgggcgccgcgcccctcgctggatcccgaagcccacatgggcccgttggtcagcaaggagcagcacgaccgcgtgatgggctacatcgaggcgggcaagcgcgatggcgccagcgtcgtcatgggcggcgattgccccagcgccgatggcgggtactacgtcaacccgacgatccttgcagacgtgaacccgcagatgtcggtcgtgcgcgaggaaatcttcggccccgtcgtcgtcgcccagcgcttcgacgatctcgatgaagtggcgaagatggccaacgacacctgcttcggcctcggcgcgggcgtgtggacgcgcgatgtcgcggtgatgcacaagcttgcctcgaagatcaaatcgggcaccgtgtggggcaactgccacgccctgatcgataccgcgctgccctttggcggctacaaggaatcgggcctgggccgcgaacaggggcgcgccggcatcgacgcctacctcgagaccaagaccgtcatcatccagatgtaa

SEQ ID NO: 14 - protein, 504 aa, Novosphingobium aromaticivorans
MNDMTTISRTQREYSEAAKAFLARKPQLFINNEWVDSSHDAVIEVEDPSNGRIVGHVVDASDKDVDRAVAAARAAFDDGRWSNLPPMVRDRTMNRLADLLEANADLFAELEAIDNGKPKGMAGAVDIPGAISQLRFMAGWASKVAGETTQPYTMPNGTVFSYTVKEPVGVCAQIVPWNFPLLMASLKIAPALAAGCTLVLKPAEQTSLTALKLADLVVEAGFPAGVINIITGNGHTAGDRMVKHPDVDKVAFTGSTEIGKLINRNATTTLKRVTLELGGKSPVVVMPDVDVAQTAPGVAGAIFFNAGQVCVAGSRLYAHRSVFDSVLEGMTQTAPFWAPRPSLDPEAHMGPLVSKEQHDRVMGYIEAGKRDGASVVMGGDCPSADGGYYVNPTILADVNPQMSVVREEIFGPVVVAQRFDDLDEVAKMANDTCFGLGAGVWTRDVAVMHKLASKIKSGTVWGNCHALIDTALPFGGYKESGLGREQGRAGIDAYLETKTVIIQM

SEQ ID NO: 15 - DNA, 741 bp, Novosphingobium aromaticivorans
  source 1..741: mol_type = genomic DNA
gtgtccgatagcaatcagattgccgcgctcgaaagccgcctgaacgacctcgaaaggcgcctgacggtgcgcgaggacgagctggacgtacgcaagctccagcatctctacggctacctgatcgacaagtgcatgtataacgagaccgtggacctgttcaccgaagatggcgaagtgcgcttcttcggcggcgtctggaagggcaaggagggcatccgccgtctctacgtcgaacgtttccagaagcgcttcacctacggcaacaacggcccgatcgacggcttcctgctcgatcacccccagcttcaggacatcatccacgtgcaggatgacggggtcaccgctctcggccgcgcgcggtcgatgatgcaggccggtcgccacaaggattacgagggcgatgccccgcacctcaaggcgcgccagtggtgggaaggcggcatctacgagaacacctacaagaaggtggacggcgtgtggcggatgcacatcctcaactacatgccgatctggcacgccgatttcgaaagcggctgggccaacaccccgcacgaatacgtgccgttccccaaggtcacctatcccgaagacccgaccggaccggacgaactgatcgccgaccactggctctggccgacccacaagctgaaccccttccacatgaagcacccggtgacgggcgaggaaatggtcgcgcagcgctggcagggcgacatcgaccgcgagaacgcgcggaaataa

SEQ ID NO: 16 - protein, 246 aa, Novosphingobium aromaticivorans
VSDSNQIAALESRLNDLERRLTVREDELDVRKLQHLYGYLIDKCMYNETVDLFTEDGEVRFFGGVWKGKEGIRRLYVERFQKRFTYGNNGPIDGFLLDHPQLQDIIHVQDDGVTALGRARSMMQAGRHKDYEGDAPHLKARQWWEGGIYENTYKKVDGVWRMHILNYMPIWHADFESGWANTPHEYVPFPKVTYPEDPTGPDELIADHWLWPTHKLNPFHMKHPVTGEEMVAQRWQGDIDRENARK

SEQ ID NO: 17 - DNA, 1485 bp, Novosphingobium aromaticivorans
  source 1..1485: mol_type = genomic DNA
atggcccaatttccgaacacccccagcttcacgggattcaacacgccgtcgcggatcgaggcggatatcgccgatctggcccacgaaggcacgattccgcaagggttaaacggcgcattctaccgcgtccagcccgacccgcagtttcctccccgcctcgacgacgacatcgccttcaacggcgacggcatgatcacccgcttccacatccacgacggccaggtcgacttccgccagcgctgggcgaagaccgacaagtggaagctggagaacgccgccggaaaggccctgttcggcgcctaccgcaacccgctgaccgacgacgaggcggtcaagggcgagatccgttcgaccgccaacaccaacgccttcgtgttcggcggcaagctgtgggcgatgaaggaggacagtcccgccctcgtcatggacccggcgacgatggaaaccttcgggttcgagaagttcggcggcaagatgaccggccagacctttaccgcccaccccaaggtcgatccgaagaccggcaacatggtcgccatcggctatgccgcaagcgggctgtgcaccgacgatgtgacctacatggaagtgagcccggagggcgagcttgtccgcgaagtgtggttcaaggtgccgtactactgcatgatgcacgacttcggcatcaccgaggattacctcgtgctgcacatcgtgccttccatcggaagctgggaaaggctggaacagggcaagccgcacttcggcttcgacacgaccatgccggtgcacctcggcatcatcccgcgccgcgacggcgtgcgccaggaagacatccgctggttcacgcgggacaactgctttgccagccatgtcctgaacgcctggcaagaggggaccaagatccacttcgtgacctgcgaggcgaagaacaacatgttcccgttcttccccgacgtccacggcgcgcccttcaacggcatggaggccatgagccatccgaccgactgggtggtcgacatggccagcaacggcgaggactttgccgggatcgtgaagctttccgacacagccgccgagttcccgcgcatcgacgaccgctttaccggccagaagacccgccatggctggttcctcgaaatggacatgaagcgcccggtggaattgcgcggcggcagcgccggcggcctgctgatgaactgcctgttccacaaggacttcgaaacgggtcgcgagcagcactggtggtgcggcccggtgtcgagccttcaggagccgtgcttcgtgccgcgcgccaaggatgcccccgaaggcgacggctggatcgtgcaggtttgcaaccggctggaagagcagcgcagcgacttgctgatcttcgacgcgctcgacatcgagaaaggcccggtggccacggtcaacatccccatccgcctgcgcttcggccttcacggcaactgggcgaatgccgacgaaatcggccttgccgagaaggtcctggccgcatga

SEQ ID NO: 18 - protein, 494 aa, Novosphingobium aromaticivorans
MAQFPNTPSFTGFNTPSRIEADIADLAHEGTIPQGLNGAFYRVQPDPQFPPRLDDDIAFNGDGMITRFHIHDGQVDFRQRWAKTDKWKLENAAGKALFGAYRNPLTDDEAVKGEIRSTANTNAFVFGGKLWAMKEDSPALVMDPATMETFGFEKFGGKMTGQTFTAHPKVDPKTGNMVAIGYAASGLCTDDVTYMEVSPEGELVREVWFKVPYYCMMHDFGITEDYLVLHIVPSIGSWERLEQGKPHFGFDTTMPVHLGIIPRRDGVRQEDIRWFTRDNCFASHVLNAWQEGTKIHFVTCEAKNNMFPFFPDVHGAPFNGMEAMSHPTDWVVDMASNGEDFAGIVKLSDTAAEFPRIDDRFTGQKTRHGWFLEMDMKRPVELRGGSAGGLLMNCLFHKDFETGREQHWWCGPVSSLQEPCFVPRAKDAPEGDGWIVQVCNRLEEQRSDLLIFDALDIEKGPVATVNIPIRLRFGLHGNWANADEIGLAEKVLAA

SEQ ID NO: 19 - DNA, 1056 bp, Novosphingobium aromaticivorans
  source 1..1056: mol_type = genomic DNA
atgacacaagaccttaagaccggcggcgagcagggctacctgcgcatcgccaccgaggaagccttcgccacgcgcgagatcatcgacgtctacctgcgcatgatccgcgatggcactgccgacaagggcatggtctcgctctggggcttctacgcccagtccccctcagagcgcgccacccagatcctcgaacgcctgctcgatcttggcgagcgccgcatcgccgacatggacgcgaccggcatcgacaaggctatcctcgcgctgacctcgcccggcgtccagccgctgcacgaccttgacgaggccaggacgctcgccacccgcgccaacgacacgcttgccgacgcgtgccaaaagtacccagaccgcttcatcggcatgggcaccgtcgccccgcaggacccggaatggtccgcgcgcgagatccatcgtggtgccagggaactgggcttcaagggcatccagatcaacagccacacgcaagggcgctacctcgacgaggagttcttcgacccgatcttccgcgccctcgttgaagtcgaccagccgctctacatccaccctgccacttcgcccgattccatgatcgacccgatgctcgaagcgggcctcgacggcgccatcttcggcttcggcgtggagacgggcatgcacctgctgcgcctcatcaccatcggcatcttcgacaagtatcccagccttcagatcatggtcggccacatgggcgaggcgctgccctactggctctaccgcctggactacatgcaccaggccggtgtccgctcgcagcgctacgaacgcatgaagcccctgaagaagaccatcgagggctacctcaagtccaacgtcctcgtcaccaattcgggcgtcgcgtgggaacctgcgatcaagttctgccagcaggtcatgggcgaggaccgcgttatgtacgcgatggactacccctaccagtacgttgccgacgaggtgcgcgcgatggacgccatggacatgagtgcgcaaacgaagaagaagttcttccagaccaacgcggagaagtggttcaagctttga

SEQ ID NO: 20 - protein, 351 aa, Novosphingobium aromaticivorans
MTQDLKTGGEQGYLRIATEEAFATREIIDVYLRMIRDGTADKGMVSLWGFYAQSPSERATQILERLLDLGERRIADMDATGIDKAILALTSPGVQPLHDLDEARTLATRANDTLADACQKYPDRFIGMGTVAPQDPEWSAREIHRGARELGFKGIQINSHTQGRYLDEEFFDPIFRALVEVDQPLYIHPATSPDSMIDPMLEAGLDGAIFGFGVETGMHLLRLITIGIFDKYPSLQIMVGHMGEALPYWLYRLDYMHQAGVRSQRYERMKPLKKTIEGYLKSNVLVTNSGVAWEPAIKFCQQVMGEDRVMYAMDYPYQYVADEVRAMDAMDMSAQTKKKFFQTNAEKWFKL

SEQ ID NO: 21 - DNA, 23 bp, synthetic construct
  source 1..23: mol_type = other DNA
ctgtcgtgccagctgcattaatg

SEQ ID NO: 22 - DNA, 29 bp, synthetic construct
  source 1..29: mol_type = other DNA
gaacatctagaaagccagtccgcagaaac

SEQ ID NO: 23 - DNA, 49 bp, synthetic construct
  source 1..49: mol_type = other DNA
cgattcattaatgcagctggcacgacagcttttcgcttctccagctcgg

SEQ ID NO: 24 - DNA, 51 bp, synthetic construct
  source 1..51: mol_type = other DNA
cccacccgcaatctcttatttccggtccaactcccatcaatttagtttgtc

SEQ ID NO: 25 - DNA, 51 bp, synthetic construct
  source 1..51: mol_type = other DNA
gtttctgcggactggctttctagatgttccttccacgatgaagcgggttgg

SEQ ID NO: 26 - DNA, 51 bp, synthetic construct
  source 1..51: mol_type = other DNA
gacaaactaaattgatgggagttggaccggaaataagagattgcgggtggg

SEQ ID NO: 27 - DNA, 52 bp, synthetic construct
  source 1..52: mol_type = other DNA
cgattcattaatgcagctggcacgacagcggctcgcgcaatttgttagtaag

SEQ ID NO: 28 - DNA, 52 bp, synthetic construct
  source 1..52: mol_type = other DNA
gtttctgcggactggctttctagatgttccggatcatgcgcaggtagacgtc

SEQ ID NO: 29 - DNA, 52 bp, synthetic construct; identical to SEQ ID NO: 28
  source 1..52: mol_type = other DNA

SEQ ID NO: 30 - DNA, 50 bp, synthetic construct
  source 1..50: mol_type = other DNA
caggcaaaaggcttccggagattaaatataattgcggtgtcggtcggcag

SEQ ID NO: 31 - DNA, 51 bp, synthetic construct
  source 1..51: mol_type = other DNA
cgattcattaatgcagctggcacgacaggaaggcgcaatccggagttctcc

SEQ ID NO: 32 - DNA, 42 bp, synthetic construct
  source 1..42: mol_type = other DNA
ccctcccggcgctggtcaaaggcaggcttccttcccgggaag

SEQ ID NO: 33 - DNA, 52 bp, synthetic construct
  source 1..52: mol_type = other DNA
gtttctgcggactggctttctagatgttctccagtggaagccgggagtgacc

SEQ ID NO: 34 - DNA, 42 bp, synthetic construct
  source 1..42: mol_type = other DNA
cttcccgggaaggaagcctgcctttgaccagcgccgggaggg

SEQ ID NO: 35 - DNA, 55 bp, synthetic construct
  source 1..55: mol_type = other DNA
cgattcattaatgcagctggcacgacaggggggctaaccgccagtctctatcttc

SEQ ID NO: 36 - DNA, 52 bp, synthetic construct
  source 1..52: mol_type = other DNA
gcaatacatacaatattgcaaggaggatgccgccgcatgatccagcccggag

SEQ ID NO: 37 - DNA, 50 bp, synthetic construct
  source 1..50: mol_type = other DNA
gtttctgcggactggctttctagatgttcccaacaggcagccgaggatag

SEQ ID NO: 38 - DNA, 52 bp, synthetic construct
  source 1..52: mol_type = other DNA
ctccgggctggatcatgcggcggcatcctccttgcaatattgtatgtattgc

SEQ ID NO: 39 - DNA, 51 bp, synthetic construct
  source 1..51: mol_type = other DNA
cgattcattaatgcagctggcacgacagctgacacggatctctcctcaacc

SEQ ID NO: 40 - DNA, 50 bp, synthetic construct
  source 1..50: mol_type = other DNA
gtaaaccgtgtaaacccgttcaggtattgctacagccctgttaaattgcg

SEQ ID NO: 41 - DNA, 50 bp, synthetic construct
  source 1..50: mol_type = other DNA
cgcaatttaacagggctgtagcaatacctgaacgggtttacacggtttac

SEQ ID NO: 42 - DNA, 50 bp, synthetic construct; identical to SEQ ID NO: 41
  source 1..50: mol_type = other DNA

SEQ ID NO: 43 - DNA, 30 bp, synthetic construct
  source 1..30: mol_type = other DNA
taacagaaagccgaaaataacaaagttagc

SEQ ID NO: 44 - DNA, 34 bp, synthetic construct
  source 1..34: mol_type = other DNA
catggttaatttctcctctttaatgaattctgtg

SEQ ID NO: 45 - DNA, 32 bp, synthetic construct
  source 1..32: mol_type = other DNA
cagaaagccgaaaataacaaagttagcctgag

SEQ ID NO: 46 - DNA, 24 bp, synthetic construct
  source 1..24: mol_type = other DNA
tgcgatcgcgctctgaaaatacag

SEQ ID NO: 47 - DNA, 43 bp, synthetic construct
  source 1..43: mol_type = other DNA
taaagaggagaaattaaccatgtccgatagcaatcagattgcc

SEQ ID NO: 48 - DNA, 39 bp, synthetic construct
  source 1..39: mol_type = other DNA
tgttattttcggctttctgttatttccgcgcattttcgc

SEQ ID NO: 49 - DNA, 41 bp, synthetic construct
  source 1..41: mol_type = other DNA
taaagaggagaaattaaccatgactgcgtacccttctctcc

SEQ ID NO: 50 - DNA, 42 bp, synthetic construct
  source 1..42: mol_type = other DNA
tgttattttcggctttctgttacccttcatgtaccgctttgg

SEQ ID NO: 51 - DNA, 42 bp, synthetic construct
  source 1..42: mol_type = other DNA
taaagaggagaaattaaccatgacacaagacctgaagaccgg

SEQ ID NO: 52 - DNA, 48 bp, synthetic construct
  source 1..48: mol_type = other DNA
tgttattttcggctttctgttaaagtttaaaccatttttcagcgttgg

SEQ ID NO: 53 - DNA, 45 bp, synthetic construct
  source 1..45: mol_type = other DNA
taaagaggagaaattaaccatggctcaatttccgaataccccaag

SEQ ID NO: 54 - DNA, 40 bp, synthetic construct
  source 1..40: mol_type = other DNA
tgttattttcggctttctgttatgcggccaggaccttttc

SEQ ID NO: 55 - DNA, 44 bp, synthetic construct
  source 1..44: mol_type = other DNA
taaagaggagaaattaaccatgctaagcgacaggcacgtcaaag

SEQ ID NO: 56 - DNA, 51 bp, synthetic construct
  source 1..51: mol_type = other DNA
tgttattttcggctttctgttagaacaccactactgaacgaatcgatttac

SEQ ID NO: 57 - DNA, 50 bp, synthetic construct
  source 1..50: mol_type = other DNA
aaatctgtattttcagagcgcgatcgcatccgatagcaatcagattgccg

SEQ ID NO: 58 - DNA, 49 bp, synthetic construct
  source 1..49: mol_type = other DNA
ggctaactttgttattttcggctttctgttatttccgcgcattttcgcg

SEQ ID NO: 59 - DNA, 52 bp, synthetic construct
  source 1..52: mol_type = other DNA
aaatctgtattttcagagcgcgatcgcaactgcgtacccttctctccacatg

SEQ ID NO: 60 - DNA, 55 bp, synthetic construct
  source 1..55: mol_type = other DNA
ggctaactttgttattttcggctttctgttacccttcatgtaccgctttggtgac

SEQ ID NO: 61 - DNA, 49 bp, synthetic construct
  source 1..49: mol_type = other DNA
cattaaagaggagaaattaaccatgcagtttgaacgtatcaatccgatg

SEQ ID NO: 62 - DNA, 48 bp, synthetic construct
  source 1..48: mol_type = other DNA
gtttaaactattaatgatgatgttaaattggatagtgacctggttggg

SEQ ID NO: 63 - DNA, 40 bp, synthetic construct
  source 1..40: mol_type = other DNA
cattaaagaggagaaattaaccatgaaagccgccgtactc

SEQ ID NO: 64 - DNA, 49 bp, synthetic construct
  source 1..49: mol_type = other DNA
gtttaaactattaatgatgatgttattgatcaaacacaataacagaacg

SEQ ID NO: 65 - DNA, 48 bp, synthetic construct
  source 1..48: mol_type = other DNA
cattaaagaggagaaattaaccatgacaatcaatacaattcgcgtacg

SEQ ID NO: 66 - DNA, 46 bp, synthetic construct
  source 1..46: mol_type = other DNA
cgtttaaactattaatgatgatttaacaaaaatgacggcagctctg

SEQ ID NO: 67 - DNA, 41 bp, synthetic construct
  source 1..41: mol_type = other DNA
cattaaagaggagaaattaaccatgttgggacgtgcatcgg

SEQ ID NO: 68 - DNA, 44 bp, synthetic construct
  source 1..44: mol_type = other DNA
gtttaaactattaatgatgatgttacgtgatcgtcggatcgatc

SEQ ID NO: 69 - DNA, 45 bp, synthetic construct
  source 1..45: mol_type = other DNA
cattaaagaggagaaattaaccatggcggcaattaatcttccccg

SEQ ID NO: 70 - DNA, 48 bp, synthetic construct
  source 1..48: mol_type = other DNA
gtttaaactattaatgatgatgttagccaaagacttcggcatagaggc

SEQ ID NO: 71 - DNA, 50 bp, synthetic construct
  source 1..50: mol_type = other DNA
cattaaagaggagaaattaaccatgcgattgaaagtactgggacttatgg

SEQ ID NO: 72 - DNA, 45 bp, synthetic construct
  source 1..45: mol_type = other DNA
gtttaaactattaatgatgatgttagccacctttggcttctaaag

SEQ ID NO: 73 - DNA, 52 bp, synthetic construct
  source 1..52: mol_type = other DNA
cattaaagaggagaaattaaccatgattccgcatggtgaacattcaatgctg

SEQ ID NO: 74 - DNA, 48 bp, synthetic construct
  source 1..48: mol_type = other DNA
gtttaaactattaatgatgatgttatggcaccaaaaccagagcgccac

SEQ ID NO: 75 - DNA, 46 bp, synthetic construct
  source 1..46: mol_type = other DNA
cattaaagaggagaaattaaccatggacgcatacgctgcaattatc

SEQ ID NO: 76 - DNA, 54 bp, synthetic construct
  source 1..54: mol_type = other DNA
gtttaaactattaatgatgatgttacattttgagaatggcttttatcgcttttc

SEQ ID NO: 77 - DNA, 50 bp, synthetic construct
  source 1..50: mol_type = other DNA
cattaaagaggagaaattaaccatgtctacacagcctgcaaccatagctg

SEQ ID NO: 78 - DNA, 46 bp, synthetic construct
  source 1..46: mol_type = other DNA
gtttaaactattaatgatgatgttatggacgagtttgcccgcttcc

SEQ ID NO: 79 - DNA, 50 bp, synthetic construct
  source 1..50: mol_type = other DNA
cattaaagaggagaaattaaccatgcgcgaacggctacagcaatacattg

SEQ ID NO: 80 - DNA, 45 bp, synthetic construct
  source 1..45: mol_type = other DNA
gtttaaactattaatgatgatgttaggcaggcaggccgctgatcg

SEQ ID NO: 81 - DNA, 40 bp, synthetic construct
  source 1..40: mol_type = other DNA
cattaaagaggagaaattaaccatgactgcccctaccgcc

SEQ ID NO: 82 - DNA, 47 bp, synthetic construct
  source 1..47: mol_type = other DNA
gtttaaactattaatgatgatgttactgctgatgacgatatacagcc

SEQ ID NO: 83 - DNA, 46 bp, synthetic construct
  source 1..46: mol_type = other DNA
cattaaagaggagaaattaaccatgggttaccgggttgtagtggtg

SEQ ID NO: 84 - DNA, 49 bp, synthetic construct; identical to SEQ ID NO: 61
  source 1..49: mol_type = other DNA

SEQ ID NO: 85 - DNA, 46 bp, synthetic construct
  source 1..46: mol_type = other DNA
cattaaagaggagaaattaaccatggcgatcaaagttgcgataaac

SEQ ID NO: 86 - DNA, 46 bp, synthetic construct
  source 1..46: mol_type = other DNA
gtttaaactattaatgatgatgttaaaggaatttcgccattgctcc

SEQ ID NO: 87 - DNA, 45 bp, synthetic construct
  source 1..45: mol_type = other DNA
cattaaagaggagaaattaaccatgaatgacatgactaccatctc

SEQ ID NO: 88 - DNA, 52 bp, synthetic construct
  source 1..52: mol_type = other DNA
gtttaaactattaatgatgatgttacatttgaataattactgttttagtctc

SEQ ID NO: 89 - DNA, 47 bp, synthetic construct
  source 1..47: mol_type = other DNA
cattaaagaggagaaattaaccatggctacgcagttgagaagtgcag

SEQ ID NO: 90 - DNA, 49 bp, synthetic construct
  source 1..49: mol_type = other DNA
gtttaaactattaatgatgatgttactgatcgaacattccggtacgacc

SEQ ID NO: 91 - DNA, 737 bp, synthetic construct
  source 1..737: mol_type = other DNA
ccgatagcaatcagattgccgcgcttgaaagtcgcctgaatgacctcgaaaggcgactgacggttagagaggacgagctggacgtacgcaaactccagcatttatacggttatctgattgataaatgcatgtataacgagacagttgacctgttcacagaagatggggaagtgcggttctttggtggcgtatggaaaggcaaggagggcatccgccgtttgtacgttgaacgttttcagaaacgtttcacctatggcaataacggcccgattgatgggttcctgttagatcatccacaacttcaagatattattcacgtgcaggatgatggggtcacggctttgggccgcgcgcgttccatgatgcaagccggtcgccacaaggattatgagggagatgcacctcatctgaaagcgcgtcagtggtgggaaggtggtatatacgaaaacacttataaaaaagtggatggcgtgtggcgtatgcatatcctaaactacatgccgatctggcacgcagattttgaaagcggctgggccaataccccgcacgaatacgttccttttcccaaagtcacctatccagaagacccgactggaccggatgaactgattgctgaccattggttatggccgacccataagctgaacccctttcacatgaaacatccggtgacgggtgaggaaatggtcgcacagcgctggcagggtgacatcgatcgcgaaaatgcgcggaaataa

SEQ ID NO: 92 - DNA, 1428 bp, synthetic construct
  source 1..1428: mol_type = other DNA
actgcgtacccttctctccacatgattattgacggtgcccgtgtcagcggcggaggacgtcgcacccacgcggtcgtcaatccggctaccggagagaccatcggtgaactgccgctggcagaagttgcagatctggatcgagcgttagaagtagcggcgaagggcttccgtatttggcgtgacagcacaccgcagcagcgcgcagccgtgttacagggcgcggcccggctgatgctggaacggcaagaggatctcgctcgcatagccacgatggaagaaggtaaaaccctgcccgaggcgcgcatcgaagttctgatgaacgtgggcctgttcaatttttacgctggagaagtatttcgtttatatggccgaaccctagtgcgccctgcgggtcagagaagcacgatcacgcatgaaccggtagggccggtggccgcctttgctccgtggaactttccgcttgggaatccaggtcgcaaactgggcgcgccaattgccgccggttgctcggtgattctaaaagcggcggaagaaacgccggcttcagcgttaggggtgctgcaatgtctgctggatgctggcctgcctaaagaagtggcccaggctgtgttcggtgtgcctgacgaggtgagtcgccacctgttgggcagttccgttatccgcaagctctcgtttacaggttctaccgtcatcggcaagcatctgatgcgacttgcagccgacaacatgttgcgtacaactatggagcttggcggccatggtcctgtcttagttttcggtgatgcagatattgacaaagcgctcgataccatggcagcttccaaatatcgtaacgcgggccaagtttgtgtttcaccaaccagatttatagtggaagaaagcgtgttcgaacgttttcgtgatggttttgcagagcgtgtcggtcggatcaaagttggaaatggtttggatcaggatgcgcagatgggaccgatggcaaatgcccgccgcccggaggcgatggatcgtctgatcggggacgccgtgactcgcggcgcaaggttgcatactgggggcgaacgtgtcggcaacgccggctatttttatgcccccacggttctgagtgaagtaccgctggacgcggctattatgaacgaagaaccgtttggcccggtagctctgattaatccattcggcggtgaggaagcgatgatcgccgaagcaaaccgtctgccgtatggcttggcagcctacgcatggacagatagcgcggcgcgggcaaaacgcttagcacgcgagattgagacggggatgctggggcttaattctaccatgattggcggcgcggattcgccattcggtggggtgaaatggtccggacacggttcagaggacggtcccgaaggtgttatggcctgccttgtcaccaaagcggtacatgaagggtaa

SEQ ID NO: 93 - DNA, 1053 bp, synthetic construct
  source 1..1053: mol_type = other DNA
acacaagacctgaagaccggcggggagcagggttacctgcgtatcgccaccgaagaagctttcgccacgcgagaaatcattgatgtctacctgcgcatgatacgcgatggaactgctgataaaggtatggtatcattgtggggcttttatgcccagtccccttcagagcgcgccacccagatcttagaacgtctgttagatcttggcgagcggcgtattgcagatatggatgcgacaggcattgacaaggctattctagcgctgacctcgccgggcgtacagccgctgcatgacttagatgaagcacggacgctcgcaacccgtgcaaatgatactcttgccgatgcgtgccaaaagtatccagaccgatttattggaatgggcaccgtggccccgcaggatccggaatggagtgcgcgcgaaattcatcgtggtgcaagggaactgggttttaagggcatccagatcaacagccacacgcaagggcgctacttggatgaggaattctttgatccgatattccgtgccctcgttgaagtcgaccagccgctgtatattcatcctgccacttcgccagattccatgatcgatccgatgttggaagcgggcctggacggtgcaatcttcggcttcggtgtggagacgggcatgcatctgctgcgcctgatcacgattgggattttcgacaaatatcccagcttgcaaattatggttgggcacatgggcgaggcgctgccctactggctctatagactggattatatgcaccaggctggtgtgcgctctcagcgctatgaacgtatgaaaccactgaaaaaaaccatcgaaggttatcttaaaagcaacgtgttagtgacaaattctggagtcgcgtgggaacctgcgattaaattttgtcagcaagtaatgggtgaggatcgggttatgtacgcgatggactacccgtatcagtacgttgcagacgaagtgcgtgcgatggatgccatggacatgagtgcgcaaacgaaaaaaaaattttttcagaccaacgctgaaaaatggtttaaactttaa

SEQ ID NO: 94 - DNA, 1554 bp, synthetic construct
  source 1..1554: mol_type = other DNA
atggctcaatttccgaataccccaagcttcacgggattcaacacgccgtctcggattgaggcggatattgcagatctggcccacgaaggtacgattccgcaagggttaaacggcgcattttatcgtgtccagcccgatccgcagtttcctccacgcctcgatgatgacattgcctttaacggagacgggatgattacccgattccatatacatgatggccaggtcgacttccgtcaacgttgggcgaaaaccgataaatggaaactggaaaacgcggccggaaaagccctgtttggtgcctaccgcaacccactgaccgatgacgaggcggttaaaggcgagatccgttcgaccgccaacactaacgccttcgttttcggtggcaaactgtgggcgatgaaagaggacagtccagcactcgtaatggatccggcgacgatggaaaccttcgggttcgaaaagttcggcggtaaaatgacaggccagacctttactgcccatccgaaggtagatccgaaaaccggcaatatggtagcgatcggttatgctgcaagcgggttgtgcacagatgatgtgacctacatggaagttagtccggagggtgaattagtacgcgaagtgtggttcaaagtgccgtattattgcatgatgcacgacttcggcattacagaggattacctcgtgctgcacattgttccttccatcggaagctgggaaagattagaacagggcaaaccgcactttggctttgatactactatgccggttcacctaggtatcattccgaggcgtgacggtgtgcgccaggaagatatccgttggttcacgcgggataattgttttgccagtcatgtactgaatgcttggcaagaagggaccaaaattcactttgtgacttgcgaagcgaaaaacaacatgtttcctttctttccagatgtccatggcgcgccctttaacggtatggaggcaatgtcacatcctacggactgggtggtcgacatggcaagcaacggcgaggactttgctgggatcgtgaagctttccgatacagctgcagaatttcctcgcatcgacgaccggtttaccggccagaaaacccgccatggttggttcttagaaatggatatgaaacgaccagtggaattgcgcggtggttcagcgggcggcctgctgatgaattgtctgtttcacaaggacttcgaaacgggtcgtgaacagcattggtggtgcggcccggtttcgtctcttcaggagccgtgttttgttccgcgcgcgaaagatgcccccgaaggtgatggatggattgtgcaagtttgtaatcgtctggaagaacagcgttccgatttgctgatatttgatgcgctggatattgagaaaggcccggtggctacggtcaatatccccatccgcctgcgctttggcttgcatggtaattgggcgaatgcagacgaaattgggcttgcggaaaaggtcctggccgcagcgatcgcaggaagcgaaaatctgtattttcagagcgcattggcacatcaccatcatcaccatcaccattaa

SEQ ID NO: 95 - DNA, 1149 bp, synthetic construct
  source 1..1149: mol_type = other DNA
ctaagcgacaggcacgtcaaagggagaccgcatgaaatgaaaacacgcgccgcagttgcgtttgcgccaaagcaaccgttggaaattgtagaactggatctggaaggtcccaaagctggggaagttctggttgagattatggcgactggagtgtgtcacaccgatgcatatacgttagacgggttcgacagcgaaggcattttccctagcgtgctgggtcatgaaggtgccggtatcgtgcgcgaagtgggccctggggtaacttccgtgaaacctggcgatcatgtgatcccgctctatacgccggaatgtcgccagtgcaaatcgtgcttgtcgggtaagaccaacctgtgcaccgctattcgcgccacgcaagggcagggcctgatgcccgatggcaccagtcgtttttcttacaaaggccagaccgtgttccactacatgggttgcagtacattctctaattttacagttctgccagagatcgcggttgcaaagattcgcgaggatgcgccgtttaaaacctcatgttatattggctgtggcgtgacgacgggtgttggcgcggtgattaacactgctaaagtacaggtcggtgacaacgtcgtggtctttggattaggcggcataggtctcaatgttattcagggagcgcggcttgccggtgcagggaaaatcattggcgtcgatatcaatccagatcgggaggaatggggccgtaaatttggcatgactgactttctgaatagtaagggcatgagccgcgaggacgtagttgctaaagtcgtcgccatgaccgatggcggtgcggactatacctttgatgccaccggtaataccgaagtgatgcgtacggcgcttgaagcatgccatcgtggttggggaacctccataatcattggtgtggcagaggcgggtaaagaaattagcacgcgtccgttccaattagttactggccgtaactggcgaggcacggccttcggaggcgccaaggggcgcacagatgttccgaaaattgtagatatgtacatgaccggaaaaatcgaaatcgatccgatgatcacccatgtcatggggctggaagagatcaacacagcatttgatctgatgcacgctggtaaatcgattcgttcagtagtggtgttctaa

SEQ ID NO: 96 - DNA, 1398 bp, synthetic construct
  source 1..1398: mol_type = other DNA
cagtttgaacgtatcaatccgatgacaggggcagtagcctcgcaggcagaggccatgaaagcgtcggacattccttccattgctgcccgcgcaggacaggcctttccggcgtgggcagcgatgggccccaacgcacgtcgcggcgtactgatgaaggcggctgcggcgttggaagcgcgggctgatgctttcgtcgaagccatgatgggcgaaatcggcgcgactagagggtgggcgctgtttaaccttggccttgcagcaagcatggtgcgcgaagccgccgcgctgaccactcaaatctctggagaggttattccatctgacaaaccggggtgtatttcgatggctctgcgcgaaccggttggtgtgattttgggcatcgcgccgtggaatgcgccgattatccttggggtgcgcgcaattgccgtgccgcttgcctgcggtaacgcggtgatattaaaagcaagcgaaacatgtccgcgaacccacgcgctcatcatcgaggcctttgctgaagcaggtttcccagaaggcgtggttaatgtagtgacgaacgcgcctgcagatgcagcggaagtggtcggggcgctgattgatgcgccggaagtgcgtcgtataaactttaccggtagtactaatgtaggcaggattatcgcaaaacgggcggccgagcatttgaaaccctgtttactcgaactgggcggtaaagcaccgttaatagttctggatgatgcggatctagacgaagcggtcaaagctgcggcttttggcgccttcatgaaccaagggcagatttgcatgtcaacggagcggatcatcgttgtagatgccgttgccgatgcattcgcagataaattcaaggccaaggtcgcctccatggctgtaggcgacccgcgtgagggtacgaccccgttgggtgcagttgtcgacgctaaaactgtcgctcattgccgtagcttaattgacgatgccctggcaaaaggtgcccgtctgctgaccggcggtgaaaccacgcacaatgtgctcatgcccgcccatgtcgtagatggcgtgacgcaggatatgaagctgttccgcgatgagagctttggcccagtggtgggcgtgattcgcgcgcgcgacgaagctcatgccattgaactggcgaacgacagtgaatatggactgtcagcggctgttttcacacgtgacacagcgcgcggcctgcgagttgcccgccagatccgtagcggtatttgccatgttaatggacctaccgtccacgatgaggcgcagatgccttttggtggagtgggtgcgtccggctacggtcgttttgggggtaaagccggcatcgatagttttaccgagctgagatggattacgatggaaacccaaccaggtcactatccaatttaa

SEQ ID NO: 97 - DNA, 1086 bp, synthetic construct
  source 1..1086: mol_type = other DNA
aaagccgccgtactcgtcgaaccgggtaaaccgctggatattcagcatttaagcgtgagtaaacccggccctcatgaagtccttatacgcacagcagcctgcgggctgtgccatagtgacttgcacttcatcgaaggtgcctatccacatccgctgccggctgtgccagggcacgaggctgctgggattgtggaagcggtaggttcagaagtgcgcacagtaaaagtgggtgacgctgttgttacctgcctgtccgcgttctgtggtcattgcgagttttgcgtgaccggccggatgtcgctgtgtcttggtggcgatactcggcgcggtgcgggtgaggcacctcgcttgacacgcaccgacgatggaagcgcagtgaaccagatgctcaacctatcggcctttgcagaacaaatgctggttcacgaacatgcctgtgttgcgatcaatcccgagatgccgctcgatagagctgcggttatcggctgtgcggtaaccactggcgcgggtgcggtgtttaatgctgcgaaactgaccccaggagagacggtatgcgttgtcggctgtggcggcgtaggcttagcaacggtcaatgccgcgaaaattgccggggcaggccgtattatcgctgtggatccgatgccggaaaaacgcgaactggccatgaaactgggtgcgaccgatgtgatggacgcgggacccgatgctgcggcacagatcgttgaaatgacgaaaggcggcgttcaccatgcgatcgaggccgtggggcgtcctgcatctggcgaccttgcggtcgcgacgctgcgtcgtgggggcaccgccacgattttaggtatgatgccgctggcacacaaggtcggattatcagcgatggatctgctgagcgataagaagctgcagggtgcaattatgggccgcaaccacttcccagtggatctgccgcgactggtcgacttctacatgcgtggcttgttggatctagacactatcattgccgaaaggattccgcttgaagggataaacgatggttttgaaaaaatgaaacagggacattccgcccgttctgttattgtgtttgatcaataa

SEQ ID NO: 98 - DNA, 1011 bp, synthetic construct
  source 1..1011: mol_type = other DNA
acaatcaatacaattcgcgtacgttcgccggccactctcgacaccttaaatttcgatacgctgacggattgtggacaaccgggaacgagcgaaatccgcattcgtctgcgcgcaacttctctgaacttccactactacgcgatgattaccagaatgctgccggctgcaacaagtcgaattcctatgtctaacggcgcctgacaggttttcggggtgtgcgatggcgtgaccaaattccaggcgcgtaacgcagttatctcgacctttttcaccgacaggaacgccggtccgccacagtcagccgcgtttacgaccgtcacggctgatgggattaatcgctacgcgcgggaagaagtggtggccccggctcattggtttacccgcgcgccgttatgctatagtcacgcaaaagccgccacgctgacctgcgcgggccttactgcatggcgtgctttgttcatagataacgctatcaagccgggcgacacggtcttggtgcagggcactggcagcgtttcggttttcgcgctgcagttaacaaaggcggcatgcgcgcgtgtcatcgcaacgagttcctcccaccagtaactgaaacgcctgcgcagccttagagcgaataaaaccataaactataaaacgcaaacctcacgggggatgcagacactagatttcactgccggtatttgtgtacactgtattgtcgagattagccggcccggtacgtttcatcaagcgatgatgtccacccgcgtgcgtgctcatatcgcgctgatcggtgttctcgcgcgttttgcgggtccagtttaaaccactttgctgatggcacagaatctgcgcgtataaggccttaccgtggcctcacgtaccaatcatctgcgaatgattcccggtatcgaggcaaaccgtatccaacctgtcattcaccgccattttccatttccgtattttgccgctgcctttcgccatcaacagagctgccgtcatttttgttaaatcgtgattgacatttga

SEQ ID NO: 99 - DNA, 1080 bp, synthetic construct
  source 1..1080: mol_type = other DNA
ttgggacgtgcatcggtgctggtaaaaccgaaccaactggagacgtgggatgttaaagtagccgatccggaaccgggcggtgccttagtttcgattgtgctgggtggggtatgcgggagcgacgtccatatattgaccggcgaggctggcgtgatgccgtttccgatcattctgggacatgagggcgtgggaaggatcgaaaaactggggcacggcgtcagcactgattacgctggtgaggaacttaaacccggcgatctggtatattggtcgccgattgctctgtgtcatcgatgttattcctgcaatgttctcgatgaaacaccttgcgaaaatacccagtttttcgaagatgcttccaagccgaactggggttcatacgcagattatgcatggctgcccaacggtatgccgttctataaactgccagcccaagcgcagcctgaagcggttgctgcgcttggctgtgcacttccaaccgccctgcgcggctttgatcgctgcggcagtgttagagtgggtgaaactgtggttgtccaaggtgcaggccctgtcggcctgtctgcagtgctcgtggcggcgcaggccggggcgcgtgacgtgattgttattgacggttcaccacttcgtcgcgaagcggctaccgcattgggtgcctctctgacgattggcttagatgtcgcgcctgaggaacggcgccggatgatttacgatcgcgttggtcgcaatggtcccaatgtagtcatcgaggcagccggagttctgccagcgtttccggaaggggtggacctgaccggtaaccacggccgttacattgtgctaggattgtggggcgcaatagggacccagccgatcagcccgcgcgacttaacaatcaaaaacctgactatcgctggtgcgaccttccctaaaccaaaacattattatcaggccttgcatttagcgacggccctgcaggaccgtgtaccgttagccggtctggtgagccaccgttttggcgtcagccaggcgggcgaagcgctgagtctcaccaagagtgggacagcgattaaggccgtgatcgatccgacgatcacgtaa

SEQ ID NO: 100 - DNA, 1155 bp, synthetic construct
  source 1..1155: mol_type = other DNA
gcggcaattaatcttccccgcgtgattcgtgctggtgggggtgcattagccgaactgcccgatgcaatggcgcagtgcggcctttcacgcccgttcgtggtgaccgatgcattcttagtgcaaagcgggatggtcgctcggatgttagaggttctggacggcgctgggattgcggccacggtcttcgatgctacggtacctgatccgactgttgctgtggtagaacaggcgcttggcgcattgcgagaggcggaatgtgattgtgtgatcgggtttggaggtggtagcccgatcgacaccagtaaagccattgccgccctggcgctggaaccgcgtgcagttcaatccatgaaggcaccagcgacgaccgacgtcccgggtctgccgatcattgccgtcccgacgaccgccggcaccggctcggaggcgactaaatttacaatcgtgaccgatgaggcgacgagtgaaaaaatgctctgcgcaggtctggccttcctgcctactatagccattgtagatttcgagctgaccatgggcaaaccggctcggctaactgccgacacaggtattgattcgctgacacatgcgattgaggcctatgtttctaagaaagccaatccgtttagtgatgctatggcgatctcggcgatgaaactgatcgcgccgaacattcgcaccgcctgcgccgaacccggaaaccgtgctgcacgcgaagcgatgatgattggcgcgcaccatgccggtattgcgttttccaacgctagcgttgcactggtgcacggtatgagccgcccaatcggcgcattctttcatgtgccgcacggattgtccaacgcaatgttgctgcctgcgattaccgcgttttccgctccgtcagcgttaccacgttacgccgattgtgcccgtgcgatgggtgtagctttggaaagcgaaggcgaccagtctgccgttgcaaggctgctcgacgaactggcggcgctgaacgcagaccttagtgtcccgacgccgcagtcgcatgggatcagcgctgatcgttggtttgaagtagtgcctgaaatggcgagacaggcaatagcatcaggctctccaggcaataatccacgcgttcctgatgcggcggaaatcgagcgcctctatgccgaagtctttggctaa

SEQ ID NO: 101 - DNA, 2115 bp, synthetic construct
  source 1..2115: mol_type = other DNA
cgattgaaagttctgggacttatggcagcactgctgccgctggcggcttgtaacatcaaaagcgagggtggaggggatgcagtcgccaacgctggagtcacagatgccctgattgcccaagcgcccgaaggcgaatggctgagctatggccgcgattatggggaacaacgcttttcaccgttgacccaaattaatgatggtaacgtcgggcagttgggtcttgcctggtttcatgacctggagactgcgcgcgggcaagaagcgacgccgctgatgcatgatggtacgttatatatctcgactgcgtggtcaatggtgaaagcgttcgatgcaaaaaccggcgcgctgaaatggagttacgatcccgaagtaccgcgtgaaacgctggtgcgcgcatgctgcgacgcggtcaatcgtggcgtcgcgctgtatggagataaagtttttgtaggtacgctcgatggtcgtctagtagcgttagatcagaagaccggaaaagtagtttggtccaaggtagtagtgcccaatcaggaggactacaccataactggtgccccgcgcgtggtgaaaggcaaagttctgattggtagcggtggctcggagtacaaagctcgaggctatattgccgcctatgacgttaacacaggcaacgaagtgtggaaattccacaccgtccctggcaatccagcggatgggtttgagaacaaagcgatggaaaatgccgctcgcacttgggctggtgaatggtggaaactcggtgggggtggcacggtgtgggattccatcacctatgatccagccaccaacctagttctgttcggcacaggcaatgcagaaccatggaacccggcagcagccgggcgggagggagacagcttgtacacgtcctctattgtagcggtgaatgccgatactggcgactatgtatggcattttcaagaaaccccggaagaccgttgggacttcgattccgcgcagcagattacgctggccgacctgacaattgatgggcagcggcgccacgtgatccttcatgcgcctaagaacggtcatgtttatgtgttggacgcaagaaccgggcagtttctgtcggcaacgccctttgtgatggtgaactgggcgaccggtattgatcctaaaacgggcaaggccactgtcaatccagaagcccgttatgaaaaaaccggcaaacctttcgttagcctgccaggtgcggtaggcgcacattcatggcagccgcagagtttcagcccgaaaaccggcctgctgtaccttccggtgaacaatgcggcatttccttatgcagccgccaaagactggaaagcaaccgatattggtttccagaccggtctcgacggctatgttaccagtatgccagccgacgcaaaggtccagggcgcagcgatgaaagcgaccactggtacgttagtggcgtgggacccggttgcgaagaaagccgcttggaaagtcgaactgccgagcccgagtaacggtggcattttatcgacagctggcaatttagtgtttcaaggtaccgcgggcggtgattttgttgcatacaacgccgataagggcaaacaattatggtcttttccggcgcagagtggcatccttgccgcgccgatgacctatgctatcgatggggaacagtacgttgcggtcatggtgggctggggaggtgtgtgggacgtcgccacaggtgtgctcgctcataaggccaaaaaacagaggaacataagccgcctggtagtgttcaaactgggcgggaaagccacgctgccggctgctcctccgatggcaaaaatggttttggatccgccgccgtttacaggtacgcccgaacaagctaaggccggtggcgaattatacggacgttactgcaacgtttgtcatggtgatgctgcggttgcgggcggcgtgaatccagatctgcgtcactcagctgcgcttaatgcaccagaggcgatccggtctgtggtgattgagggggcgctgcagcacaacgggatggtctcgttcaaatctgcgctgaagcctgaggatgcggataatatccgccactacttgatcaaacgtgcaaatgaagacaaagctctcgaagccaaaggaggctaa

SEQ ID NO: 102 - DNA, 1002 bp, synthetic construct
  source 1..1002: mol_type = other DNA
attccgcatggtgaacattcaatgctggcaatgcagttggatggtccaggcaaacggctgcacccagtcgtgcgccctctgccgttaccggggcgaggtgaagtgcgggtaaaagtgcatgcctgtggtgtttgccgtacggacctgcacgttgcagatggcgatattcacggtctgctacctattgtgccggggcacgaagtgataggcgttgtcgatgcactggggccgggggtgacggatgttgaacctggtgcgcgtgtaggtgtcccgtggctcggccatgcctgtggcacctgcccatattgcgacagcgggagggaaaacctttgtgatgcgccgctgttcaccggttttactcgcgatggcggatacgctacccatgtgattgcagatgcgcgcttttgctttcctattccagagggttttgacgatctgcacgcggcgccgctcctgtgcgcgggcttgatcggctatcgcgctcttcggcttgccggcgatgcacctgtactcggattctatggttttggagcggcggcgcatattttagctcaggtggccctgtggcagggtagaacggtttacgcgtttactcgcgatggcgacgctaaggcccaggcctttgctcgtgacatcggttgccaatgggccggaccctctggcgctgcgccgccgcaagctctggacgcagcgatcatcttcgcctccgcgggagaattggtgccgacagccctgcgtgcagtgcgcaaaggcgggcgtgttgtctgtgccggtattcatatgagcgatatcccggcattcccctacgccgatttatgggaggaacgtcagatcctgtcggtagcgaatttaacccgacgcgatggcgtagaattcctgccccttgcagcgcgtgcaggcgttcgcacacatgtcgaggccatgccgttaatgaaagcgaacgaggccctggaccgcctgcgtcgtggcgacgtcagtggcgctctggttttggtgccataa

SEQ ID NO: 103 - DNA, 1095 bp, synthetic construct
  source 1..1095: mol_type = other DNA
gacgcatacgctgcaattatcgagcgtcagggtggagaattcgttctggataacgtatctatcgaggatccgcgcgatggcgaagtgctggttaaggttgccgcagctggcatgtgtcataccgatctgacggttcgcgatcaatattacccgacgccgcttccggcggtgctgggccacgaaggtagcggcgttgttgaaaaagtgggacgtggcgtcaccactgtcaaaccaggtgacaaagtagtgttatccttcagctattgcggtacttgtccttcgtgcctcaaagggcatcaggcatactgtccgagcctgttcccgttaaatttcatgggccgtcgcctggatggttcaacgcccattacacgcaacggtcaagaggtcaacgcctgctttttcgggcaatcctcttttgcgacctatagtattgcgtcagaaaacaattgcgtcaaggttgccgacgatgcacagattgaacttttgggcccactgggctgcggcattcagaccggtgcgggaagtattttaaatgctctttgtcccgaacctggttcctctatagcgatctttggggtggggagtgtaggcttaagcgccgtgatggctgctaaagcatcgggctgcttgaagatcatcgcggttgacagaaatgcaggtcgcttggaactggcgcgtgaactgggcgccaccgatgtgattgacgccaacacggtcaatgctcaggaagcgatcgtcgcgatgactggtggcggcgccgactatgcaatggataccacagccattccagcggtgctgcggagtgcggtggatagcacgcacaatatgggtgaaacagcagtggtgggcggggcgaaactgggtaccgagttttcactagacatgaataacatgctgtttggtcgaaaattgcgtggcgtagtcgaaggatcgagcacgcctcaggtgttcatcccgcaactgattgcgatgcagaaagccgggctgtttccgtttgagaaactctgtaccttttatgatctggatcagatcaaccaggccgtagaggataccgaaaagactggaaaagcgataaaagccattctcaaaatgtaa

SEQ ID NO: 104 - DNA, 1275 bp, synthetic construct
  source 1..1275: mol_type = other DNA
tctacacagcctgcaaccatagctgattccgcgaccgatctggttgagggtcttgcacgtgcagcccgttctgcgcagcgccagttggcgcggatggattcaccggtaaaagaacgcgcgctgacgttagccgctgcagcgctgcgtgccgctgaggccgaaattttagccgctaacgcgcaggatatggcgaatggcgcagcaaacggcctgtcctcggccatgctcgaccggctgaagttaacgccagagcgtctggccggcattgccgatgctgtggcgcaagtcgccgggctggccgatccggtcggcgaggtgatcagtgaagctgcgcgtccgaatggcatggtgctgcagagagtgcgtattccggtcggagttatcggcatcatttacgaaagccgccccaacgttaccgccgatgcagcagcgctctgcgtgcgttcaggtaatgcggcgattctgcgcggtggctcggaagcggttcatagtaaccgtgcgatccataaagcgctggttgctgggcttgccgaaggcggagtgccggcagaagcggtgcagcttgtacctacgcaggaccgtgctgccgtaggggcaatgctaggtgccgcgggactgatcgacatgatcgttccgcgcggcggaaaaagccttgtcgctcgcgtccaggcagatgcccgcgtgccggtgttagcacacttggacggtatcaaccacacgtttgttcatgccagtgcagatccggcgatggcccaagcgatagtgttgaatgccaaaatgcgtcgcaccggcgtttgtggtgcgatggaaaccctgctgattgacgcgacttatccagatccccacggcctggtcgaaccgctgctagacgccggttgcgagctgcgcggcgatgctcgagcgagagcaattgatccgaggattgcgccagctgccgacaacgactgggatacagaatatttggaagcgattctttcggttgcagtggtcgacggtttggatgaagcgctcgcccacatcgcgcgccatgcctctggtcataccgatgcaatcgtcgcggcggaccaagatgtggcagaccgattcttagctgaagtagatagcgcaattgtaatgcataatgcatccagccagtttgctgatggcggtgagttcggcctgggtgctgagattggtattgccacggggcggctgcacgcgcgcggccctgtagcgctcgaagggctgactacctacaaatggctggtgcgcggaagcgggcaaactcgtccataa

SEQ ID NO: 105 - DNA, 1422 bp, synthetic construct
  source 1..1422: mol_type = other DNA
cgcgaacggctacagcaatacattgatggaaagtgggtagacagtgaaggtggcaaacgtcacgaagtcattaatccgactacagaggaaccctgttgtgtgattacgctgggcacgcaagcagatgtcgacaaagcagtggccgcggcacagcgcgcctttaaaaccttcagcaaaacgacgcgtgaggaacgactggcgctgcttgaacgcatcgtagaagaatacaagaagcgtgtccctgatttagccgccgcgatggccgaggaaatgggagctccggtaagctttgccagcaccgcgcaagttggcgccggaatcggagcatttctgggcaccatggccgcgctccgtaatttctcctttgttgaggacaacggtgcgtttaaagtggcctacgaaccgataggtgttgtgggtatgattacgccatggaactggccactgaatcagatagctctgaaagtagcaccggcgctggccgcggggaataccatgatcctgaaaccgtccgaggaatgcccaaccaacgcagcgatctttaccgaaattttggatgccgcaggggttccgccaggggtttttaacctgattcagggcgatggtcctggtgtaggcactgcgatcagtagtcatccgggcattgatatggttagtttcaccggttcgacccgtgcgggcatcctcgtggcgaaagctgcggccgataccgtcaagcgggtgcatcaggaacttggcggtaaatctcccaatgtggtgctgcccgatgcagacttcgcaaaatatctgccgtctaccgcgtcaggcccgttggtgaacagcggccagagctgcatttcgccaacccgtattttagtaccaagagaacgcgaagcagaagccgcggcttttgtttctgcgatgtactccgcaacaccggtcggggatccgatgcaagaaggtgcgcacattgggccggtggttaacaaagctcagtttgacaagatccgcggtctgattcaatcggcaatagacgaaggcgcgaaactcgagacaggcgggcccgacttaccggccaatgtgaaccgcggctattatatcaaaccaacggtcttttcaggcgttactcctgatatgcgcattgctcaggaagaaatcttcggcccggtggcgacgattatggcgtacgattcattagaggaggccattgagatcgcaaatgatacagcctatggactgtcggcctgcattactggtgatccggcgaaagcggctgaagtcgctcctgagcttcgtgcaggtatggtggctatcaataactggggccctactccgggtgctccgttcggtggctataaacagtccggtaacggtagggagggagggttgtatgggttgaaagacttcatggaaatgaaagcgatcagcggcctgcctgcctaa

SEQ ID NO: 106 - DNA, 1344 bp, synthetic construct
  source 1..1344: mol_type = other DNA
actgcccctaccgccgcagacctttccgccgatattgcacgggtttttgcactgcaacaagcgcacatgtgggaggccaaggcgtccaccgcggcggagcgcaaagaaaaattggcgcgtctgaaggccgcggttgaagcacacgcggatgacattgtggccgcggttctggaagatacgcgcaaacctgttggtgaaataagggtgaccgaagttctgaatgtaaccgccaatatccagcgaaacatcgataatctcgatgaatggatgaaaccggtcgaggtcgctacctcactgaatccagcggaccgcgcgcagataattcatgaagcgcgcggcgtatgcctgattcttggcccatggaatttccccttaggtctggcgctgggtccggtcgccgctgctatcgccgcaggcaatacttgtatcgtgaaattaacggacttgtgtccagcgaccgcaagagtggcatcggtgatcgtgcgtgaagcgttcgatgaaaaagatgtggctctgtttgagggagacgttagtgtagctaccgcgcttttggatctgccgtttaatcatgtattttttacaggctctccacgtgtaggcaaaattgtgatggctgctgcggcaaagcatctgaccagcgtcacgttagagcttggtgggaagtctcccgttattgtcgatgatagcgcagatatcgatcaagttgctgcccagttagccgcggccaaacaattcaacggcgggcaggcctgcatttccccggactatgtgtttgtgaaagaagacaaaaaagctgcgctggtagaaggtttccgtgccaatgtgcagaaaaacttgtatgatgatgcaggcaacctgaaaaaagacagtattgcacaggtggtcaacaaagcgaactttgatcgtgtgaaagccatgttcgacgatgcagtcgcaaaaggcgcgaccgtcgccgctggtggaacgtttgaagcggatgacttgactattcatccgacaatgctgacaggcgtaaccccgcagatgactattctccaggatgagatctttgcccctgtcattccggtgatgacctacgacacgctggatcaagcgatcgggtatatcgaagcacgcgacaaaccgctagcactctatgtttacagtaaagatgaagcgaacgttgaaaaggtcttagcccgcacgtcatcgggtggtgttacggtgaatggtgtgttctcgcactacctggaaaacaacctgccgttcgggggggttaacacaagcggtatgggcagctaccatggcgtgttcggatttaagtgctttagccacgagcgggctgtatatcgtcatcagcagtaa

SEQ ID NO: 107 - DNA, 1023 bp, synthetic construct
  source 1..1023: mol_type = other DNA
ggttaccgggttgtagtggtgggtgcgactgggaatgtggggcgtgaaatgctgaacattctggcagaacgcgagtttccttgtgacgagatcgcagcggttgctagctctcgttcgcagggcaccgaaatagaatttggcgaaactggccggaagctgaaagtacagaatgttgaaaattttgattttaccggatgggacattgcactgtttgcggcgggatcaggcccgacgcagatccatgctccacgtgccgcttctcagggctgcgtggtgatcgataacagtagcttataccgcatggacccggacgtgcctctgatcgtgcccgaggtgaatccggatgcgattgatggctataccaaaaaaaacattattgccaatccaaactgttccaccgcgcaaatggtcgtggcgctgaaaccgttacatgatgccgccaaaattaaaagagttgtcgtctccacgtatcaaagcgtttccggcgcgggtaaagaagggatggatgaactgttcgaacaaagccgcgcgatatttgtcggggacccggtggaaccgaaaaaattcaccaaacagatcgcattcaacgtgatccctcatatcgatgtattcctagacgatggttcgactaaagaagagtggaaaatggtcgccgaaaccaaaaaaattttggaccccaaggttaaggtaacggcaacctgcgtgcgtgtgccggtgttcatcggccactcggaagcgttaaacattgagttcgagaatgaaattagtgccgaggaagcgcagaatatcctgcgcgaagcaccaggtgtgatgctcgtcgataagcgcgagaacggcggatatgttacgccggtcgaatgcgttggtgattttgccacatttgttagccgcgtacgtgaggattcaacagttgataacggccttaatatttggtgtgtcagtgataacctgaggaaaggtgctgccttgaacgctgtacagattgcagaactgctcggtcgtcgacaccttaaaaagggttaa

SEQ ID NO: 108 - DNA, 1005 bp, synthetic construct
  source 1..1005: mol_type = other DNA
gcgatcaaagttgcgataaacggttttggacgtatcgggaggaatgtggcccgcgccattttagaacgtcccgattgtgggttagaactggttagcattaacgacctggctgatgccaaggctaacgccctgctgtttaaacgcgacagcgttcatggcgcgttcagtggcgaagtatcagtggatggcaatgatctgattgtgaatggcaagcgcattcaggtgactgcagagcgcgatcctgctaacctgccacacggagccaatggtattgacattgcgctggaatgcacgggctttttcaccaatcgtgatggtggccagaaacacttggacgcgggcgccaaacgcgttctgatttccgctccggcaaaaaacgtagacctgacggtcgtctatggtgtgaaccacgacaaactgaccggcgatcataagatcgtgtccaacgcgagttgcacgaccaactgtttggcgccgatggcaaaagtcctgcatgaatctatcgggattgagcgtggtctaatgacaacgattcattcgtataccaatgatcaaaaaatactcgaccagatccatagcgatcctagacgggctcgggcagcggcgatgaatatgatccccacaagcaccggggccgcagttgcagtgggtgaagttctgccagacttaaaagggaaacttgatggttcgtcgattcgagtcccgaccccgaacgtatctgtcgtggatcttactttcacgccgaagcgtgataccagcgtagaggaagtaaatggtctcttgaaagcggctgccgaaggcgcattgaaaggcgtgttaggttacaccgacgaaccgctggtttcaatcgattttaaccacgatccgcatagttcaacaatcgacagccttgagactgccgtgctcgaaggtaaactggtgcgcgtcctgtcttggtacgataatgagtggggcttttccaaccgtatgctggatacggcgggagcaatggcgaaattcctttaa

SEQ ID NO: 109 - DNA, 1512 bp, synthetic construct
  source 1..1512: mol_type = other DNA
aatgacatgactaccatctcacgcacgcagcgtgaatactccgaggccgcaaaagctttcctcgcgagaaagccgcaattgtttattaataacgagtgggtcgatagcagtcacgatgcagtgatcgaagtggaagacccctcgaatgggaggattgtaggtcatgtcgttgatgcctcggacaaagacgttgaccgggcggttgccgctgcgcgggccgctttcgatgatggtcgttggtccaacctgccgccaatggtacgcgatcgtaccatgaatcgcctggccgacctgcttgaagcaaacgcagatctctttgcagagctggaagcgattgataatggtaaaccgaagggtatggccggcgccgttgatattccaggtgcgataagccaactacgcttcatggcaggatgggccagcaaggtagctggcgaaacgacgcagccttacacgatgccgaatggcaccgtgtttagttacaccgtcaaagaacccgtcggtgtctgcgcgcagattgtgccgtggaacttcccgctgctgatggcatcattgaagatcgccccggcgctggcggctggatgtacactggtgctgaaacctgccgaacagacatcgcttaccgcgttaaaactggcagatttggtggttgaggctggctttcctgcgggagtgatcaacattatcacagggaacggccacaccgcaggtgatcgcatggtcaaacatcccgacgtagacaaagtcgcctttactggctccaccgaaatcgggaaactgataaatcgaaacgcaaccaccacgcttaaacgggttacgctcgaactgggcgggaaaagtcccgtagtggttatgccagacgtagatgtggcgcagaccgcgcctggcgttgccggtgcgatttttttcaacgctggccaggtttgtgttgccggtagtcgtttatatgcgcaccgttcggtgttcgattccgtgttagaaggtatgacccagactgcgccgttttgggcgccgcgcccgagcctggatccagaagcacacatgggaccgttggtcagcaaagagcaacatgaccgtgtgatgggatatatcgaggcgggcaagcgtgatggcgccagcgtagtgatgggcggtgattgcccaagcgctgatggagggtactatgttaatccgacgattctggcagacgtgaatccgcagatgtctgtcgtgcgcgaggaaatttttggtccggttgtcgtcgcccaacgcttcgacgatttagatgaagtggcgaaaatggcaaacgacacctgttttggcttaggtgcgggcgtgtggacgcgcgatgttgcggtgatgcataaacttgcttcaaagatcaaatctggcactgtgtggggcaactgccatgccctgatcgatacagcgctgccttttggcggctataaagaatctgggctgggtcgagaacaggggcgtgccggtattgatgcttatttggagactaaaacagtaattattcaaatgtaa

SEQ ID NO: 110 - DNA, 1503 bp, synthetic construct
  source 1..1503: mol_type = other DNA
gctacgcagttgagaagtgcagaaaatgaatatgggatcaaatccgagtatggtcattatataggaggtgagtggattgcaggggatagcggcaagaccatagatttactaaatccctctaccggtaaagtgctgaccaaaattcaagccggcaacgcaaaagatattgaacgcgcgattgccgctgcaaaagcggcgtttccgaagtggagccagagcctgccaggggagcgccaagaaatcctgatagaggttgcgcgtcgtctgaaagcacgccattcgcactatgcaaccttagaaacgctcaataacggtaaaccgatgcgcgaatcaatgtatttcgatatgcctcaaacgatcgggcaatttgagctgttcgccggtgccgcctatggcctgcatggccagacgctggattatccagacgcgattggcatcgtccaccgtgaaccgttaggcgtatgcgcgcagattattccatggaacgtgccgatgttgatgatggcgtgcaaaatcgcgcccgcgctggcctctggcaacactgtcgttctgaaaccggccgaaacggtgtgcctttctgtgattgaatttttcgtggaaatggctgatctgttgcctccgggtgtgatcaacgttgttaccgggtatggtgctgacgttggcgaggcgcttgtaacaagccctgatgtagctaaagtggcctttaccggttcgattgctacggcgcgccggattattcagtatgcctcggccaatatcattccacagacgctcgagttgggcggtaaatcagcgcatatcgtgtgtggcgatgccgatattgacgcggcggtggaaagtgcgactatgtccaccgttttaaataaaggtgaagtctgtctggctggttcacgcctgtttctgcatcagtccatccaggatgaattcctggccaaatttaaaacagcgcttgaaggcattcgccaaggcgacccgctagatatggcgactcaacttggagcgcaggcatcgaagatgcagtttgacaaggtgcaaagctacttaaggctggctacagaggaaggggcagaggtactgaccggcggtagtcgttcagatgccgcagatctggcagatggcaattttatcaaaccgacggtttttactaacgtcaataactccatgcggatcgcgcaggaagagattttcggaccggttaccagcgtaattacatggagcgacgaagacgacatgatgaaacaggccaacaatacaacttacggcttggctggcggtgtctggaccaaggacatcgcacgagcacaccgtattgcgcgtaaactcgaaactggcacggtctggatcaatcgctactacaacctgaaagccaacatgccgctgggaggttacaagcaaagtggctttgggcgtgaattcagccatgaagtgctgaatcactacacccagaccaaatctgtggttgtcaacctccaggaaggtcgtaccggaatgttcgatcagtaa

(19) United States
(12) Patent Application Publication
(10) Pub. No.: US 2025/0376706 A1
(43) Pub. Date: Dec. 11, 2025

(54) RECOMBINANT MICROORGANISMS THAT CATABOLIZE LIGNIN AROMATICS AND METHODS OF USING SAME

(71) Applicant: Wisconsin Alumni Research Foundation, Madison, WI (US)

(72) Inventors: Timothy Donohue, Middleton, WI (US); Fletcher Metz, Madison, WI (US); Kevin Myers, Madison, WI (US); Fachuang Lu, Madison, WI (US); Daniel Noguera, Madison, WI (US)

(73) Assignee: Wisconsin Alumni Research Foundation, Madison, WI (US)

(21) Appl. No.: 18/737,647

(22) Filed: Jun. 7, 2024

(51) Int. Cl. C12P 17/04; C12N 9/02; C12N 9/88

(52) U.S. Cl. CPC C12P 17/04; C12N 9/0004; C12N 9/0069; C12N 9/0093; C12N 9/88; C12Y 102/01071; C12Y 113/11043; C12Y 117/01; C12Y 401/01028; C12Y 402/01

(57) ABSTRACT

Recombinant microorganisms that catabolize lignin aromatics, such as β-5 linked lignin aromatics, and methods of using same to catabolize the lignin aromatics.

### STATEMENT REGARDING FEDERALLY SPONSORED RESEARCH

This invention was made with government support under DE-SC0018409 awarded by the US Department of Energy. The government has certain rights in the invention.

### SEQUENCE LISTING

The instant application contains a Sequence Listing which has been submitted in XML format and is hereby incorporated by reference in its entirety. The XML copy, created on May 31, 2024, is named USPTO-24607-09824544-P240270US01-SEQ_LIST.xml and is 140,384 bytes in size.

### FIELD OF THE INVENTION

The invention is directed to recombinant microorganisms that catabolize lignin aromatics, such as 3-5 linked lignin aromatics, and methods of using same to catabolize the lignin aromatics.

### BACKGROUND

Over the past century, aromatic compounds have proven integral to industries that generate critical chemicals and materials for society. For example, aromatic compounds are precursors for the production of plastics, adhesives, medicinal compounds, and flavorings. Most of today's industrial aromatics are derived from fossil fuels. However, there is increasing interest in identifying renewable raw materials that can serve as alternative sources of these valuable chemicals.

The plant polymer lignin can comprise up to 40% of the dry weight of plant biomass, making it the second most abundant biopolymer on the planet (1) and an attractive source of renewable aromatics for producing chemicals. Lignin is a heteropolymer composed of syringyl (S), guaiacyl (G), and p-hydroxyphenyl (H) aromatic subunits which differ in the number of methoxy groups attached to the aromatic ring (two, one, or zero, respectively) (2, 3). Since lignin polymers are synthesized via radical chemistry in plants, the aromatic subunits are joined by a variety of interunit bonds (FIG. 1 (A)) (4-6). The chemical heterogeneity of its inter-aromatic linkages makes lignin recalcitrant to break down, so it has traditionally been burned for fuel (1, 7, 8). However, strategies are emerging to convert the aromatic subunits of lignin to commodity chemicals and materials that are needed by society (2, 8).

One promising strategy is to use the aromatic compounds resulting from depolymerization of lignin as carbon sources that microbes can funnel into valuable products (9-12). Microbes suitable for this purpose are needed.

### SUMMARY OF THE INVENTION

One aspect of the invention is directed recombinant microorganisms. The recombinant microorganisms can comprise any one or more, any two or more, any three or more, any four or more, or each of: one or more recombinant alcohol dehydrogenase genes; one or more recombinant aldehyde dehydrogenase genes; a recombinant T-formaldehyde lyase gene; a recombinant lignostilbene dioxygenase gene; and a recombinant aromatic acid decarboxylase gene.

In some versions, the recombinant microorganism comprises any two or more, any three or more, any four or more, or each of: the one or more recombinant alcohol dehydrogenase genes; the one or more recombinant aldehyde dehydrogenase genes; the recombinant T-formaldehyde lyase gene; the recombinant lignostilbene dioxygenase gene; and the recombinant aromatic acid decarboxylase gene. In some versions, the recombinant microorganism comprises any three or more, any four or more, or each of: the one or more recombinant alcohol dehydrogenase genes; the one or more recombinant aldehyde dehydrogenase genes; the recombinant T-formaldehyde lyase gene; the recombinant lignostilbene dioxygenase gene; and the recombinant aromatic acid decarboxylase gene. In some versions, the recombinant microorganism comprises any four or more or each of: the one or more recombinant alcohol dehydrogenase genes; the one or more recombinant aldehyde dehydrogenase genes; the recombinant T-formaldehyde lyase gene; the recombinant lignostilbene dioxygenase gene; and the recombinant aromatic acid decarboxylase gene. In some versions, the recombinant microorganism comprises each of: the one or more recombinant alcohol dehydrogenase genes; the one or more recombinant aldehyde dehydrogenase genes; the recombinant T-formaldehyde lyase gene; the recombinant lignostilbene dioxygenase gene; and the recombinant aromatic acid decarboxylase gene.

In some versions, the one or more recombinant alcohol dehydrogenase genes encode FdhA of Novosphingobium aromaticivorans (SEQ ID NO:2) or a homolog thereof. In some versions, the one or more recombinant alcohol dehydrogenase genes encode FdhA of Novosphingobium aromaticivorans (SEQ ID NO:2), a protein comprising a sequence at least 80%, at least 85%, at least 90%, at least 95%, or at least 99% identical to SEQ ID NO:2, an ortholog of FdhA of Novosphingobium aromaticivorans, or a recombinant variant of the ortholog of FdhA of Novosphingobium aromaticivorans.

In some versions, the one or more recombinant alcohol dehydrogenase genes encode Saro_0995 of Novosphingobium aromaticivorans (SEQ ID NO:4) or a homolog thereof. In some versions, the one or more recombinant alcohol dehydrogenase genes encode Saro_0995 of Novosphingobium aromaticivorans (SEQ ID NO:4), a protein comprising a sequence at least 80%, at least 85%, at least 90%, at least 95%, or at least 99% identical to SEQ ID NO:4, an ortholog of Saro_0995 of Novosphingobium aromaticivorans, or a recombinant variant of the ortholog of Saro_0995 of Novosphingobium aromaticivorans.

In some versions, the one or more recombinant alcohol dehydrogenase genes encode Saro_3899 of Novosphingobium aromaticivorans (SEQ ID NO:6) or a homolog thereof. In some versions, the one or more recombinant alcohol dehydrogenase genes encode Saro_3899 of Novosphingobium aromaticivorans (SEQ ID NO:6), a protein comprising a sequence at least 80%, at least 85%, at least 90%, at least 95%, or at least 99% identical to SEQ ID NO:6, an ortholog of Saro_3899 of Novosphingobium aromaticivorans, or a recombinant variant of the ortholog of Saro_3899 of Novosphingobium aromaticivorans.

In some versions, the one or more recombinant aldehyde dehydrogenase genes encode FerD of Novosphingobium aromaticivorans (SEQ ID NO:8) or a homolog thereof. In some versions, the one or more recombinant aldehyde dehydrogenase genes encode FerD of Novosphingobium aromaticivorans (SEQ ID NO:8), a protein comprising a sequence at least 80%, at least 85%, at least 90%, at least 95%, or at least 99% identical to SEQ ID NO:8, an ortholog of FerD of Novosphingobium aromaticivorans, or a recombinant variant of the ortholog of FerD of Novosphingobium aromaticivorans.

In some versions, the one or more recombinant aldehyde dehydrogenase genes encode Saro_1104 of Novosphingobium aromaticivorans (SEQ ID NO:10) or a homolog thereof. In some versions, the one or more recombinant aldehyde dehydrogenase genes encode Saro_1104 of Novosphingobium aromaticivorans (SEQ ID NO:10), a protein comprising a sequence at least 80%, at least 85%, at least 90%, at least 95%, or at least 99% identical to SEQ ID NO:10, an ortholog of Saro_1104 of Novosphingobium aromaticivorans, or a recombinant variant of the ortholog of Saro_1104 of Novosphingobium aromaticivorans.

In some versions, the one or more recombinant aldehyde dehydrogenase genes encode Saro_1197 of Novosphingobium aromaticivorans (SEQ ID NO:12) or a homolog thereof. In some versions, the one or more recombinant aldehyde dehydrogenase genes encode Saro_1197 of Novosphingobium aromaticivorans (SEQ ID NO:12), a protein comprising a sequence at least 80%, at least 85%, at least 90%, at least 95%, or at least 99% identical to SEQ ID NO:12, an ortholog of Saro_1197 of Novosphingobium aromaticivorans, or a recombinant variant of the ortholog of Saro_1197 of Novosphingobium aromaticivorans.

In some versions, the one or more recombinant aldehyde dehydrogenase genes encode Saro_2869 of Novosphingobium aromaticivorans (SEQ ID NO:14) or a homolog thereof. In some versions, the one or more recombinant aldehyde dehydrogenase genes encode Saro_2869 of Novosphingobium aromaticivorans (SEQ ID NO:14), a protein comprising a sequence at least 80%, at least 85%, at least 90%, at least 95%, or at least 99% identical to SEQ ID NO:14, an ortholog of Saro_2869 of Novosphingobium aromaticivorans, or a recombinant variant of the ortholog of Saro_2869 of Novosphingobium aromaticivorans.

In some versions, the recombinant y-formaldehyde lyase gene encodes PcfL of Novosphingobium aromaticivorans (SEQ ID NO:16) or a homolog thereof. In some versions, the recombinant y-formaldehyde lyase gene encodes PcfL of Novosphingobium aromaticivorans (SEQ ID NO:16), a protein comprising a sequence at least 80%, at least 85%, at least 90%, at least 95%, or at least 99% identical to SEQ ID NO:16, an ortholog of PcfL of Novosphingobium aromaticivorans, a recombinant variant of the ortholog of PcfL of Novosphingobium aromaticivorans.

In some versions, the recombinant lignostilbene dioxygenase gene encodes LsdD of Novosphingobium aromaticivorans (SEQ ID NO:18) or a homolog thereof. In some versions, the recombinant lignostilbene dioxygenase gene encodes LsdD of Novosphingobium aromaticivorans (SEQ ID NO:18), a protein comprising a sequence at least 80% identical, at least 85% identical, at least 90% identical, at least 95% identical, or at least 99% identical to SEQ ID NO:18, an ortholog of LsdD of Novosphingobium aromaticivorans, or a recombinant variant of the ortholog of LsdD of Novosphingobium aromaticivorans.

In some versions, the recombinant aromatic acid decarboxylase gene encodes LigW of Novosphingobium aromaticivorans (SEQ ID NO:20) or a homolog thereof. In some versions, the recombinant aromatic acid decarboxylase gene encodes LigW of Novosphingobium aromaticivorans (SEQ ID NO:20), a protein comprising a sequence at least 80% identical, at least 85% identical, at least 90% identical, at least 95% identical, or at least 99% identical to SEQ ID NO:20, an ortholog of LigW of Novosphingobium aromaticivorans, or a recombinant variant of the ortholog of LigW of Novosphingobium aromaticivorans.

In some versions, the orthologs of FdhA, Saro_0995, Saro_3899, FerD, Saro_1104, Saro_1197, Saro_2869, PcfL, LsdD, and/or LigW are from a bacterium. In some versions, the orthologs of FdhA, Saro_0995, Saro_3899, FerD, Saro_1104, Saro_1197, Saro_2869, PcfL, LsdD, and/or LigW are from an Alphaproteobacterium. In some versions, the orthologs of FdhA, Saro_0995, Saro_3899, FerD, Saro_1104, Saro_1197, Saro_2869, PcfL, LsdD, and/or LigW are from an order selected from the group consisting of Sphingomonadales, Actinomyces, Gammaproteobacteria, Betaproteobacteria, and Bacilli. In some versions, the orthologs of FdhA, Saro_0995, Saro_3899, FerD, Saro_1104, Saro_1197, Saro_2869, PcfL, LsdD, and/or LigW are from the group consisting of Novosphingobium, Erythrobacteraceae, Sphingobium, and Sphingomonas.

In some versions, the recombinant microorganism is a bacterium. In some versions, the recombinant microorganism is an Alphaproteobacterium. In some versions, the recombinant microorganism is from an order selected from the group consisting of Sphingomonadales, Actinomyces, Gammaproteobacteria, Betaproteobacteria, and Bacilli. In some versions, the recombinant microorganism is from the group consisting of Novosphingobium, Erythrobacteraceae, Sphingobium, and Sphingomonas.

Another aspect of the invention is directed to methods of catabolizing a lignin aromatic. The methods can comprise culturing the recombinant microorganism of the invention in a medium comprising the lignin aromatic to thereby catabolize the lignin aromatic. In some versions, the lignin aromatic comprises a β-5 linked lignin aromatic. In some versions, the lignin aromatic comprises one or more of dehydrodiconiferyl alcohol (DC-A), dehydrodiconiferyl aldehyde (DC-L), dehydrodiconiferyl carboxylic acid (DC-C), dehydrodiconiferyl stilbene carboxylic acid (DC-S-C), 5-formyl ferulate (5-FF), 5-carboxyferulate (5-CF), and 4-hydroxyphenyl and syringyl analogs thereof.

The objects and advantages of the invention will appear more fully from the following detailed description of the preferred embodiment of the invention made in conjunction with the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. DC-A models β-5 linked lignin aromatics. A) Model lignin polymer that illustrates major interunit linkages and aromatic subunits. B) Structure of dehydrodiconiferyl alcohol (DC-A), a β-5 linked aromatic dimer composed of two G-family aromatic subunits. The β-5 bond is highlighted in red.

FIG. 2. N. aromaticivorans funnels DC-A into central aromatic metabolism. A) Growth of WT N. aromaticivorans in SMB minimal medium with DC-A as the sole carbon source. B) Growth of 12444PDC in SMB minimal medium containing either DC-A plus glucose or glucose alone as carbon sources. C) Metabolite concentrations in extracellular medium of 12444PDC grown in SMB minimal medium with DC-A plus glucose as carbon sources. Error bars represent standard deviation across biological triplicates.

FIG. 3. Genome-wide screens identify candidate genes for DC-A catabolism. A) Dot plot (log₂ scale) of RNA-Seq (y-axis) and RB-TnSeq (x-axis) data sets, with each dot representing a single gene. The horizontal and vertical red lines mark a 2-fold increase in transcript abundance when N. aromaticivorans PDC12444 is grown on DC-A compared to vanillin and a 2-fold abundance reduction of a disrupted gene when a N. aromaticivorans DSM12444 RB-TnSeq library is grown on DC-A compared to glucose, respectively. The five candidate genes investigated in this study are labeled in red. B) Genomic region containing four of the five candidate genes. Candidate genes are labeled in red. Experimentally determined transcription start sites (TSS) are labeled (34).

FIG. 4. Proposed catabolic pathway for DC-A in N. aromaticivorans. The allylic alcohol side chain of DC-A is oxidized to DC-L and then to DC-C by dehydrogenases. The five-member ring of DC-C is opened by PcfL to form DC-S-C, which is then cleaved by LsdD into vanillin and 5-FF. 5-FF is oxidized to 5-CF by FerD and other dehydrogenases before it is decarboxylated by LigW to form ferulic acid. Metabolism of ferulic acid and vanillin to PDC by N. aromaticivorans has been previously described (10, 21). The gene products predicted to be involved in metabolism of formaldehyde following oxidation by FdhA are based on homology of N. aromaticivorans gene products with known S-glutathione hydrolases (Saro_2822) (35) and the subunits of a formate dehydrogenase complex (Saro_0732, Saro_0733, and Saro_0735) (36).

FIGS. 5A-5C. PcfL converts DC-C to DC-S-C. FIG. 5A) Metabolite concentrations in extracellular medium of 12444PDCΔpcfL grown in SMB minimal medium with DC-A plus glucose as carbon sources. Error bars represent standard deviation across biological triplicates. FIG. 5 B) Representative HPLC chromatograms of in vitro reactions containing DC-C and either control E. coli B834 cell extract or cell extract from E. coli B834 expressing recombinant PcfL. FIG. 5C) Conversion of DC-C to DC-S-C by PcfL.

FIGS. 6A-6C. LsdD cleaves DC-S-C to form 5-FF and vanillin. FIG. 6A) Metabolite concentrations in extracellular medium of 12444PDCΔlsdD grown in SMB minimal medium with DC-A plus glucose as carbon sources. Error bars represent standard deviation across biological triplicates. FIG. 6B) Representative HPLC chromatograms of in vitro reactions containing DC-S-C and either control E. coli cell extract or cell extract from E. coli expressing recombinant LsdD. FIG. 6C) Cleavage of DC-S-C to 5-FF and vanillin by LsdD and abiotic dimerization of DC-S-C to DC-T-C.

FIGS. 7A-7C. FerD and LigW convert 5-FF to 5-CF and then ferulic acid. FIG. 7A) Metabolite concentrations in extracellular medium of 12444PDCΔferD and 12444PDCΔligW grown in SMB minimal medium with DC-A plus glucose as carbon sources. Error bars represent standard deviation across biological triplicates. FIG. 7B) Representative HPLC chromatograms of in vitro reactions (left) containing 5-FF plus NAD⁺ and either control E. coli B834 cell extract or cell extract of E. coli B834 expressing recombinant FerD or reactions (right) containing 5-CF and either control E. coli B834 cell extract or cell extract of E. coli B834 expressing recombinant LigW. FIG. 7C) Oxidation of 5-FF to 5-CF by FerD and decarboxylation of 5-CF to ferulic acid by LigW.

FIG. 8. Multiple partially redundant ADHs and ALDHs can oxidize the allylic side chain of DC-A. Concentration of DC-L over the course of 1 hour long in vitro assays containing A) DC-A, NAD⁺, and a control E. coli B834 cell extract or cell extracts of E. coli B834 expressing recombinant candidate ADHs or B) DC-L, NAD⁺, and control E. coli B834 cell extract or cell extracts of E. coli B834 expressing recombinant candidate ALDHs. For clarity of presentation, only dehydrogenases exhibiting activity on the tested substrates are shown. Error bars represent standard deviation across triplicates.

FIG. 9. The proposed catabolic pathway enzymes can convert DC-A to ferulic acid and vanillic acid in vitro. Representative HPLC chromatograms of in vitro reactions containing DC-A plus NAD⁺ and either control E. coli B834 cell extract or cell extracts from E. coli B834 expressing recombinant Saro_0995, PcfL, LsdD, FerD, and LigW.

FIGS. 10A-10G. Order Sphingomonadales contains two pathways for conversion of DC-C to DC-S-C and a conserved pathway for DC-S-C catabolism. Phylogeny constructed based on the bacterial reference genes of Alphaproteobacteria containing homologs (>50% amino acid identity, >70% query coverage) of at least two enzymes found in the β-5 linked aromatic catabolic pathways characterized in N. aromaticivorans or Sphingobium sp. SYK-6. Homologs found in each species are marked by colored boxes. Clades are labeled and color-coded. The scale bar indicates the number of nucleotide substitutions per sequence site. The gap in the outgroup corresponds to 1.5 on the scale bar. A simplified diagram of the DC-A catabolic pathways in N. aromaticivorans and Sphingobium sp. SYK-6 is shown. Phylogeny presented in FIG. 10A represents the bacteria from left to right as they appear in the order in which they appear in FIGS. 10B-10G.

FIG. 11 Trace amounts of DC-L transiently accumulate during DC-A catabolism. DC-L concentration in extracellular medium of 12444PDC grown in SMB minimal medium with DC-A plus glucose as carbon sources. Error bars represent standard deviation across biological triplicates.

FIG. 12. Genome-wide screens identify candidate genes for DC-A catabolism. Dot plot (log₂ scale) of RNA-Seq (y-axis) and RB-TnSeq (x-axis) data sets, with each dot representing a single gene. The horizontal and vertical red lines mark a 2-fold increase in transcript abundance when N. aromaticivorans PDC12444 is grown on DC-A compared to A) glucose or B) ferulic acid and a 2-fold abundance reduction of a disrupted gene when a N. aromaticivorans DSM12444 RB-TnSeq library is grown on DC-A compared to glucose, respectively. The five candidate genes investigated in this study are labeled in red.

FIG. 13. Formaldehyde is released when PcfL converts DC-C to DC-S-C. Concentration of formaldehyde after 6 hours of incubating in vitro reactions containing DC-C and either cell extract of E. coli B834 expressing recombinant PcfL or control E. coli B834 cell extract. Error bars represent standard deviation across triplicates.

FIG. 14. FdhA acts on formaldehyde released during DC-A catabolism. A) Metabolite concentrations in extracellular medium of 12444PDCΔfdhA grown in SMB minimal medium with DC-A plus glucose as carbon sources. B) Formaldehyde concentration in extracellular medium of 12444PDC or 12444PDCΔfdhA grown in SMB minimal medium with DC-A plus glucose as carbon sources. Error bars represent standard deviation across biological triplicates.

FIGS. 15A and 15B. DC-S-C abiotically homodimerizes in aqueous solutions to form DC-T-C. FIG. 15A)¹³C NMR spectrum of the product obtained when DC-S-C is incubated in SMB minimal medium supplemented with 1 g/L glucose. The structure of the resulting compound, DC-T-C, is shown. FIG. 15B) Loss of DC-S-C over time in various solutions. Note that some DC-S-C visually precipitated in the water condition. Error bars represent standard deviation across triplicates.

FIGS. 16A and 16B. FerD is an NAD⁺-dependent aldehyde dehydrogenase. FIG. 16A) Representative HPLC chromatograms of in vitro reactions containing 5-FF and either control E. coli B834 cell extract or cell extract of E. coli B834 expressing recombinant FerD without added NAD⁺. FIG. 16B) Ratio of NAD⁺ to NADH after 6 hours incubating in vitro reactions containing 5-FF and NAD⁺ along with purified FerD, cell extract of E. coli B834 expressing recombinant FerD, or control E. coli B834 cell extract. Error bars represent standard deviation across triplicates.

FIG. 17. Differences in DC-A, DC-L, and DC-C absorbance can be leveraged in colorimetric assays. UV-Vis traces of 0.2 mM solutions of DC-A, DC-L, and DC-C in S30 buffer.

FIG. 18. FerD converts vanillin to vanillic acid. Representative HPLC chromatograms of in vitro reactions containing vanillin and either control E. coli B834 cell extract or cell extract of E. coli B834 expressing recombinant FerD.

FIGS. 19A-19C. PcfL exhibits activity on DC-A and DC-L in vitro. Representative HPLC chromatograms of in vitro reactions containing DC-A (FIG. 19A) or DC-L (FIG. 19B) and either control E. coli B834 cell extract or cell extract of E. coli B834 expressing recombinant PcfL. FIG. 19C) Structures of proposed stilbene compounds based on m/z of the in vitro reaction products.

FIG. 20. Proposed N. aromaticivorans catabolic pathway for DC-A, accounting for the ability of PcfL to act on DC-A, DC-L, and DC-C. The allylic alcohol is oxidized to an aldehyde and then to a carboxylic acid by dehydrogenases. The five-member ring of DC-C is opened by PcfL to form DC-S-C, which is then cleaved by LsdD into vanillin and 5-FF. 5-FF is oxidized to 5-CF by FerD and other dehydrogenases before it is decarboxylated by LigW to form ferulic acid. Metabolism of ferulic acid and vanillin to PDC by N. aromaticivorans has been previously described (10, 21). The gene products involved in metabolism of formaldehyde following oxidation by FdhA represent a hypothetical pathway based on homology with known S-glutathione hydrolases (Saro_2822) (35) and the subunits of a formate dehydrogenase complex (Saro_0732, Saro_0733, and Saro_0735) (36). Steps that differ from those proposed in FIG. 4 are marked with blue arrows.

FIGS. 21A-21C. The full N. aromaticivorans DC-A catabolic pathway is exclusive to Alphaproteobacteria. Phylogeny constructed based on the bacterial reference genes of bacteria containing homologs (>50% amino acid identity, >70% query coverage) of at least two enzymes found in the N. aromaticivorans β-5 linked aromatic pathway. The bacterial species are sorted by class. The colored bars to the right of the tree indicate the proportion of each class containing a homolog of each enzyme. The scale bar indicates the number of nucleotide substitutions per sequence site. A simplified diagram of the DC-A catabolic pathway in N. aromaticivorans is shown in FIG. 21A. FIGS. 21B and 21C show a closeups of FIG. 21A with relevant percentages.

FIGS. 22A-22E. DC-A, DC-L, DC-C, and DC-S-C synthesis. FIG. 22A) Synthetic routes to DC-A, DC-L, DC-C, and DC-S-C. FIGS. 22B-22E)¹³C NMR (acetone-d₆) spectra and structures of synthetic DC-A (FIG. 22B), DC-L (FIG. 22C), DC-C (FIG. 22D), and DC-S-C (FIG. 22E).

FIGS. 23A-23C. DC-S-C and DC-T-C synthesis. FIG. A) Synthetic routes to 5-FF and 5-CF. B-C)¹³C NMR (acetone-d₆) spectra and structures of synthetic FIG. B) 5-FF and FIG. C) 5-CF.

FIG. 24. Growth of 12444PDC and 12444PDC mutant strains. Growth curves of 12444PDC and 12444PDC mutant strains in SMB minimal medium containing 0.5 mM DC-A and 1 g/L glucose as carbon sources. Error bars represent standard deviation across biological triplicates.

FIG. 25. Solvent B (MeOH) percent protocol for HPLC method. Trace of percent solvent B over time. Solvent A was 0.2% formic acid in water.

FIG. 26. Differences in DC-S-C and DC-T-C can be leveraged in colorimetric assays. UV-Vis traces of 0.2 mM solutions of DC-S-C and DC-T-C in S30 buffer.

### DETAILED DESCRIPTION OF THE INVENTION

The recombinant microorganisms of the invention can comprise one or more recombinant genes. The recombinant genes can comprise one or more recombinant alcohol dehydrogenase genes, one or more recombinant aldehyde dehydrogenase genes, a recombinant 7-formaldehyde lyase gene, a recombinant lignostilbene dioxygenase gene, and/or a recombinant aromatic acid decarboxylase gene.

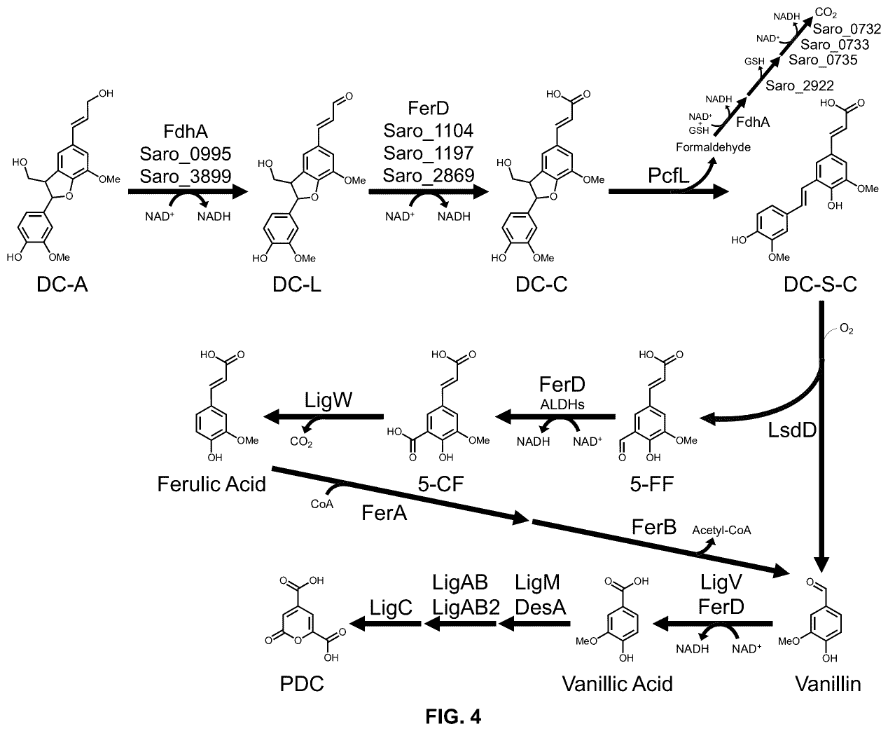

The recombinant alcohol dehydrogenase genes of the invention are preferably capable of catalyzing the conversion of dehydrodiconiferyl alcohol (DC-A) to dehydrodiconiferyl aldehyde (DC-L). See, e.g., FIG. 4. The recombinant alcohol dehydrogenase genes of the invention may also be capable of catalyzing the conversion of phenolic analogs (such as 4-hydroxyphenyl or syringyl analogs) of dehydrodiconiferyl alcohol (DC-A) (a guaiacyl aromatic) to phenolic analogs (such as 4-hydroxyphenyl or syringyl analogs) of dehydrodiconiferyl aldehyde (DC-L) (a guaiacyl aromatic). Exemplary recombinant alcohol dehydrogenase genes include those encoding FdhA of Novosphingobium aromaticivorans (Saro_0874) (SEQ ID NO:2 (exemplary coding sequence is SEQ ID NO:1)) or a homolog thereof, Saro_0995 of Novosphingobium aromaticivorans (SEQ ID NO:4 (exemplary coding sequence is SEQ ID NO:3)) or a homolog thereof, and Saro_3899 of Novosphingobium aromaticivorans (SEQ ID NO:6 (exemplary coding sequence is SEQ ID NO:5)) or a homolog thereof. The homolog of FdhA can comprise a protein comprising a sequence at least 80%, at least 85%, at least 90%, at least 95%, or at least 99% identical to SEQ ID NO:2, an ortholog of FdhA, or a recombinant variant of the ortholog of FdhA. The homolog of Saro_0995 can comprise a protein comprising a sequence at least 80%, at least 85%, at least 90%, at least 95%, or at least 99% identical to SEQ ID NO:4, an ortholog of Saro_0995, or a recombinant variant of the ortholog of Saro_0995. The homolog of Saro_3899 can comprise a sequence at least 80%, at least 85%, at least 90%, at least 95%, or at least 99% identical to SEQ ID NO:6, an ortholog of Saro_3899, or a recombinant variant of the ortholog of Saro_3899.

The recombinant aldehyde dehydrogenase genes of the invention are preferably capable of catalyzing the conversion of dehydrodiconiferyl aldehyde (DC-L) (a guaiacyl aromatic) or a 4-hydroxyphenyl or syringyl analog thereof to dehydrodiconiferyl carboxylic acid (DC-C) (a guaiacyl aromatic) or a 4-hydroxyphenyl or syringyl analog thereof. See, e.g., FIG. 4. The recombinant aldehyde dehydrogenase genes of the invention may also be capable of catalyzing the conversion of phenolic analogs (such as 4-hydroxyphenyl or syringyl analogs) of dehydrodiconiferyl aldehyde (DC-L) (a guaiacyl aromatic) to phenolic analogs (such as 4-hydroxyphenyl or syringyl analogs) of dehydrodiconiferyl carboxylic acid (DC-C) (a guaiacyl aromatic). Exemplary recombinant aldehyde dehydrogenase genes include those encoding FerD of Novosphingobium aromaticivorans (Saro_0797) (SEQ ID NO:8 (exemplary coding sequence is SEQ ID NO:7)) or a homolog thereof, Saro_1104 of Novosphingobium aromaticivorans (SEQ ID NO:10 (exemplary coding sequence is SEQ ID NO:9)) or a homolog thereof, Saro_1197 of Novosphingobium aromaticivorans (SEQ ID NO:12 (exemplary coding sequence is SEQ ID NO:11)) or a homolog thereof, and Saro_2869 of Novosphingobium aromaticivorans (SEQ ID NO:14 (exemplary coding sequence is SEQ ID NO:13)) or a homolog thereof. The homolog of FerD can comprise a protein comprising a sequence at least 80%, at least 85%, at least 90%, at least 95%, or at least 99% identical to SEQ ID NO:8, an ortholog of FerD, or a recombinant variant of the ortholog of FerD. The homolog of Saro_1104 can comprise a protein comprising a sequence at least 80%, at least 85%, at least 90%, at least 95%, or at least 99% identical to SEQ ID NO:10, an ortholog of Saro_1104, or a recombinant variant of the ortholog of Saro_1104. The homolog of Saro_1197 can comprise a protein comprising a sequence at least 80%, at least 85%, at least 90%, at least 95%, or at least 99% identical to SEQ ID NO:12, an ortholog of Saro_1197, or a recombinant variant of the ortholog of Saro_1197. The homolog of Saro_2869 can comprise a protein comprising a sequence at least 80%, at least 85%, at least 90%, at least 95%, or at least 99% identical to SEQ ID NO:14, an ortholog of Saro_2869, or a recombinant variant of the ortholog of Saro_2869. The FerD of Novosphingobium aromaticivorans (Saro_0797) can also convert 5-formyl ferulate (5-FF) to 5-carboxyferulate (5-CF) and vanillin to vanillic acid.

The recombinant γ-formaldehyde lyase genes of the invention are preferably capable of catalyzing the conversion of dehydrodiconiferyl carboxylic acid (DC-C) to dehydrodiconiferyl stilbene carboxylic acid (DC-S-C). See, e.g., FIG. 4. The recombinant γ-formaldehyde lyase genes of the invention may also be capable of catalyzing the conversion of phenolic analogs (such as 4-hydroxyphenyl or syringyl analogs) of dehydrodiconiferyl carboxylic acid (DC-C) (a guaiacyl aromatic) to phenolic analogs (such as 4-hydroxyphenyl or syringyl analogs) of dehydrodiconiferyl stilbene carboxylic acid (DC-S-C) (a guaiacyl aromatic). Exemplary recombinant aldehyde dehydrogenase genes include those encoding PcfL of Novosphingobium aromaticivorans (Saro_0796) (SEQ ID NO:16 (exemplary coding sequence is SEQ ID NO:15)) or a homolog thereof. The homolog of PcfL can comprise a protein comprising a sequence at least 80%, at least 85%, at least 90%, at least 95%, or at least 99% identical to SEQ ID NO:16, an ortholog of PcfL, a recombinant variant of the ortholog of PcfL.

The recombinant lignostilbene dioxygenase genes of the invention are preferably capable of catalyzing the conversion of dehydrodiconiferyl stilbene carboxylic acid (DC-S-C) to 5-formyl ferulate (5-FF) and/or vanillin. See, e.g., FIG. 4. The recombinant lignostilbene dioxygenase genes of the invention may also be capable of catalyzing the conversion of phenolic analogs (such as a 4-hydroxyphenyl analog) of dehydrodiconiferyl stilbene carboxylic acid (DC-S-C) to phenolic analogs (such as a 4-hydroxyphenyl analog) of dehydrodiconiferyl stilbene carboxylic acid (DC-S-C) (a guaiacyl aromatic). Exemplary recombinant lignostilbene dioxygenase genes include those encoding LsdD of Novosphingobium aromaticivorans (Saro_0802) (SEQ ID NO:18 (exemplary coding sequence is SEQ ID NO:17)) or a homolog thereof. The homolog of LsdD can comprise a protein comprising a sequence at least 80%, at least 85%, at least 90%, at least 95%, or at least 99% identical to SEQ ID NO:18, an ortholog of LsdD, a recombinant variant of the ortholog of LsdD.

The recombinant aromatic acid decarboxylase genes of the invention are preferably capable of catalyzing the conversion of 5-carboxyferulate (5-CF) to ferulic acid. See, e.g., FIG. 4. The recombinant aromatic acid decarboxylase genes of the invention may also be capable of catalyzing the conversion of phenolic analogs (such as a 4-hydroxyphenyl analog) of 5-carboxyferulate (5-CF) to phenolic analogs (such as a 4-hydroxyphenyl analog) of ferulic acid. Exemplary recombinant aromatic acid decarboxylase genes include those encoding LigW of Novosphingobium aromaticivorans (Saro_0799) (SEQ ID NO:20 (exemplary coding sequence is SEQ ID NO:19)) or a homolog thereof. The homolog of LigW can comprise a protein comprising a sequence at least 80%, at least 85%, at least 90%, at least 95%, or at least 99% identical to SEQ ID NO:20, an ortholog of LigW, a recombinant variant of the ortholog of LigW.

The recombinant genes of the invention can be configured to be expressed or overexpressed in the microorganism. If a microorganism endogenously comprises a particular gene, the gene may be modified to exchange or optimize promoters, exchange or optimize enhancers, or exchange or optimize any other genetic element to result in increased expression of the gene. Alternatively, one or more additional copies of the gene or coding sequence thereof may be introduced to the cell for enhanced expression of the gene product. If a microorganism does not endogenously comprise a particular gene, the gene or coding sequence thereof may be introduced to the microorganism for heterologous expression of the gene product. The gene or coding sequence may be incorporated into the genome of the microorganism or may be contained on an extra-chromosomal plasmid. The gene or coding sequence may be introduced to the microorganism individually or may be included on an operon. Techniques for genetic manipulation are described in further detail below.

The recombinant microorganisms of the invention may be genetically altered to express or overexpress any of the specific genes or gene products explicitly described herein or homologs thereof. Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Nucleic acid or gene product (amino acid) sequences of any known gene, including the genes or gene products described herein, can be determined by searching any sequence databases known in the art using the gene name or accession number as a search term. Common sequence databases include GenBank (www.ncbi.nlm.nih.gov), ExPASy (expasy.org), KEGG (www.genome.jp), among others. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity (e.g., identity) over 50, 100, 150 or more residues (nucleotides or amino acids) is routinely used to establish homology (e.g., over the full length of the two sequences to be compared). Higher levels of sequence similarity (e.g., identity), e.g., 30%, 35% 40%, 45% 50%, 55%, 60%, 65%, 70%, 75%, 80%, 85%, 90%, 95%, or 99% or more, can also be used to establish homology. Accordingly, homologs of the genes or gene products described herein include genes or gene products having at least about 30%, 35%, 40%, 45%, 50%, 55%, 60%, 65%, 70%, 75%, 80%, 85%, 90%, 95%, or 99% identity to the genes or gene products described herein. Methods for determining sequence similarity percentages (e.g., BLASTP and BLASTN using default parameters) are described herein and are generally available. The homologous proteins should demonstrate comparable activities and, if an enzyme, participate in the same or analogous pathways. Homologs include orthologs and paralogs. “Orthologs” are genes and products thereof in different species that evolved from a common ancestral gene by speciation. Normally, orthologs retain the same or similar function in the course of evolution. Paralogs are genes and products thereof related by duplication within a genome. As used herein, “orthologs” and “paralogs” are included in the term “homologs.”

For sequence comparison and homology determination, one sequence typically acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence based on the designated program parameters. A typical reference sequence of the invention is a nucleic acid or amino acid sequence corresponding to the genes or gene products described herein.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see Current Protocols in Molecular Biology, F. M. Ausubel et al., eds., Current Protocols, a joint venture between Greene Publishing Associates, Inc. and John Wiley & Sons, Inc., (supplemented through 2008)).

One example of an algorithm that is suitable for determining percent sequence identity and sequence similarity for purposes of defining homologs is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001. The above-described techniques are useful in identifying homologous sequences for use in the methods described herein.

The terms “identical” or “percent identity”, in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described above (or other algorithms available to persons of skill) or by visual inspection.

The phrase “substantially identical” in the context of two nucleic acids or polypeptides refers to two or more sequences or subsequences that have at least about 60%, about 65%, about 70%, about 75%, about 80%, about 85%, about 90, about 95%, about 98%, or about 99% or more nucleotide or amino acid residue identity, when compared and aligned for maximum correspondence, as measured using a sequence comparison algorithm or by visual inspection. Such “substantially identical” sequences are typically considered to be “homologous,” without reference to actual ancestry. Preferably, the “substantial identity” exists over a region of the sequences that is at least about 50 residues in length, more preferably over a region of at least about 100 residues, and most preferably, the sequences are substantially identical over at least about 150 residues, at least about 250 residues, or over the full length of the two sequences to be compared.

Derived: When used with reference to a nucleic acid or protein, “derived” means that the nucleic acid or polypeptide is isolated from a described source or is at least 70%, 80%, 90%, 95%, 99%, or more identical to a nucleic acid or polypeptide included in the described source.

Endogenous: As used herein with reference to a nucleic acid molecule, genetic element (e.g., gene, promoter, etc.), or polypeptide in a particular cell, “endogenous” refers to a nucleic acid molecule, genetic element, or polypeptide that is in the cell and was not introduced into the cell or transferred within the genome of the cell using recombinant engineering techniques. For example, an endogenous genetic element is a genetic element that was present in a cell in its particular locus in the genome when the cell was originally isolated from nature.

Exogenous: As used herein with reference to a nucleic acid molecule, genetic element (e.g., gene, promoter, etc.), or polypeptide in a particular cell, “exogenous” refers to any nucleic acid molecule, genetic element, or polypeptide that was introduced into the cell or transferred within the genome of the cell using recombinant engineering techniques. For example, an exogenous genetic element is a genetic element that was not present in its particular locus in the genome when the cell was originally isolated from nature.

Expression: The process by which a gene's coded information is converted into the structures and functions of a cell, such as a protein, transfer RNA, or ribosomal RNA. Expressed genes include those that are transcribed into mRNA and then translated into protein and those that are transcribed into RNA but not translated into protein (for example, transfer and ribosomal RNAs).

Introduce: When used with reference to genetic material, such as a nucleic acid, and a cell, “introduce” refers to the delivery of the genetic material to the cell in a manner such that the genetic material is capable of being expressed within the cell. Introduction of genetic material includes both transformation and transfection. Transformation encompasses techniques by which a nucleic acid molecule can be introduced into cells such as prokaryotic cells or non-animal eukaryotic cells. Transfection encompasses techniques by which a nucleic acid molecule can be introduced into cells such as animal cells. These techniques include but are not limited to introduction of a nucleic acid via conjugation, electroporation, lipofection, infection, and particle gun acceleration.

Isolated: An “isolated” biological component (such as a nucleic acid molecule, polypeptide, or cell) has been substantially separated or purified away from other biological components in which the component naturally occurs, such as other chromosomal and extrachromosomal DNA and RNA and proteins. Nucleic acid molecules and polypeptides that have been “isolated” include nucleic acid molecules and polypeptides purified by standard purification methods. The term also includes nucleic acid molecules and polypeptides prepared by recombinant expression in a cell as well as chemically synthesized nucleic acid molecules and polypeptides. In one example, “isolated” refers to a naturally occurring nucleic acid molecule that is not immediately contiguous with both of the sequences with which it is immediately contiguous (one on the 5′ end and one on the 3′ end) in the naturally-occurring genome of the organism from which it is derived.

Gene: Genes minmally include a promoter operationally linked to a coding sequence, and can include other elements that facilitate or regulate the transcription and/or translation of the coding sequence.

Heterologous: The term “heterologous” refers to an element in an arrangement with another element that does not occur in nature. For example, a gene or protein that is heterologous to a given cell is a gene or protein that does not occur in the cell in nature. A promoter that is heterologous to a given coding sequence is a promoter that is not operably linked to the coding sequence in nature.

Nucleic acid: Encompasses both RNA and DNA molecules including, without limitation, cDNA, genomic DNA, and mRNA. Nucleic acids also include synthetic nucleic acid molecules, such as those that are chemically synthesized or recombinantly produced. The nucleic acid can be double-stranded or single-stranded. Where single-stranded, the nucleic acid molecule can be the sense strand, the antisense strand, or both. In addition, the nucleic acid can be circular or linear.

Operably linked: A first element is operably linked with a second element when the first element is placed in a functional relationship with the second element. For instance, a promoter is operably linked to a coding sequence if the promoter affects the transcription or expression of the coding sequence. A secretion signal sequence is operably linked to a protein (such as an enzyme) when the secretion signal sequence affects secretion of the protein from a cell.

Overexpress: When a gene is caused to be transcribed at an elevated rate compared to the endogenous or basal transcription rate for that gene. In some examples, overexpression additionally includes an elevated rate of translation of the gene compared to the endogenous translation rate for that gene. Methods of testing for overexpression are well known in the art, for example transcribed RNA levels can be assessed using RT-PCR and protein levels can be assessed using SDS-PAGE gel analysis.

Recombinant: A recombinant nucleic acid or polypeptide is one comprising a sequence that is not naturally occurring. A recombinant gene is a gene that comprises a recombinant nucleic acid sequence, is present within a cell in which it does not naturally occur, and/or is present in a different locus (e.g., genetic locus or on an extrachromosomal plasmid) within a particular cell than in a corresponding native cell. A recombinant cell (such as a recombinant microorganism) is one that comprises a recombinant nucleic acid, a recombinant gene, or a recombinant polypeptide. An example of a recombinant gene is a gene that has a coding sequence operably linked to a heterologous promoter.

Recombinant variant: Used with reference to an ortholog, “recombinant variant” refers to a variant of the ortholog that comprises one or more modifications to amino acid sequence of the ortholog. Exemplary modifications include substitutions, deletions, and insertions. The recombinant variant preferably comprises an amino acid sequence at least 95% identical to the amino acid sequence of the ortholog.

Another aspect of the invention is directed to methods of catabolizing a lignin aromatic. The methods can comprise culturing the recombinant microorganism of the invention in a medium comprising the lignin aromatic to thereby catabolize the lignin aromatic.

“Lignin aromatic” as used herein refers to an aromatic present in or derived from lignin. The lignin aromatics can be a monomer, a dimer, an oligomer, or a polymer. The lignin aromatics can comprise syringyl aromatics, guaiacyl aromatics, p-hydroxyphenyl aromatics, or any combinations thereof. Syringyl, guaiacyl, and p-hydroxyphenyl aromatics differ in their degree of methoxilation of the aromatic ring. Syringyl aromatics comprise methoxy groups at the 3 and 5 positions of the aromatic ring. Guaiacyl aromatics comprise a methoxy group on only one of the 3 and 5 positions on the aromatic ring. p-Hydroxyphenyl aromatics are devoid of methoxy groups on either of the 3 and 5 positions of the aromatic ring.

In some versions, the lignin aromatic comprises a β-5 linked lignin aromatic. β-5 linked lignin aromatics include lignin aromatics that comprise at least one β-5 linkage.

In some versions, the lignin aromatic comprises one or more of dehydrodiconiferyl alcohol (DC-A), dehydrodiconiferyl aldehyde (DC-L), dehydrodiconiferyl carboxylic acid (DC-C), dehydrodiconiferyl stilbene carboxylic acid (DC-S-C), 5-formyl ferulate (5-FF), 5-carboxyferulate (5-CF) or a 4-hydroxyphenyl or syringyl analog thereof. The 4-hydroxyphenyl or syringyl analogs of these compounds lack methoxy groups at both of the 3 and 5 positions of the aromatic ring or comprise methoxy groups at both of the 3 and 5 positions of the aromatic ring, respectively.

In some versions, the lignin aromatic can be derived from (and optionally isolated from) and/or provided in the form of depolymerized lignin, such as chemically depolymerized lignin. Methods of depolymerizing lignin are well known in the art. See Pandey et al. 2010 (Pandey M P, Kim C S. Lignin Depolymerization and Conversion: A Review of Thermochemical Methods. Chemical & Engineering Technology, 2010, Vol. 34, Issue 1, pp. 3-145) and Wang et al. 2013 (Wang H, Tucker M, Ji Y. Recent Development in Chemical Depolymerization of Lignin: A Review. Journal of Applied Chemistry, 2013, Volume 2013, Article ID 838645).

The depolymerized lignin can be derived from pretreated lignocellulosic biomass. Methods of pretreating lignocellulosic biomass are well known in the art. See Kumar et al. 2017 (Kumar A K and Sharma S. Recent Updates on Different Methods of Pretreatment of Lignocellulosic Feedstocks: A Review. Bioresour. Bioprocess. (2017) 4:7); Kumar et al. 2009 (Kumar, P.; Barrett, D. M.; Delwiche, M. J.; Stroeve, P., Methods for Pretreatment of lignocellulosic Biomass for Efficient Hydrolysis and Biofuel Production. Industrial & Engineering Chemistry Research 2009, 48, (8), 3713-3729); Wang et al. 2013 (Wang H, Tucker M, Ji Y. Recent Development in Chemical Depolymerization of Lignin: A Review. (2013) Journal of Applied Chemistry. 2013:1-9), and Karlen et al. 2020 (Karlen S D, Fasahati P, Mazaheri M, Serate J, Smith R A, Sirobhushanam S, Chen M, Tymkhin V I, Cass C L, Liu S, Padmakshan D, Xie D, Zhang Y, McGee M A, Russell J D, Coon J J, Kaeppler H F, de Leon N, Maravelias C T, Runge T M, Kaeppler S M, Sedbrook J C, Ralph J. Assessing the viability of recovering hydroxycinnamic acids from lignocellulosic biorefinery alkaline pretreatment waste streams. ChemSusChem. 2020 Jan. 26). Examples include chipping, grinding, milling, steam pretreatment, ammonia fiber expansion (AFEX, also referred to as ammonia fiber explosion), ammonia recycle percolation (ARP), CO₂ explosion, steam explosion, ozonolysis, wet oxidation, acid hydrolysis, dilute-acid hydrolysis, alkaline hydrolysis, organosolv, ionic liquids, gamma-valerolactone, enzymatic pretreatment, biological pretreatment, and pulsed electrical field treatment, among others.

The lignocellulosic biomass can be derived from any source, such as corn cobs, corn stover, cotton seed hairs, grasses, hardwood stems, leaves, newspaper, nut shells, paper, softwood stems, sorghum, switchgrass, waste papers from chemical pulps, wheat straw, wood, woody residues, mixed biomass species such as those produced by native prairie, and other sources. Sources that maintain β-5 bonds in lignin are preferred.

It is noted that the aromatic analogs of the compounds described herein will have modifications to aromatic groups only at positions on the aromatic groups where they are chemically possible. For example, only one of the two aromatic groups in DC-A, DC-L, DC-C, and DC-S-C permit the presence of syringyl analogs due to the β-5 bonds or other bonding at the relevant position on the aromatic ring. Similarly, 5-FF and 5-CF do not permit the presence of syringyl analogs due to the presence of the aldehyde and carboxy groups, respectively, at the relevant position on the aromatic ring. Mixed type β-5 aromatics (e.g., those containing one syringyl type aromatic and one 4-hydroxyphenyl type aromatic) are contemplated as examples of aromatic analogs of the compounds herein.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### Examples

Catabolism of β-5 Linked Aromatics by Novosphingobium aromaticivorans

### Summary

Aromatic compounds are an important source of commodity chemicals traditionally produced from fossil fuels. Aromatics derived from plant lignin can potentially be converted into commodity chemicals through depolymerization followed by microbial funneling of monomers and low molecular weight oligomers. This study investigates the catabolism of the β-5 linked aromatic dimer dehydrodiconiferyl alcohol (DC-A) by the bacterium Novosphingobium aromaticivorans. We used genome-wide screens to identify candidate genes involved in DC-A catabolism. Subsequent in vivo and in vitro analyses of these candidate genes elucidated a catabolic pathway composed of four required gene products and several partially redundant dehydrogenases that convert DC-A to aromatic monomers that can be funneled into the central aromatic metabolic pathway of N. aromaticivorans. Specifically, a newly identified γ-formaldehyde lyase, PcfL, opens the phenylcoumaran ring to form a stilbene and formaldehyde. A lignostilbene dioxygenase, LsdD, then cleaves the stilbene to generate the aromatic monomers vanillin and 5-formylferulate (5-FF). We also show that the aldehyde dehydrogenase FerD oxidizes 5-FF before it is decarboxylated by LigW, yielding ferulic acid. We found that some enzymes involved in the β-5 catabolism pathway can act on multiple substrates and that some steps in the pathway can be mediated by multiple enzymes, providing new insights into the robust flexibility of aromatic catabolism in N. aromaticivorans. A comparative genomic analysis predicted that the newly discovered β-5 aromatic catabolic pathway is common within the order Sphingomonadales.

In the transition to a circular bioeconomy, the plant polymer lignin holds promise as a renewable source of industrially important aromatic chemicals. However, since lignin contains aromatic subunits joined by various chemical linkages, producing single chemical products from this polymer can be challenging. One strategy to overcome this challenge is using microbes to funnel a mixture of lignin-derived aromatics into target chemical products. This approach requires strategies to cleave the major inter-unit linkages of lignin to release monomers for funneling into valuable products. In this study, we report newly discovered aspects of a pathway by which the Novosphingobium aromaticivorans DSM12444 catabolizes aromatics joined by the second most common inter-unit linkage in lignin, the β-5 linkage. This work advances our knowledge of aromatic catabolic pathways, laying the groundwork for future metabolic engineering of this and other microbes for optimized conversion of lignin into products.

### Introduction

Novosphingobium aromaticivorans DSM12444 is an Alphaproteobacterium with properties that make it a potential microbial chassis for lignin valorization. N. aromaticivorans can metabolize a variety of natural and chemically modified aromatic monomers and oligomers and it can co-metabolize aromatic compounds with other carbon sources (13, 14). Additionally, native metabolic pathways enable engineered strains of this bacterium to funnel the products of depolymerized lignin into commodity chemicals such as 2-pyrone-4,6-dicarboxylic acid (PDC) (10, 15), cis-cis-muconic acid (16), and carotenoids (17). This study uses a previously engineered strain of N. aromaticivorans (12444PDC), in which ligI, desC, and desD have been deleted so that it converts S-, G- and H-aromatics into PDC (10), which is a potential platform chemical for industrial valorization (18, 19).

While metabolic pathways by which N. aromaticivorans funnels aromatic monomers into central aromatic metabolism have been characterized (10, 20, 21), less is known about how it catabolizes aromatics joined by the various interunit bonds present in lignin. To date, only the pathways for catabolism of the most abundant interunit bond, the 3-O-4 linkage (22, 23), as well as the R-1 linkage (24) have been elucidated in N. aromaticivorans. Catabolic pathways for aromatic oligomers containing other abundant interunit linkages have been reported in some organisms, but knowledge gaps remain in the pathways used by this bacterium.

This work sought to investigate the ability of N. aromaticivorans to catabolize β-5 (phenylcoumaran) linked aromatics. β-5 linked aromatics represent the second most abundant interunit linkage in lignin, accounting for up to 12% of the total interunit bonds depending on the biomass source (25, 26). The only pathway for the catabolism of β-5 linked aromatics has been proposed in Sphingomonas paucimobilis TMY10009 (27) and characterized in Sphingobium sp. SYK-6 (28-32), while one enzyme with activity on β-5 linked aromatics has been identified in Agrobacterium sp. (33). However, there are reports of significant differences in either the ability to catabolize aromatic compounds or the enzymes involved in the catabolic pathways of members of the order Sphingomonadales (11, 12, 20). Thus, it is important to identify similarities and differences in aromatic catabolism among different bacteria when developing strategies to valorize lignin.

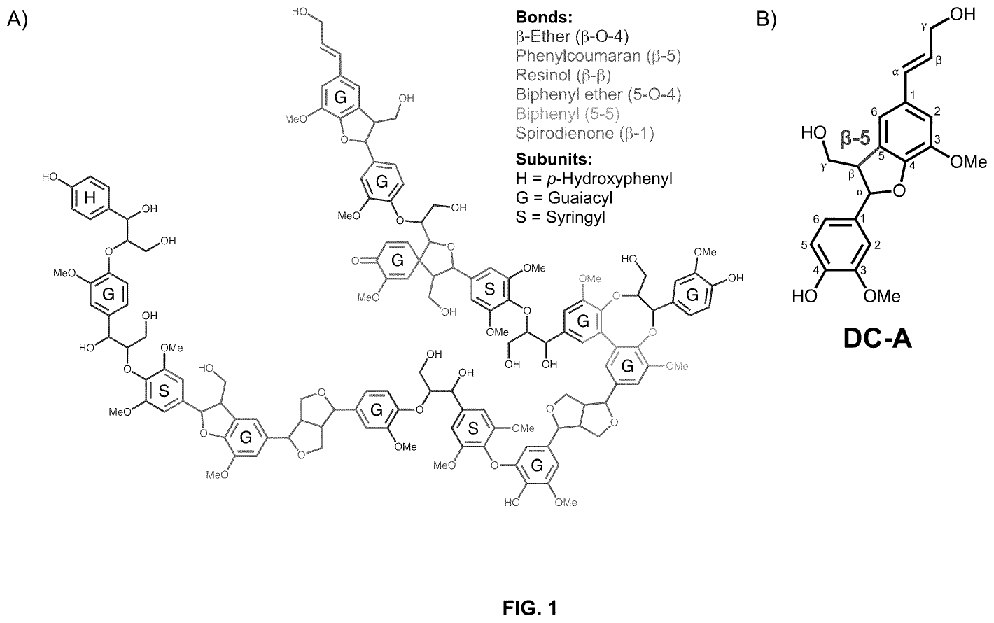

The goal of this study was to determine if and how N. aromaticivorans catabolizes aromatics joined by a β-5 linkage. To do this, we synthesized dehydrodiconiferyl alcohol (DC-A), a dimer composed of two G-aromatic monomers connected by a β-5 interunit linkage (FIG. 1 (B)). We found that N. aromaticivorans can grow on DC-A and funnel it through its central aromatic metabolism. We combined data from two genome-wide screens to identify candidate genes involved in DC-A catabolism, followed by in vivo analysis of defined mutants and in vitro enzyme activity assays to test the roles of candidate genes and proteins in catabolism of this β-5 linked aromatic dimer. This approach defined a pathway for N. aromaticivorans DC-A catabolism that contains enzymes not previously known to be involved in aromatic dimer catabolism. Furthermore, comparative genomic analysis allows us to predict that gene products involved in this catabolic pathway are widespread among the order Sphingomonadales.

### Results

N. aromaticivorans Catabolizes DC-A

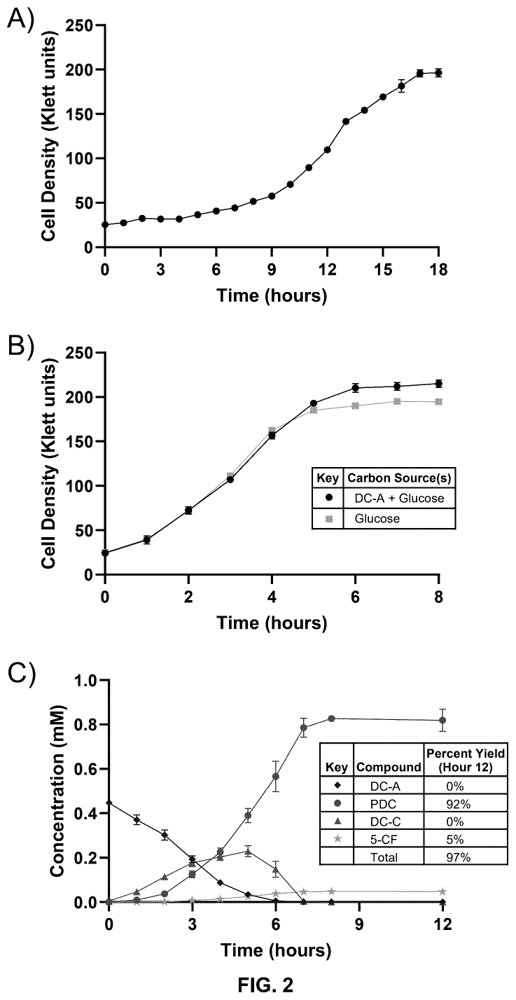

To test whether N. aromaticivorans can catabolize the β-5 linked dimer DC-A, we used a sacB− strain (23) as the wild-type (WT) and grew it in standard mineral base (SMB) minimal medium with DC-A as the sole carbon source. We found that WT N. aromaticivorans grows on DC-A under these conditions (FIG. 2 (A)). This led us to predict that the N. aromaticivorans genome encodes enzymes that cleave the β-5 linkage and metabolize the resulting G-family aromatic monomers.

We then asked whether N. aromaticivorans funnels these monomers through the known central aromatic metabolic pathway. To answer this question, we took advantage of the properties of N. aromaticivorans strain 12444PDC, which contains mutations in the central aromatic catabolic pathway that allow it to produce PDC when grown in the presence of many G-family aromatics (10). However, since G-aromatics are funneled into PDC in this strain, glucose or another alternative carbon source is required for growth. 12444PDC grown in the presence of 1 g/L glucose and 0.4 mM DC-A grows at a similar rate but to a slightly higher density than when it uses glucose as a sole carbon source (FIG. 2 (B)), suggesting that both the glucose and some of the DC-A are used to produce biomass.

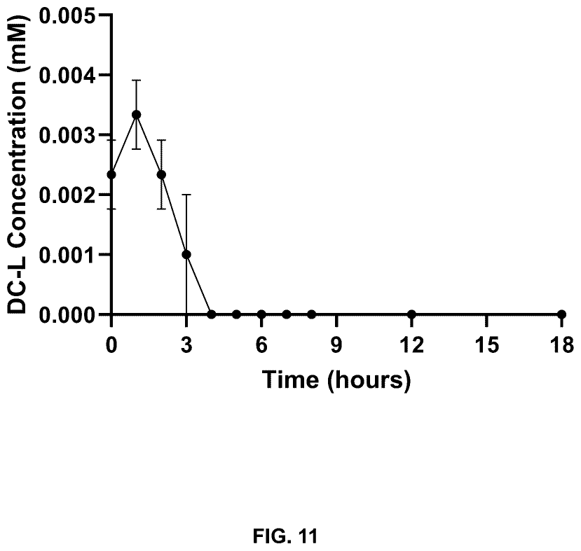

We used high pressure liquid chromatography-mass spectrometry (HPLC-MS) to analyze the culture medium of 12444PDC grown in the presence of DC-A and glucose for consumption of DC-A and accumulation of PDC or other aromatic intermediates (see FIG. 4 for chemical structures). We found that DC-A disappears from the culture medium and PDC accumulates at 92% of the expected yield, assuming that one mole of DC-A would generate two moles of PDC (FIG. 2 (C)). We used HPLC-MS to identify unknown aromatics (Table 1), including 5-carboxyferulate (5-CF), which represents 5% of the aromatics present in the medium at the end of the incubation period (FIG. 2 (C)). Finally, we observed the transient extracellular accumulation of trace amounts of a compound that was subsequently identified as dehydrodiconiferyl aldehyde (DC-L) (FIG. 11) and the accumulation of a compound identified as dehydrodiconiferyl carboxylic acid (DC-C), suggesting the side chain of DC-A is oxidized from an alcohol to an aldehyde and then to a carboxylic acid. These results led us to conclude that N. aromaticivorans possesses the ability to funnel both G-family monomers of the β-5 linked DC-A dimer through its central aromatic metabolic pathway.

TABLE 1
HPLC-MS multiple reaction monitoring conditions and elution
times for the compounds analyzed in this study.

| | | Parent | | | | Elution |
|---|---|---|---|---|---|---|
| | MW | Ion (—) | Transition | Transition | Transition | Time |
| Compound | (g/mol) | m/z | 1 m/z | 2 m/z | 3 m/z | (min)¹ |
| PDC | 184.10 | 183.30 | 111.00 | 139.05 | 95.00 | 1.11 |
| Vanillic Acid | 168.14 | 167.25 | 152.05 | 108.05 | 123.05 | 2.13 |
| Vanillin | 152.15 | 151.15 | 136.00 | 92.00 | 108.00 | 2.41 |
| Ferulic Acid | 194.18 | 193.25 | 134.15 | 178.00 | 149.10 | 2.99 |
| 5-carboxyferulate | 238.19 | 237.10 | 134.10 | 178.10 | 149.15 | 3.36 |
| 5-formylferulate | 222.19 | 221.10 | 206.10 | 134.10 | 162.10 | 3.87 |
| DC-A | 358.38 | 357.15 | 203.10 | 339.15 | 221.20 | 5.25 |
| DC-C | 372.37 | 371.15 | 352.30 | 341.20 | 191.05 | 5.62 |
| DC-L | 356.37 | 355.15 | 337.15 | 219.05 | 190.05 | 5.97 |
| DC-S-C | 342.34 | 341.15 | 267.15 | 326.15 | 282.10 | 6.72 |
| DC-T-C | 682.68 | 681.25 | 339.20 | 637.25 | 324.15 | 6.84 |

¹Elution times can differ when measurements are taken on different days. The elution times listed are those that are found in the HPLC chromatograms shown in this study.

### Genome-Wide Screens Identify Candidate Genes Involved in DC-A Catabolism

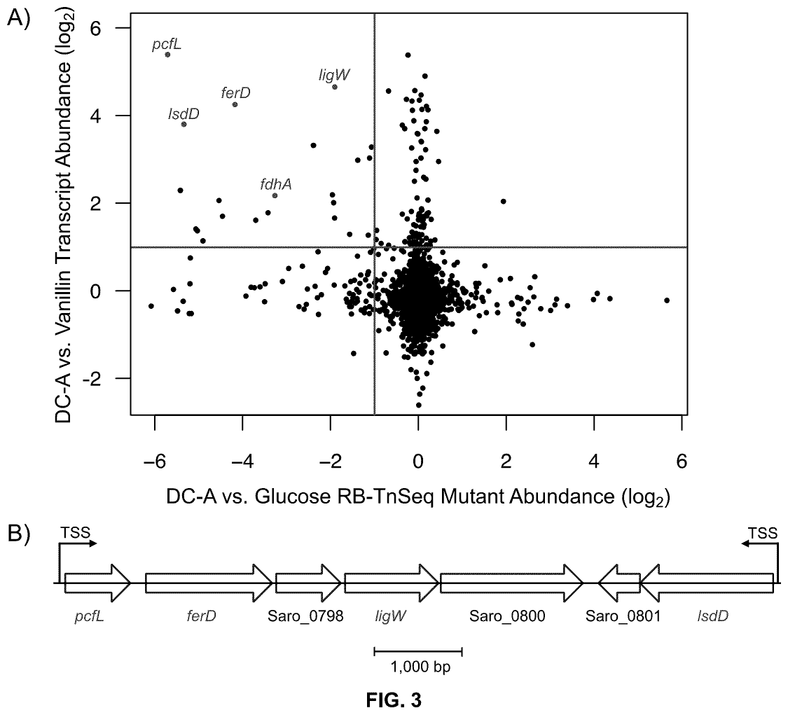

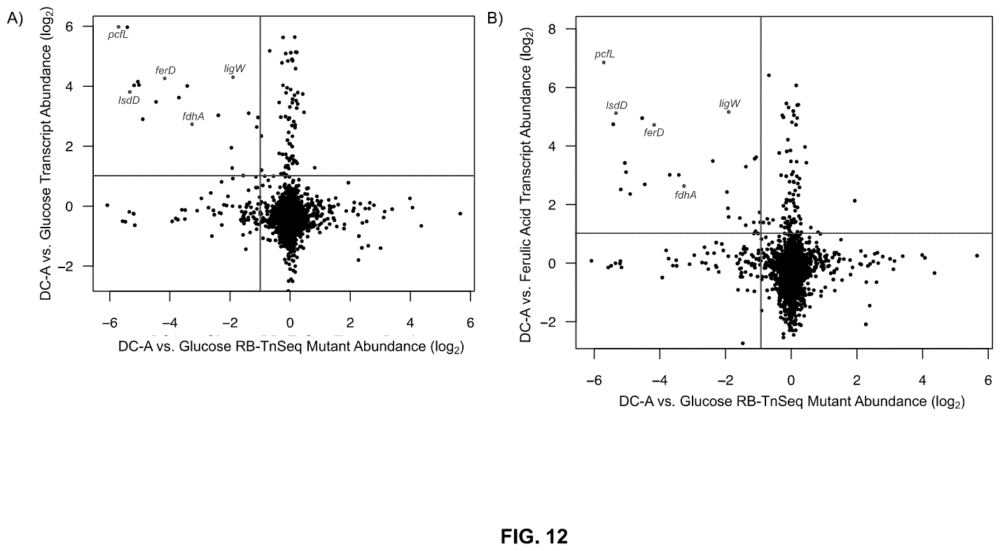

Based on the above results, we sought to identify potential gene products involved in the catabolic pathway for β-5 linked aromatics in N. aromaticivorans. To do this, we integrated data from a pair of genome-wide screens. In one approach, we used RNA-Seq to compare mid-log phase transcript abundances of N. aromaticivorans 12444PDC grown on glucose plus either DC-A or the G-family aromatic monomer vanillin, which was used as a control because we predicted this aromatic monomer to be a product of DC-A catabolism that is further metabolized by known pathways (20, 21). We focused on the 126 transcripts that exhibited a greater than 2-fold, statistically significant increase in abundance when grown in the presence of DC-A compared to cells grown in the presence of vanillin (FIG. 3 (A)). Additionally, we performed RNA-Seq experiments using glucose alone (FIG. 12 (A)) and glucose plus the G-family monomer ferulic acid (FIG. 12 (B)) as controls, which yielded similar results.

In a second genome-wide screen, we used an existing N. aromaticivorans randomly barcoded transposon insertion sequencing (RB-TnSeq) library (21) to identify insertions that led to fitness defects when cells were grown on DC-A as a sole carbon source compared to those grown on glucose alone. In this screen, we found 91 genes for which transposon insertions led to a greater than 2-fold reduced abundance (>50% fitness decrease) after ˜6.5 doublings when using DC-A compared to glucose as sole carbon sources (FIG. 3 (A)).

Of the 91 transposon insertions that met the 2-fold abundance reduction threshold in the RB-TnSeq screen, 22 were also among the candidates from the DC-A vs. vanillin RNA-Seq screen. Subsequent analysis centered on five candidate genes annotated as encoding proteins with predicted enzymatic activity (Table 2). Four of these five genes are found in two adjacent predicted transcription units (FIG. 3 (B)), leading us to hypothesize that the gene products encoded by this region of the genome play a key role in DC-A catabolism.

Below, we present data from in vivo and in vitro experiments used to test this hypothesis. Combined, the data from these experiments identify dehydrogenases that can oxidize the allylic side chain of DC-A in a stepwise manner as well as gene products that open the phenylcoumaran ring in the β-5 interunit linkage of DC-C, cleave the resulting dehydrodiconiferyl stilbene carboxylic acid (DC-S-C), and funnel the monomeric G-family cleavage product 5-formyl ferulate (5-FF) into the N. aromaticivorans central aromatic metabolic pathway (FIG. 4).

TABLE 2
DC-A catabolismcandidate genes identified from RNA-Seq and RB-TnSeq data.

| | | Transcript | Abundance | | Function in DC-A |
|---|---|---|---|---|---|
| Name | Locus Tag | Increase¹ | Reduction² | Annotation | Catabolism |
| pcfL | Saro_0796 | 5.39 | −5.71 | Nuclear transport factor | Phenylcoumaran ring |
| | | | | 2 family protein | opening |
| fdhA | Saro_0874 | 2.17 | −3.27 | S-(hydroxymethyl) | Formaldehyde |
| | | | | glutathione | metabolism; |
| | | | | dehydrogenase | Allylic alcohol oxidation |
| lsdD | Saro_0802 | 3.80 | −5.34 | Carotenoid oxygenase | Stilbene cleavage |
| | | | | family protein | |
| ferD | Saro_0797 | 4.25 | −4.18 | NAD⁺-dependent succinate-semialdehyde | Allylic aldehyde |
| | | | | dehydrogenase | 5-FF oxidation; |
| | | | | | oxidation |
| ligW | Saro_0799 | 4.65 | −1.90 | Amidohydrolase | 5-CF decarboxylation |

¹log₂ comparing transcript abundance when N. aromaticivorans PDC12444 is grown on DC-A plus glucose compared and vanillin plus glucose.
²log₂ comparing abundance of N. aromaticivorans DSM12444 transposon mutants grown on DC-A to those grown on glucose.

### PcfL Opens the DC-A Phenylcoumaran Ring

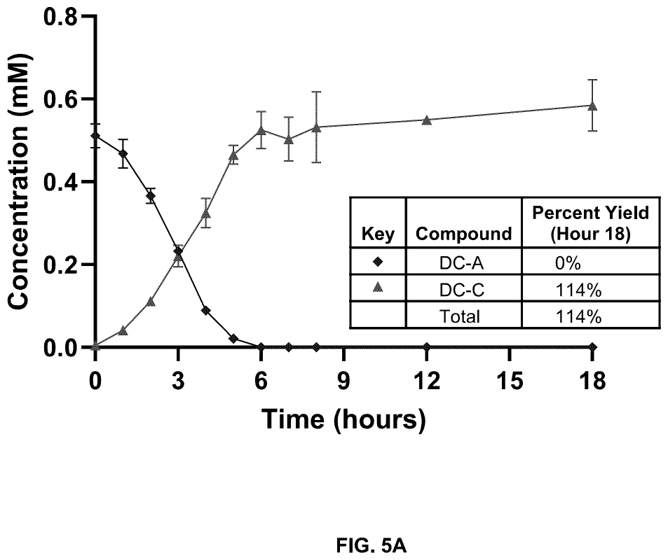

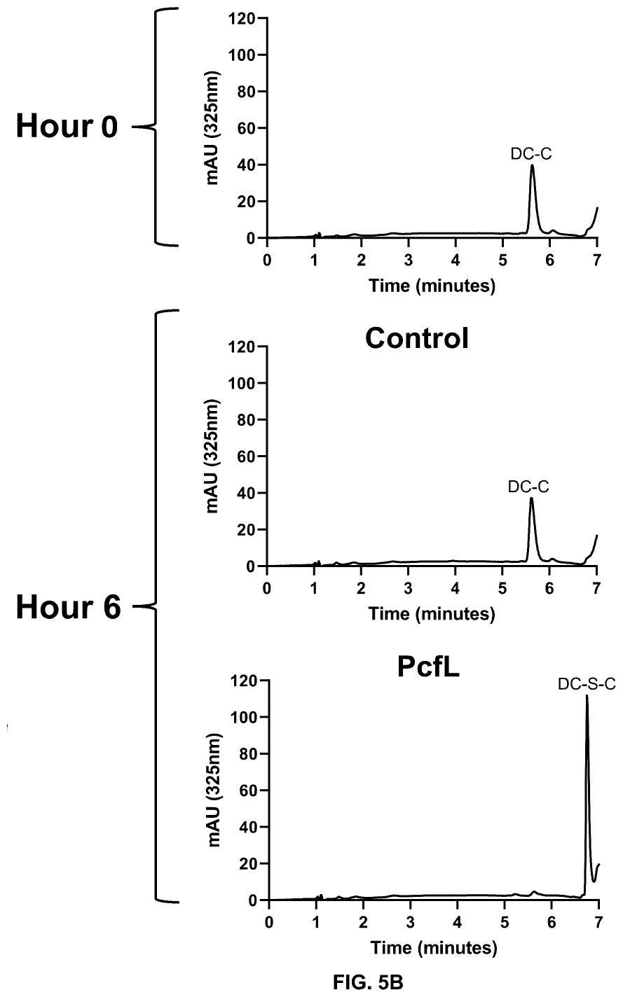

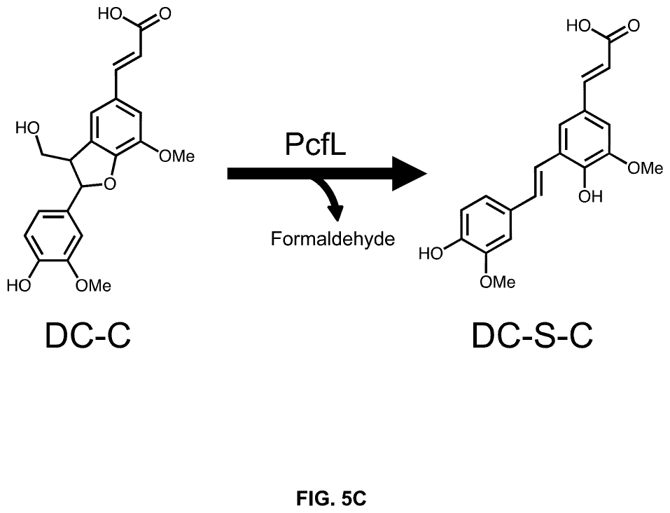

We examined the role of PcfL (Saro_0796) in DC-A catabolism by comparing metabolism of this β-5 linked aromatic dimer in the 12444PDC strain with a ΔpcfL in-frame deletion strain (12444PDCΔpcfL). We found that DC-A disappears from the growth medium of this mutant (FIG. 5A), but unlike the parent strain (FIG. 2 (C)), it does not accumulate PDC. Instead, when grown in the presence of DC-A and glucose, 12444PDCΔpcfL accumulates a compound which we were able to identify as DC-C using a synthetic DC-C standard. In addition, when we quantified DC-C in the 12444PDCΔpcfL medium, we found that one mole of DC-C accumulates per mole of DC-A. Since DC-A catabolism does not progress past DC-C in cells that lack pcfL, we proposed that DC-C is a substrate for this enzyme.

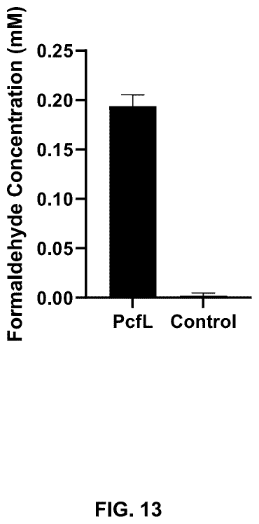

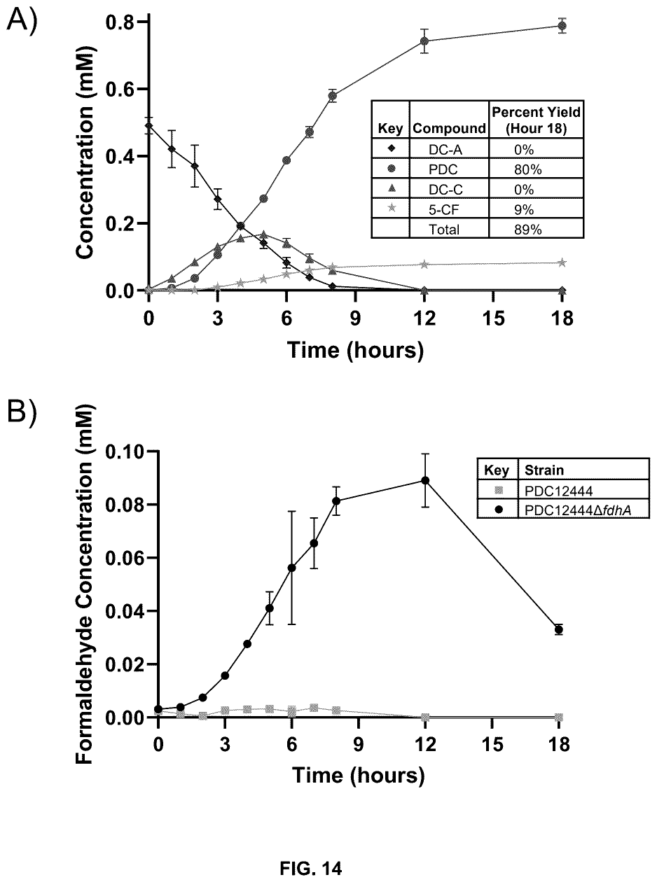

To evaluate this hypothesis, we incubated E. coli cell extracts containing a recombinant PcfL enzyme with pure DC-C. We found that PcfL-containing cell extract converts DC-C to another compound that matches synthetic DC-S-C, while a control extract exhibits no detectable conversion of DC-C under the same conditions (FIG. 5B). Based on these data and the 44% amino acid identity between PcfL and the γ-formaldehyde lyase LdpA that contributes to 3-1 linked aromatic catabolism in N. aromaticivorans (24, 37), we proposed that PcfL removes formaldehyde from DC-C to form the stilbene DC-S-C. We further predicted that the formaldehyde released during this reaction is oxidized by the putative glutathione-dependent dehydrogenase Saro_0874, which we named FdhA (formaldehyde dehydrogenase A), based on homology with an enzyme found in Rhodobacter sphaeroides (38, 39). Upon testing these hypotheses, we found that PcfL produces formaldehyde from DC-C in vitro (FIG. 13) and that a 12444PDCΔfdhA mutant accumulates more extracellular formaldehyde than the parent strain when grown in the presence of DC-A and glucose (FIG. 14). In sum, our data indicate that PcfL is a newly identified γ-formaldehyde lyase that deformylates DC-C, yielding DC-S-C and formaldehyde (FIG. 5C). Based on these results, we named this gene product PcfL to denote its activity as a phenylcoumaran γ-formaldehyde lyase.

LsdD Cleaves DC-S-C into Two Aromatic Monomers

Our results suggest that N. aromaticivorans contains one or more gene products that use the stilbene DC-S-C as a substrate. LsdD (Saro_0802) is a candidate for cleavage of DC-S-C since this gene product shares 80% amino acid identity with the Sphingobium sp. SYK-6 enzyme LsdD, which has been reported to convert DC-S-C into vanillin and 5-FF (30). Furthermore, N. aromaticivorans LsdD (named NOV1 in other work) has been shown to be an iron-dependent dioxygenase that cleaves stilbenes such as resveratrol in vitro (40, 41).

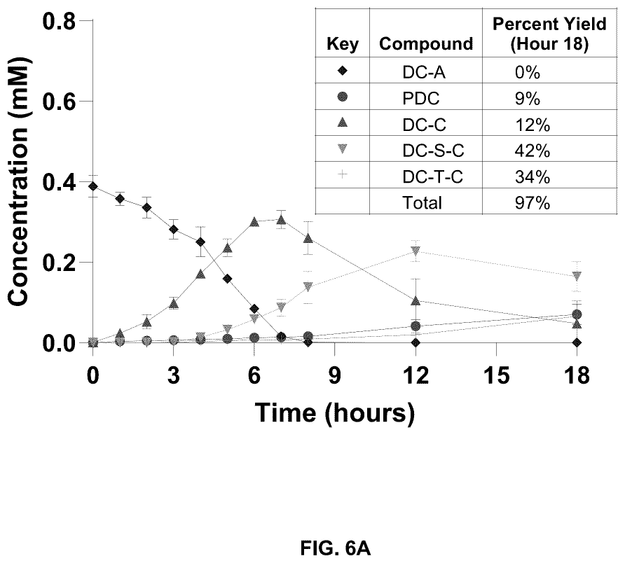

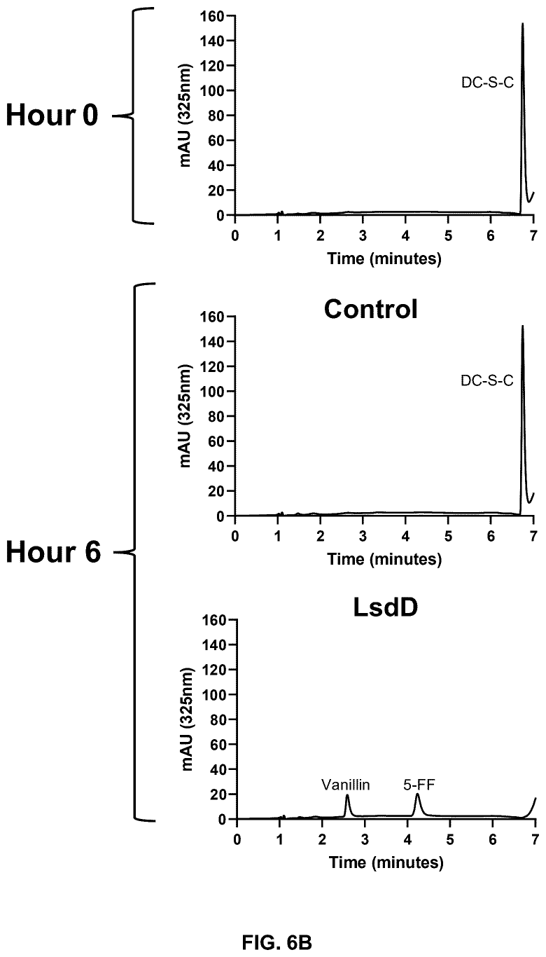

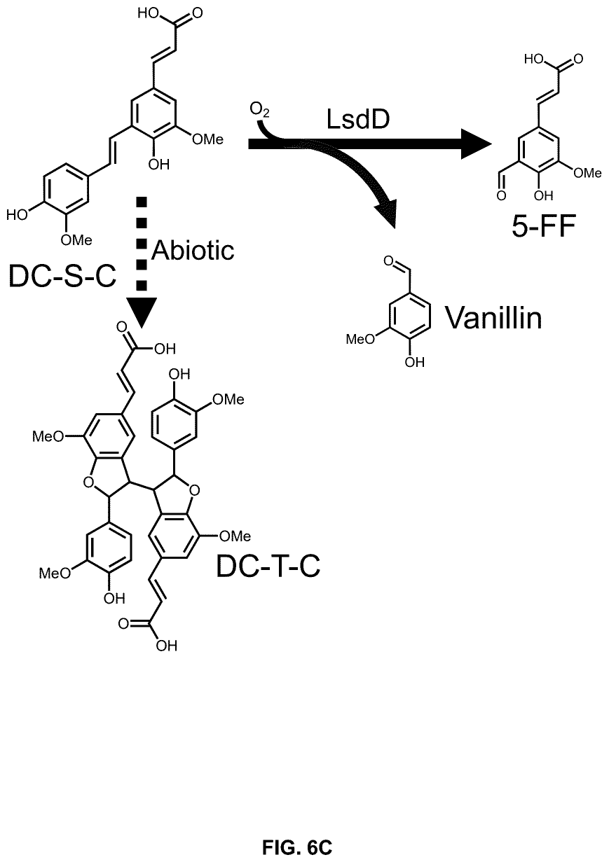

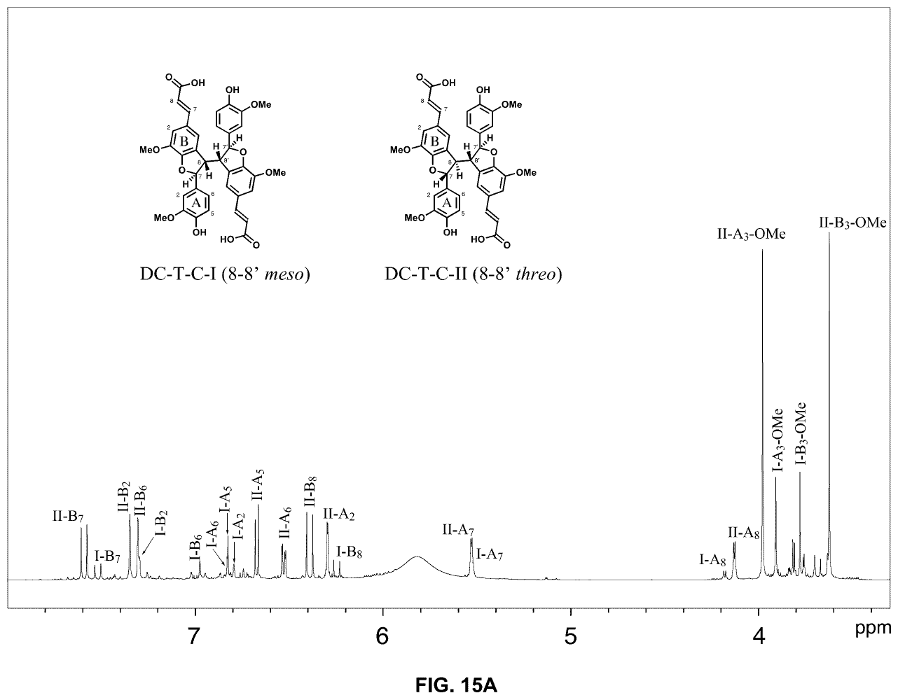

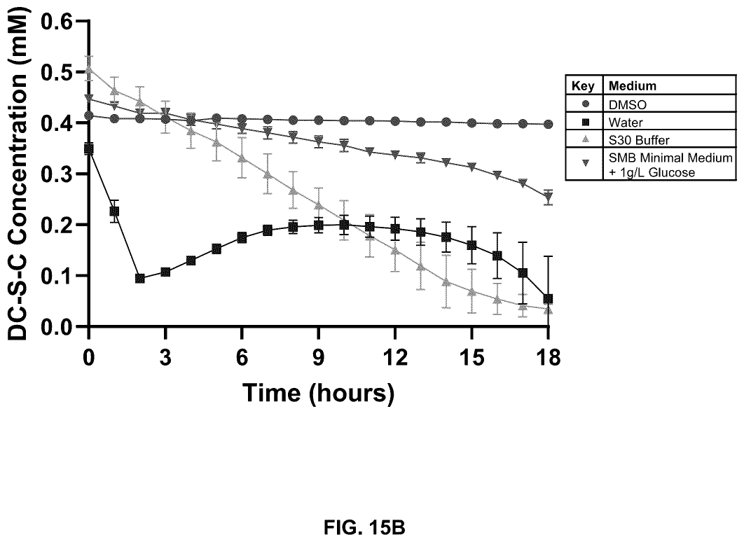

As predicted by this hypothesis, we found that 12444PDCΔlsdD grown in the presence of DC-A and glucose accumulates DC-S-C in the medium (FIG. 6A). This strain also accumulates more DC-C than the parent strain (FIG. 2 (B)) before it is metabolized to DC-S-C, with a detectable amount of DC-C still present in the medium after the 18-hour incubation. In addition, HPLC-MS analysis of extracellular compounds in the 12444PDCΔlsdD strain indicated the presence of another unknown aromatic compound in the medium. In control experiments, we found that DC-S-C is subject to abiotic homodimerization to form the dehydroconiferyl tetramer carboxylic acid DC-T-C when incubated in SMB minimal medium (FIG. 15 (A,B)). At the end of the incubation, 76% of the extracellular aromatics produced from DC-A by 12444PDCΔlsdD are found in the sum of DC-S-C and DC-T-C, while only 9% are converted into PDC. We propose that the low amount of PDC excreted by this strain is derived from the activity of one or more enzymes besides LsdD in cleaving DC-S-C (see Discussion).

We tested the predicted activity of LsdD by incubating E. coli cell extracts containing a recombinant LsdD enzyme with synthetic DC-S-C. When incubated with DC-S-C in the absence of any cofactors, LsdD converts this substrate to 5-FF and vanillin (FIG. 6B). Therefore, we concluded that LsdD cleaves the β-5 linked stilbene DC-S-C into two G-family monomers (FIG. 6C) that can then be funneled into the central pathway for aromatic metabolism.

### FerD and LigW Convert 5-FF to Ferulic Acid

Our data indicate that the two monomeric products of DC-A catabolism are the G-aromatic monomers vanillin and 5-FF. In N. aromaticivorans, vanillin is known to be oxidized to vanillic acid by LigV before entering central G-aromatic metabolism (21). However, the enzymes that metabolize 5-FF have not been identified in this organism. Based on the data from our genome-wide screens, we hypothesized that the putative pyridine nucleotide-dependent ALDH FerD (Saro_0797) oxidizes 5-FF to 5-CF, which is then decarboxylated by LigW (Saro_0799) to form ferulic acid. Ferulic acid is known to be converted into vanillin via a previously described pathway in N. aromaticivorans (21).

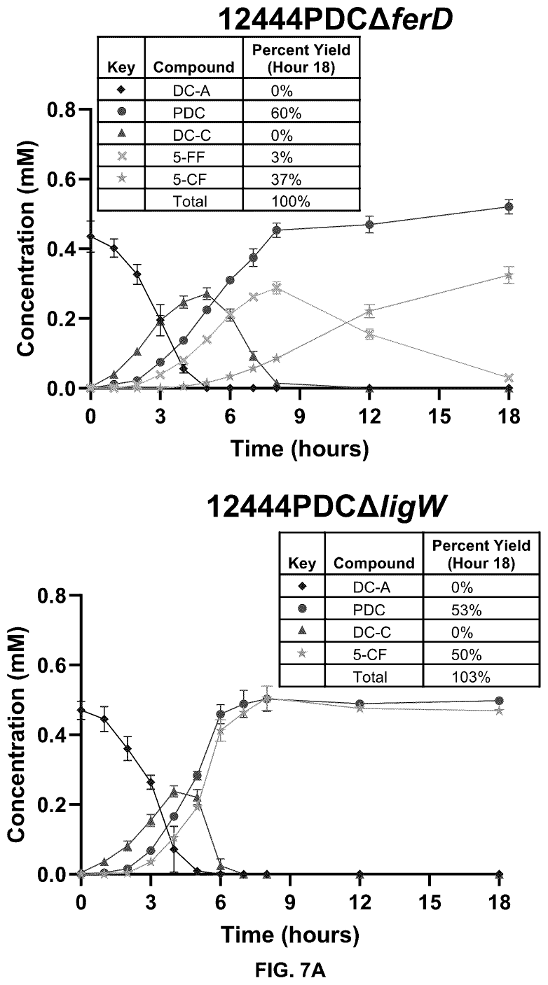

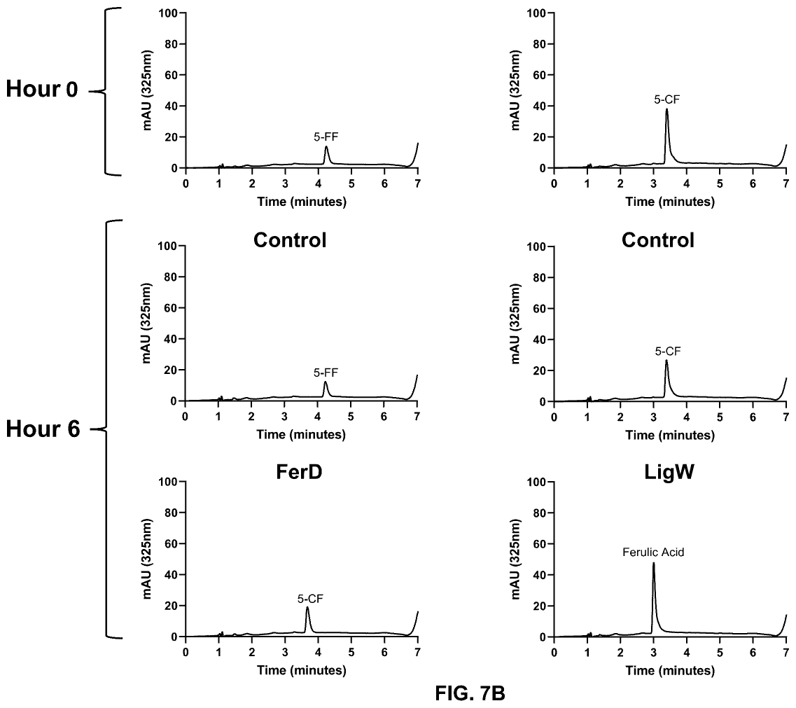

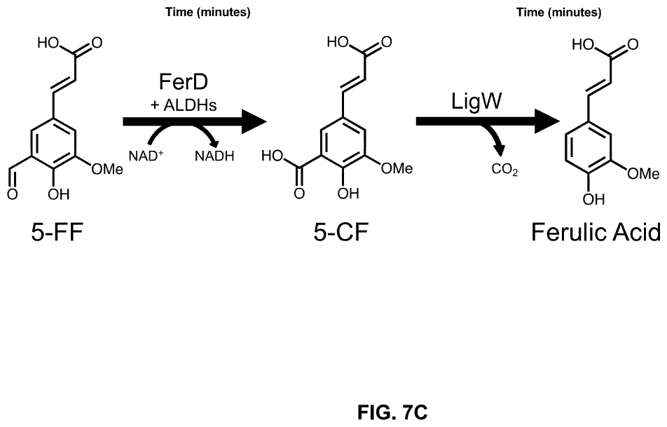

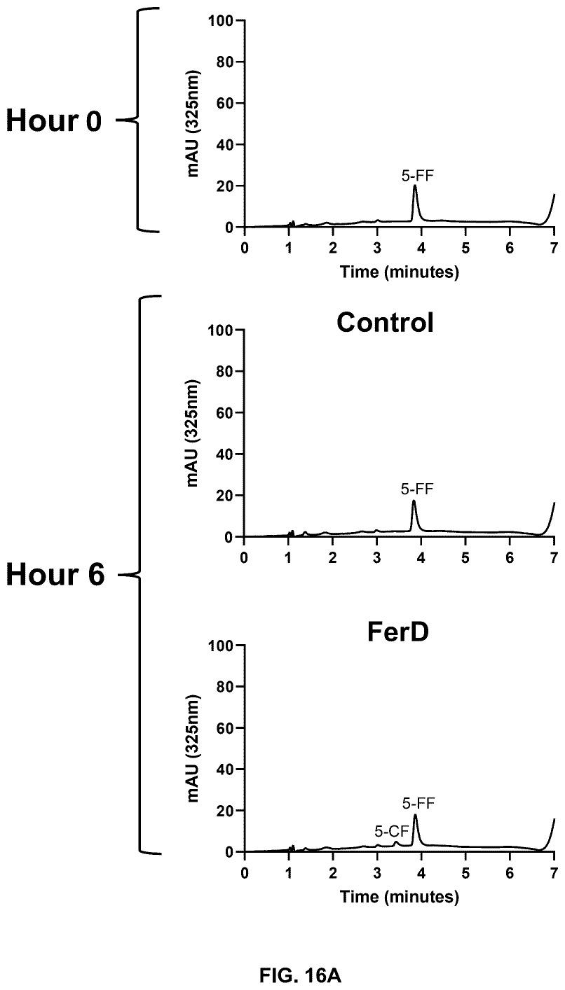

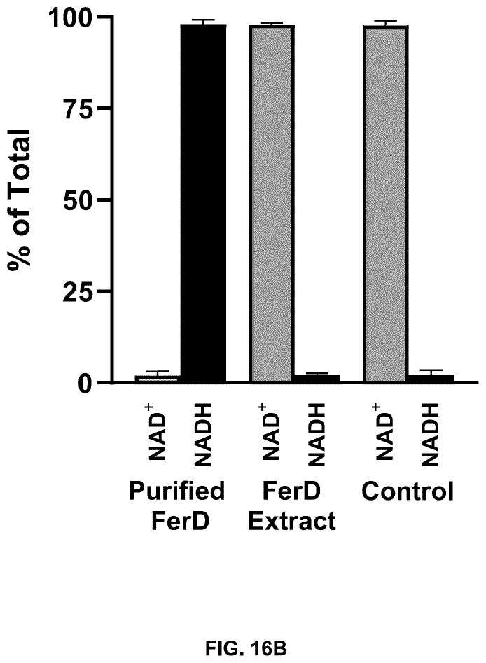

Since the conversion of 5-FF to 5-CF occurs after DC-S-C cleavage, we predicted that growing 12444PDCΔferD in the presence of DC-A and glucose would result in the accumulation of one mole of both 5-FF and PDC per mole of DC-A. We found that 12444PDCΔferD cells transiently accumulate 5-FF in the medium. However, at later time points, as the concentration of 5-FF decreases, the concentration of 5-CF increases. 5-CF can then be funneled into PDC production, leading to the accumulation of 1.17 moles of PDC per mole of DC-A by the end of the incubation (FIG. 7A). To explain these results, we hypothesize that one or more other N. aromaticivorans dehydrogenases can oxidize 5-FF to 5-CF, albeit at a slower rate than FerD. Additionally, E. coli cell extract containing recombinant FerD converts 5-FF into 5-CF (FIG. 7B). As expected, FerD-containing cell extract requires NAD⁺ to convert 5-FF to 5-CF (FIG. 16A) and a purified recombinant FerD protein reduces NAD⁺ to NADH during this reaction (FIG. 16B). From these data, we propose that the NAD⁺-dependent dehydrogenase FerD is the major gene product responsible for 5-FF to 5-CF conversion (FIG. 7C) when cells are grown on DC-A, but that other yet uncharacterized enzymes can also catalyze this reaction.

We investigated the predicted role of LigW in decarboxylation of 5-CF to ferulic acid by growing a 12444PDCΔligW strain in medium containing DC-A and glucose. Under these conditions, we found that cells lacking ligW accumulate ˜1 mole of both PDC and 5-CF per mole of DC-A (FIG. 7A), suggesting that this gene product is responsible for decarboxylation of 5-CF. As predicted, we found that E. coli cell extracts expressing recombinant LigW are able to convert 5-CF into ferulic acid in vitro (FIG. 7B). We therefore concluded that LigW decarboxylates 5-CF in N. aromaticivorans (FIG. 7C).

### Multiple Dehydrogenases can Oxidize the DC-A Allylic Alcohol Side Chain

Given the predicted intermediates of DC-A catabolism (FIG. 4), we hypothesized that N. aromaticivorans contains enzymes that oxidize the allylic alcohol to an aldehyde and then to a carboxylic acid. The only proteins annotated as either alcohol dehydrogenases (ADH) or aldehyde dehydrogenases (ALDH) that were identified as candidates in our genome-wide screens were FdhA and FerD, respectively. However, in the 12444PDCΔferD and 12444PDCΔfdhA strains, the DC-A allylic side chain was still oxidized to a carboxylic acid (FIG. 7A, FIG. 14 (A)). Based on these findings, we hypothesized that N. aromaticivorans contains multiple partially redundant ADHs and ALDHs that convert DC-A to DC-L and DC-L to DC-C.

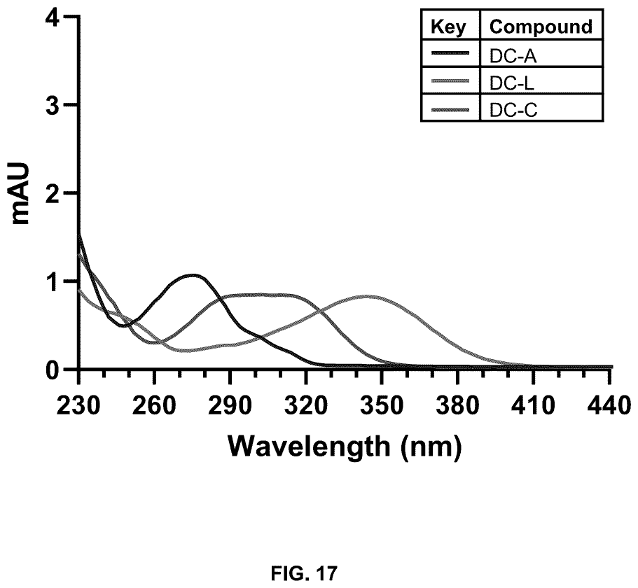

We tested this hypothesis by analyzing the activity of 8 putative ADHs and 9 putative ALDHs for which transcripts represented >2% of the total RNA coding for ADHs or ALDHs when N. aromaticivorans is grown in the presence of DC-A (Table 3). We performed enzyme assays to determine the activity of these gene products by expressing recombinant versions of the proteins in E. coli and incubating cell extracts normalized to the same protein concentration with either DC-A or DC-L with and without NAD⁺ (or PQQ for Saro_2870). We used differences in absorption spectra (FIG. 17) to monitor conversion from DC-A to DC-L and DC-L to DC-C. Control experiments show that none of the cell extracts containing recombinant ADHs or ALDHs were active on these substrates in the absence of NAD⁺.

TABLE 3
Candidate ADHs and ALDHs identified from RNA-Seq data.

| Name/ | Enzyme | Percent of Total ADH | Activity on DC-A |
|---|---|---|---|
| Locus Tag | Class | or ALDH Transcripts¹ | or DC-L |
| FdhA | ADH | 46.65% | Yes |
| Saro_0995 | ADH | 2.16% | Yes |
| Saro_1431 | ADH | 2.95% | No |
| Saro_1476 | ADH | 2.38% | No |
| Saro_2795 | ADH | 2.17% | No |
| Saro_2870 | ADH | 30.89% | No |
| Saro_3899 | ADH | 3.41% | Yes |
| Saro_3463 | ADH | 3.84% | No |
| Saro_0060 | ALDH | 2.36% | No |
| FerD | ALDH | 7.43% | Yes |
| Saro_1104 | ALDH | 16.02% | Yes |
| Saro_1197 | ALDH | 12.16% | Yes |
| Saro_1410 | ALDH | 10.16% | No |
| LigV | ALDH | 2.04% | No |
| Saro_1967 | ALDH | 22.20% | No |
| Saro_2869 | ALDH | 14.74% | Yes |
| Saro_3848 | ALDH | 4.76% | No |

¹Percent of total putative ADH or ALDH transcripts when N. aromaticivorans 12444PDC is grown in the presence of DC-A.

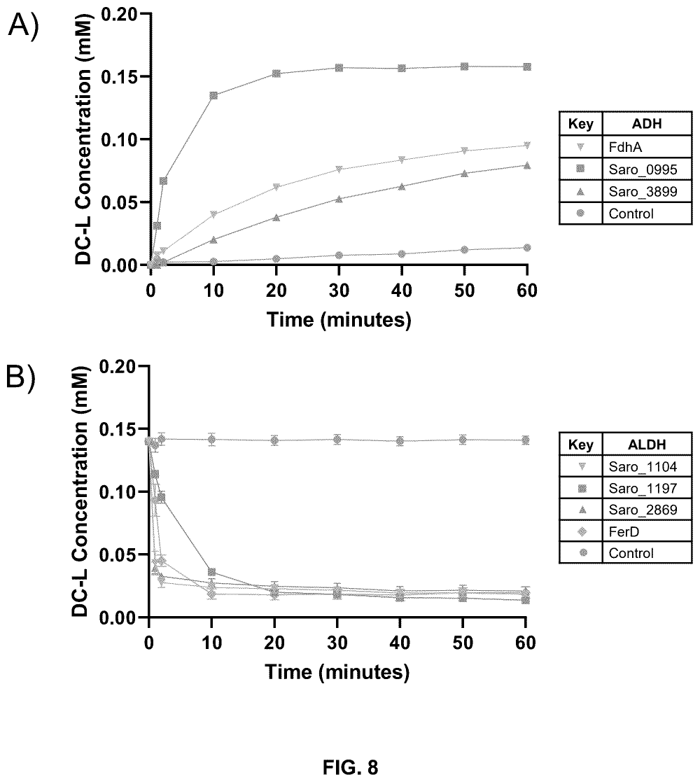

We found that the putative ADHs FdhA, Saro_0995, and Saro_3899 convert DC-A to DC-L in vitro, with Saro_0995 exhibiting the highest activity under our assay conditions (FIG. 8 (A)). There was some conversion of DC-A to DC-L when a control E. coli extract was incubated with DC-A, suggesting that one or more native E. coli enzymes have limited activity on DC-A. However, the conversion of DC-A to DC-L was much faster when using extracts prepared from cells expressing the ADHs listed above.

Using the same approach, we found that the cell extracts containing recombinant versions of the putative ALDHs FerD, Saro_1104, Saro_1197, and Saro_2869 are able to convert DC-L to DC-C in vitro (FIG. 8 (B)). The similar activity of extracts containing these ALDHs on DC-L suggests that they could each make a significant contribution to the metabolism of DC-L in vivo. Combined, the results of these experiments predict that multiple N. aromaticivorans enzymes can oxidize the DC-A allylic alcohol side chain to an aldehyde and then to a carboxylic acid.

### Reconstructing the DC-A Catabolic Pathway In Vitro

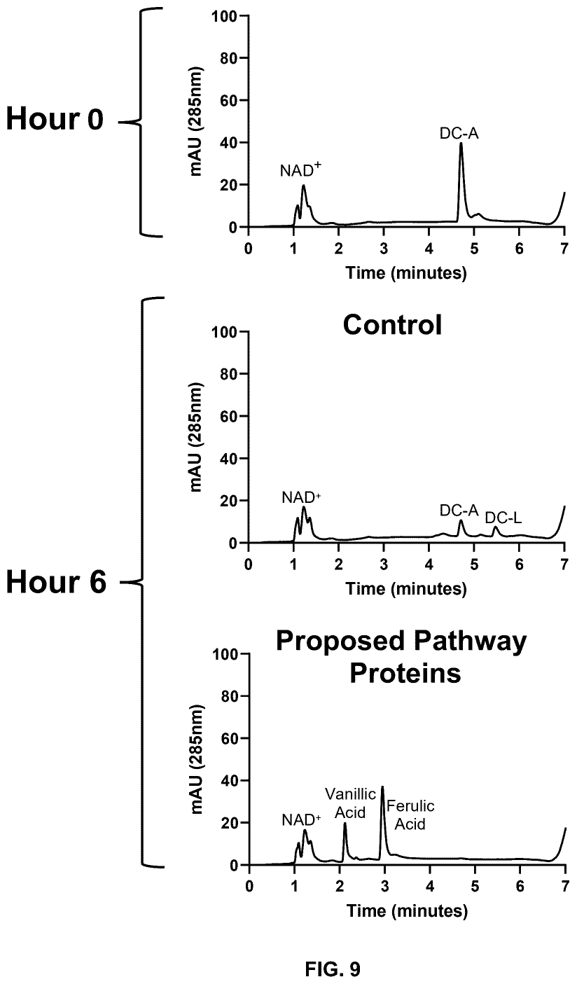

As an independent test of whether the enzymes described above are sufficient for the catabolism of DC-A to G-family aromatic monomers, we sought to reconstruct the entire N. aromaticivorans DC-A catabolic pathway in vitro. Based on the above results, we predicted that a mixture of cell extracts containing NAD⁺, the γ-formaldehyde lyase PcfL, the stilbene cleaving dioxygenase LsdD, the ALDH FerD, the decarboxylase LigW, and the ADH Saro_0995 would be able to convert DC-A to G-family aromatics. After incubating DC-A with these five cell extracts and NAD⁺, we observed complete conversion of DC-A to ferulic and vanillic acid (FIG. 9). When incubated with a control E. coli cell extract containing none of these N. aromaticivorans enzymes, ferulic acid and vanillic acid do not accumulate. However, DC-A is slowly converted to DC-L by the control extract, resulting in a mixture of DC-A and DC-L, in agreement with observations that some native E. coli enzymes have limited activity on DC-A (FIG. 8A). Overall, this experiment confirms that the N. aromaticivorans enzymes we identified are sufficient for the catabolism of DC-A to aromatic monomers that are funneled through known pathways into N. aromaticivorans central aromatic metabolism.

### Discussion

Aromatic compounds are an important source of industrial products and there is increasing interest in renewable sources of these compounds. The abundant plant polymer lignin is a potential source of aromatics that could be used in the production of commodity chemicals. To valorize lignin, the various interunit linkages between aromatic subunits of this polymer must be cleaved and the resulting mixture of monomers funneled into products (9, 10, 12). Recently, progress has been made in the biological funneling of aromatics into valuable chemicals using the Alphaproteobacterium N. aromaticivorans (15). In this study, we found that N. aromaticivorans contains enzymes capable of catabolizing aromatic dimers with β-5 linkages, which is the second most abundant interunit linkage in lignin (25, 26).

Specifically, we showed that N. aromaticivorans can grow on the model β-5 linked G-family aromatic dimer DC-A and that the engineered 12444PDC strain funnels both of its aromatic monomers into PDC production. By combining genomic, genetic, and biochemical assays, we identified gene products that are necessary and sufficient for catabolism of DC-A. Based on these studies, we proposed a catabolic pathway for conversion of DC-A to intermediates in the known N. aromaticivorans central aromatic metabolic pathway.

### Oxidation of the DC-A Allylic Side Chain

We identified enzymes that oxidize the allylic alcohol side chain of DC-A to an aldehyde and the aldehyde to a carboxylic acid. Our data show that three N. aromaticivorans pyridine nucleotide-dependent ADHs (FdhA, Saro_0995, and Saro_3899) can oxidize the allylic alcohol side chain of DC-A, producing the aldehyde DC-L. We also identified four pyridine nucleotide-dependent ALDHs (FerD, Saro_1104, Saro_1197, and Saro_2869) that can oxidize the aldehyde side chain of DC-L to generate the carboxylic acid DC-C. These findings are consistent with RNA-Seq and RB-TnSeq data that indicate increased transcript abundance for multiple ADHs and ALDHs but small or no fitness defects when these dehydrogenases are mutated, suggesting that oxidization of the allylic alcohol side chain of DC-A could be performed by multiple ADHs and ALDHs in vivo (FIG. 3A). Additional biochemical and genetic analyses would be needed to quantify the activity of each ADH and ALDH enzyme on DC-A or DC-L and their relative contribution to catabolism of these and other β-5 linked aromatics in vivo.

### Cleavage of the β-5 Linkage

We found that the phenylcoumaran DC-C is converted to the stilbene DC-S-C and formaldehyde by the newly identified γ-formaldehyde lyase PcfL. This strategy for catabolism of a phenylcoumaran by N. aromaticivorans diverges from the one reported in another aromatic metabolizing member of the order Sphingomonadales, Sphingobium sp. SYK-6 (28, 29). In this bacterium, a pair of enantiospecific oxidoreductases, PhcC and PhcD, as well as other partially redundant dehydrogenases, were shown to sequentially oxidize the phenylcoumaran alcohol to an aldehyde and then a carboxylic acid (28). Next, a pair of enantiospecific decarboxylases, PhcF and PhcG, decarboxylate and open the phenylcoumaran ring on DC-C to produce DC-S-C and CO₂ (29). By comparison, the N. aromaticivorans pathway for generating a stilbene from DC-C requires only a single enzyme as PcfL opens the phenylcoumaran ring and releases formaldehyde in a single step. In addition, our finding that recombinant PcfL can completely convert DC-C into DC-S-C indicates that this enzyme is agnostic to the enantiomeric state of its substrate. Additionally, an Agrobacterium sp. enzyme catalyzes a similar reaction in which it converts a phenylcoumaran to a stilbene, but this enzyme is a glutathione-dependent LigE family enzyme rather than a γ-formaldehyde lyase like PclF.

To our knowledge, the only homolog of PcfL that has been characterized is LdpA, which is another N. aromaticivorans gene product that converts a dimeric aromatic substrate into a stilbene and releases formaldehyde (24, 37). While we found that PcfL has activity with a phenylcoumaran substrate, LdpA acts on a diarylpropane dimer which is a reported intermediate in the N. aromaticivorans β-1 linked aromatic catabolic pathway (24). Since PcfL shares eight of the eleven active site residues of LdpA, future work should test if and how these amino acid differences contribute to the substrate preferences of these two enzymes.

Once DC-S-C forms, our data show this aromatic dimer is cleaved to form 5-FF and vanillin by the lignostilbene dioxygenase LsdD, a homolog of an enzyme previously reported in Sphingobium sp. SYK-6 (30). Cleavage of this β-5 linked stilbene by N. aromaticivorans mirrors the process in 3-1 aromatic dimer metabolism, in which the stilbene produced by LdpA is then cleaved by the dioxygenase NOV2. This combination of a γ-formaldehyde lyase followed by a lignostilbene dioxygenase is a newly described strategy for breaking both β-5 and 3-1 interunit linkages in lignin.

Funneling of Monomers into Central Aromatic Metabolism

Once the β-5 linked dimer DC-A is cleaved into monomeric products, vanillin and 5-FF are funneled into the N. aromaticivorans central G-aromatic metabolic pathway and can be converted into PDC. While vanillin is metabolized through a known pathway (21), our experiments identified enzymes involved in the conversion of 5-FF to 5-CF and then to ferulic acid. We found that 5-FF is oxidized to 5-CF by FerD with minor contributions from one or more uncharacterized ALDHs. We also found that LigW decarboxylates 5-CF to ferulic acid, which is metabolized to vanillin through a known pathway (21). A recently published analysis of 5-FF metabolism in Sphingobium sp. SYK-6 reports the same functions for FerD and LigW (31). N. aromaticivorans LigW has previously been shown to decarboxylate 5-carboxyvanillate (5-CV) (42), which contains a simple carboxylic acid in place of the allylic acid side chain of 5-CF. Thus, it appears that N. aromaticivorans LigW is a relatively broad specificity manganese-dependent aromatic decarboxylase that can function in the metabolism of both the β-5 linked aromatic catabolic pathway intermediate 5-CF and the predicted 5-5 linked aromatic catabolic pathway intermediate 5-CV (43).

### Redundant Enzymes in Catabolism of β-5 Linked Aromatics

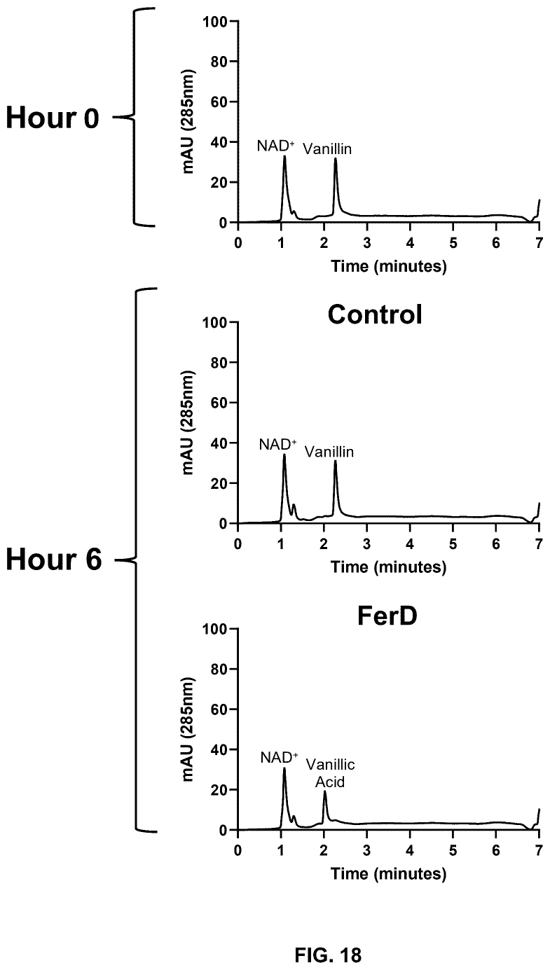

N. aromaticivorans is known to contain several enzymes with multiple functions in aromatic metabolism (20, 44), so it is not surprising for us to find that LigW is not the only enzyme in this pathway with activity on multiple aromatics. We also showed that the dehydrogenases FerD and FdhA display activity on multiple intermediates in the DC-A catabolic pathway. While FdhA is active in conversion of DC-A to DC-L and in the catabolism of formaldehyde, FerD is a promiscuous ALDH that plays a crucial role in the oxidation of 5-FF to 5-CF but is also able to oxidize both DC-L to DC-C and vanillin to vanillic acid (FIG. 18).

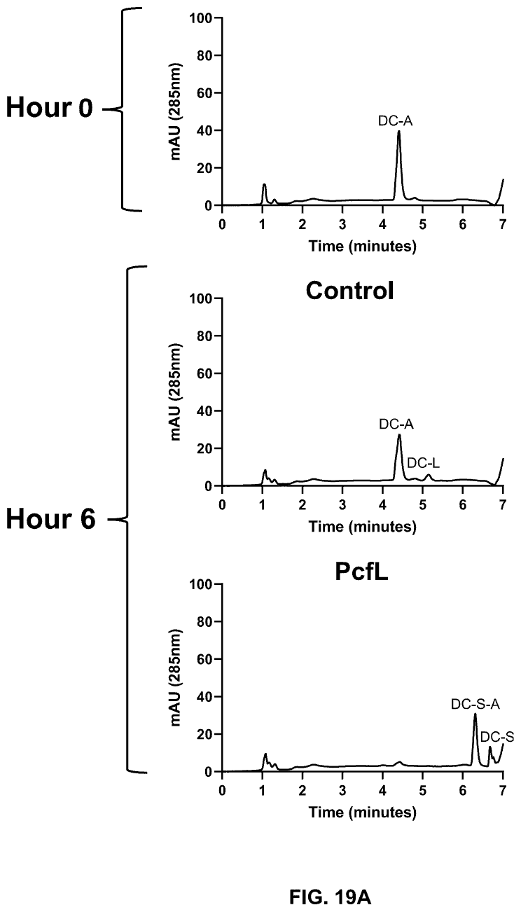

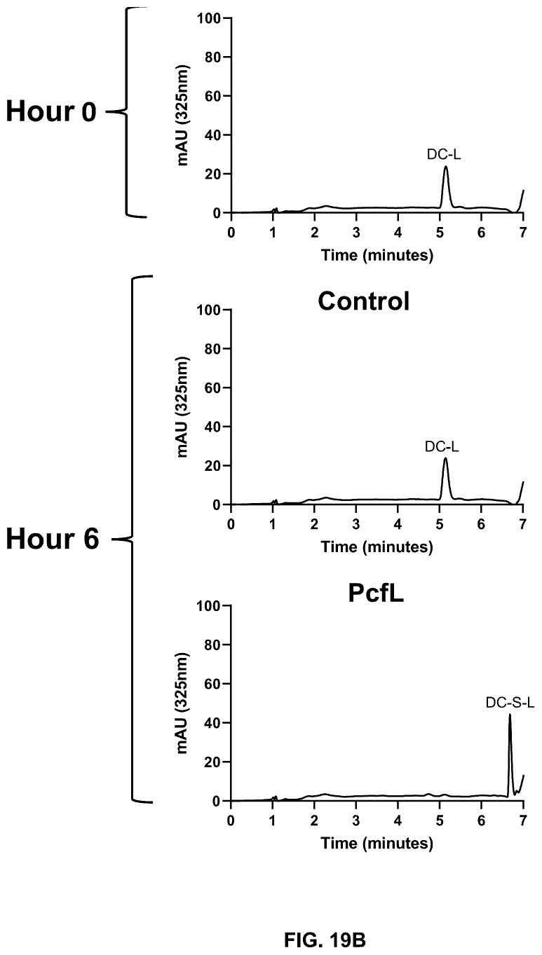

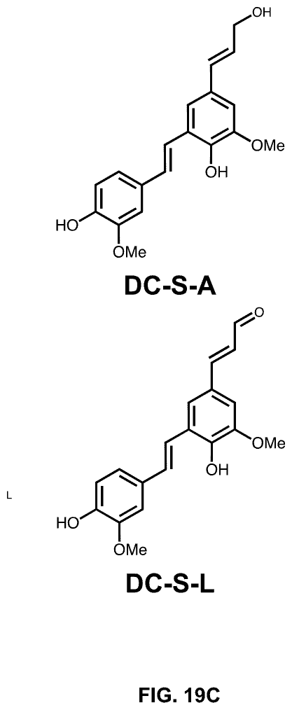

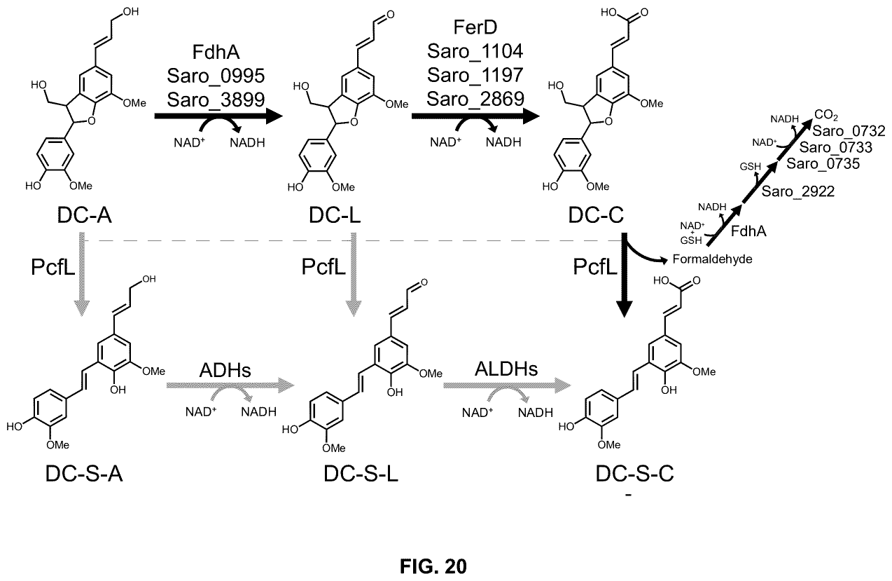

In addition, PcfL deformylates not only DC-C, but also DC-A and DC-L in vitro (FIGS. 19A and 19B), forming products that match the m/z of predicted allylic alcohol and allylic aldehyde stilbenes (FIG. 19C). While we propose that side chain oxidation precedes conversion of the phenylcoumaran to a stilbene based on the transient accumulation of DC-C in the medium when 12444PDC is grown on DC-A (FIG. 2B), it is possible that PcfL converts some DC-A or DC-L to a stilbene prior to side chain oxidation (FIG. 20).

In addition to N. aromaticivorans enzymes acting on multiple aromatic substrates, it is known that multiple enzymes often mediate the same reaction in aromatic metabolism. Consistent with this, we found that allylic side chain oxidation of DC-A and oxidation of 5-FF are performed by multiple dehydrogenases. While our data indicate that LsdD plays a major role in cleavage of DC-S-C into monomers, it is possible that one or both of two other N. aromaticivorans homologs of this dioxygenase (NOV2 (Saro_2809) and Saro_3580) can also perform this reaction. Overall, our findings showcase the robust and flexible strategies N. aromaticivorans uses for funneling a range of aromatics into a central metabolic pathway.

### Conservation of β-5 Linked Aromatic Catabolic Pathways in the Order Sphingomonadales

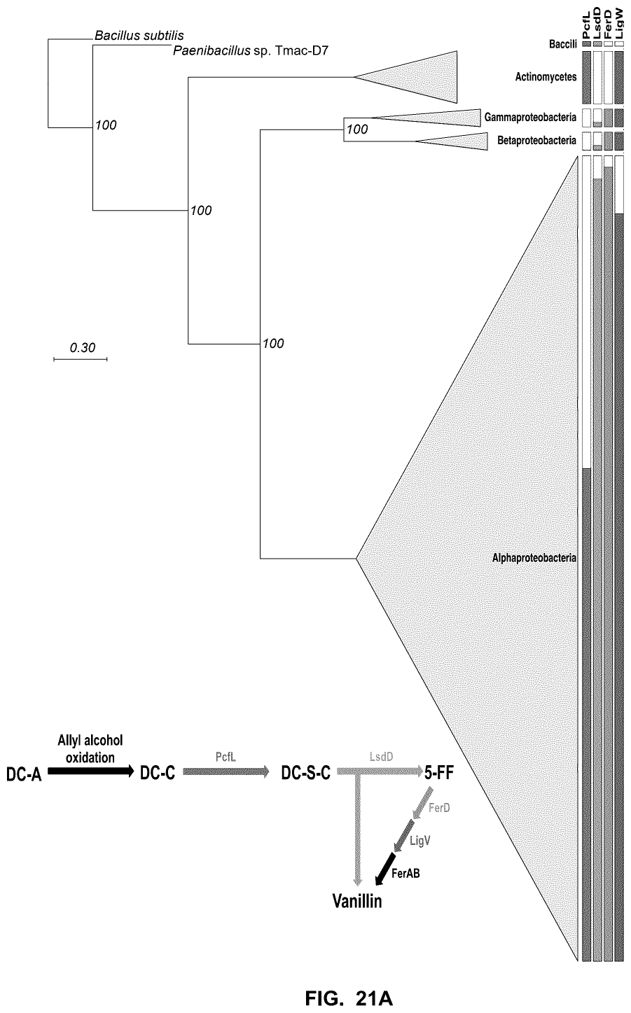

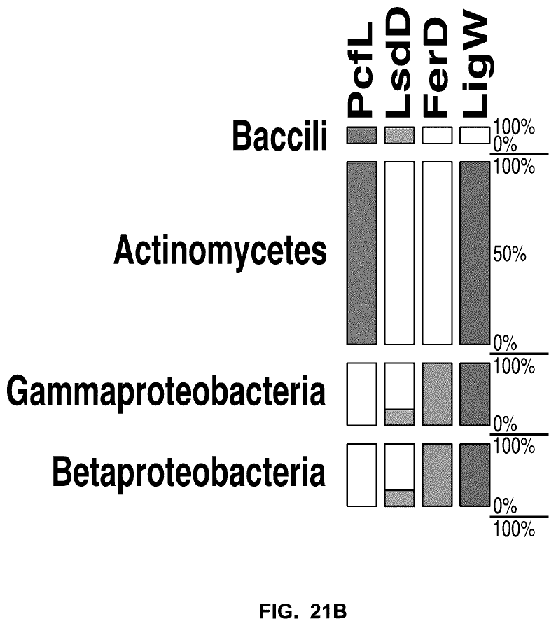

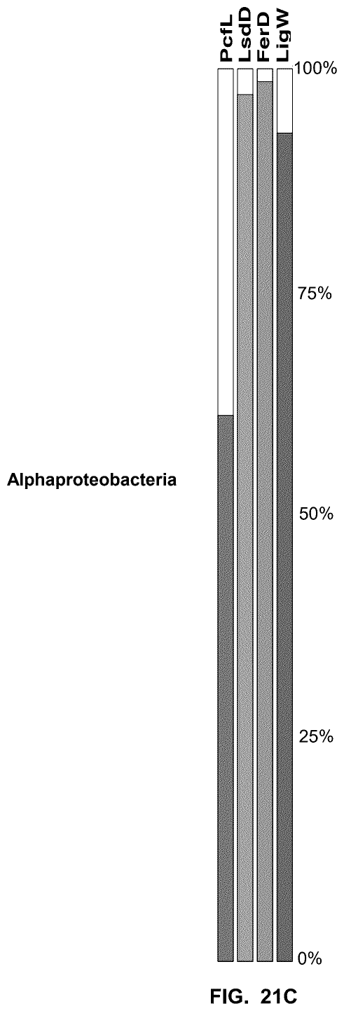

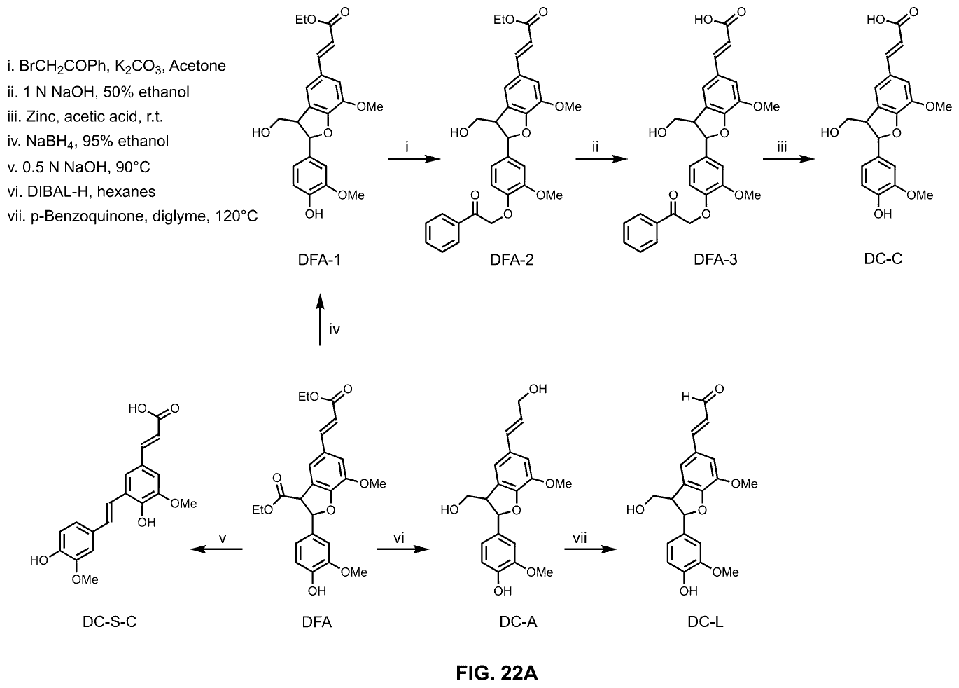

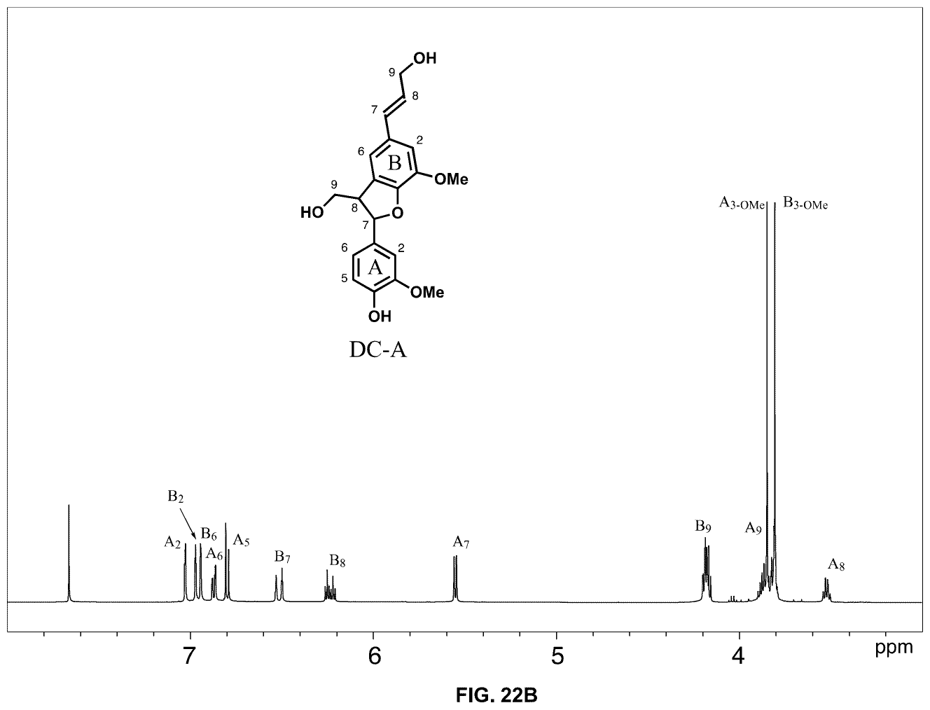

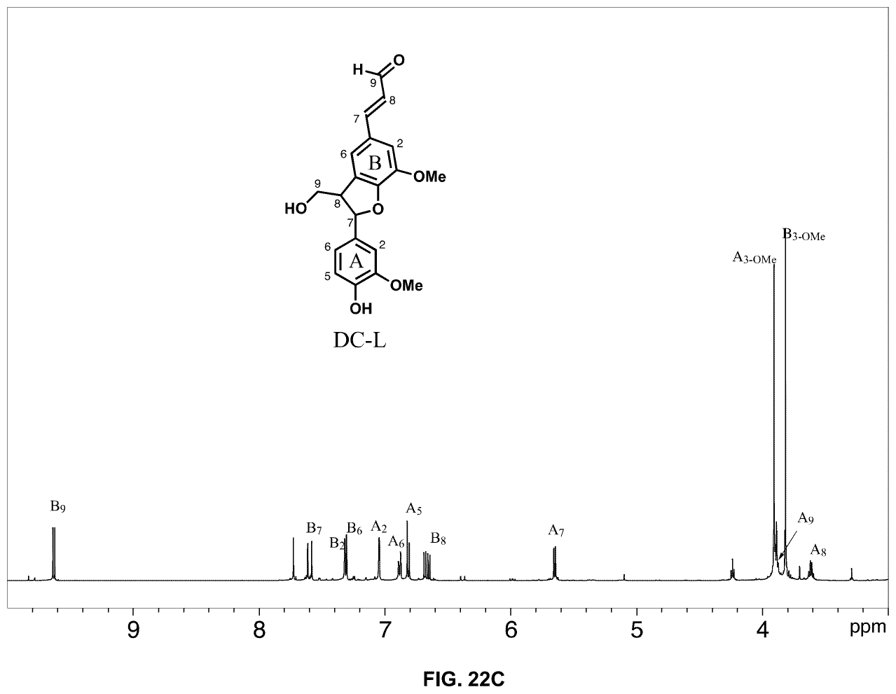

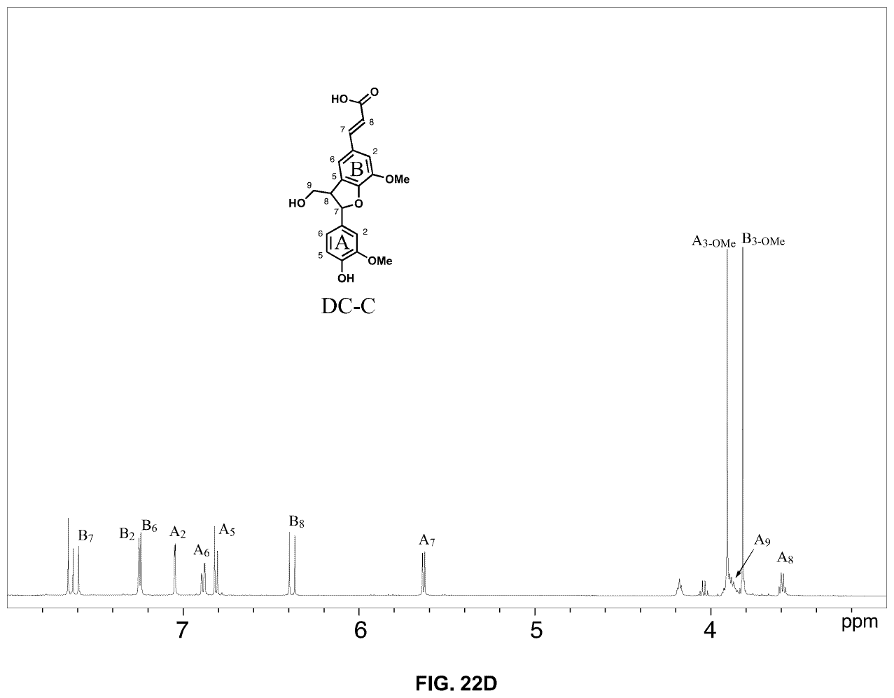

After uncovering the pathway for β-5 linked aromatic catabolism in N. aromaticivorans, we asked whether other organisms contain enzymes predicted to function in this pathway. To do so, we searched for homologs (>50% amino acid identity, >70% query coverage) of PcfL, LsdD, FerD, and LigW across all bacteria. We found that 82 organisms, all Alphaproteobacteria, are predicted to contain all four of these enzymes. Of those 82, all but Maricaulis flavus are members of the order Sphingomonadales. We also identified organisms with at least two homologs of β-5 linked aromatic catabolism enzymes, which are distributed across both gram-negative and gram-positive bacteria, including members of the orders Actinomyces, Gammaproteobacteria, Betaproteobacteria, and Bacilli (FIGS. 21A-21C). Thus, we concluded that the complete N. aromaticivorans pathway for β-5 linked aromatics is almost exclusively found in Sphingomonadales, but that other bacteria are predicted to contain some of the enzymes described in this study.

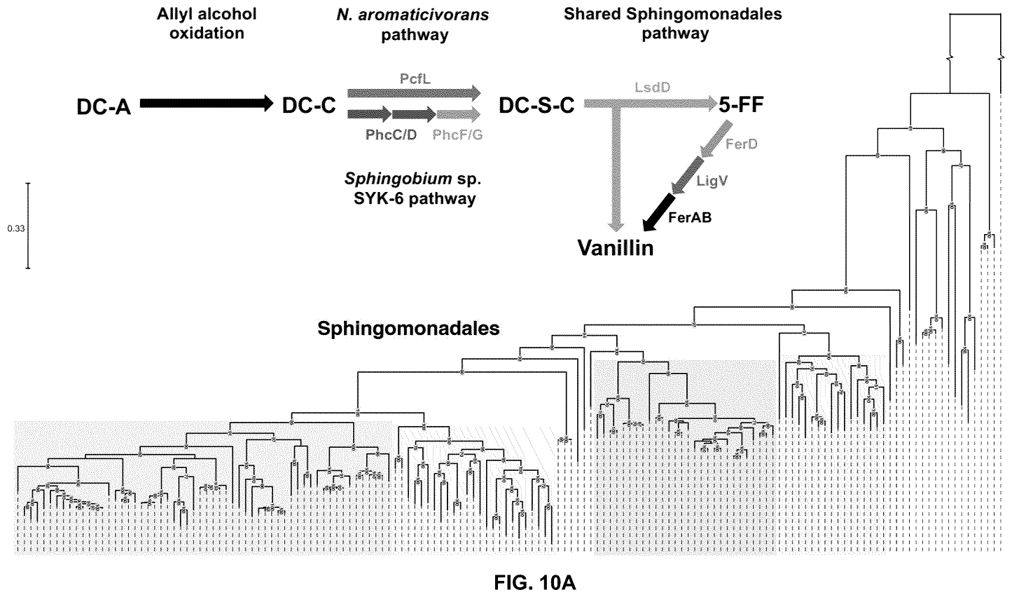

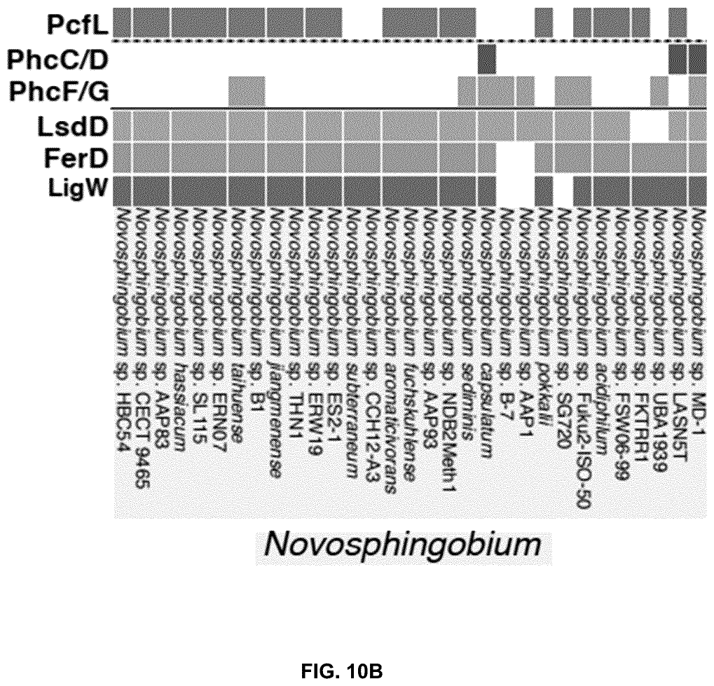

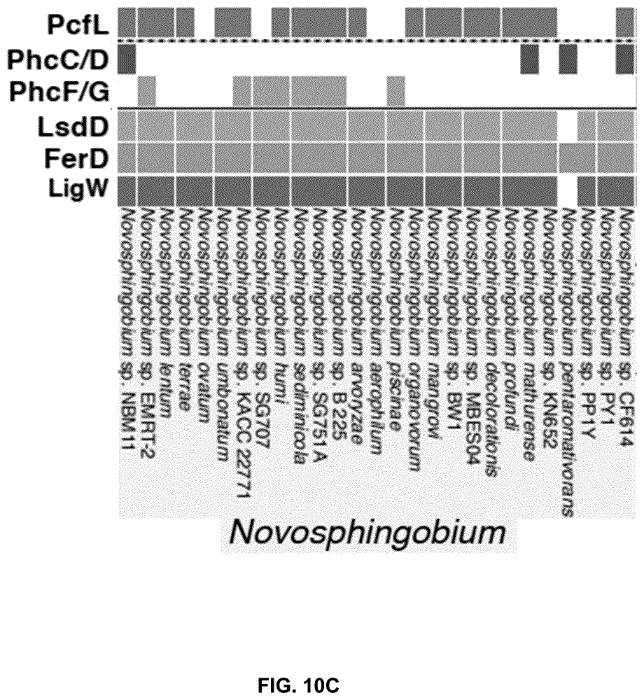

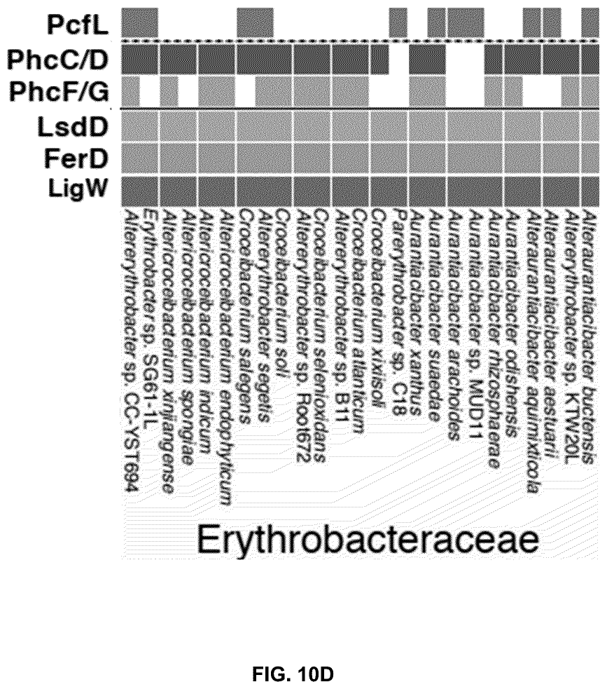

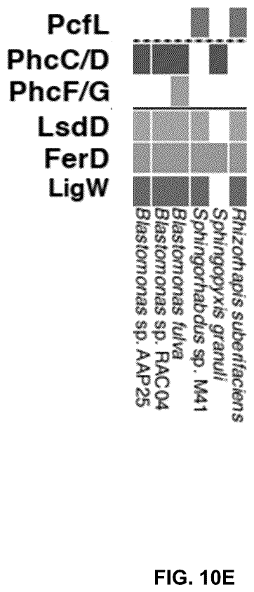

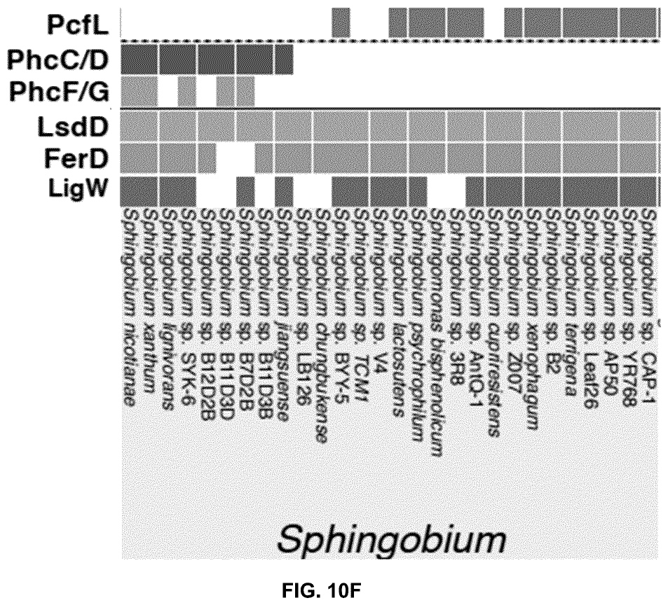

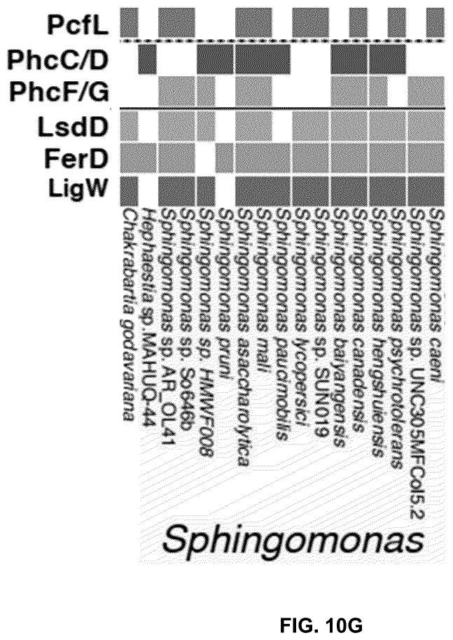

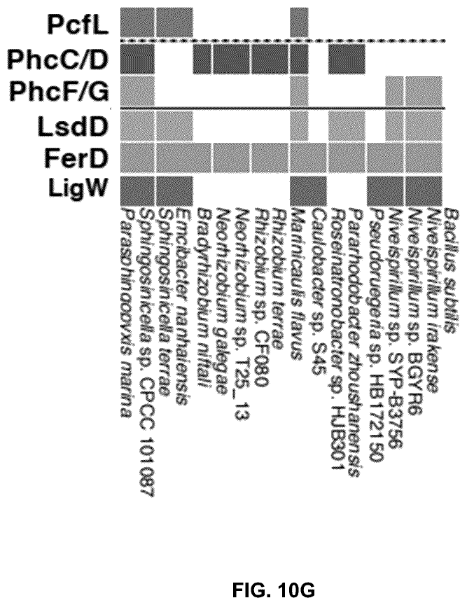

We also used comparative genomics to analyze the distribution of the β-5 linked aromatic catabolic pathways found in N. aromaticivorans and Sphingobium sp. SYK-6 (FIG. 10). For this analysis, we included the two pairs of enantiospecific enzymes (PhcC/PhcD and PhcF/PhcG) from the Sphingobium sp. SYK-6 pathway that are not shared by N. aromaticivorans. We found that most species predicted to have the enzymes needed for β-5 linked aromatic catabolism contain homologs of LsdD, FerD, and LigW, but they differ in whether they are predicted to convert DC-C to DC-S-C using a PcfL homolog (N. aromaticivorans pathway) or through oxidation and decarboxylation of DC-C (Sphingobium sp. SYK-6 pathway). Most of the organisms identified by our search contain homologs of either PcfL or PhcC/PhcD and/or PhcF/PhcG, but ten species contain homologs of all of these enzymes, suggesting they can convert a phenylcoumaran to a stilbene via both of these pathways.

The largest clades of Alphaproteobacteria with predicted β-5 catabolism capabilities are members of the genera Novosphingobium, Sphingobium, and Sphingomonas, and other members of the family Erythrobacteraceae aside from Novosphingobium. Our analysis predicts that the PcfL-dependent formaldehyde releasing pathway found in N. aromaticivorans is common in the genus Novosphingobium, while the phenylcoumaran oxidation and decarboxylation pathway discovered in Sphingobium sp. SYK-6 is common in other Erythrobacteraceae. The Sphingobium clade can be split into two groups, one of which is predicted to use either pathway. By contrast, the Sphingomonas clade is comprised of organisms predicted to contain either or both pathways for β-5 linked aromatic catabolism. In total, while the PcfL-dependent pathway is found in 82 Alphaproteobacteria, homologs of both PhcC/PhcD and PhcF/PhcG are found in 32 organisms. Overall, this analysis has revealed a conserved core pathway among the Sphingomonadales for metabolism of a β-5 linked stilbene and a pair of diverging pathways for the conversion of a phenylcoumaran to a stilbene.

In sum, we identified a catabolic pathway for β-5 linked aromatics in N. aromaticivorans that uses four conserved enzymes in addition to several partially redundant enzymes to funnel each monomeric unit into the N. aromaticivorans central aromatic pathway. Notably, this work showed that N. aromaticivorans uses a heretofore undescribed γ-formaldehyde lyase, PcfL, for converting phenylcoumarans to stilbenes. Future studies should focus on biochemically and mechanistically characterizing PcfL, as well as comparing it to its homolog, LdpA (24, 37), which is reported to generate a stilbene from a R-1 linked aromatic dimer.

The results of this analysis have expanded our knowledge of the aromatic metabolism of N. aromaticivorans and the order Sphingomonadales, laying the groundwork for future metabolic engineering to optimize the production of commodity chemicals from additional major components of deconstructed lignin. This N. aromaticivorans pathway holds promise for industrial applications since its catabolism of β-5 linked aromatics to vanillic acid and ferulic acid requires a minimal set of five gene products, as we demonstrated in vitro. These five genes could confer β-5 linked aromatic catabolism on other industrially relevant species. To increase the impact of our findings, future work is needed to assess whether β-5 linked aromatics that have been subjected to different pretreatment conditions are catabolized by N. aromaticivorans through a similar pathway to the one elucidated in this study.

### Methods

### Chemicals

Other than those noted below, all chemicals used were analytical grade and were purchased commercially.

(E)-4-(3-(hydroxymethyl)-5-(3-hydroxyprop-1-en-1-yl)-7-methoxy-2,3-dihydrobenzofuran-2-yl)-2-methoxyphenol (DC-A) was synthesized in 65% yield by DIBAL-H reduction of 8-5-coupled diferulate (DFA) (45), which was synthesized from ethyl ferulate through peroxidase-H₂O₂ oxidative coupling reaction (46). (E)-3-(2-(4-hydroxy-3-methoxyphenyl)-3-(hydroxymethyl)-7-methoxy-2,3-dihydrobenzofuran-5-yl)acrylaldehyde (DC-L) was synthesized in 80% yield from DC-A by p-benzoquinone oxidation as previously described (47). (E)-3-(4-hydroxy-3-((E)-4-hydroxy-3-methoxystyryl)-5-methoxyphenyl)acrylic acid (DC-S-C) was synthesized in 23% yield from DFA by alkali hydrolysis at 90° C. as previously described (48). To synthesize (E)-3-(2-(4-hydroxy-3-methoxyphenyl)-3-(hydroxymethyl)-7-methoxy-2, 3-dihydrobenzofuran-5-yl)acrylic acid (DC-C), DFA was selectively reduced in 95% ethanol by NaBH₄ to produce the alcohol DFA-1 (32% yield). Protection of phenolic hydroxyl in DFA-1 by phenacyl ether was accomplished in 90% yield. Alkali hydrolysis of the ester group in DFA-2 was performed in 1N NaOH/ethanol (1/1, v/v) solution, producing the acid DFA-3 in 85% yield. Finally, deprotection of the phenacyl ether in DFA-3 by Zinc dust in acetic acid resulted in DC-C in 70% yield. The synthesis of DC-A, DC-L, DC-C, and DC-S-C is depicted in FIG. 12 (A). Each product was confirmed by NMR (FIGS. 12B-12E, Table 4).

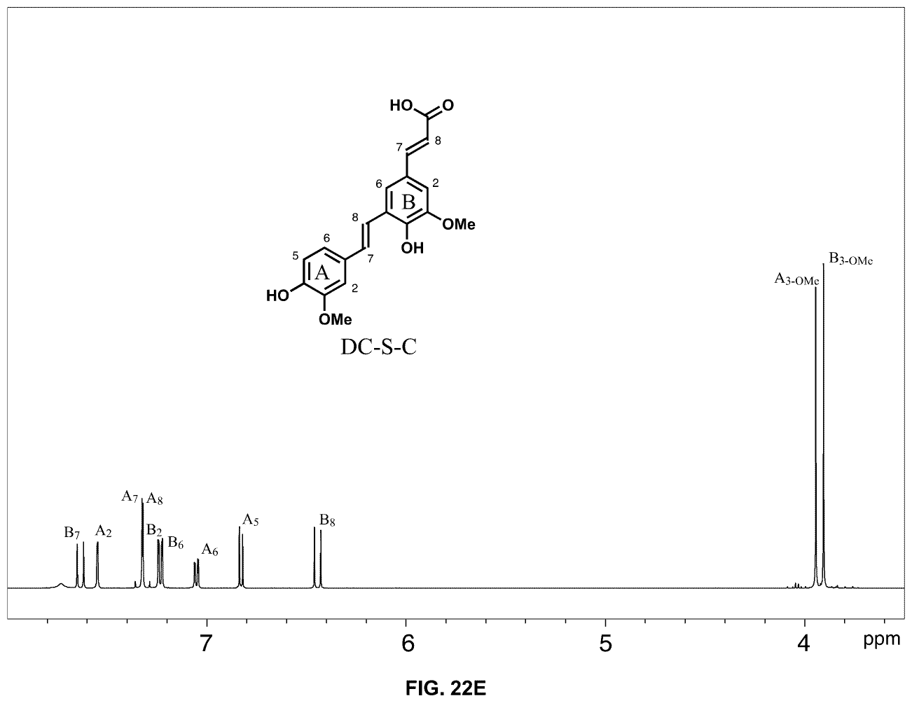

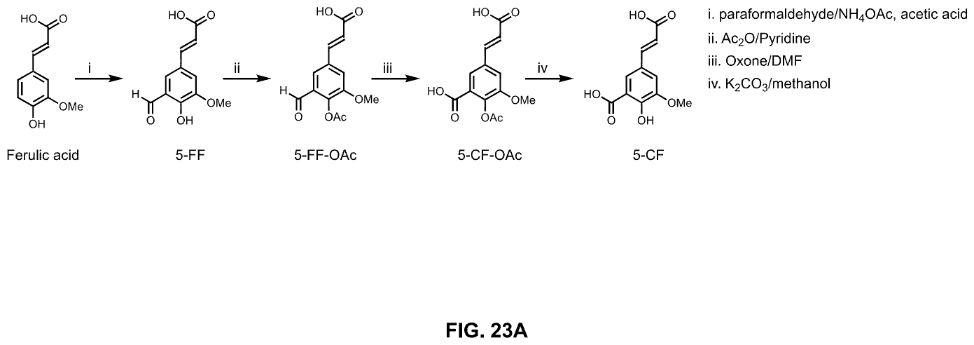

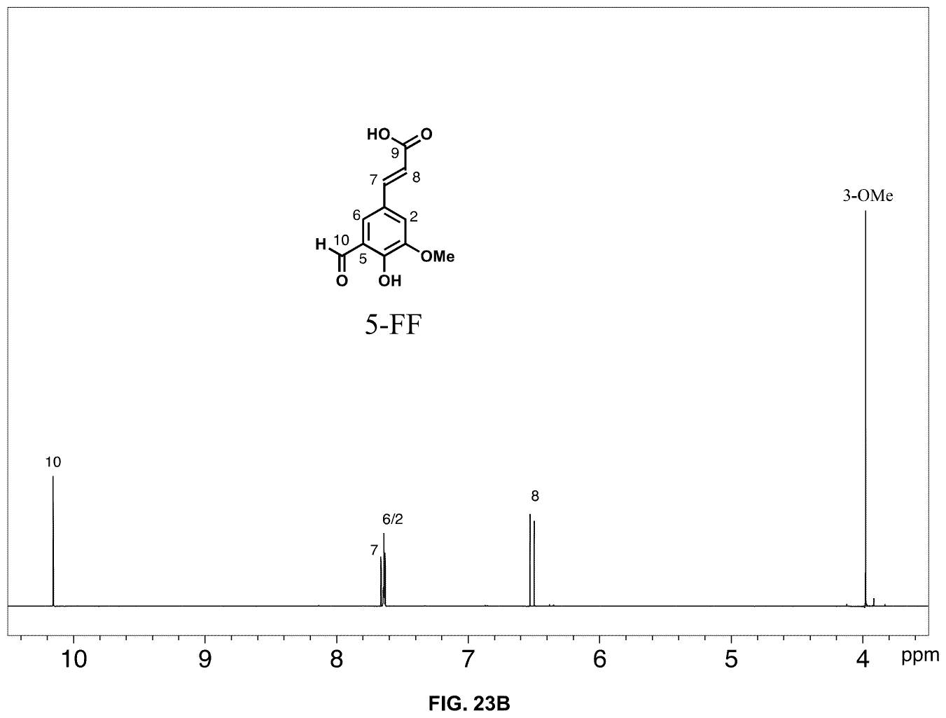

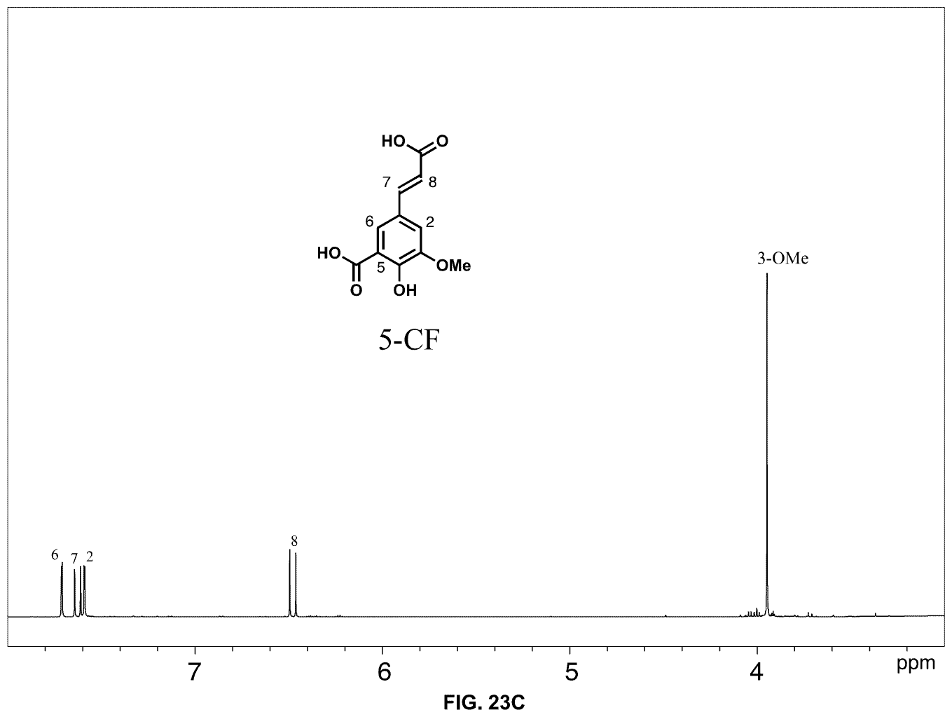

(E)-3-(3-formyl-4-hydroxy-5-methoxyphenyl)acrylic acid (5-FF) was synthesized in 38% yield from ferulic acid by ortho formylation with paraformaldehyde and ammonium acetate in acetic acid as previously described (49). To synthesize (E)-5-(2-carboxyvinyl)-2-hydroxy-3-methoxybenzoic acid (5-CF), the phenolic hydroxyl of 5-FF was protected by acetylation in acetic anhydride/pyridine (1/1, v/v) to produce acetylated 5-FF. The aldehyde group was then converted to carboxylic acid in 85% yield by Oxone oxidation in DMF as previously described (50). Finally, the acetylated 5-CF was transferred in 95% yield to 5-CF by hydrolysis of the acetate with K₂CO₃ in 60% aqueous ethanol. The synthesis of 5-FF and 5-CF is depicted in FIG. 23A. Each product was confirmed by NMR (FIGS. 23B and 23C), Table 4).

To generate DC-T-C, DC-S-C was incubated under abiotic conditions in SMB minimal medium supplemented with 1 g/L glucose at 30° C. for 2 weeks. DMSO was then added to a 30% final concentration (v/v). The resulting product was recovered by ethyl acetate extraction of the SMB buffer solution. After removing the solvent, the crude residue was directly examined by NMR. It was found that the DC-S-C was completely converted and the majority of products were two stereoisomers of 8-8-coupled dimer DC-T-C, which was identified by comparison of their NMR data with those published (FIG. 15A, Table 4) (51). This material was used as a 1 mM DC-T-C standard. All other standards were created by dissolving the appropriate compound in DMSO at a final concentration of 100 mM.

TABLE 4
¹H and ¹³C NMR (acetone-d₆) analysis of indicated compounds.

| Compound | ¹H NMR Data | ¹³C NMR Data |
|---|---|---|
| DC-A | 3.52, 3.78-3.88, 3.81,3.85, 4.19, 5.56, | 54.70, 56.13, 56.21, 63.33, 64.49, 88.45, |
| | 6.23, 6.52, 6.80, 6.87, 6.94, 6.97, 7.03 | 110.30, 111.41, 115.58, 115.96, 119.51, |
| | | 128.28, 130.29, 130.42, 131.82, 134.28, |
| | | 145.09, 147.19, 148.28, 148.82 |
| DC-L | 3.61, 3.82, 3.91, 3.87-3.91, 5.65, 6.65, | 54.25, 56.29, 56.46, 64.32, 89.39, 110.59, |
| | 6.81, 6.88, 7.04, 7.29, 7.32, 7.59, 9.63 | 113.56, 115.76, 119.64, 119.73, 127.14, |
| | | 129.00, 131.24, 133.75, 145.65, 147.55, |
| | | 148.46, 152.41, 154.10, 193.77 |
| DC-C | 3.59 (m, 1H), 3.82 (s, 3H, —OMe), 3.83- | 54.36, 56.20, 56.33, 64.28, 89.14, 110.45, |
| | 3.92 (m, 2H), 3.90 (s, 3H, —OMe), 4.18, | 113.12, 115.67, 116.00, 118.73, 119.67, |
| | 5.63, 6.38 (d, J = 15.92 Hz), 6.81 (d, J = | 129.01, 130.88, 133.86, 145.46, 145.98, |
| | 8.15 Hz), 6.88 (dd, J = 8.15, 1.93 Hz), | 147.41, 148.38, 151.54, 168.04. |
| | 7.05 (d, J = 1.93 Hz), 7.23 (br-s), 7.25 | |
| | (br-s), 7.61(d, J = 15.92 Hz) | |
| DC-S-C | 3.91 (s, OMe), 3.95 (s, OMe), 6.44 (d, | 56.10, 56.44, 108.96, 109.89, 115.90, |
| | J = 15.9 Hz),6.83(d, J = 8.1 Hz), 7.05 | 116.18, 120.41, 120.82, 121.10, 125.33, |
| | (dd, J = 8.1, 2.0, ), 7.22 (d, J = 2.0 Hz), | 126.83, 130.57, 130.77, 146.21, 146.88, |
| | 7.23 (d, J = 1.9 Hz), 7.31 and 7.33 | 147.46, 148.49, 148.71, 168.35 |
| | (ABqt, AVAB = 7.39 Hz, JAB = 16.5 Hz), | |
| | 7.54 (d, J = 1.9 Hz), 7.63 (1 H, d, | |
| | J = 15.9 Hz) | |
| 5-FF | 3.98 (s, 3H, OMe), 6.52 (d, J = 16.0 | 56.68, 116.36, 118.06, 122.11, 125.31, |
| | Hz), 7.64 (d, J = 16.0 Hz), 7.64 and 7.64 | 127.39, 144.34, 149.74, 154.02, 167.70, |
| | (ABqt, AVAB = 3.56 Hz, JAB = 2.15 Hz), | 196.04 (—CHO) |
| | 10.15 (s, —CHO) | |
| 5-CF | 3.95 (s, OMe), 6.48 (d, J = 15.95 Hz), | 56.50 (OMe), 113.17, 115.43, 117.60, |
| | 7.59 (d, J = 2.0 Hz), 7.62 (d, J = 15.95 | 123.87, 126.30, 144.75, 150.12, 155.52, |
| | Hz), 7.71 (d, J = 2.0 Hz) | 167.78, 172.64 |
| DC-T-C | 3.62(s), 3.98 (s), 4.13 (d, J = 3.64 Hz), | 55.76, 55.98, 56.48, 87.12, 109.10, 113.15, |
| (threo | 5.53 (d, J = 3.64 Hz), 6.30 (d, J = 1.90 | 115.59, 117.72, 118.56, 118.77, 129.60, |
| isomer) | Hz), 6.39 (d, J = 15.90 Hz), 6.53 (dd, J = | 130.13, 133.63, 144.20, 145.65, 146.96, |
| | 8.15, 1.90 Hz), 6.67 (d, J = 8.15 Hz), | 148.30, 151.41, 169.60 |
| | 7.30 (d, J = 1.50 Hz), 7.35 (d, J = 1.50 | |
| | Hz), 7.59 (d, J = 15.90 Hz) | |
| DC-T-C | 3.78 (s, OMe), 3.91 (s, OMe), 4.18 (d, | 53.50 (C-8), 56.22, 56.38, 88.67, 110.83, |
| (meso | J = 6.15 Hz), 5.52 (d, J = 6.15 Hz), 6.25 | 113.57, 115.85, 116.43, 118.48, 120.12, |
| isomer) | (d, J = 15.90 Hz), 6.80 (d, J = 1.2 Hz), | 129.35, 130.11, 132.91, 145.65, 145.70, |
| | 6.82 (d, J = 8.10 Hz), 6.84 (dd, J = 8.10, | 147.81, 148.50, 151.92, 167.93 |
| | 1.36 Hz), 6.98 (d, J = 1.56 Hz), 7.30 (d, | |
| | J = 1.56 Hz), 7.52 (d, J = 15.90 Hz) | |

### Bacterial Strains and Growth Media

N. aromaticivorans strain 12444A1879 is referred to as the wild-type elsewhere in this paper. In 12444A1879, a putative sacB homolog (Saro_1879) has been deleted (23) to allow for genomic modifications to be made using the pK18mobsacB plasmid system (52). The 12444PDC strain harbors several gene deletions that allow it to funnel aromatics into production of the aromatic metabolic pathway intermediate PDC (10). 12444PDC was used as a parent strain for the construction of the deletion mutants used to study DC-A catabolism. All N. aromaticivorans strains (Table 5) were grown at 30° C. and shaking at 200 rpm in SMB minimal medium supplemented with 1 g/L glucose, except where noted. SMB minimal medium was prepared as previously described (23).

E. coli NEB5a (New England Biolabs, Ipswich, MA) was used as a plasmid host. E. coli WM6026 (53) was used as a conjugal donor for mobilizing plasmids into N. aromaticivorans while E. coli B834 (54) was used to express recombinant proteins. All E. coli strains (Table 5) were grown in lysogeny broth (LB) at 37° C. and shaking at 200 rpm, except where noted below.

TABLE 5
Bacterial strains used in this study.

| Strain | Relevant Characteristics | Source |
|---|---|---|
| 12444Δ1879 | WT N. aromaticivorans Δ1879 (sacB-) | (23) |
| 12444PDC | 1244441879 Δ2819 (ligI) Δ2864 (desC) Δ2865 (desD) | (10) |
| 12444PDCΔpcfL | 12444PDC Δ0796 (pcfL) | This study |
| 12444PDCΔferD | 12444PDC Δ0797 (ferD) | This study |
| 12444PDCΔligW | 12444PDC Δ0799 (lig W) | This study |
| 12444PDCΔlsdD | 12444PDC Δ0802 (lsdD) | This study |
| 12444PDCΔfdhA | 12444PDC Δ0874 (fdhA) | This study |
| E. coli NEB5α | fhuA2 Δ(argF-lacZ)U169 phoA glnV44 Φ80 Δ(lacZ)M15 | New England |
| | gyrA96 recA1 relA1 endAl thi-1 hsdR17 | Biolabs |
| E. coli WM6026 | lacI^{q}, rrnB3, ΔlacZ4787, hsdR514, ΔaraBAD567, | (53) |
| | ΔrhaBAD568, rph-1, attλ::pAE12(ΔoriR6K-cat::Frt5), | |
| | ΔendA::Frt, uidA(ΔMluI)::pir, attHK::pJK1006D(oriR6K- | |
| | cat::Frt5; trfA::Frt) dap | |
| E. coli B834 | F⁻ hsdS metE gal ompT | (54) |

### RNA-Seq Analysis

Four isolated N. aromaticivorans PDC12444 colonies were cultured and grown overnight. The next day, the overnight cultures were diluted 1:1 with SMB minimal medium supplemented with 1 g/L glucose and grown for one hour. The cultures were then diluted 1:100 into separate cultures of SMB minimal medium supplemented with 1 g/L glucose, 1 g/L glucose plus 0.5 mM DC-A, 1 g/L glucose plus 0.5 mM vanillin, or 1 g/L glucose plus 0.5 mM ferulic acid. These cultures were grown until they reached mid-exponential growth phase, at which point growth was stopped by the 1:8 addition of ice cold 5% acid phenol:chloroform (5:1) in ethanol. The cells were pelleted by centrifugation (4,300×g for 10 minutes) at 4° C. and stored at −80° C. RNA was extracted using hot acid phenol:chloroform (5:1), as previously described (55). RNA was purified using the RNeasy Kit (Qiagen, Germantown, MD), checked for purity by NanoDrop spectrophotometry (OD 260:280 ratio >2.0, OD 260:230 ratio >2.0), visualized after electrophoresis on a 1% agarose gel, and quantified with a Qubit fluorometer.

RNA-Seq library preparation and sequencing was performed by the Joint Genome Institute (JGI) using default parameters. rRNA in the samples was depleted using the QIAseq FastSelect kit (Qiagen, Germantown, MD). Libraries were constructed using the TruSeq stranded mRNA kit (Illumina, San Diego, CA) following standard JGI protocols. The libraries were sequenced on an Illumina NovaSeq to produce 2×150 reads. All paired-end FASTQ files were processed through the same pipeline. Reads were trimmed using Trimmomatic version 0.3 with the default settings except for a HEADCROP of 5, LEADING of 3, TRAILING of 3, SLIDINGWINDOW of 3:30, and MINLEN of 36 (56). After trimming, the reads were aligned to the N. aromaticivorans DSM12444 genome sequence (GenBank accession GCF_000013325.1) using bwa-mem (version 0.7.17-h5bf99c6_8) with default settings (57). Alignment files were further processed with Picard-tools (version 2.26.10) (https://broadinstitute.github.io/picard/) (CleanSAM and AddOrReplaceReadGroups commands) and samtools (version 1.2) (sort and index commands) (58). Paired aligned reads were mapped to gene locations using HTSeq version 0.6.0 (59). The R package edgeR (version 3.30.3) (60) with default settings was used to identify significantly differentially expressed genes from pairwise analyses, using Benjamini and Hochberg false discovery rate (FDR) less than 0.05 as a significance threshold (61). Raw sequencing reads were normalized using the fragments per kilobase per million mapped reads method (FPKM). Fold change, FPKM, and FDR for all genes are described elsewhere herein.

### Screening a Genome-Scale RB-TnSeq Library

A previously generated RB-TnSeq library in wild-type N. aromaticivorans was used to screen for fitness (21). An aliquot of the library was thawed and cultured in LB supplemented with 50 mg/L kanamycin and grown overnight. The culture was diluted 1:100 into three flasks containing 2 g/L glucose in SMB minimal medium and grown to saturation (˜6.5 doublings). Each culture was then diluted to a starting cell density of 40 Klett units in SMB minimal medium with 1 g/L glucose or 1 g/L DC-A as the sole carbon source. The cultures were grown to saturation (˜6.5 doublings), split into 0.6 mL aliquots, frozen, and stored at −80° C. The cells were harvested by centrifugation (2,300×g for 5 minutes) at 4° C., resuspended in lysis buffer (0.16 mM EDTA and 2% SDS), and incubated at 65° C. for 5 minutes. Genomic DNA was extracted using 25:24:1 phenol:chloroform:isoamyl alcohol. Barcode DNA sequences were amplified from the genome using custom indexing primers BarSeq_P1 and BarSeq_P2_ITO01 to BarSeq_P2_IT009 (62). Barcode amplicons were quantified using a Qubit fluorometer and pooled before being sequenced at Azenta/GENEWIZ on an Illumina MiSeq with paired-end 150 bp reads (Illumina, San Diego, CA). Barcode frequencies and fitness values were calculated as previously described (62).

### Heterologous Protein Expression

To express recombinant proteins, a single isolated colony of each E. coli B834 expression strain was cultured in LB medium containing kanamycin (50 mg/L). The next day, the overnight cultures were diluted 1:1 in LB medium and grown for one hour at 37° C. Next, flasks containing either 48 ml, 2×YPTG medium (16 g/L, tryptone, 10 g/L yeast extract, 5 g/L NaCl, 7 g/L, KH₂PO₄, 3 g/L K₂HPO₄, 18 g/L glucose) or 49.5 mL ZMS-80155 auto-inducing medium (63) were inoculated with 2_mL or 0.5 mL of E. coli B834 culture, respectively. The 2×YPTG cultures were allowed to grow until their OD600 reached 0.6-0.8, at which point expression of the recombinant protein was induced via addition of 1 mM isopropyl β-D-1-thiogalactopyranosid (IPTG). Since significant recombinant FdhA was present in inclusion bodies, we added 0.5 M sorbitol and 0.2 M arginine to its culture at the same time we added IPTG (64). 2×YPTG and ZMS-801555 cultures were both grown overnight at room temperature (˜24 hours). The cultures were washed twice with cold S30 buffer supplemented with 2 mM dithiothreitol (DTT) (65) and the cells were harvested by centrifugation (3000×g for 10 minutes) at 4° C. The cell pellets were flash frozen in a dry ice-ethanol bath and stored at −80° C. Heterologous expression of His-tagged proteins for purification was performed as described above except the cultures contained 990 mL ZMS-80155 auto-inducing medium and were inoculated with 10 mL E. coli B834 culture.

### Harvesting Cell Extracts

Harvested E. coli B834 cells containing the recombinant proteins were resuspended in 12 mL ice-cold S30 buffer supplemented with 2 mM DTT for untagged constructs or in 2.5 mL/g pellet lysis buffer (50 mM NatPO₄*H₂O, 0.5 mM tris(2-carboxyethyl)phosphine, 5 mM imidazole, 100 mM NaCl, 10% glycerol, and 1% Triton-X-100, pH 8.0) for His-tagged constructs. Cells were sonicated on ice using a QSonic sonicator set to amplitude 40 with 20 seconds on and 40 seconds off cycles for 15 minutes. The sonicated solutions were then centrifuged (7,600×g for 20 minutes) at 4° C. and the supernatant was collected as a crude cell extract, flash frozen in a dry ice-ethanol bath, and stored at −80° C.

### Growth Experiments

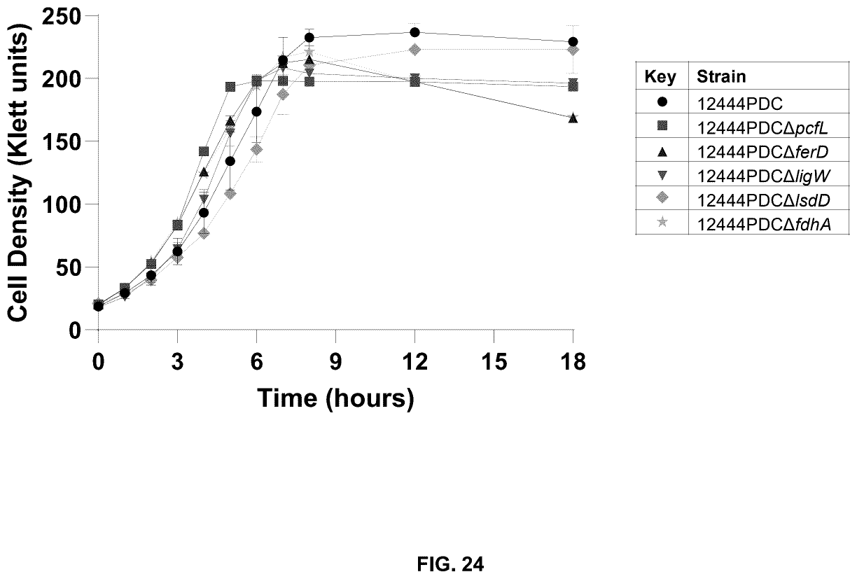

All N. aromaticivorans strains were cultured in triplicate from three isolated colonies and grown overnight. The next day, the cultures were diluted 1:1 in SMB minimal medium supplemented with 1 g/L glucose and incubated for one hour before being diluted with additional 1 g/L glucose in SMB minimal medium to the same cell density. A portion of these cultures were centrifuged (2,300×g for 5 minutes), the supernatant was discarded, and the cell pellets were diluted in the appropriate growth medium (SMB minimal medium with 1 g/L glucose and with or without 0.5 mM DC-A). One mL aliquots of the resuspended cells were used to inoculate triplicate flasks containing 19 mL of the appropriate medium, giving a starting cell density of 20-25 Klett units. The cultures were grown for 18 hours and growth was monitored using a Klett-Summerson colorimeter (FIG. 24). At indicated time points, 0.8 mL of the cultures were removed, the cells were pelleted by centrifugation (2,300×g for 5 minutes) at 4° C., and the supernatants were passed through a 0.22 m PVDF syringe filter to collect extracellular samples that were stored at −80° C. for subsequent analysis.

Since DC-A has low solubility in SMB minimal medium, a 100 mM DC-A stock in DMSO was added to SMB minimal medium that was heated to 65° C. to achieve final concentrations of ˜0.45 mM DC-A and 0.5% DMSO after filtering the medium.

### Analysis of Extracellular Aromatic Metabolites

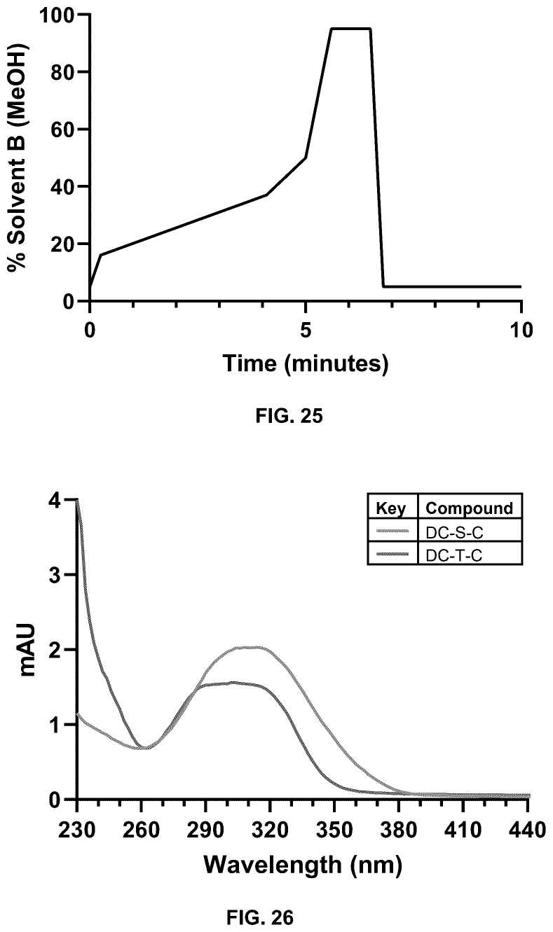

The aromatics in extracellular samples were analyzed on a Shimadzu triple quadrupole liquid chromatography mass spectrometer (Nexera XR HPLC-8045 MS/MS). The mobile phase was a binary gradient with solvent A (0.2% formic acid in water) and solvent B (methanol) using the protocol in FIG. 25 and flowing at a rate of 0.4 mL/min. The stationary phase was a Phemonenex Kinetex F5 column (2.6 μm pore size, 2.1 mm ID, 150 mm length, P/N: H18-105937). The m/z of peaks was determined using a negative ion mode scan. Aromatic compound standards were generated as described above and used to confirm the identity of unknown chemicals through elution and multiple-reaction monitoring (MRM).

A series of 2-fold dilutions were performed to create a standard curve of eight concentrations of each compound. The standard curves were then used to quantify extracellular concentrations of aromatics via MRM (Table 2). The percent yields of individual compounds were calculated using equation (1).

$$percent yield = \frac{( {[ aromatic ]}_{final} \times n )}{( {[ DC - A ]}_{initial} \times 2 )} \times 100 \qquad Equation ( 1 )$$

$$Where n = number of aromatic rings in the compound$$

### In Vitro Enzyme Activity Assays

Crude cell extracts containing individual recombinant proteins were prepared as described above. The cell extracts expressing candidate DC-A catabolism proteins and control E. coli B834 cell extract or control extract alone were added to 3 separate reaction mixtures containing S30 buffer (pH 8.2) supplemented with aromatic substrate and NAD⁺, where appropriate. In candidate test conditions, candidate protein and control extracts each comprised 15% of the final volume and the aromatic and NAD⁺ (where appropriate) concentrations were 0.25 mM and 1 mM, respectively. For the in vitro reconstruction of the DC-A catabolic pathway experiment, each of the five protein expression cell extracts made up 5% of the final reaction volume instead. For control reactions, the crude extract from E. coli B834 comprised 30% of the final mixture. These reactions were incubated at 30° C. for 6 hours and then diluted 1:1 with 40% acetonitrile, 40% methanol, and 100 mM formic acid in water to terminate enzyme activity. The samples were centrifuged (21,000×g for 5 minutes) at 4° C. and the supernatants were passed through a 0.22 m PVDF syringe filter and stored at −80° C. for further analysis. Experiments testing in vitro activity of purified PcfL and FerD were performed in the same fashion, except HEPES buffer (pH 7.66) was used in placed of S30 buffer and control experiments were conducted by adding additional HEPES buffer instead of crude E. coli B834 cell extract.

Analysis of the in vitro reaction products was performed on a Shimadzu triple quadrupole liquid chromatography mass spectrometer as described above. LC traces were collected and reaction products were identified using MRM methods developed from synthetic standards (Table 2).

To assay the relative rate of conversion of substrates to products by candidate ADHs and ALDHs, absorbance at 370 nm was used for measuring DC-L concentration since DC-L absorbs at this wavelength while DC-A and DC-C do not (FIG. 17). E. coli B834 cell extracts expressing candidate ADHs or ALDHs as well as control extracts were collected as described above and diluted with S30 buffer plus 2 mM DTT to a total protein concentration of 2 mg/mL. The dehydrogenase and control E. coli B834 cell extracts were each added to triplicate wells of a 96-well plate containing S30 buffer (pH 8.2) supplemented with 0.15 mM DC-A or 0.15 mM DC-L, as well as 1 mM electron acceptor (NAD⁺ or PQQ, where appropriate). The diluted extracts comprised 5% of the final reaction volume. Each enzyme was tested for activity in assays with and without added electron acceptor. After addition of cell extract to the wells, the 96-well plate was immediately placed in a Tecan Infinite M1000 reader set to maintain a temperature of 30° C. At indicated timepoints over the course of one hour, absorbance of DC-L was measured at 370 nm. Control experiments show that NADH does not accumulate significantly in this cell extract system, potentially due to the activity of native E. coli dehydrogenases (FIG. 16B). A series of standards created by 2-fold dilutions of DC-L in S30 buffer plus 2 mM DTT were used to generate an 8-point standard curve and quantify the concentration of DC-L in the reactions based on absorbance at 370 nm.

Due to absorbance of PQQ at 370 nm, the activity assay for the putative PQQ-dependent ALDH Saro_2870 was performed as described above except 15 L samples were collected from the reaction at each indicated time point and diluted 1:1 with 40% acetonitrile, 40% methanol, and 100 mM formic acid in water to terminate enzyme activity. These samples were then diluted 5:1 with S30 buffer and analyzed by LC-MS as described above.

Formaldehyde was measured as a product of PcfL activity by using small aliquots of the cell extract reaction mixtures and the Invitrogen Formaldehyde Fluorescent Detection Kit (Invitrogen, Carlsbad, CA). To test for conversion of NAD⁺ to NADH by FerD, assays were performed as described above for both the purified FerD and FerD-containing cell extract, except the S30 or HEPES buffer was supplemented with 0.4 mM NAD⁺ and 0.4 mM 5-FF. NAD⁺ and NADH were quantified using small aliquots of the reactions and the Sigma Aldrich NAD/NADH Quantitation Kit (Sigma Aldrich, St. Louis, MO).

### Phylogenetic Analysis

Predicted homologs of DC-A catabolism genes were identified using NCBI protein-protein BLAST to search all genomes in the NCBI database as of July 2023, excluding uncultured/environmental sample sequences and using cut-offs of 50% amino acid identity and 70% query coverage. All bacteria containing homologs of at least two N. aromaticivorans DC-A catabolism enzymes (PcfL, FerD, LigW, and LsdD) were used to create a phylogenetic tree. Alphaproteobacteria containing homologs of at least two N. aromaticivorans DC-A catabolism enzymes (PcfL, FerD, LigW, and LsdD) and/or Sphingobium sp. SYK-6 DC-A catabolism enzymes that differ from N. aromaticivorans (PhcC/PhcD and PhcF/PhcG) were used to create an additional phylogenetic tree.

Phylogenetic analysis was performed on genomes identified in these BLAST searches (Table 6) using GDTB-Tk (version 2.1.1, release 207_v2) to identify and align the bacterial reference genes using default parameters (66). The multiple sequence alignment file was used to construct maximum likelihood trees using RAxML-ng (version 0.9.0) using model LG+G8+F and default parameters (67). Bacillus subtilis subsp. subtilis str. 168 was used as an outgroup. Trees were visualized in TreeViewer (version 2.2.0) (68).

TABLE 6
Organisms included in the phylogenetic analyses
in FIGS. 10A-10G and FIGS. 21A-21C.

| | Assembly Accession | |
|---|---|---|
| Scientific Name | Number | Class |
| Alteraurantiacibacter aestuarii | GCF_009827405.1 | Alphaproteobacteria |
| Alteraurantiacibacter aquimixticola | GCF_004965515.1 | Alphaproteobacteria |
| Alteraurantiacibacter buctensis | GCF_009827655.1 | Alphaproteobacteria |
| Altererythrobacter segetis | GCF_011320115.1 | Alphaproteobacteria |
| Altererythrobacter sp. B11 | GCF_003569745.1 | Alphaproteobacteria |
| Altererythrobacter sp. CC-YST694 | GCF_020539485.1 | Alphaproteobacteria |
| Altererythrobacter sp. KTW20L | GCF_023501975.1 | Alphaproteobacteria |
| Altererythrobacter sp. Root672 | GCF_001427865.1 | Alphaproteobacteria |
| Altericroceibacterium endophyticum | GCF_009827595.1 | Alphaproteobacteria |
| Altericroceibacterium indicum | GCF_009828105.1 | Alphaproteobacteria |
| Altericroceibacterium spongiae | GCF_003610805.1 | Alphaproteobacteria |
| Altericroceibacterium xinjiangense | GCF_003958635.1 | Alphaproteobacteria |
| Aurantiacibacter arachoides | GCF_009827335.1 | Alphaproteobacteria |
| Aurantiacibacter odishensis | GCF_003605195.1 | Alphaproteobacteria |
| Aurantiacibacter rhizosphaerae | GCF_009807005.1 | Alphaproteobacteria |
| Aurantiacibacter sp. MUD11 | GCF_026967575.1 | Alphaproteobacteria |
| Aurantiacibacter suaedae | GCF_005434915.1 | Alphaproteobacteria |
| Aurantiacibacter xanthus | GCF_003584015.1 | Alphaproteobacteria |
| Blastomonas fulva | GCF_003431825.1 | Alphaproteobacteria |
| Blastomonas sp. AAP25 | GCF_001295965.1 | Alphaproteobacteria |
| Blastomonas sp. RAC04 | GCF_001713435.1 | Alphaproteobacteria |
| Bradyrhizobium niftali | GCF_004571025.1 | Alphaproteobacteria |
| Caulobacter sp. S45 | GCF_009765965.1 | Alphaproteobacteria |
| Chakrabartia godavariana | GCA 023260075.1 | Alphaproteobacteria |
| Croceibacterium atlanticum | GCF_001008165.2 | Alphaproteobacteria |
| Croceibacterium salegens | GCF_009827435.1 | Alphaproteobacteria |
| Croceibacterium selenioxidans | GCF_018599195.1 | Alphaproteobacteria |
| Croceibacterium soli | GCF_009828065.1 | Alphaproteobacteria |
| Croceibacterium xixiisoli | GCF_009827305.1 | Alphaproteobacteria |
| Emcibacter nanhaiensis | GCF_006385175.1 | Alphaproteobacteria |
| Erythrobacter sp. SG61-1L | GCF_001305965.1 | Alphaproteobacteria |
| Hephaestia sp. MAHUQ-44 | GCF_023806085.1 | Alphaproteobacteria |
| Marinicaulis flavus | GCF_002943565.1 | Alphaproteobacteria |
| Neorhizobium galegae | GCF_008806425.1 | Alphaproteobacteria |
| Neorhizobium sp. T25_13 | GCF_002968675.1 | Alphaproteobacteria |
| Niveispirillum irakense | GCF_000429645.1 | Alphaproteobacteria |
| Niveispirillum sp. BGYR6 | GCF_027568365.1 | Alphaproteobacteria |
| Niveispirillum sp. SYP-B3756 | GCF_009495745.1 | Alphaproteobacteria |
| Novosphingobium acidiphilum | GCF_000429005.1 | Alphaproteobacteria |
| Novosphingobium aerophilum | GCF_014230345.1 | Alphaproteobacteria |
| Novosphingobium aromaticivorans | GCF_900102455.1 | Alphaproteobacteria |
| Novosphingobium arvoryzae | GCF_014652615.1 | Alphaproteobacteria |
| Novosphingobium capsulatum | GCF_031454595.1 | Alphaproteobacteria |
| Novosphingobium decolorationis | GCF_018417475.1 | Alphaproteobacteria |
| Novosphingobium fuchskuhlense | GCF_001519075.1 | Alphaproteobacteria |
| Novosphingobium hassiacum | GCF_014196055.1 | Alphaproteobacteria |
| Novosphingobium humi | GCF_028607105.1 | Alphaproteobacteria |
| Novosphingobium jiangmenense | GCF_015694345.1 | Alphaproteobacteria |
| Novosphingobium lentum | GCF_001590965.1 | Alphaproteobacteria |
| Novosphingobium mangrovi | GCF_022818885.1 | Alphaproteobacteria |
| Novosphingobium mathurense | GCF_900168325.1 | Alphaproteobacteria |
| Novosphingobium organovorum | GCF_022832435.1 | Alphaproteobacteria |
| Novosphingobium ovatum | GCF_009909235.1 | Alphaproteobacteria |
| Novosphingobium pentaromativorans | GCA 003241455.1 | Alphaproteobacteria |
| Novosphingobium piscinae | GCF_014230355.1 | Alphaproteobacteria |
| Novosphingobium pokkalii | GCF_014652855.1 | Alphaproteobacteria |
| Novosphingobium profundi | GCF_018491765.1 | Alphaproteobacteria |
| Novosphingobium sediminicola | GCF_014196525.1 | Alphaproteobacteria |
| Novosphingobium sediminis | GCF_007991615.1 | Alphaproteobacteria |
| Novosphingobium sp. AAP1 | GCF_001295765.1 | Alphaproteobacteria |
| Novosphingobium sp. AAP83 | GCF_001295795.1 | Alphaproteobacteria |
| Novosphingobium sp. AAP93 | GCF_001296055.1 | Alphaproteobacteria |
| Novosphingobium sp. B 225 | GCF_002198665.1 | Alphaproteobacteria |
| Novosphingobium sp. B-7 | GCF_000410615.1 | Alphaproteobacteria |
| Novosphingobium sp. B1 | GCF_900176395.1 | Alphaproteobacteria |
| Novosphingobium sp. BW1 | GCF_008107685.1 | Alphaproteobacteria |
| Novosphingobium sp. CCH12-A3 | GCF_001556015.1 | Alphaproteobacteria |
| Novosphingobium sp. CECT 9465 | GCF_920987055.1 | Alphaproteobacteria |
| Novosphingobium sp. CF614 | GCF_900113255.1 | Alphaproteobacteria |
| Novosphingobium sp. EMRT-2 | GCF_005145025.1 | Alphaproteobacteria |
| Novosphingobium sp. ERN07 | GCF_012641335.1 | Alphaproteobacteria |
| Novosphingobium sp. ERW19 | GCF_012641315.1 | Alphaproteobacteria |
| Novosphingobium sp. ES2-1 | GCF_015169775.1 | Alphaproteobacteria |
| Novosphingobium sp. FKTRR1 | GCF_020404405.1 | Alphaproteobacteria |
| Novosphingobium sp. FSW06-99 | GCF_001519065.1 | Alphaproteobacteria |
| Novosphingobium sp. Fuku2-ISO-50 | GCF_001519055.1 | Alphaproteobacteria |
| Novosphingobium sp. HBC54 | GCF_029436685.1 | Alphaproteobacteria |
| Novosphingobium sp. KACC 22771 | GCF_028736195.1 | Alphaproteobacteria |
| Novosphingobium sp. KN65.2 | GCF_001368935.1 | Alphaproteobacteria |
| Novosphingobium sp. LASN5T | GCF_003856955.1 | Alphaproteobacteria |
| Novosphingobium sp. MBES04 | GCF_000813185.1 | Alphaproteobacteria |
| Novosphingobium sp. MD-1 | GCF_001014975.1 | Alphaproteobacteria |
| Novosphingobium sp. NBM11 | GCF_015390225.1 | Alphaproteobacteria |
| Novosphingobium sp. NDB2Meth1 | GCF_900117425.1 | Alphaproteobacteria |
| Novosphingobium sp. PP1Y | GCF_000253255.1 | Alphaproteobacteria |
| Novosphingobium sp. PY1 | GCF_017312445.1 | Alphaproteobacteria |
| Novosphingobium sp. SG707 | GCF_012275515.1 | Alphaproteobacteria |
| Novosphingobium sp. SG720 | GCF_012275365.1 | Alphaproteobacteria |
| Novosphingobium sp. SG751A | GCF_013149295.1 | Alphaproteobacteria |
| Novosphingobium sp. SL115 | GCF_026672515.1 | Alphaproteobacteria |
| Novosphingobium sp. THN1 | GCF_003454795.1 | Alphaproteobacteria |
| Novosphingobium sp. UBA1939 | GCF_002336885.1 | Alphaproteobacteria |
| Novosphingobium subterraneum | GCF_000807925.1 | Alphaproteobacteria |
| Novosphingobium taihuense | GCF_007830315.1 | Alphaproteobacteria |
| Novosphingobium terrae | GCF_017163935.1 | Alphaproteobacteria |
| Novosphingobium umbonatum | GCF_004005905.1 | Alphaproteobacteria |
| Pararhodobacter zhoushanensis | GCF_003990445.1 | Alphaproteobacteria |
| Parasphingopyxis marina | GCF_014237875.1 | Alphaproteobacteria |
| Parerythrobacter sp. C18 | GCF_030140925.1 | Alphaproteobacteria |
| Pseudoruegeria sp. HB172150 | GCF_013184805.1 | Alphaproteobacteria |
| Rhizobium sp. CF080 | GCF_000282095.2 | Alphaproteobacteria |
| Rhizobium terrae | GCF_003425685.1 | Alphaproteobacteria |
| Rhizorhapis suberifaciens | GCF_014200045.1 | Alphaproteobacteria |
| Roseinatronobacter sp. HJB301 | GCF_028745735.1 | Alphaproteobacteria |
| Sphingobium chungbukense | GCF_001005725.1 | Alphaproteobacteria |
| Sphingobium cupriresistens | GCF_004152865.1 | Alphaproteobacteria |
| Sphingobium jiangsuense | GCF_014196495.1 | Alphaproteobacteria |
| Sphingobium lactosutens | GCF_013393185.1 | Alphaproteobacteria |
| Sphingobium lignivorans | GCF_014203955.1 | Alphaproteobacteria |
| Sphingobium nicotianae | GCF_018603885.1 | Alphaproteobacteria |
| Sphingobium psychrophilum | GCF_012927105.1 | Alphaproteobacteria |
| Sphingobium sp. 3R8 | GCF_020166615.1 | Alphaproteobacteria |
| Sphingobium sp. AntQ-1 | GCF_028538045.1 | Alphaproteobacteria |
| Sphingobium sp. AP50 | GCF_900109095.1 | Alphaproteobacteria |
| Sphingobium sp. B11D3B | GCF_025961735.1 | Alphaproteobacteria |
| Sphingobium sp. B11D3D | GCF_025961755.1 | Alphaproteobacteria |
| Sphingobium sp. B12D2B | GCF_025961775.1 | Alphaproteobacteria |
| Sphingobium sp. B2 | GCF_007693735.1 | Alphaproteobacteria |
| Sphingobium sp. B7D2B | GCF_025961895.1 | Alphaproteobacteria |
| Sphingobium sp. BYY-5 | GCF_022758885.1 | Alphaproteobacteria |
| Sphingobium sp. CAP-1 | GCF_009720145.1 | Alphaproteobacteria |
| Sphingobium sp. LB126 | GCF_002795205.1 | Alphaproteobacteria |
| Sphingobium sp. Leaf26 | GCF_001421665.1 | Alphaproteobacteria |
| Sphingobium sp. SYK-6 | GCF_000283515.1 | Alphaproteobacteria |
| Sphingobium sp. TCM1 | GCF_001650725.1 | Alphaproteobacteria |
| Sphingobium sp. V4 | GCF_029590555.1 | Alphaproteobacteria |
| Sphingobium sp. YR768 | GCF_900111125.1 | Alphaproteobacteria |
| Sphingobium sp. Z007 | GCF_900013445.1 | Alphaproteobacteria |
| Sphingobium terrigena | GCF_003591655.1 | Alphaproteobacteria |
| Sphingobium xanthum | GCF_019737615.1 | Alphaproteobacteria |
| Sphingobium xenophagum | GCF_002288285.1 | Alphaproteobacteria |
| Sphingomonas asaccharolytica | GCF_001598355.1 | Alphaproteobacteria |
| Sphingomonas baiyangensis | GCF_005144715.1 | Alphaproteobacteria |
| Sphingomonas bisphenolicum | GCF_024349785.1 | Alphaproteobacteria |
| Sphingomonas caeni | GCF_026013415.1 | Alphaproteobacteria |
| Sphingomonas canadensis | GCF_026013525.1 | Alphaproteobacteria |
| Sphingomonas hengshuiensis | GCF_000935025.1 | Alphaproteobacteria |
| Sphingomonas lycopersici | GCF_026130585.1 | Alphaproteobacteria |
| Sphingomonas mali | GCF_001598415.1 | Alphaproteobacteria |
| Sphingomonas paucimobilis | GCF_001029575.1 | Alphaproteobacteria |
| Sphingomonas pruni | GCF_001598455.1 | Alphaproteobacteria |
| Sphingomonas psychrotolerans | GCF_002796605.1 | Alphaproteobacteria |
| Sphingomonas sp. AR_OL41 | GCF_029911635.1 | Alphaproteobacteria |
| Sphingomonas sp. HMWF008 | GCA 003061185.1 | Alphaproteobacteria |
| Sphingomonas sp. So64.6b | GCF_014171475.1 | Alphaproteobacteria |
| Sphingomonas sp. SUN019 | GCF_024758705.1 | Alphaproteobacteria |
| Sphingomonas sp. UNC305MFCol5.2 | GCF_000712135.1 | Alphaproteobacteria |
| Sphingopyxis granuli | GCF_001956775.1 | Alphaproteobacteria |
| Sphingorhabdus sp. M41 | GCF_001586275.1 | Alphaproteobacteria |
| Sphingosinicella sp. CPCC 101087 | GCF_004151485.1 | Alphaproteobacteria |
| Sphingosinicella terrae | GCF_003347635.1 | Alphaproteobacteria |
| Caldimonas tepidiphila | GCF_003569765.1 | Betaproteobacteria |
| Glaciimonas soli | GCF_009497155.1 | Betaproteobacteria |
| Massilia cavernae | GCF_003590855.1 | Betaproteobacteria |
| Noviherbaspirillum humi | GCF_900188095.1 | Betaproteobacteria |
| Luteimonas sp. BDR2-5 | GCF_021191695.1 | Gammaproteobacteria |
| Pseudomonas capeferrum | GCF_000731675.1 | Gammaproteobacteria |
| Pseudomonas sp. LS1212 | GCF_024741815.1 | Gammaproteobacteria |
| Pseudomonas sp. R5(2019) | GCF_009905435.1 | Gammaproteobacteria |
| Geodermatophilus sabuli | GCF_900215145.1 | Actinomycetes |
| Lipingzhangella halophila | GCF_014203805.1 | Actinomycetes |
| Pseudonocardia sp. CNS-004 | GCF_001942185.1 | Actinomycetes |
| Pseudonocardia sp. DSM 110487 | GCF_019468565.1 | Actinomycetes |
| Pseudonocardia hierapolitana | GCF_007994075.1 | Actinomycetes |
| Rhodococcus jostii | GCF_900105375.1 | Actinomycetes |
| Rhodococcus opacus | GCF_019856255.1 | Actinomycetes |
| Streptomyces sp. NRRL S-813 | GCF_000718945.1 | Actinomycetes |
| Streptomyces spiralis | GCF_014654675.1 | Actinomycetes |
| Thermopolyspora flexuosa | GCF_006716785.1 | Actinomycetes |
| Bacillus subtilis subsp. subtilis str. 168 | GCF_000155325.1 | Bacilli |
| Paenibacillus sp. tmac-D7 | GCF_006519665.1 | Bacilli |

Construction of in-Frame Deletion Mutants

Gene deletion mutants were constructed using 12444PDC as a parent strain and the pK18mobsacB suicide plasmid. This plasmid was linearized via polymerase chain reaction (PCR) as previously described (23). Regions of N. aromaticivorans genomic DNA ˜1,000 bp upstream and downstream of each gene of interest (Table 7) were amplified via PCR using the primers listed in Table 8 that contain overhanging regions complementary to the ends of linearized pK18mobsacB. NEBuilder HiFi Assembly system (New England Biolabs, Ipswich, MA) was used to insert the amplified fragments into the linearized plasmid, creating a construct in which the genomic regions upstream and downstream of the gene to be deleted are adjacent to each other with no coding region between them. All plasmids used are listed in Table 9.

TABLE 7
N. aromaticivorans genes analyzed in this study and their
associated locus tags. Unnamed alcohol dehydrogenase gene
products (ADHs) and aldehyde dehydrogenase gene products
(ALDHs) investigated are labeled by enzyme class.

| N. aromaticivorans gene | Saro_Locus Tag | SARO_RS Locus Tag |
|---|---|---|
| PcfL | Saro_0796 | SARO_RS03975 |
| FerD | Saro_0797 | SARO_RS03980 |
| LigW | Saro_0799 | SARO_RS03990 |
| LsdD | Saro_0802 | SARO_RS04005 |
| FdhA | Saro_0874 | SARO_RS04375 |
| LigV | Saro_1668 | SARO_RS08360 |
| Putative ADH | Saro_0995 | SARO_RS04970 |
| Putative ADH | Saro_1431 | SARO_RS07175 |
| Putative ADH | Saro_1476 | SARO_RS07405 |
| Putative ADH | Saro_2795 | SARO_RS14810 |
| Putative ADH | Saro_2870 | SARO_RS14555 |
| Putative ADH | Saro_3463 | SARO_RS18190 |
| Putative ADH | Saro_3899 | SARO_RS17300 |
| Putative ALDH | Saro_0060 | SARO_RS02990 |
| Putative ALDH | Saro_1104 | SARO_RS05510 |
| Putative ALDH | Saro_1197 | SARO_RS05980 |
| Putative ALDH | Saro_1410 | SARO RS07070 |
| Putative ALDH | Saro_1967 | SARO_RS09870 |
| Putative ALDH | Saro_2869 | SARO_RS14550 |
| Putative ALDH | Saro_3848 | SARO_RS17045 |

TABLE 8
Primers used to create gcne deletion mutants. Capitalized regions are complementary to
the end of linearized pK18mobsacB. Underlined bases do not match template.

| PCR Reaction | Primers |
|---|---|
| Linearize | pK18msB AseI ampl F: |
| pK18mobsacB | ctgtcgtgccagctgcattaatg (SEQ ID NO: 21) |
| | pK18msB -MCS XbaI R: |
| | gaacatctagaaagccagtccgcagaaac (SEQ ID NO: 22) |
| Amplify region | PcfL pk18 F: |
| upstream of | CGATTCATTAATGCAGCTGGCACGACAGcttttcgcttctccagctcgg (SEQ |
| pcfL | ID NO: 23) |
| | PcfL Del R.2: |
| | cccacccgcaatctcttatttccggtccaactcccatcaatttagtttgtc (SEQ ID NO: 24) |
| Amplify region | PcfL pk18 R.2: |
| downstream of | GTTTCTGCGGACTGGCTTTCTAGATGTTCcttccacgatgaagcgggttgg |
| pcfL | (SEQ ID NO: 25) |
| | PcfL Del F.2: |
| | gacaaactaaattgatgggagttggaccggaaataagagattgcgggtggg (SEQ ID NO: 26) |
| Amplify region | FerD pk18 F: |
| upstream of | CGATTCATTAATGCAGCTGGCACGACAGcggctcgcgcaatttgttagtaag |
| ferD | (SEQ ID NO: 27) |
| | FerD Del R.3: |
| | ctgccgaccgacaccgcaattatatttaatctccggaagccttttgcctg (SEQ ID NO: 28) |
| Amplify region | FerD pk18 R.2: |
| downstream of | GTTTCTGCGGACTGGCTTTCTAGATGTTCcggatcatgcgcaggtagacgtc |
| ferD | (SEQ ID NO: 29) |
| | FerD Del F.3: |
| | caggcaaaaggcttccggagattaaatataattgcggtgtcggtcggcag (SEQ ID NO: 30) |
| Amplify region | LigW pk18 F: |
| upstream of | CGATTCATTAATGCAGCTGGCACGACAGgaaggcgcaatccggagttctcc |
| ligW | (SEQ ID NO: 31) |
| | LigW Del R: |
| | ccctcccggcgctggtcaaaggcaggcttccttcccgggaag (SEQ ID NO: 32) |
| Amplify region | LigW pk18 R: |
| downstream of | GTTTCTGCGGACTGGCTTTCTAGATGTTCtccagtggaagccgggagtgacc |
| ligW | (SEQ ID NO: 33) |
| | LigW Del F: |
| | cttcccgggaaggaagcctgcctttgaccagcgccgggaggg (SEQ ID NO: 34) |
| Amplify region | LsdD pk18 F.4: |
| upstream of | CGATTCATTAATGCAGCTGGCACGACAGgggggctaaccgccagtctctatcttc |
| lsdD | (SEQ ID NO: 35) |
| | LsdD Del R.4: |
| | gcaatacatacaatattgcaaggaggatgccgccgcatgatccagcccggag (SEQ ID NO: 36) |
| Amplify region | LsdD pk18 R.3: |
| downstream of | GTTTCTGCGGACTGGCTTTCTAGATGTTCccaacaggcagccgaggatag |
| lsdD | (SEQ ID NO: 37) |
| | LsdD Del F.4: |
| | ctccgggctggatcatgcggcggcatcctccttgcaatattgtatgtattgc (SEQ ID NO: 38) |
| Amplify region | FdhA pk18 F: |
| upstream of | CGATTCATTAATGCAGCTGGCACGACAGctgacacggatotctcctcaacc |
| fdhA | (SEQ ID NO: 39) |
| | FdhA Del R: |
| | gtaaaccgtgtaaacccgttcaggtattgctacagccctgttaaattgcg (SEQ ID NO: 40) |
| Amplify region | FdhA pk18 R: |
| downstream of | cgcaatttaacagggctgtagcaatacctgaacgggtttacacggtttac (SEQ ID NO: 41) |
| fdhA | FdhA Del F: |
| | cgcaatttaacagggctgtagcaatacctgaacgggtttacacggtttac (SEQ ID NO: 42) |

TABLE 9
Plasmids used in this study.

| Plasmid | Relevant Characteristics | Source |
|---|---|---|
| pK18mobsacB | pMB1ori sacB kan^{R} mobT oriT(RP4) lacZa | (52) |
| PVP302K | lac promoter lacI, Tev site rtxA (V. cholera) kan^{R}; | (8) |
| | coding sequence for 8 × His-tag | |
| pK18mobsacBΔpcfL | pK18mobsacB containing genomic regions flanking | This study |
| | pcfL | |
| pK18mobsacBΔlsdD | pK18mobsacB containing genomic regions flanking | This study |
| | lsdD | |
| pK18mobsacBΔferD | pK18mobsacB containing genomic regions flanking | This study |
| | ferD | |
| pK18mobsacBΔligW | pK18mobsacB containing genomic regions flanking | This study |
| | ligW | |
| pK18mobsacBΔfdhA | pK18mobsacB containing genomic regions flanking | This study |
| | fdhA | |
| PVP302K-PcfL | pVP302K containing codon optimized PcfL | This study |
| PVP302K-PcfL-NTag | pVP302K containing codon optimized PcfL | This study |
| | downstream of His-tag coding sequence and Tev | |
| | protease site | |
| PVP302K-LsdD | pVP302K containing codon optimized LsdD | This study |
| PVP302K-FerD | pVP302K containing codon optimized FerD | This study |
| PVP302K-FerD-NTag | pVP302K containing codon optimized FerD | This study |
| | downstream of His-tag coding sequence and Tev | |
| | protease site | |
| PVP302K-LigW | PVP302K containing codon optimized LigW | This study |
| PVP302K-FdhA | pVP302K containing codon optimized FdhA | This study |
| pVP302K-LigV | pVP302K containing codon optimized LigV | This study |
| PVP302K-0995 | pVP302K containing codon optimized Saro_0995 | This study |
| PVP302K-1431 | pVP302K containing codon optimized Saro_1431 | This study |
| PVP302K-1476 | pVP302K containing codon optimized Saro_1476 | This study |
| PVP302K-2795 | pVP302K containing codon optimized Saro_2795 | This study |
| pVP302K-2870 | pVP302K containing codon optimized Saro_2870 | This study |
| pVP302K-3463 | pVP302K containing codon optimized Saro_3463 | This study |
| PVP302K-3899 | pVP302K containing codon optimized Saro_3899 | This study |
| pVP302K-0060 | pVP302K containing codon optimized Saro_0060 | This study |
| PVP302K-1104 | pVP302K containing codon optimized Saro_1104 | This study |
| PVP302K-1197 | pVP302K containing codon optimized Saro_1197 | This study |
| PVP302K-1410 | pVP302K containing codon optimized Saro_1410 | This study |
| PVP302K-1967 | pVP302K containing codon optimized Saro_1967 | This study |
| PVP302K-2869 | pVP302K containing codon optimized Saro_2869 | This study |
| PVP302K-3848 | pVP302K containing codon optimized Saro_3848 | This study |

These plasmids were transformed into E. coli NEB5α by heat shock. Plasmids were isolated from NEB5αcultures using the QIAprep Miniprep Kit (Q)iagen, Germantown, NID) and the insert regions of the plasmids were amplified and submitted for Sanger sequencing at Functional Biosciences (Madison, WI) or the, University of Wisconsin-Madison DNA Sequencing core facility. Once the sequences of these plasmids were verified, they were transformed via heat shock into E. coli WM46026, which served as a conjugal donor to mobilize the plasmids into N. aromaticivorans as previously described (16), except that the SMB minimal medium contained 1 g/L glucose.

### Construction of Protein Expression Strains

Plasmids for recombinant protein expression were constructed using pVP302K, which was linearized via PCR using the primers listed in Table 10. Codon optimized (Benchling Biological Software) gBlocks (Table 11) of genes of interest (Table 7) for heterologous recombinant protein expression were obtained from Integrated DNA Technologies (San Diego, California) and amplified by PCR using the primers in Table 9 that contain overhanging regions complementary to the ends of linearized pVP302K. NEBuilder HiFi Assembly system was used to insert the amplified gBlocks into the linearized plasmid, yielding untagged expression plasmids for all genes as well as N-terminal His-tagged constructs with a TEV-protease cleavage site between the tag and the protein for PcfL and FerD. All plasmids used are listed in Table 9.

These pVP302K derivatives were transformed into E. coli NEB5α and their sequences were verified as described above. They were then transformed into E. coli B834 by heat shock.

TABLE 10
Primers used to create recombinant protein expression plasmids. Capitalized
DNA sequences are complementary to the end of linearized pVP302K.

| PCR Reaction | Primers |
|---|---|
| Linearize | PVP302K No His Lin F: |
| PVP302K with | taacagaaagccgaaaataacaaagttagc (SEQ ID NO: 43) |
| no His-tag | PVP302K No His Lin R: |
| | catggttaatttctcctctttaatgaattctgtg (SEQ ID NO: 44) |
| Linearize | PVP302K N-Term Lin F: |
| PVP302K with | cagaaagccgaaaataacaaagttagcctgag (SEQ ID NO: 45) |
| an N-terminal | PVP302K N-Term Lin R: |
| His-tag | tgcgatcgcgctctgaaaatacag (SEQ ID NO: 46) |
| Amplify PcfL | pVP302K No His PcfL HiFi F: |
| gBlock (no His- | TAAAGAGGAGAAATTAACCATGtccgatagcaatcagattgcc (SEQ ID |
| tag construct) | NO: 47) |
| | PVP302K No His PcfL HiFi R: |
| | TGTTATTTTCGGCTTTCTGTTAtttccgcgcattttcgc (SEQ ID NO: 48) |
| Amplify FerD | PVP302K No His FerD HiFi F: |
| gBlock (no His- | TAAAGAGGAGAAATTAACCATGactgcgtacccttctctcc (SEQ ID |
| tag construct) | NO: 49) |
| | pVP302K No His FerD HiFi R: |
| | TGTTATTTTCGGCTTTCTGTTAcccttcatgtaccgctttgg (SEQ ID NO: 50) |
| Amplify LigW | PVP302K No His LigW HiFi F: |
| gBlock | TAAAGAGGAGAAATTAACCATGacacaagacctgaagaccgg (SEQ ID |
| | NO: 51) |
| | pVP302K No His LigW HiFi R: |
| | TGTTATTTTCGGCTTTCTGTTAaagtttaaaccatttttcagcgttgg (SEQ ID |
| | NO: 52) |
| Amplify LsdD | PVP302K No His LsdD HiFi F: |
| gBlock | TAAAGAGGAGAAATTAACCATGgctcaatttccgaataccccaag (SEQ ID |
| | NO: 53) |
| | PVP302K No His LsdD HiFi R: |
| | TGTTATTTTCGGCTTTCTGTTAtgcggccaggaccttttc (SEQ ID NO: 54) |
| Amplify FdhA | PVP302K No His LsdD HiFi F: |
| gBlock | TAAAGAGGAGAAATTAACCATGctaagcgacaggcacgtcaaag (SEQ ID |
| | NO: 55) |
| | PVP302K No His LsdD HiFi R: |
| | TGTTATTTTCGGCTTTCTGTTAgaacaccactactgaacgaatcgatttac (SEQ |
| | ID NO: 56) |
| Amplify PcfL | pVP302K-N PcfL HiFi F: |
| gBlock (N- | AAATCTGTATTTTCAGAGCGCGATCGCAtccgatagcaatcagattgccg |
| terminal His-tag | (SEQ ID NO: 57) |
| construct) | PVP302K-N PcfL HiFi R: |
| | GGCTAACTTTGTTATTTTCGGCTTTCTGttatttccgcgcattttcgcg (SEQ |
| | ID NO: 58) |
| Amplify FerD | PVP302K-N FerD HiFi F: |
| gBlock (N- | AAATCTGTATTTTCAGAGCGCGATCGCAactgcgtacccttctctccacatg |
| terminal His-tag | (SEQ ID NO: 59) |
| construct) | PVP302K-N FerD HiFi R: |
| | GGCTAACTTTGTTATTTTCGGCTTTCTGttacccttcatgtaccgctttggtgac |
| | (SEQ ID NO: 60) |
| Amplify LigV | LigV Exp LigV F: |
| gBlock | CATTAAAGAGGAGAAATTAACCatgcagtttgaacgtatcaatccgatg (SEQ |
| | ID NO: 61) |
| | Exp LigV R: |
| | GTTTAAACTATTAATGATGATGttaaattggatagtgacctggttggg (SEQ |
| | ID NO: 62) |
| Amplify | 0995 Exp F: |
| Saro_0995 | CATTAAAGAGGAGAAATTAACCatgaaagccgccgtactc (SEQ ID |
| gBlock | NO: 63) |
| | 0995 Exp R: |
| | GTTTAAACTATTAATGATGATGttattgatcaaacacaataacagaacg (SEQ |
| | ID NO: 64) |
| Amplify | 1431 Exp F: |
| Saro_1431 | CATTAAAGAGGAGAAATTAACCatgacaatcaatacaattcgcgtacg (SEQ |
| gBlock | ID NO: 65) |
| | 1431 Exp R: |
| | CGTTTAAACTATTAATGATGATttaacaaaaatgacggcagctctg (SEQ ID |
| | NO: 66) |
| Amplify | 1476 Exp F: |
| Saro_1476 | CATTAAAGAGGAGAAATTAACCatgttgggacgtgcatcgg (SEQ ID |
| gBlock | NO: 67) |
| | 1476 Exp R: |
| | GTTTAAACTATTAATGATGATGttacgtgatcgtoggatcgatc (SEQ ID |
| | NO: 68) |
| Amplify | Exp 2795 F: |
| Saro_2795 | CATTAAAGAGGAGAAATTAACCatggcggcaattaatcttccccg (SEQ ID |
| gBlock | NO: 69) |
| | Exp 2795 R: |
| | GTTTAAACTATTAATGATGATGttagccaaagacttcggcatagaggc (SEQ |
| | ID NO: 70) |
| Amplify | Exp 2870x F: |
| Saro_2870 | CATTAAAGAGGAGAAATTAACCatgcgattgaaagtactgggacttatgg |
| gBlock | (SEQ ID NO: 71) |
| | Exp 2870 R: |
| | GTTTAAACTATTAATGATGATGttagccacctttggcttctaaag (SEQ ID |
| | NO: 72) |
| Amplify | Exp 3463 F: |
| Saro_3463 | CATTAAAGAGGAGAAATTAACCatgattccgcatggtgaacattcaatgctg |
| gBlock | (SEQ ID NO: 73) |
| | Exp 3463 R: |
| | GTTTAAACTATTAATGATGATGttatggcaccaaaaccagagcgccac (SEQ |
| | ID NO: 74) |
| Amplify | Exp 3899 F: |
| Saro_3899 | CATTAAAGAGGAGAAATTAACCatggacgcatacgctgcaattatc (SEQ ID |
| gBlock | NO: 75) |
| | Exp 3899 R: |
| | GTTTAAACTATTAATGATGATGttacattttgagaatggcttttatcgcttttc |
| | (SEQ ID NO: 76) |
| Amplify | Exp 0060 F: |
| Saro_0060 | CATTAAAGAGGAGAAATTAACCatgtctacacagcctgcaaccatagctg |
| gBlock | (SEQ ID NO: 77) |
| | Exp 0060 R: |
| | GTTTAAACTATTAATGATGATGttatggacgagtttgcccgcttcc (SEQ ID |
| | NO: 78) |
| Amplify | Exp 1104 F: |
| Saro_1104 | CATTAAAGAGGAGAAATTAACCatgcgcgaacggctacagcaatacattg |
| gBlock | (SEQ ID NO: 79) |
| | Exp 1104 R: |
| | GTTTAAACTATTAATGATGATGttaggcaggcaggccgctgatcg (SEQ ID |
| | NO: 80) |
| Amplify | Exp 1197 F: |
| Saro_1197 | CATTAAAGAGGAGAAATTAACCatgactgcccctaccgcc (SEQ ID |
| gBlock | NO: 81) |
| | Exp 1197 R: |
| | GTTTAAACTATTAATGATGATGttactgctgatgacgatatacagcc (SEQ ID |
| | NO: 82) |
| Amplify | Exp 1410 F: |
| Saro_1410 | CATTAAAGAGGAGAAATTAACCatgggttaccgggttgtagtggtg (SEQ ID |
| gBlock | NO: 83) |
| | Exp 1410 R: |
| | CATTAAAGAGGAGAAATTAACCatgcagtttgaacgtatcaatccgatg (SEQ |
| | ID NO: 84) |
| Amplify | Exp 1967 F: |
| Saro_1967 | CATTAAAGAGGAGAAATTAACCatggcgatcaaagttgcgataaac (SEQ |
| gBlock | ID NO: 85) |
| | Exp 1967 R: |
| | GTTTAAACTATTAATGATGATGttaaaggaatttcgccattgctcc (SEQ ID |
| | NO: 86) |
| Amplify | Exp 2869 F: |
| Saro_2869 | CATTAAAGAGGAGAAATTAACCatgaatgacatgactaccatctc (SEQ ID |
| gBlock | NO: 87) |
| | Exp 2869 R: |
| | GTTTAAACTATTAATGATGATGttacatttgaataattactgttttagtctc (SEQ |
| | ID NO: 88) |
| Amplify | Exp 3848 F: |
| Saro_3848 | CATTAAAGAGGAGAAATTAACCatggctacgcagttgagaagtgcag (SEQ |
| gBlock | ID NO: 89) |
| | Exp 3848 R: |
| | GTTTAAACTATTAATGATGATGttactgatcgaacattccggtacgacc (SEQ |
| | ID NO: 90) |

TABLE 11
gBlocks of N. aromaticivorans genes codon optimized for E. coli and
used to create heterologous protein expression constructs.

| gBlock | Sequence |
|---|---|
| PcfL | ccgatagcaatcagattgccgcgcttgaaagtcgcctgaatgacctcgaa |
| gBlock | aggcgactgacggttagagaggacgagctggacgtacgcaaactccagca |
| | tttatacggttatctgattgataaatgcatgtataacgagacagttgacc |
| | tgttcacagaagatggggaagtgcggttctttggtggcgtatggaaaggc |
| | aaggagggcatccgccgtttgtacgttgaacgttttcagaaacgtttcac |
| | ctatggcaataacggcccgattgatgggttcctgttagatcatccacaac |
| | ttcaagatattattcacgtgcaggatgatggggtcacggctttgggccgc |
| | gcgcgttccatgatgcaagccggtcgccacaaggattatgagggagatgc |
| | acctcatctgaaagcgcgtcagtggtgggaaggtggtatatacgaaaaca |
| | cttataaaaaagtggatggcgtgtggcgtatgcatatcctaaactacatg |
| | ccgatctggcacgcagattttgaaagcggctgggccaataccccgcacga |
| | atacgttccttttcccaaagtcacctatccagaagacccgactggaccgg |
| | atgaactgattgctgaccattggttatggccgacccataagctgaacccc |
| | tttcacatgaaacatccggtgacgggtgaggaaatggtcgcacagcgctg |
| | gcagggtgacatcgatcgcgaaaatgcgcggaaataa |
| | (SEQ ID NO: 91) |
| FerD | actgcgtacccttctctccacatgattattgacggtgcccgtgtcagcgg |
| gBlock | cggaggacgtcgcacccacgcggtcgtcaatccggctaccggagagacca |
| | tcggtgaactgccgctggcagaagttgcagatctggatcgagcgttagaa |
| | gtagcggcgaagggcttccgtatttggcgtgacagcacaccgcagcagcg |
| | cgcagccgtgttacagggcgcggcccggctgatgctggaacggcaagagg |
| | atctcgctcgcatagccacgatggaagaaggtaaaaccctgcccgaggcg |
| | cgcatcgaagttctgatgaacgtgggcctgttcaatttttacgctggaga |
| | agtatttcgtttatatggccgaaccctagtgcgccctgcgggtcagagaa |
| | gcacgatcacgcatgaaccggtagggccggtggccgcctttgctccgtgg |
| | aactttccgcttgggaatccaggtcgcaaactgggcgcgccaattgccgc |
| | cggttgctcggtgattctaaaagcggcggaagaaacgccggcttcagcgt |
| | taggggtgctgcaatgtctgctggatgctggcctgcctaaagaagtggcc |
| | caggctgtgttcggtgtgcctgacgaggtgagtcgccacctgttgggcag |
| | ttccgttatccgcaagctctcgtttacaggttctaccgtcatcggcaagc |
| | atctgatgcgacttgcagccgacaacatgttgcgtacaactatggagctt |
| | ggcggccatggtcctgtcttagttttcggtgatgcagatattgacaaagc |
| | gctcgataccatggcagcttccaaatatcgtaacgcgggccaagtttgtg |
| | tttcaccaaccagatttatagtggaagaaagcgtgttcgaacgttttcgt |
| | gatggttttgcagagcgtgtcggtcggatcaaagttggaaatggtttgga |
| | tcaggatgcgcagatgggaccgatggcaaatgcccgccgcccggaggcga |
| | tggatcgtctgatcggggacgccgtgactcgcggcgcaaggttgcatact |
| | gggggcgaacgtgtcggcaacgccggctatttttatgcccccacggttct |
| | gagtgaagtaccgctggacgcggctattatgaacgaagaaccgtttggcc |
| | cggtagctctgattaatccattcggcggtgaggaagcgatgatcgccgaa |
| | gcaaaccgtctgccgtatggcttggcagcctacgcatggacagatagcgc |
| | ggcgcgggcaaaacgcttagcacgcgagattgagacggggatgctggggc |
| | ttaattctaccatgattggcggcgcggattcgccattcggtggggtgaaa |
| | tggtccggacacggttcagaggacggtcccgaaggtgttatggcctgcct |
| | tgtcaccaaagcggtacatgaagggtaa (SEQ ID NO: 92) |
| LigW | acacaagacctgaagaccggcggggagcagggttacctgcgtatcgccac |
| gBlock | cgaagaagctttcgccacgcgagaaatcattgatgtctacctgcgcatga |
| | tacgcgatggaactgctgataaaggtatggtatcattgtggggcttttat |
| | gcccagtccccttcagagcgcgccacccagatcttagaacgtctgttaga |
| | tcttggcgagcggcgtattgcagatatggatgcgacaggcattgacaagg |
| | ctattctagcgctgacctcgccgggcgtacagccgctgcatgacttagat |
| | gaagcacggacgctcgcaacccgtgcaaatgatactcttgccgatgcgtg |
| | ccaaaagtatccagaccgatttattggaatgggcaccgtggccccgcagg |
| | atccggaatggagtgcgcgcgaaattcatcgtggtgcaagggaactgggt |
| | tttaagggcatccagatcaacagccacacgcaagggcgctacttggatga |
| | ggaattctttgatccgatattccgtgccctcgttgaagtcgaccagccgc |
| | tgtatattcatcctgccacttcgccagattccatgatcgatccgatgttg |
| | gaagcgggcctggacggtgcaatcttcggcttcggtgtggagacgggcat |
| | gcatctgctgcgcctgatcacgattgggattttcgacaaatatcccagct |
| | tgcaaattatggttgggcacatgggcgaggcgctgccctactggctctat |
| | agactggattatatgcaccaggctggtgtgcgctctcagcgctatgaacg |
| | tatgaaaccactgaaaaaaaccatcgaaggttatcttaaaagcaacgtgt |
| | tagtgacaaattctggagtcgcgtgggaacctgcgattaaattttgtcag |
| | caagtaatgggtgaggatcgggttatgtacgcgatggactacccgtatca |
| | gtacgttgcagacgaagtgcgtgcgatggatgccatggacatgagtgcgc |
| | aaacgaaaaaaaaattttttcagaccaacgctgaaaaatggtttaaactt |
| | taa (SEQ ID NO: 93) |
| LsdD | atggctcaatttccgaataccccaagcttcacgggattcaacacgccgtc |
| gBlock | tcggattgaggcggatattgcagatctggcccacgaaggtacgattccgc |
| | aagggttaaacggcgcattttatcgtgtccagcccgatccgcagtttcct |
| | ccacgcctcgatgatgacattgcctttaacggagacgggatgattacccg |
| | attccatatacatgatggccaggtcgacttccgtcaacgttgggcgaaaa |
| | ccgataaatggaaactggaaaacgcggccggaaaagccctgtttggtgcc |
| | taccgcaacccactgaccgatgacgaggcggttaaaggcgagatccgttc |
| | gaccgccaacactaacgccttcgttttcggtggcaaactgtgggcgatga |
| | aagaggacagtccagcactcgtaatggatccggcgacgatggaaaccttc |
| | gggttcgaaaagttcggcggtaaaatgacaggccagacctttactgccca |
| | tccgaaggtagatccgaaaaccggcaatatggtagcgatcggttatgctg |
| | caagcgggttgtgcacagatgatgtgacctacatggaagttagtccggag |
| | ggtgaattagtacgcgaagtgtggttcaaagtgccgtattattgcatgat |
| | gcacgacttcggcattacagaggattacctcgtgctgcacattgttcctt |
| | ccatcggaagctgggaaagattagaacagggcaaaccgcactttggcttt |
| | gatactactatgccggttcacctaggtatcattccgaggcgtgacggtgt |
| | gcgccaggaagatatccgttggttcacgcgggataattgttttgccagtc |
| | atgtactgaatgcttggcaagaagggaccaaaattcactttgtgacttgc |
| | gaagcgaaaaacaacatgtttcctttctttccagatgtccatggcgcgcc |
| | ctttaacggtatggaggcaatgtcacatcctacggactgggtggtcgaca |
| | tggcaagcaacggcgaggactttgctgggatcgtgaagctttccgataca |
| | gctgcagaatttcctcgcatcgacgaccggtttaccggccagaaaacccg |
| | ccatggttggttcttagaaatggatatgaaacgaccagtggaattgcgcg |
| | gtggttcagcgggcggcctgctgatgaattgtctgtttcacaaggacttc |
| | gaaacgggtcgtgaacagcattggtggtgcggcccggtttcgtctcttca |
| | ggagccgtgttttgttccgcgcgcgaaagatgcccccgaaggtgatggat |
| | ggattgtgcaagtttgtaatcgtctggaagaacagcgttccgatttgctg |
| | atatttgatgcgctggatattgagaaaggcccggtggctacggtcaatat |
| | ccccatccgcctgcgctttggcttgcatggtaattgggcgaatgcagacg |
| | aaattgggcttgcggaaaaggtcctggccgcagcgatcgcaggaagcgaa |
| | aatctgtattttcagagcgcattggcacatcaccatcatcaccatcacca |
| | ttaa (SEQ ID NO: 94) |
| FdhA | ctaagcgacaggcacgtcaaagggagaccgcatgaaatgaaaacacgcgc |
| gBlock | cgcagttgcgtttgcgccaaagcaaccgttggaaattgtagaactggatc |
| | tggaaggtcccaaagctggggaagttctggttgagattatggcgactgga |
| | gtgtgtcacaccgatgcatatacgttagacgggttcgacagcgaaggcat |
| | tttccctagcgtgctgggtcatgaaggtgccggtatcgtgcgcgaagtgg |
| | gccctggggtaacttccgtgaaacctggcgatcatgtgatcccgctctat |
| | acgccggaatgtcgccagtgcaaatcgtgcttgtcgggtaagaccaacct |
| | gtgcaccgctattcgcgccacgcaagggcagggcctgatgcccgatggca |
| | ccagtcgtttttcttacaaaggccagaccgtgttccactacatgggttgc |
| | agtacattctctaattttacagttctgccagagatcgcggttgcaaagat |
| | tcgcgaggatgcgccgtttaaaacctcatgttatattggctgtggcgtga |
| | cgacgggtgttggcgcggtgattaacactgctaaagtacaggtcggtgac |
| | aacgtcgtggtctttggattaggcggcataggtctcaatgttattcaggg |
| | agcgcggcttgccggtgcagggaaaatcattggcgtcgatatcaatccag |
| | atcgggaggaatggggccgtaaatttggcatgactgactttctgaatagt |
| | aagggcatgagccgcgaggacgtagttgctaaagtcgtcgccatgaccga |
| | tggcggtgcggactatacctttgatgccaccggtaataccgaagtgatgc |
| | gtacggcgcttgaagcatgccatcgtggttggggaacctccataatcatt |
| | ggtgtggcagaggcgggtaaagaaattagcacgcgtccgttccaattagt |
| | tactggccgtaactggcgaggcacggccttcggaggcgccaaggggcgca |
| | cagatgttccgaaaattgtagatatgtacatgaccggaaaaatcgaaatc |
| | gatccgatgatcacccatgtcatggggctggaagagatcaacacagcatt |
| | tgatctgatgcacgctggtaaatcgattcgttcagtagtggtgttctaa |
| | (SEQ ID NO: 95) |
| LigV | cagtttgaacgtatcaatccgatgacaggggcagtagcctcgcaggcaga |
| gBlock | ggccatgaaagcgtcggacattccttccattgctgcccgcgcaggacagg |
| | cctttccggcgtgggcagcgatgggccccaacgcacgtcgcggcgtactg |
| | atgaaggggctgcggcgttggaagcgcgggctgatgctttcgtcgaagcc |
| | atgatgggcgaaatcggcgcgactagagggtgggcgctgtttaaccttgg |
| | ccttgcagcaagcatggtgcgcgaagccgccgcgctgaccactcaaatct |
| | ctggagaggttattccatctgacaaaccggggtgtatttcgatggctctg |
| | cgcgaaccggttggtgtgattttgggcatcgcgccgtggaatgcgccgat |
| | tatccttggggtgcgcgcaattgccgtgccgcttgcctgcggtaacgcgg |
| | tgatattaaaagcaagcgaaacatgtccgcgaacccacgcgctcatcatc |
| | gaggcctttgctgaagcaggtttcccagaaggcgtggttaatgtagtgac |
| | gaacgcgcctgcagatgcagcggaagtggtcggggcgctgattgatgcgc |
| | cggaagtgcgtcgtataaactttaccggtagtactaatgtaggcaggatt |
| | atcgcaaaacgggggccgagcatttgaaaccctgtttactcgaactgggc |
| | ggtaaagcaccgttaatagttctggatgatgcggatctagacgaagcggt |
| | caaagctgcggcttttggcgccttcatgaaccaagggcagatttgcatgt |
| | caacggagcggatcatcgttgtagatgccgttgccgatgcattcgcagat |
| | aaattcaaggccaaggtcgcctccatggctgtaggcgacccgcgtgaggg |
| | tacgaccccgttgggtgcagttgtcgacgctaaaactgtcgctcattgcc |
| | gtagcttaattgacgatgccctggcaaaaggtgcccgtctgctgaccggc |
| | ggtgaaaccacgcacaatgtgctcatgcccgcccatgtcgtagatggcgt |
| | gacgcaggatatgaagctgttccgcgatgagagctttggcccagtggtgg |
| | gcgtgattcgcgcgcgcgacgaagctcatgccattgaactggcgaacgac |
| | agtgaatatggactgtcagcggctgttttcacacgtgacacagcgcgcgg |
| | cctgcgagttgcccgccagatccgtagcggtatttgccatgttaatggac |
| | ctaccgtccacgatgaggcgcagatgccttttggtggagtgggtgcgtcc |
| | ggctacggtcgttttgggggtaaagccggcatcgatagttttaccgagct |
| | gagatggattacgatggaaacccaaccaggtcactatccaatttaa |
| | (SEQ ID NO: 96) |
| Saro_0995 | aaagccgccgtactcgtcgaaccgggtaaaccgctggatattcagcattt |
| gBlock | aagcgtgagtaaacccggccctcatgaagtccttatacgcacagcagcct |
| | gcgggctgtgccatagtgacttgcacttcatcgaaggtgcctatccacat |
| | ccgctgccggctgtgccagggcacgaggctgctgggattgtggaagcggt |
| | aggttcagaagtgcgcacagtaaaagtgggtgacgctgttgttacctgcc |
| | tgtccgcgttctgtggtcattgcgagttttgcgtgaccggccggatgtcg |
| | ctgtgtcttggtggcgatactcggcgcggtgcgggtgaggcacctcgctt |
| | gacacgcaccgacgatggaagcgcagtgaaccagatgctcaacctatcgg |
| | cctttgcagaacaaatgctggttcacgaacatgcctgtgttgcgatcaat |
| | cccgagatgccgctcgatagagctgcggttatcggctgtgcggtaaccac |
| | tggcgcgggtgcggtgtttaatgctgcgaaactgaccccaggagagacgg |
| | tatgcgttgtcggctgtggcggcgtaggcttagcaacggtcaatgccgcg |
| | aaaattgccggggcaggccgtattatcgctgtggatccgatgccggaaaa |
| | acgcgaactggccatgaaactgggtgcgaccgatgtgatggacgcgggac |
| | ccgatgctgcggcacagatcgttgaaatgacgaaaggcggcgttcaccat |
| | gcgatcgaggccgtggggcgtcctgcatctggcgaccttgcggtcgcgac |
| | gctgcgtcgtgggggcaccgccacgattttaggtatgatgccgctggcac |
| | acaaggtcggattatcagcgatggatctgctgagcgataagaagctgcag |
| | ggtgcaattatgggccgcaaccacttcccagtggatctgccgcgactggt |
| | cgacttctacatgcgtggcttgttggatctagacactatcattgccgaaa |
| | ggattccgcttgaagggataaacgatggttttgaaaaaatgaaacaggga |
| | cattccgcccgttctgttattgtgtttgatcaataa |
| | (SEQ ID NO: 97) |
| Saro_1431 | acaatcaatacaattcgcgtacgttcgccggccactctcgacaccttaaa |
| gBlock | tttcgatacgctgacggattgtggacaaccgggaacgagcgaaatccgca |
| | ttcgtctgcgcgcaacttctctgaacttccactactacgcgatgattacc |
| | agaatgctgccggctgcaacaagtcgaattcctatgtctaacggcgcctg |
| | acaggttttcggggtgtgcgatggcgtgaccaaattccaggcgcgtaacg |
| | cagttatctcgacctttttcaccgacaggaacgccggtccgccacagtca |
| | gccgcgtttacgaccgtcacggctgatgggattaatcgctacgcgcggga |
| | agaagtggtggccccggctcattggtttacccgcgcgccgttatgctata |
| | gtcacgcaaaagccgccacgctgacctgcgcgggccttactgcatggcgt |
| | gctttgttcatagataacgctatcaagccgggcgacacggtcttggtgca |
| | gggcactggcagcgtttcggttttcgcgctgcagttaacaaaggcggcat |
| | gcgcgcgtgtcatcgcaacgagttcctcccaccagtaactgaaacgcctg |
| | cgcagccttagagcgaataaaaccataaactataaaacgcaaacctcacg |
| | ggggatgcagacactagatttcactgccggtatttgtgtacactgtattg |
| | tcgagattagccggcccggtacgtttcatcaagcgatgatgtccacccgc |
| | gtgcgtgctcatatcgcgctgatcggtgttctcgcgcgttttgcgggtcc |
| | agtttaaaccactttgctgatggcacagaatctgcgcgtataaggcctta |
| | ccgtggcctcacgtaccaatcatctgcgaatgattcccggtatcgaggca |
| | aaccgtatccaacctgtcattcaccgccattttccatttccgtattttgc |
| | cgctgcctttcgccatcaacagagctgccgtcatttttgttaaatcgtga |
| | ttgacatttga (SEQ ID NO: 98) |
| Saro_1476 | ttgggacgtgcatcggtgctggtaaaaccgaaccaactggagacgtggga |
| gBlock | tgttaaagtagccgatccggaaccgggcggtgccttagtttcgattgtgc |
| | tgggtggggtatgcgggagcgacgtccatatattgaccggcgaggctggc |
| | gtgatgccgtttccgatcattctgggacatgagggcgtgggaaggatcga |
| | aaaactggggcacggcgtcagcactgattacgctggtgaggaacttaaac |
| | ccggcgatctggtatattggtcgccgattgctctgtgtcatcgatgttat |
| | tcctgcaatgttctcgatgaaacaccttgcgaaaatacccagtttttcga |
| | agatgcttccaagccgaactggggttcatacgcagattatgcatggctgc |
| | ccaacggtatgccgttctataaactgccagcccaagcgcagcctgaagcg |
| | gttgctgcgcttggctgtgcacttccaaccgccctgcgcggctttgatcg |
| | ctgcggcagtgttagagtgggtgaaactgtggttgtccaaggtgcaggcc |
| | ctgtcggcctgtctgcagtgctcgtggcggcgcaggccggggcgcgtgac |
| | gtgattgttattgacggttcaccacttcgtcgcgaagcggctaccgcatt |
| | gggtgcctctctgacgattggcttagatgtcgcgcctgaggaacggcgcc |
| | ggatgatttacgatcgcgttggtcgcaatggtcccaatgtagtcatcgag |
| | gcagccggagttctgccagcgtttccggaaggggtggacctgaccggtaa |
| | ccacggccgttacattgtgctaggattgtggggcgcaatagggacccagc |
| | cgatcagcccgcgcgacttaacaatcaaaaacctgactatcgctggtgcg |
| | accttccctaaaccaaaacattattatcaggccttgcatttagcgacggc |
| | cctgcaggaccgtgtaccgttagccggtctggtgagccaccgttttggcg |
| | tcagccaggcgggcgaagcgctgagtctcaccaagagtgggacagcgatt |
| | aaggccgtgatcgatccgacgatcacgtaa (SEQ ID NO: 99) |
| Saro_2795 | gcggcaattaatcttccccgcgtgattcgtgctggtgggggtgcattagc |
| gBlock | cgaactgcccgatgcaatggcgcagtgcggcctttcacgcccgttcgtgg |
| | tgaccgatgcattcttagtgcaaagcgggatggtcgctcggatgttagag |
| | gttctggacggcgctgggattgcggccacggtcttcgatgctacggtacc |
| | tgatccgactgttgctgtggtagaacaggcgcttggcgcattgcgagagg |
| | cggaatgtgattgtgtgatcgggtttggaggtggtagcccgatcgacacc |
| | agtaaagccattgccgccctggcgctggaaccgcgtgcagttcaatccat |
| | gaaggcaccagcgacgaccgacgtcccgggtctgccgatcattgccgtcc |
| | cgacgaccgccggcaccggctcggaggcgactaaatttacaatcgtgacc |
| | gatgaggcgacgagtgaaaaaatgctctgcgcaggtctggccttcctgcc |
| | tactatagccattgtagatttcgagctgaccatgggcaaaccggctcggc |
| | taactgccgacacaggtattgattcgctgacacatgcgattgaggcctat |
| | gtttctaagaaagccaatccgtttagtgatgctatggcgatctcggcgat |
| | gaaactgatcgcgccgaacattcgcaccgcctgcgccgaacccggaaacc |
| | gtgctgcacgcgaagcgatgatgattggcgcgcaccatgccggtattgcg |
| | ttttccaacgctagcgttgcactggtgcacggtatgagccgcccaatcgg |
| | cgcattctttcatgtgccgcacggattgtccaacgcaatgttgctgcctg |
| | cgattaccgcgttttccgctccgtcagcgttaccacgttacgccgattgt |
| | gcccgtgcgatgggtgtagctttggaaagcgaaggcgaccagtctgccgt |
| | tgcaaggctgctcgacgaactggcggcgctgaacgcagaccttagtgtcc |
| | cgacgccgcagtcgcatgggatcagcgctgatcgttggtttgaagtagtg |
| | cctgaaatggcgagacaggcaatagcatcaggctctccaggcaataatcc |
| | acgcgttcctgatgcggcggaaatcgagcgcctctatgccgaagtctttg |
| | gctaa (SEQ ID NO: 100) |
| Saro_2870 | cgattgaaagttctgggacttatggcagcactgctgccgctggcggcttg |
| gBlock | taacatcaaaagcgagggtggaggggatgcagtcgccaacgctggagtca |
| | cagatgccctgattgcccaagcgcccgaaggcgaatggctgagctatggc |
| | cgcgattatggggaacaacgcttttcaccgttgacccaaattaatgatgg |
| | taacgtcgggcagttgggtcttgcctggtttcatgacctggagactgcgc |
| | gcgggcaagaagcgacgccgctgatgcatgatggtacgttatatatctcg |
| | actgcgtggtcaatggtgaaagcgttcgatgcaaaaaccggcgcgctgaa |
| | atggagttacgatcccgaagtaccgcgtgaaacgctggtgcgcgcatgct |
| | gcgacgcggtcaatcgtggcgtcgcgctgtatggagataaagtttttgta |
| | ggtacgctcgatggtcgtctagtagcgttagatcagaagaccggaaaagt |
| | agtttggtccaaggtagtagtgcccaatcaggaggactacaccataactg |
| | gtgccccgcgcgtggtgaaaggcaaagttctgattggtagcggtggctcg |
| | gagtacaaagctcgaggctatattgccgcctatgacgttaacacaggcaa |
| | cgaagtgtggaaattccacaccgtccctggcaatccagcggatgggtttg |
| | agaacaaagcgatggaaaatgccgctcgcacttgggctggtgaatggtgg |
| | aaactcggtgggggtggcacggtgtgggattccatcacctatgatccagc |
| | caccaacctagttctgttcggcacaggcaatgcagaaccatggaacccgg |
| | cagcagccggggggagggagacagcttgtacacgtcctctattgtagcgg |
| | tgaatgccgatactggcgactatgtatggcattttcaagaaaccccggaa |
| | gaccgttgggacttcgattccgcgcagcagattacgctggccgacctgac |
| | aattgatgggcagcggcgccacgtgatccttcatgcgcctaagaacggtc |
| | atgtttatgtgttggacgcaagaaccgggcagtttctgtcggcaacgccc |
| | tttgtgatggtgaactgggcgaccggtattgatcctaaaacgggcaaggc |
| | cactgtcaatccagaagcccgttatgaaaaaaccggcaaacctttcgtta |
| | gcctgccaggtgcggtaggcgcacattcatggcagccgcagagtttcagc |
| | ccgaaaaccggcctgctgtaccttccggtgaacaatgcggcatttcctta |
| | tgcagccgccaaagactggaaagcaaccgatattggtttccagaccggtc |
| | tcgacggctatgttaccagtatgccagccgacgcaaaggtccagggcgca |
| | gcgatgaaagcgaccactggtacgttagtggcgtgggacccggttgcgaa |
| | gaaagccgcttggaaagtcgaactgccgagcccgagtaacggtggcattt |
| | tatcgacagctggcaatttagtgtttcaaggtaccgcgggcggtgatttt |
| | gttgcatacaacgccgataagggcaaacaattatggtcttttccggcgca |
| | gagtggcatccttgccgcgccgatgacctatgctatcgatggggaacagt |
| | acgttgcggtcatggtgggctggggaggtgtgtgggacgtcgccacaggt |
| | gtgctcgctcataaggccaaaaaacagaggaacataagccgcctggtagt |
| | gttcaaactgggcgggaaagccacgctgccggctgctcctccgatggcaa |
| | aaatggttttggatccgccgccgtttacaggtacgcccgaacaagctaag |
| | gccggtggcgaattatacggacgttactgcaacgtttgtcatggtgatgc |
| | tgcggttgcgggcggcgtgaatccagatctgcgtcactcagctgcgctta |
| | atgcaccagaggcgatccggtctgtggtgattgagggggcgctgcagcac |
| | aacgggatggtctcgttcaaatctgcgctgaagcctgaggatgcggataa |
| | tatccgccactacttgatcaaacgtgcaaatgaagacaaagctctcgaag |
| | ccaaaggaggctaa (SEQ ID NO: 101) |
| Saro_3463 | attccgcatggtgaacattcaatgctggcaatgcagttggatggtccagg |
| gBlock | caaacggctgcacccagtcgtgcgccctctgccgttaccggggcgaggtg |
| | aagtgcgggtaaaagtgcatgcctgtggtgtttgccgtacggacctgcac |
| | gttgcagatggcgatattcacggtctgctacctattgtgccggggcacga |
| | agtgataggcgttgtcgatgcactggggccgggggtgacggatgttgaac |
| | ctggtgcgcgtgtaggtgtcccgtggctcggccatgcctgtggcacctgc |
| | ccatattgcgacagcgggagggaaaacctttgtgatgcgccgctgttcac |
| | cggttttactcgcgatggcggatacgctacccatgtgattgcagatgcgc |
| | gcttttgctttcctattccagagggttttgacgatctgcacgcggcgccg |
| | ctcctgtgcgcgggcttgatcggctatcgcgctcttcggcttgccggcga |
| | tgcacctgtactcggattctatggttttggagcggcggcgcatattttag |
| | ctcaggtggccctgtggcagggtagaacggtttacgcgtttactcgcgat |
| | ggcgacgctaaggcccaggcctttgctcgtgacatcggttgccaatgggc |
| | cggaccctctggcgctgcgccgccgcaagctctggacgcagcgatcatct |
| | tcgcctccgcgggagaattggtgccgacagccctgcgtgcagtgcgcaaa |
| | ggcgggcgtgttgtctgtgccggtattcatatgagcgatatcccggcatt |
| | cccctacgccgatttatgggaggaacgtcagatcctgtcggtagcgaatt |
| | taacccgacgcgatggcgtagaattcctgccccttgcagcgcgtgcaggc |
| | gttcgcacacatgtcgaggccatgccgttaatgaaagcgaacgaggccct |
| | ggaccgcctgcgtcgtggcgacgtcagtggcgctctggttttggtgccat |
| | aa (SEQ ID NO: 102) |
| Saro_3899 | gacgcatacgctgcaattatcgagcgtcagggtggagaattcgttctgga |
| gBlock | taacgtatctatcgaggatccgcgcgatggcgaagtgctggttaaggttg |
| | ccgcagctggcatgtgtcataccgatctgacggttcgcgatcaatattac |
| | ccgacgccgcttccggcggtgctgggccacgaaggtagcggcgttgttga |
| | aaaagtgggacgtggcgtcaccactgtcaaaccaggtgacaaagtagtgt |
| | tatccttcagctattgcggtacttgtccttcgtgcctcaaagggcatcag |
| | gcatactgtccgagcctgttcccgttaaatttcatgggccgtcgcctgga |
| | tggttcaacgcccattacacgcaacggtcaagaggtcaacgcctgctttt |
| | tcgggcaatcctcttttgcgacctatagtattgcgtcagaaaacaattgc |
| | gtcaaggttgccgacgatgcacagattgaacttttgggcccactgggctg |
| | cggcattcagaccggtgcgggaagtattttaaatgctctttgtcccgaac |
| | ctggttcctctatagcgatctttggggggggagtgtaggcttaagcgccg |
| | tgatggctgctaaagcatcgggctgcttgaagatcatcgcggttgacaga |
| | aatgcaggtcgcttggaactggcgcgtgaactgggcgccaccgatgtgat |
| | tgacgccaacacggtcaatgctcaggaagcgatcgtcgcgatgactggtg |
| | gcggcgccgactatgcaatggataccacagccattccagcggtgctgcgg |
| | agtgcggtggatagcacgcacaatatgggtgaaacagcagtggtgggcgg |
| | ggcgaaactgggtaccgagttttcactagacatgaataacatgctgtttg |
| | gtcgaaaattgcgtggcgtagtcgaaggatcgagcacgcctcaggtgttc |
| | atcccgcaactgattgcgatgcagaaagccgggctgtttccgtttgagaa |
| | actctgtaccttttatgatctggatcagatcaaccaggccgtagaggata |
| | ccgaaaagactggaaaagcgataaaagccattctcaaaatgtaa |
| | (SEQ ID NO: 103) |
| Saro_0060 | tctacacagcctgcaaccatagctgattccgcgaccgatctggttgaggg |
| gBlock | tcttgcacgtgcagcccgttctgcgcagcgccagttggcgcggatggatt |
| | caccggtaaaagaacgcgcgctgacgttagccgctgcagcgctgcgtgcc |
| | gctgaggccgaaattttagccgctaacgcgcaggatatggcgaatggcgc |
| | agcaaacggcctgtcctcggccatgctcgaccggctgaagttaacgccag |
| | agcgtctggccggcattgccgatgctgtggcgcaagtcgccgggctggcc |
| | gatccggtcggcgaggtgatcagtgaagctgcgcgtccgaatggcatggt |
| | gctgcagagagtgcgtattccggtcggagttatcggcatcatttacgaaa |
| | gccgccccaacgttaccgccgatgcagcagcgctctgcgtgcgttcaggt |
| | aatgcggcgattctgcgcggtggctcggaagcggttcatagtaaccgtgc |
| | gatccataaagcgctggttgctgggcttgccgaaggcggagtgccggcag |
| | aagcggtgcagcttgtacctacgcaggaccgtgctgccgtaggggcaatg |
| | etaggtgccgcgggactgatcgacatgatcgttccgcgcggcggaaaaag |
| | ccttgtcgctcgcgtccaggcagatgcccgcgtgccggtgttagcacact |
| | tggacggtatcaaccacacgtttgttcatgccagtgcagatccggcgatg |
| | gcccaagcgatagtgttgaatgccaaaatgcgtcgcaccggcgtttgtgg |
| | tgcgatggaaaccctgctgattgacgcgacttatccagatccccacggcc |
| | tggtcgaaccgctgctagacgccggttgcgagctgcgcggcgatgctcga |
| | gcgagagcaattgatccgaggattgcgccagctgccgacaacgactggga |
| | tacagaatatttggaagcgattctttcggttgcagtggtcgacggtttgg |
| | atgaagcgctcgcccacatcgcgcgccatgcctctggtcataccgatgca |
| | atcgtcgcggcggaccaagatgtggcagaccgattcttagctgaagtaga |
| | tagcgcaattgtaatgcataatgcatccagccagtttgctgatggcggtg |
| | agttcggcctgggtgctgagattggtattgccacggggggctgcacgcgc |
| | gcggccctgtagcgctcgaagggctgactacctacaaatggctggtgcgc |
| | ggaagcgggcaaactcgtccataa (SEQ ID NO: 104) |
| Saro_1104 | cgcgaacggctacagcaatacattgatggaaagtgggtagacagtgaagg |
| gBlock | tggcaaacgtcacgaagtcattaatccgactacagaggaaccctgttgtg |
| | tgattacgctgggcacgcaagcagatgtcgacaaagcagtggccgcggca |
| | cagcgcgcctttaaaaccttcagcaaaacgacgcgtgaggaacgactggc |
| | gctgcttgaacgcatcgtagaagaatacaagaagcgtgtccctgatttag |
| | ccgccgcgatggccgaggaaatgggagctccggtaagctttgccagcacc |
| | gcgcaagttggcgccggaatcggagcatttctgggcaccatggccgcgct |
| | ccgtaatttctcctttgttgaggacaacggtgcgtttaaagtggcctacg |
| | aaccgataggtgttgtgggtatgattacgccatggaactggccactgaat |
| | cagatagctctgaaagtagcaccggcgctggccgcggggaataccatgat |
| | cctgaaaccgtccgaggaatgcccaaccaacgcagcgatctttaccgaaa |
| | ttttggatgccgcaggggttccgccaggggtttttaacctgattcagggc |
| | gatggtcctggtgtaggcactgcgatcagtagtcatccgggcattgatat |
| | ggttagtttcaccggttcgacccgtgcgggcatcctcgtggcgaaagctg |
| | cggccgataccgtcaagcgggtgcatcaggaacttggcggtaaatctccc |
| | aatgtggtgctgcccgatgcagacttcgcaaaatatctgccgtctaccgc |
| | gtcaggcccgttggtgaacagcggccagagctgcatttcgccaacccgta |
| | ttttagtaccaagagaacgcgaagcagaagccgcggcttttgtttctgcg |
| | atgtactccgcaacaccggtcggggatccgatgcaagaaggtgcgcacat |
| | tgggccggtggttaacaaagctcagtttgacaagatccgcggtctgattc |
| | aatcggcaatagacgaaggcgcgaaactcgagacagggggcccgacttac |
| | cggccaatgtgaaccgcggctattatatcaaaccaacggtcttttcaggc |
| | gttactcctgatatgcgcattgctcaggaagaaatcttcggcccggtggc |
| | gacgattatggcgtacgattcattagaggaggccattgagatcgcaaatg |
| | atacagcctatggactgtcggcctgcattactggtgatccggcgaaagcg |
| | gctgaagtcgctcctgagcttcgtgcaggtatggtggctatcaataactg |
| | gggccctactccgggtgctccgttcggtggctataaacagtccggtaacg |
| | gtaggggggagggttgtatgggttgaaagacttcatggaaatgaaagcga |
| | tcagcggcctgcctgcctaa (SEQ ID NO: 105) |
| Saro_1197 | actgcccctaccgccgcagacctttccgccgatattgcacgggtttttgc |
| gBlock | actgcaacaagcgcacatgtgggaggccaaggcgtccaccgcggcggagc |
| | gcaaagaaaaattggcgcgtctgaaggccgcggttgaagcacacgcggat |
| | gacattgtggccgcggttctggaagatacgcgcaaacctgttggtgaaat |
| | aagggtgaccgaagttctgaatgtaaccgccaatatccagcgaaacatcg |
| | ataatctcgatgaatggatgaaaccggtcgaggtcgctacctcactgaat |
| | ccagcggaccgcgcgcagataattcatgaagcgcgcggcgtatgcctgat |
| | tcttggcccatggaatttccccttaggtctggcgctgggtccggtcgccg |
| | ctgctatcgccgcaggcaatacttgtatcgtgaaattaacggacttgtgt |
| | ccagcgaccgcaagagtggcatcggtgatcgtgcgtgaagcgttcgatga |
| | aaaagatgtggctctgtttgagggagacgttagtgtagctaccgcgcttt |
| | tggatctgccgtttaatcatgtattttttacaggctctccacgtgtaggc |
| | aaaattgtgatggctgctgcggcaaagcatctgaccagcgtcacgttaga |
| | gcttggtgggaagtctcccgttattgtcgatgatagcgcagatatcgatc |
| | aagttgctgcccagttagccgcggccaaacaattcaacggcgggcaggcc |
| | tgcatttccccggactatgtgtttgtgaaagaagacaaaaaagctgcgct |
| | ggtagaaggtttccgtgccaatgtgcagaaaaacttgtatgatgatgcag |
| | gcaacctgaaaaaagacagtattgcacaggtggtcaacaaagcgaacttt |
| | gatcgtgtgaaagccatgttcgacgatgcagtcgcaaaaggcgcgaccgt |
| | cgccgctggtggaacgtttgaagcggatgacttgactattcatccgacaa |
| | tgctgacaggcgtaaccccgcagatgactattctccaggatgagatcttt |
| | gcccctgtcattccggtgatgacctacgacacgctggatcaagcgatcgg |
| | gtatatcgaagcacgcgacaaaccgctagcactctatgtttacagtaaag |
| | atgaagcgaacgttgaaaaggtcttagcccgcacgtcatcgggtggtgtt |
| | acggtgaatggtgtgttctcgcactacctggaaaacaacctgccgttcgg |
| | gggggttaacacaagcggtatgggcagctaccatggcgtgttcggattta |
| | agtgctttagccacgagcgggctgtatatcgtcatcagcagtaa |
| | (SEQ ID NO: 106) |
| Saro_1410 | ggttaccgggttgtagtggtgggtgcgactgggaatgtggggcgtgaaat |
| gBlock | gctgaacattctggcagaacgcgagtttccttgtgacgagatcgcagcgg |
| | ttgctagctctcgttcgcagggcaccgaaatagaatttggcgaaactggc |
| | cggaagctgaaagtacagaatgttgaaaattttgattttaccggatggga |
| | cattgcactgtttgcggcgggatcaggcccgacgcagatccatgctccac |
| | gtgccgcttctcagggctgcgtggtgatcgataacagtagcttataccgc |
| | atggacccggacgtgcctctgatcgtgcccgaggtgaatccggatgcgat |
| | tgatggctataccaaaaaaaacattattgccaatccaaactgttccaccg |
| | cgcaaatggtcgtggcgctgaaaccgttacatgatgccgccaaaattaaa |
| | agagttgtcgtctccacgtatcaaagcgtttccggcgcgggtaaagaagg |
| | gatggatgaactgttcgaacaaagccgcgcgatatttgtcggggacccgg |
| | tggaaccgaaaaaattcaccaaacagatcgcattcaacgtgatccctcat |
| | atcgatgtattcctagacgatggttcgactaaagaagagtggaaaatggt |
| | cgccgaaaccaaaaaaattttggaccccaaggttaaggtaacggcaacct |
| | gcgtgcgtgtgccggtgttcatcggccactcggaagcgttaaacattgag |
| | ttcgagaatgaaattagtgccgaggaagcgcagaatatcctgcgcgaagc |
| | accaggtgtgatgctcgtcgataagcgcgagaacggcggatatgttacgc |
| | cggtcgaatgcgttggtgattttgccacatttgttagccgcgtacgtgag |
| | gattcaacagttgataacggccttaatatttggtgtgtcagtgataacct |
| | gaggaaaggtgctgccttgaacgctgtacagattgcagaactgctcggtc |
| | gtcgacaccttaaaaagggttaa (SEQ ID NO: 107) |
| Saro_1967 | gcgatcaaagttgcgataaacggttttggacgtatcgggaggaatgtggc |
| gBlock | ccgcgccattttagaacgtcccgattgtgggttagaactggttagcatta |
| | acgacctggctgatgccaaggctaacgccctgctgtttaaacgcgacagc |
| | gttcatggcgcgttcagtggcgaagtatcagtggatggcaatgatctgat |
| | tgtgaatggcaagcgcattcaggtgactgcagagcgcgatcctgctaacc |
| | tgccacacggagccaatggtattgacattgcgctggaatgcacgggcttt |
| | ttcaccaatcgtgatggtggccagaaacacttggacgcgggcgccaaacg |
| | cgttctgatttccgctccggcaaaaaacgtagacctgacggtcgtctatg |
| | gtgtgaaccacgacaaactgaccggcgatcataagatcgtgtccaacgcg |
| | agttgcacgaccaactgtttggcgccgatggcaaaagtcctgcatgaatc |
| | tatcgggattgagcgtggtctaatgacaacgattcattcgtataccaatg |
| | atcaaaaaatactcgaccagatccatagcgatcctagacgggctcgggca |
| | gcggcgatgaatatgatccccacaagcaccggggccgcagttgcagtggg |
| | tgaagttctgccagacttaaaagggaaacttgatggttcgtcgattcgag |
| | tcccgaccccgaacgtatctgtcgtggatcttactttcacgccgaagcgt |
| | gataccagcgtagaggaagtaaatggtctcttgaaagcggctgccgaagg |
| | cgcattgaaaggcgtgttaggttacaccgacgaaccgctggtttcaatcg |
| | attttaaccacgatccgcatagttcaacaatcgacagccttgagactgcc |
| | gtgctcgaaggtaaactggtgcgcgtcctgtcttggtacgataatgagtg |
| | gggcttttccaaccgtatgctggatacggcgggagcaatggcgaaattcc |
| | tttaa (SEQ ID NO: 108) |
| Saro_2869 | aatgacatgactaccatctcacgcacgcagcgtgaatactccgaggccgc |
| gBlock | aaaagctttcctcgcgagaaagccgcaattgtttattaataacgagtggg |
| | tcgatagcagtcacgatgcagtgatcgaagtggaagacccctcgaatggg |
| | aggattgtaggtcatgtcgttgatgcctcggacaaagacgttgaccgggc |
| | ggttgccgctgcgcgggccgctttcgatgatggtcgttggtccaacctgc |
| | cgccaatggtacgcgatcgtaccatgaatcgcctggccgacctgcttgaa |
| | gcaaacgcagatctctttgcagagctggaagcgattgataatggtaaacc |
| | gaagggtatggccggcgccgttgatattccaggtgcgataagccaactac |
| | gcttcatggcaggatgggccagcaaggtagctggcgaaacgacgcagcct |
| | tacacgatgccgaatggcaccgtgtttagttacaccgtcaaagaacccgt |
| | cggtgtctgcgcgcagattgtgccgtggaacttcccgctgctgatggcat |
| | cattgaagatcgccccggcgctggcggctggatgtacactggtgctgaaa |
| | cctgccgaacagacatcgcttaccgcgttaaaactggcagatttggtggt |
| | tgaggctggctttcctgcgggagtgatcaacattatcacagggaacggcc |
| | acaccgcaggtgatcgcatggtcaaacatcccgacgtagacaaagtcgcc |
| | tttactggctccaccgaaatcgggaaactgataaatcgaaacgcaaccac |
| | cacgcttaaacgggttacgctcgaactggggggaaaagtcccgtagtggt |
| | tatgccagacgtagatgtggcgcagaccgcgcctggcgttgccggtgcga |
| | tttttttcaacgctggccaggtttgtgttgccggtagtcgtttatatgcg |
| | caccgttcggtgttcgattccgtgttagaaggtatgacccagactgcgcc |
| | gttttgggcgccgcgcccgagcctggatccagaagcacacatgggaccgt |
| | tggtcagcaaagagcaacatgaccgtgtgatgggatatatcgaggcgggc |
| | aagcgtgatggcgccagcgtagtgatgggcggtgattgcccaagcgctga |
| | tggagggtactatgttaatccgacgattctggcagacgtgaatccgcaga |
| | tgtctgtcgtgcgcgaggaaatttttggtccggttgtcgtcgcccaacgc |
| | ttcgacgatttagatgaagtggcgaaaatggcaaacgacacctgttttgg |
| | cttaggtgcgggcgtgtggacgcgcgatgttgcggtgatgcataaacttg |
| | cttcaaagatcaaatctggcactgtgtggggcaactgccatgccctgatc |
| | gatacagcgctgccttttggcggctataaagaatctgggctgggtcgaga |
| | acaggggcgtgccggtattgatgcttatttggagactaaaacagtaatta |
| | ttcaaatgtaa (SEQ ID NO: 109) |
| Saro_3848 | gctacgcagttgagaagtgcagaaaatgaatatgggatcaaatccgagta |
| gBlock | tggtcattatataggaggtgagtggattgcaggggatagcggcaagacca |
| | tagatttactaaatccctctaccggtaaagtgctgaccaaaattcaagcc |
| | ggcaacgcaaaagatattgaacgcgcgattgccgctgcaaaagcggcgtt |
| | tccgaagtggagccagagcctgccaggggagcgccaagaaatcctgatag |
| | aggttgcgcgtcgtctgaaagcacgccattcgcactatgcaaccttagaa |
| | acgctcaataacggtaaaccgatgcgcgaatcaatgtatttcgatatgcc |
| | tcaaacgatcgggcaatttgagctgttcgccggtgccgcctatggcctgc |
| | atggccagacgctggattatccagacgcgattggcatcgtccaccgtgaa |
| | ccgttaggcgtatgcgcgcagattattccatggaacgtgccgatgttgat |
| | gatggcgtgcaaaatcgcgcccgcgctggcctctggcaacactgtcgttc |
| | tgaaaccggccgaaacggtgtgcctttctgtgattgaatttttcgtggaa |
| | atggctgatctgttgcctccgggtgtgatcaacgttgttaccgggtatgg |
| | tgctgacgttggcgaggcgcttgtaacaagccctgatgtagctaaagtgg |
| | cctttaccggttcgattgctacggcgcgccggattattcagtatgcctcg |
| | gccaatatcattccacagacgctcgagttgggcggtaaatcagcgcatat |
| | cgtgtgtggcgatgccgatattgacgcggcggtggaaagtgcgactatgt |
| | ccaccgttttaaataaaggtgaagtctgtctggctggttcacgcctgttt |
| | ctgcatcagtccatccaggatgaattcctggccaaatttaaaacagcgct |
| | tgaaggcattcgccaaggcgacccgctagatatggcgactcaacttggag |
| | cgcaggcatcgaagatgcagtttgacaaggtgcaaagctacttaaggctg |
| | gctacagaggaaggggcagaggtactgaccggcggtagtcgttcagatgc |
| | cgcagatctggcagatggcaattttatcaaaccgacggtttttactaacg |
| | tcaataactccatgcggatcgcgcaggaagagattttcggaccggttacc |
| | agcgtaattacatggagcgacgaagacgacatgatgaaacaggccaacaa |
| | tacaacttacggcttggctggcggtgtctggaccaaggacatcgcacgag |
| | cacaccgtattgcgcgtaaactcgaaactggcacggtctggatcaatcgc |
| | tactacaacctgaaagccaacatgccgctgggaggttacaagcaaagtgg |
| | ctttgggcgtgaattcagccatgaagtgctgaatcactacacccagacca |
| | aatctgtggttgtcaacctccaggaaggtcgtaccggaatgttcgatcag |
| | taa (SEQ ID NO: 110) |

### Protein Purification

PcfL and FerD were purified from the crude cell extract by fast protein liquid chromatography. The crude cell extracts were applied directly to a Ni-NTA column and washed with buffer A (50 mM NaH₂PO₄*H₂O, 0.5 mM tris(2-carboxyethyl) phosphine, 25 mM imidazole, and 200 mM NaCl, pH 7.5). The His-tagged proteins bound to the resin were eluted with Buffer B (50 mM NaH₂PO₄*H₂O, 0.5 mM tris(2-carboxyethyl) phosphine, 500 mM imidazole, and 300 mM NaCl, pH 7.5). The eluted proteins were collected and concentrated in Buffer C (50 mM NaH₂PO₄*H₂O, 0.5 mM tris(2-carboxyethyl) phosphine, 10 mM imidazole, and 100 mM NaCl, pH 7.5) using a 10 kDA MWCO centrifugal filter and hanging basket centrifugation (3,000×g) at 4° C. Protein concentration was quantified by Bradford protein assay measuring absorbance at 595 nm and the purified proteins were diluted to ˜2 mg/mL protein by addition of buffer C. They were then treated overnight at 4° C. with 1 mg TEV-protease per ˜30 mg of protein. The protease-treated samples were applied to a Ni-NTA column and the proteins were eluted with buffer C and the high imidazole buffer B was used afterwards to elute any remaining protein. A 10 kDA MWCO centrifugal filter and hanging basket centrifugation (3,000×g) at 4° C. was used to concentrate the proteins, wash them twice with HEPES buffer (50 mM HEPES, 20 mM NaCl, pH 7.5), and concentrate them again, Fractions were saved throughout the purification process and protein content in each fraction was analyzed by sodium dodecyl sulfate polyacrylamide gel electrophoresis. Glycerol was added to the purified, concentrated proteins to a final concentration of 20% before they were flash frozen in a dry ice-ethanol bath and stored at −80° C. A Bradford protein assay measuring absorbance at 595 nm was used to determine the final protein concentration.

### Analysis of Extracellular Formaldehyde

Extracellular medium samples were collected as described in the Materials and Methods and analyzed for extracellular formaldehyde by the Great Lakes Bioenergy Research Center Metabolomics Lab. Formaldehyde concentrations were measured by headspace analysis using an Agilent 7890 Gas Chromatogram equipped with a LECO Pegasus BT time-of-flight mass spectrometry and controlled using LECO's ChromTOF software v4.72.0.0. The samples were prepared in 20 mL headspace vials (Restek, Cat #23082) by diluting 100 μL of filtered medium into 5 mL of water containing p-TSA as the internal standard. The diluted samples were loaded onto a L-PAL 3 auto-sampler equipped with a 2.5 mL headspace syringe (PAL system, Cat #PAL3-Sys-008655). Prior to injection, each sample was transferred to an agitator preheated to 70° C. and incubated for 40 minutes at 350 rpm prior to loading 500 μL of the headspace gas into the syringe. The sample was injected into a 120° C. inlet with a 50:1 split ratio onto a Stabilwax-DA column (Restek, 30 m×0.25 mm×0.5 μm, Cat #11038) with helium as the mobile phase flowing at a constant 1 mL/min. The temperature program was set at 40° C. for 4.20 minutes, followed by a 40° C./minute ramp up to 200° C. The transfer line to the MS was set to 210° C. The MS source was set to 200° C. and had an acquisition delay of 135 seconds. The chromatogram data was collected from 135-55 seconds at 10 spectra/see covering the mass range of 10-350 m/z. Quantification was performed using p-TSA as the internal standard with a 10-point calibration curve.

### DC-S-C Abiotic Dimerization Assay

The time-dependent abiotic conversion of DC-S-C to DC-T-C was measured in water, DMSO, S30 buffer, and SMB minimal medium supplemented with 1 g/L glucose in a 96-well plate. DC-S-C was added in triplicate to each medium to a concentration of 0.2 mM and the 96-well plate was immediately placed in a Tecan Infinite M1000 reader set to maintain a temperature of 30° C. Every hour for 18 hours, absorbance of DC-S-C was measured at 370 nm since DC-S-C absorbs at 370 nm while DC-T-C does not (FIG. 26). A series of 2-fold dilutions were performed to create a standard curve of eight concentrations of DC-S-C and of DC-T-C in each medium. The standard curves were then used to quantify extracellular concentrations of these aromatics based on absorbance at 370 nm.

### Absorbance Spectra of Standards

To identify the wavelengths at which to measure absorbance in the ADH and ALDH in vitro assays and DC-S-C abiotic dimerization assay, the absorbance of standards was determined with the goal of identifying wavelengths at which either solely a substrate or solely a product absorbs. Triplicate 0.2 mM mixtures of DC-A, DC-L, and DC-C in S30 buffer and 0.2 mM standards of DC-S-C and DC-T-C in SMB minimal medium supplemented with 1 g/L glucose were created and their absorbance was measured from 230 nm to 500 nm in a Tecan Infinite M1000 reader.

### REFERENCES

1. Ragauskas A J, Beckham G T, Biddy M J, Chandra R, Chen F, Davis M F, Davison B H, Dixon R A, Gilna P, Keller M, Langan P, Naskar A K, Saddler J N, Tschaplinski T J, Tuskan G A, Wyman C E. 2014. Lignin valorization: improving lignin processing in the biorefinery. Science 344:1246843.
2. Sun Z, Fridrich B, de Santi A, Elangovan S, Barta K. 2018. Bright Side of Lignin Depolymerization: Toward New Platform Chemicals. Chem Rev 118:614-678.
3. Abu-Omar M M, Barta K, Beckham G T, Luterbacher J S, Ralph J, Rinaldi R, Romin-Leshkov Y, Samec J S M, Sels B F, Wang F. 2021. Guidelines for performing lignin-first biorefining. Energy & Environmental Science 14:262-292.
4. Ralph J, Lapierre C, Boerjan W. 2019. Lignin structure and its engineering. Curr Opin Biotechnol 56:240-249.
5. Vanholme R, De Meester B, Ralph J, Boerjan W. 2019. Lignin biosynthesis and its integration into metabolism. Curr Opin Biotechnol 56:230-239.
6. Sangha A K, Parks J M, Standaert R F, Ziebell A, Davis M, Smith J C. 2012. Radical coupling reactions in lignin synthesis: a density functional theory study. J Phys Chem B 116:4760-8.
7. Zakzeski J, Jongerius A L, Bruijnincx P C, Weckhuysen B M. 2012. Catalytic lignin valorization process for the production of aromatic chemicals and hydrogen. ChemSusChem 5:1602-9.
8. Gall D L, Ralph J, Donohue T J, Noguera D R. 2017. Biochemical transformation of lignin for deriving valued commodities from lignocellulose. Current Opinion in Biotechnology 45:120-126.
9. Linger J G, Vardon D R, Guarnieri M T, Karp E M, Hunsinger G B, Franden M A, Johnson C W, Chupka G, Strathmann T J, Pienkos P T, Beckham G T. 2014. Lignin valorization through integrated biological funneling and chemical catalysis. Proc Natl Acad Sci USA 111:12013-8.
10. Perez J M, Kontur W S, Alherech M, Coplien J, Karlen S D, Stahl S S, Donohue T J, Noguera D R. 2019. Funneling aromatic products of chemically depolymerized lignin into 2-pyrone-4-6-dicarboxylic acid with. Green Chemistry 21:1340-1350.
11. Kamimura N, Takahashi K, Mori K, Araki T, Fujita M, Higuchi Y, Masai E. 2017. Bacterial catabolism of lignin-derived aromatics: New findings in a recent decade: Update on bacterial lignin catabolism. Environ Microbiol Rep 9:679-705.
12. Becker J, Wittmann C. 2019. A field of dreams: Lignin valorization into chemicals, materials, fuels, and health-care products. Biotechnol Adv 37:107360.
13. Fredrickson J K, Brockman F J, Workman D J, Li S W, Stevens T O. 1991. Isolation and characterization of a subsurface bacterium capable of growth on toluene, naphthalene, and other aromatic compounds. Appl Environ Microbiol 57:796-803.
14. Fredrickson J K, Balkwill D L, Drake G R, Romine M F, Ringelberg D B, White D C. 1995. Aromatic-degrading Sphingomonas isolates from the deep subsurface. Appl Environ Microbiol 61:1917-22.
15. Perez J M, Sener C, Misra S, Umana G E, Coplien J, Haak D, Li Y D, Maravelias C T, Karlen S D, Ralph J, Donohue T J, Noguera D R. 2022. Integrating lignin depolymerization with microbial funneling processes using agronomically relevant feedstocks. Green Chemistry 24:2795-2811.
16. Vilbert A C, Kontur W S, Gille D, Noguera D R, Donohue T J. 2024. Engineering Novosphingobium aromaticivorans to produce cis,cis-muconic acid from biomass aromatics. Appl Environ Microbiol 90:e0166023.
17. Hall B W, Kontur W S, Neri J C, Gille D M, Noguera D R, Donohue T J. 2023. Production of carotenoids from aromatics and pretreated lignocellulosic biomass by Novosphingobium aromaticivorans. Appl Environ Microbiol 89:e0126823.
18. Otsuka Y, Nakamura M, Shigehara K, Sugimura K, Masai E, Ohara S, Katayama Y. 2006. Efficient production of 2-pyrone 4,6-dicarboxylic acid as a novel polymer-based material from protocatechuate by microbial function. Appl Microbiol Biotechnol 71:608-14.
19. Shikinaka K, Otsuka Y, Nakamura M, Masai E, Katayama Y. 2018. Utilization of Lignocellulosic Biomass via Novel Sustainable Process. J Oleo Sci 67:1059-1070.
20. Perez J M, Kontur W S, Gehl C, Gille D M, Ma Y, Niles A V, Umana G, Donohue T J, Noguera D R. 2021. Redundancy in aromatic O-demethylation and ring opening reactions in Novosphingobium aromaticivorans and their impact in the metabolism of plant derived phenolics. Appl Environ Microbiol 87.
21. Cecil J H, Garcia D C, Giannone R J, Michener J K. 2018. Rapid, Parallel Identification of Catabolism Pathways of Lignin-Derived Aromatic Compounds in Novosphingobium aromaticivorans. Appl Environ Microbiol 84.
22. Gall D L, Ralph J, Donohue T J, Noguera D R. 2014. A group of sequence-related sphingomonad enzymes catalyzes cleavage of beta-aryl ether linkages in lignin beta-guaiacyl and beta-syringyl ether dimers. Environ Sci Technol 48:12454-63.
23. Kontur W S, Bingman C A, Olmsted C N, Wassarman D R, Ulbrich A, Gall D L, Smith R W, Yusko L M, Fox B G, Noguera D R, Coon J J, Donohue T J. 2018. Novosphingobium aromaticivorans uses a Nu-class glutathione S-transferase as a glutathione lyase in breaking the beta-aryl ether bond of lignin. J Biol Chem 293:4955-4968.
24. Presley G N, Werner A Z, Katahira R, Garcia D C, Haugen S J, Ramirez K J, Giannone R J, Beckham G T, Michener J K. 2021. Pathway discovery and engineering for cleavage of a β-1 lignin-derived biaryl compound. Metabolic Engineering 65:1-10.
25. Chen Z, Wan C X. 2017. Biological valorization strategies for converting lignin into fuels and chemicals. Renewable & Sustainable Energy Reviews 73:610-621.
26. Guadix-Montero S, Sankar M. 2018. Review on Catalytic Cleavage of C-C Inter-unit Linkages in Lignin Model Compounds: Towards Lignin Depolymerisation. Topics in Catalysis 61:183-198.
27. Habu N, Samejima M, Yoshimoto T. 1988. Metabolic Pathway of Dehydrodiconiferyl Alcohol by Pseudomonas Sp Tmy1009. Mokuzai Gakkaishi 34:1026-1034.
28. Takahashi K, Hirose Y, Kamimura N, Hishiyama S, Hara H, Araki T, Kasai D, Kajita S, Katayama Y, Fukuda M, Masai E. 2015. Membrane-Associated Glucose-Methanol-Choline Oxidoreductase Family Enzymes PhcC and PhcD Are Essential for Enantioselective Catabolism of Dehydrodiconiferyl Alcohol. Applied and Environmental Microbiology 81:8022-8036.
29. Takahashi K, Miyake K, Hishiyama S, Kamimura N, Masai E. 2018. Two novel decarboxylase genes play a key role in the stereospecific catabolism of dehydrodiconiferyl alcohol in sp strain SYK-6. Environmental Microbiology 20:1739-1750.
30. Kamimura N, Hirose Y, Masuba R, Kato R, Takahashi K, Higuchi Y, Hishiyama S, Masai E. 2021. LsdD has a critical role in the dehydrodiconiferyl alcohol catabolism among eight lignostilbene α,β-dioxygenase isozymes in sp. strain SYK-6. International Biodeterioration & Biodegradation 159.
31. Kawazoe M, Takahashi K, Tokue Y, Hishiyama S, Seki H, Higuchi Y, Kamimura N, Masai E. 2023. Catabolic System of 5-Formylferulic Acid, a Downstream Metabolite of a-5-Type Lignin-Derived Dimer, in SYK-6. Journal of Agricultural and Food Chemistry 71:19663-19671.
32. Takahashi K, Kamimura N, Hishiyama S, Hara H, Kasai D, Katayama Y, Fukuda M, Kajita S, Masai E. 2014. Characterization of the catabolic pathway for a phenylcoumaran-type lignin-derived biaryl in Sphingobium sp. strain SYK-6. Biodegradation 25:735-45.
33. Rashid G M M, Riviere G, Cottyn-Boitte B, Majira A, Cezard L, Sodre V, Lam R, Fairbairn J A, Baumberger S, Bugg T D. 2024. Ether Bond Cleavage of a Phenylcoumaran beta-5 Lignin Model Compound and Polymeric Lignin Catalysed by a LigE-type Etherase from Agrobacterium sp. Chembiochem doi:10.1002/cbic.202400132:e202400132.
34. Myers K S, Vera J M, Lemmer K C, Linz A M, Landick R, Noguera D R, Donohue T J. 2020. Genome-Wide Identification of Transcription Start Sites in Two Alphaproteobacteria, Rhodobacter sphaeroides 2.4.1 and Novosphingobium aromaticivorans DSM 12444. Microbiol Resour Announc 9.
35. Gonzalez C F, Proudfoot M, Brown G, Korniyenko Y, Mori H, Savchenko A V, Yakunin A F. 2006. Molecular basis of formaldehyde detoxification. Characterization of two S-formylglutathione hydrolases from Escherichia coli, FrmB and YeiG. J Biol Chem 281:14514-22.
36. Leonhartsberger S, Korsa I, Bock A. 2002. The molecular biology of formate metabolism in enterobacteria. J Mol Microbiol Biotechnol 4:269-76.
37. Kuatsjah E, Zahn M, Chen X, Kato R, Hinchen D J, Konev M O, Katahira R, Orr C, Wagner A, Zou Y, Haugen S J, Ramirez K J, Michener J K, Pickford A R, Kamimura N, Masai E, Houk K N, McGeehan J E, Beckham G T. 2023. Biochemical and structural characterization of a sphingomonad diarylpropane lyase for cofactorless deformylation. Proc Natl Acad Sci USA 120:e2212246120.
38. Barber R D, Rott M A, Donohue T J. 1996. Characterization of a glutathione-dependent formaldehyde dehydrogenase from Rhodobacter sphaeroides. J Bacteriol 178:1386-93.
39. Barber R D, Donohue T J. 1998. Function of a glutathione-dependent formaldehyde dehydrogenase in Rhodobacter sphaeroides formaldehyde oxidation and assimilation. Biochemistry 37:530-7.
40. Marasco E K, Schmidt-Dannert C. 2008. Identification of bacterial carotenoid cleavage dioxygenase homologues that cleave the interphenyl alpha,beta double bond of stilbene derivatives via a monooxygenase reaction. Chembiochem 9:1450-61.
41. McAndrew R P, Sathitsuksanoh N, Mbughuni M M, Heins R A, Pereira J H, George A, Sale K L, Fox B G, Simmons B A, Adams P D. 2016. Structure and mechanism of NOV1, a resveratrol-cleaving dioxygenase. Proc Natl Acad Sci USA 113:14324-14329.
42. Vladimirova A, Patskovsky Y, Fedorov A A, Bonanno J B, Fedorov E V, Toro R, Hillerich B, Seidel R D, Richards N G, Almo S C, Raushel F M. 2016. Substrate Distortion and the Catalytic Reaction Mechanism of 5-Carboxyvanillate Decarboxylase. J Am Chem Soc 138:826-36.
43. Peng X, Masai E, Kitayama H, Harada K, Katayama Y, Fukuda M. 2002. Characterization of the 5-carboxyvanillate decarboxylase gene and its role in lignin-related biphenyl catabolism in Sphingomonas paucimobilis SYK-6. Appl Environ Microbiol 68:4407-15.
44. Linz A M, Ma Y, Perez J M, Myers K S, Kontur W S, Noguera D R, Donohue T J. 2021.

Aromatic Dimer Dehydrogenases from Novosphingobium aromaticivorans Reduce Monoaromatic Diketones. Appl Environ Microbiol 87:e0174221.
45. Quideau S, Ralph J. 1992. Facile Large-Scale Synthesis of Coniferyl, Sinapyl, and Para-Coumaryl Alcohol. Journal of Agricultural and Food Chemistry 40:1108-1110.
46. Ralph J, Conesa MTG, Williamson G. 1998. Simple preparation of 8-5-coupled diferulate. Journal of Agricultural and Food Chemistry 46:2531-2532.
47. Kulkarni M G, Mathew S. 1990. 1,4-Benzoquinone—a New Selective Reagent for Oxidation of Alcohols. Tetrahedron Letters 31:4497-4500.
48. Yue F X, Gao R L, Piotrowski J S, Kabbage M, Lu F C, Ralph J. 2017. Scaled-up production of poacic acid, a plant-derived antifungal agent. Industrial Crops and Products 103:240-243.
49. Li Q, Li Y, Liu W, Wang T Y, Zhu Y J, Du Z Y. 2021. Formylation of Phenols and Paraformaldehyde Catalyzed by Ammonium Acetate. Chinese Journal of Organic Chemistry 41:2038-2044.
50. Travis B R, Sivakumar M, Hollist G O, Borhan B. 2003. Facile oxidation of aldehydes to acids and esters with Oxone. Org Lett 5:1031-4.
51. Huber R, Marcourt L, Koval A, Schnee S, Righi D, Michellod E, Katanaev V L, Wolfender J L, Gindro K, Queiroz E F. 2021. Chemoenzymatic Synthesis of Complex Phenylpropanoid Derivatives by the Botrytis cinerea Secretome and Evaluation of Their Wnt Inhibition Activity. Front Plant Sci 12:805610.
52. Schafer A, Tauch A, Jager W, Kalinowski J, Thierbach G, Puhler A. 1994. Small mobilizable multi-purpose cloning vectors derived from the Escherichia coli plasmids pK18 and pK19: selection of defined deletions in the chromosome of Corynebacterium glutamicum. Gene 145:69-73.
53. Blodgett J A, Thomas P M, Li G, Velasquez J E, van der Donk W A, Kelleher N L, Metcalf W W. 2007. Unusual transformations in the biosynthesis of the antibiotic phosphinothricin tripeptide. Nat Chem Biol 3:480-5.
54. Doherty A J, Ashford S R, Brannigan J A, Wigley D B. 1995. A superior host strain for the over-expression of cloned genes using the T7 promoter based vectors. Nucleic Acids Res 23:2074-5.
55. Lakey B D, Myers K S, Alberge F, Mettert E L, Kiley P J, Noguera D R, Donohue T J. 2022. The essential Rhodobacter sphaeroides CenKR two-component system regulates cell division and envelope biosynthesis. PLoS Genet 18:e1010270.
56. Bolger A M, Lohse M, Usadel B. 2014. Trimmomatic: a flexible trimmer for Illumina sequence data. Bioinformatics 30:2114-20.
57. Li H, Durbin R. 2009. Fast and accurate short read alignment with Burrows-Wheeler transform. Bioinformatics 25:1754-60.
58. Li H, Handsaker B, Wysoker A, Fennell T, Ruan J, Homer N, Marth G, Abecasis G, Durbin R, Genome Project Data Processing S. 2009. The Sequence Alignment/Map format and SAMtools. Bioinformatics 25:2078-9.
59. Anders S, Pyl P T, Huber W. 2015. HTSeq—a Python framework to work with high-throughput sequencing data. Bioinformatics 31:166-9.
60. Robinson M D, McCarthy D J, Smyth G K. 2010. edgeR: a Bioconductor package for differential expression analysis of digital gene expression data. Bioinformatics 26:139-40.
61. Benjamini Y, Hochberg Y. 1995. Controlling the False Discovery Rate—a Practical and Powerful Approach to Multiple Testing. Journal of the Royal Statistical Society Series B-Statistical Methodology 57:289-300.
62. Wetmore K M, Price M N, Waters R J, Lamson J S, He J, Hoover C A, Blow M J, Bristow J, Butland G, Arkin A P, Deutschbauer A. 2015. Rapid quantification of mutant fitness in diverse bacteria by sequencing randomly bar-coded transposons. mBio 6:e00306-15.
63. Studier F W. 2005. Protein production by auto-induction in high density shaking cultures. Protein Expr Purif 41:207-34.
64. Prasad S, Khadatare P B, Roy I. 2011. Effect of chemical chaperones in improving the solubility of recombinant proteins in Escherichia coli. Appl Environ Microbiol 77:4603-9.
65. Kigawa T, Yabuki T, Matsuda N, Matsuda T, Nakajima R, Tanaka A, Yokoyama S. 2004. Preparation of Escherichia coli cell extract for highly productive cell-free protein expression. J Struct Funct Genomics 5:63-8.
66. Chaumeil P A, Mussig A J, Hugenholtz P, Parks D H. 2019. GTDB-Tk: a toolkit to classify genomes with the Genome Taxonomy Database. Bioinformatics 36:1925-7.
67. Kozlov A M, Darriba D, Flouri T, Morel B, Stamatakis A. 2019. RAxML-N G: a fast, scalable and user-friendly tool for maximum likelihood phylogenetic inference. Bioinformatics 35:4453-4455.
68. Bianchini G, Sanchez-Baracaldo P. 2024. TreeViewer: Flexible, modular software to visualise and manipulate phylogenetic trees. Ecol Evol 14:e10873.

### Enzyme Sequences

FdhA (Saro_0874) Coding Sequence
(SEQ ID NO: 1)
Atgctatcggaccgccacgtcaaagggagaccgcacgaaatgaag
acccgcgccgcagttgcgttcgcgcccaagcagccgctcgagatc
gtcgaactggacctcgaaggccccaaggctggcgaagtgctggtc
gagatcatggcgaccggcgtgtgccacaccgatgcctacacgctc
gacgggttcgacagcgaaggcatcttccccagcgtgctgggccac
gaaggcgccggtatcgtgcgcgaggtgggccctggggtcacttcg
gtgaagcccggcgatcacgtgatcccgctctacacgccggaatgc
cgccagtgcaaatcgtgcctctcgggcaagaccaacctgtgcacc
gcgatccgcgccacgcaagggcagggcctgatgcccgacggcacc
agccgcttttcgtacaagggccagaccgtgttccactacatgggc
tgctcgaccttctctaacttcaccgtcctgcccgagatcgcggtt
gccaagatccgcgaggacgcgccgttcaagacctcgtgctatatc
ggctgcggcgtgacgacgggcgtcggcgcggtgatcaacaccgcc
aaggtccaggtcggtgacaacgtcgtggtcttcggcctcggcggc
atcggcctcaacgtgatccagggcgcgcggcttgccggtgccggc
aagatcatcggcgtcgacatcaaccccgaccgcgaggaatggggc
cgcaagttcggcatgaccgacttcctcaacagcaagggcatgagc
cgcgaggacgtcgtcgccaaggtcgtcgccatgaccgacggcggc
gcggactacaccttcgacgccaccggcaacaccgaagtgatgcgc
acggcgcttgaagcctgccatcgcggctggggcacctccatcatc
atcggcgtggccgaggcgggcaaggaaatcagcacgcgtccgttc
cagctcgtcaccggccgcaactggcgcggcacggccttcggcggc
gccaagggccgcaccgacgtgcccaagatcgtcgacatgtacatg
accggcaagatcgagatcgacccgatgatcacccatgtcatgggc
ctggaagagatcaacaccgccttcgacctgatgcacgccggcaag
tcgatccgttcagtcgtggtgttctga
FdhA (Saro_0874) Protein Sequence
(SEQ ID NO: 2)
MLSDRHVKGRPHEMKTRAAVAFAPKQPLEIVELDLEGPKAGEVLV
EIMATGVCHTDAYTLDGFDSEGIFPSVLGHEGAGIVREVGPGVTS
VKPGDHVIPLYTPECRQCKSCLSGKTNLCTAIRATQGQGLMPDGT
SRFSYKGQTVFHYMGCSTFSNFTVLPEIAVAKIREDAPFKTSCYI
GCGVTTGVGAVINTAKVQVGDNVVVFGLGGIGLNVIQGARLAGAG
KIIGVDINPDREEWGRKFGMTDFLNSKGMSREDVVAKVVAMTDGG
ADYTFDATGNTEVMRTALEACHRGWGTSIIIGVAEAGKEISTRPF
QLVTGRNWRGTAFGGAKGRTDVPKIVDMYMTGKIEIDPMITHVMG
LEEINTAFDLMHAGKSIRSVVVF*
Saro_0995 Coding Sequence
(SEQ ID NO: 3)
Atgaaagccgccgtactcgtcgaaccgggcaagccgctggatatt
cagcatctcagcgtgtccaagcccggcccgcatgaagtccttatc
cgcaccgcagcctgcgggctgtgccattcggacttgcacttcatc
gaaggtgcctatccccatccgctgcccgcggtgccggggcacgag
gcggcggggatcgtcgaggcggtcggctcggaagtgcgcacggtc
aaggtgggtgacgcggtcgtcacctgcctgtccgcgttctgcggt
cattgcgagttctgcgtgaccggccggatgtcgctgtgccttggc
ggcgacacccggcgcggcgcgggcgaggcacctcgccttacccgc
accgacgacggcagcgccgtgaaccagatgctcaacctctcggcc
tttgccgaacagatgctggtgcacgaacatgcctgcgtggcgatc
aatcccgagatgccgctcgaccgcgcggcggtgatcggctgcgcg
gtcaccactggcgcgggtgcggtgttcaacgcggcgaagctgacc
ccgggcgagacggtctgcgtggtcggctgtggcggcgtcggcctt
gccacggtcaacgccgcgaagatcgccggcgcaggccggatcatc
gcggtggacccgatgccggaaaagcgcgaactggccatgaagctg
ggcgcgaccgatgtgatggacgcgggacccgatgcggcggcacag
atcgtcgagatgacgaaaggcggcgtccaccatgcgatcgaggcc
gtggggcgtccggcatcgggcgaccttgcggtcgcgacgctgcgc
cgcggcggcaccgccacgatccttggcatgatgccgctggcacac
aaggtcggactttccgcgatggacctgctgtcggacaagaagctg
cagggcgccatcatgggccgcaaccacttcccggtggacctgccg
cgcctggtcgacttctacatgcgcggcttgctcgatctcgacacg
atcattgccgaacgcatcccgctcgaagggatcaacgatggcttc
gagaagatgaagcagggccattccgcccgctctgtcatcgtgttc
gaccaatga
Saro_0995 Protein Sequence
(SEQ ID NO: 4)
MKAAVLVEPGKPLDIQHLSVSKPGPHEVLIRTAACGLCHSDLHFI
EGAYPHPLPAVPGHEAAGIVEAVGSEVRTVKVGDAVVTCLSAFCG
HCEFCVTGRMSLCLGGDTRRGAGEAPRLTRTDDGSAVNQMLNLSA
FAEQMLVHEHACVAINPEMPLDRAAVIGCAVTTGAGAVENAAKLT
PGETVCVVGCGGVGLATVNAAKIAGAGRIIAVDPMPEKRELAMKL
GATDVMDAGPDAAAQIVEMTKGGVHHAIEAVGRPASGDLAVATLR
RGGTATILGMMPLAHKVGLSAMDLLSDKKLQGAIMGRNHFPVDLP
RLVDFYMRGLLDLDTIIAERIPLEGINDGFEKMKQGHSARSVIVF
DQ*
Saro_3899 Coding Sequence
(SEQ ID NO: 5)
Atggacgcatacgcggcaattatcgagcgtcaaggcggcgaattc
gttctggataacgtctctatcgaggatccgcgcgacggcgaagtg
ctggtcaaggttgccgcagctggcatgtgtcataccgacctgacg
gttcgcgatcaatattacccgacgccgctgccggcggtgctgggc
catgaaggttcgggcgttgtcgaaaaggtcggacgtggcgtcacc
actgtcaagccaggcgacaaggtcgtgctctccttcagctattgc
ggcacctgtccatcgtgcctcaaggggcatcaggcctattgtccg
agcctgttcccgctcaatttcatgggccgccgcctggatggttcg
acgccgattacccgcaacggccaagaggtcaacgcctgcttcttc
gggcaatcctcgttcgcgacctattcgatcgcgtcggaaaacaac
tgcgtcaaggttgccgacgacgcacagatcgaacttttgggccca
ctgggctgcggcatccagaccggggcgggcagcatcctcaatgcg
ctttgtcccgaacctggctcctcgatcgcgatcttcggggtcggg
tcggtcggcctcagcgccgtgatggccgccaaggcctcgggctgc
ctcaagatcatcgcggttgaccgcaacgcaggccgcttggaactg
gcgcgtgaactgggcgccaccgatgtgatcgacgccaacacggtc
aacgctcaggaagcgatcgtcgcgatgaccggtggcggcgccgac
tatgccatggataccaccgccattccagcggtgctgcgctcggcg
gtggacagcacgcacaacatgggtgaaaccgcagtggtcggcggg
gcgaagctgggcaccgagttttcgctagacatgaacaacatgctg
tttggccgcaagttgcgcggcgtagtcgaaggatcgagcaccccg
caggtcttcatcccgcaactgattgcgatgcagaaggccgggctg
ttcccgttcgagaagctctgcaccttctatgatctcgaccagatc
aaccaggccgtcgaggataccgaaaagaccggcaaggcgatcaag
gccattctcaaaatgtag
Saro_3899 Protein Sequence
(SEQ ID NO: 6)
MDAYAAIIERQGGEFVLDNVSIEDPRDGEVLVKVAAAGMCHTDLT
VRDQYYPTPLPAVLGHEGSGVVEKVGRGVTTVKPGDKVVLSFSYC
GTCPSCLKGHQAYCPSLFPLNFMGRRLDGSTPITRNGQEVNACFF
GQSSFATYSIASENNCVKVADDAQIELLGPLGCGIQTGAGSILNA
LCPEPGSSIAIFGVGSVGLSAVMAAKASGCLKIIAVDRNAGRLEL
ARELGATDVIDANTVNAQEAIVAMTGGGADYAMDTTAIPAVLRSA
VDSTHNMGETAVVGGAKLGTEFSLDMNNMLFGRKLRGVVEGSSTP
QVFIPQLIAMQKAGLFPFEKLCTFYDLDQINQAVEDTEKTGKAIK
AILKM*
FerD (Saro_0797) Coding Sequence
(SEQ ID NO: 7)
gtgactgcgtacccttcgctccacatgatcatcgacggcgcccgc
gtcagcggcggcggacgtcgcacccacgcggtcgtcaatcccgct
accggagagaccatcggcgaactgccgctggccgaagtcgccgat
ctcgaccgcgcgctcgaagtcgcggcgaagggcttccgcatctgg
cgcgacagcacgccgcagcagcgcgcagccgtgctccagggcgcg
gcccgcctgatgctggaacggcaggaggacctcgcccgcatcgcc
acgatggaagaaggcaagaccctgcccgaggcgcgcatcgaagtc
ctgatgaacgtgggcctgttcaacttctacgccggcgaggtattc
cggctctatggccgcaccctcgtgcgccctgcgggtcagcgcagc
acgatcacgcatgaaccggtcgggcccgtggccgcctttgcgccg
tggaactttccgctcggcaaccccggccgcaagctcggcgcgccc
attgccgccggttgctcggtgatcctcaaggcggcggaagaaacg
ccggcctccgcgctcggggtgctgcaatgcctgctcgatgcgggc
ctgcccaaggaagtggcccaggccgtgttcggtgtgcctgacgag
gtgagtcgccacctgctcggctcgtccgtcatccgcaagctctcg
ttcaccggctcgaccgtcatcggcaagcacctcatgcgccttgcc
gccgacaacatgttgcgcacaacgatggagcttggcggccacggc
cctgtcctcgtcttcggcgatgccgatatcgacaaggcgctcgat
accatggccgcgtccaagtatcgcaacgcgggccaggtctgcgtc
tcgccaacccgcttcatcgtggaagagagcgtgttcgaacgcttc
cgcgacggttttgccgagcgcgtcggccggatcaaggtcggcaac
ggcctcgatcaggatgcgcagatgggccccatggccaacgcccgc
cgccccgaggcgatggatcgcctgatcggggacgccgtgacccgc
ggcgcaaggctccacaccgggggcgagcgcgtcggcaacgccggc
tatttctacgcccccacggtcctgtccgaagtcccgctcgacgcg
gcgatcatgaacgaggagccgttcggcccggtcgcgctgatcaat
cccttcggcggcgaggaagcgatgatcgccgaggccaaccgcctg
ccctacggcctcgccgcctacgcctggaccgacagcgcggcgcgg
gccaagcgcctcgcccgcgagatcgagacggggatgctcgggctt
aactcgaccatgatcggcggcgcggattcgcccttcggcggggtc
aagtggtccggccacggctccgaggacggtcccgaaggcgtcatg
gcctgccttgtcaccaaggcggtccacgaagggtaa
FerD (Saro_0797) Protein Sequence
(SEQ ID NO: 8)
VTAYPSLHMIIDGARVSGGGRRTHAVVNPATGETIGELPLAEVAD
LDRALEVAAKGFRIWRDSTPQQRAAVLQGAARLMLERQEDLARIA
TMEEGKTLPEARIEVLMNVGLFNFYAGEVFRLYGRTLVRPAGQRS
TITHEPVGPVAAFAPWNFPLGNPGRKLGAPIAAGCSVILKAAEET
PASALGVLQCLLDAGLPKEVAQAVFGVPDEVSRHLLGSSVIRKLS
FTGSTVIGKHLMRLAADNMLRTTMELGGHGPVLVFGDADIDKALD
TMAASKYRNAGQVCVSPTRFIVEESVFERFRDGFAERVGRIKVGN
GLDQDAQMGPMANARRPEAMDRLIGDAVTRGARLHTGGERVGNAG
YFYAPTVLSEVPLDAAIMNEEPFGPVALINPFGGEEAMIAEANRL
PYGLAAYAWTDSAARAKRLAREIETGMLGLNSTMIGGADSPFGGV
KWSGHGSEDGPEGVMACLVTKAVHEG*
Saro_1104 Coding Sequence
(SEQ ID NO: 9)
atgcgcgaacggctacagcaatacattgatggcaagtgggtagac
agcgagggtggcaagcgccacgaggtcatcaatccgacgaccgag
gaaccctgctgcgtcatcacgctgggcacgcaggccgatgtcgac
aaggcagtggccgcggcccagcgcgccttcaagaccttcagcaag
acgacgcgcgaggagcgactcgcgctgcttgaacgcatcgtcgag
gaatacaagaagcgcgtccccgatctcgccgccgcgatggccgag
gaaatgggcgctccggtaagcttcgccagcaccgcgcaggtcggc
gccggcatcggcgccttcctcggcaccatggccgcgctccgcaac
ttctccttcgtcgaggacaacggtgcgttcaaggtcgcctacgaa
ccgatcggcgtcgtcggcatgatcacgccatggaactggcccctc
aaccagatcgcgctcaaggtcgcaccggcgctggccgcgggcaac
accatgatcctcaagccgtccgaggaatgccccaccaacgccgcg
atctttaccgagatcctcgatgccgccggcgtcccgccaggcgtc
ttcaacctcatccagggcgatggtcccggcgtcggcactgcgatc
agctcgcacccgggcatcgacatggtcagcttcaccggctcgacc
cgcgcgggcatcctcgtggcgaaggctgcggccgataccgtcaag
cgcgtccatcaggagcttggcggcaagtcgcccaacgtcgtcctg
cccgatgcagacttcgccaagtacctgccgtcgaccgcgtccggc
ccgttggtcaacagcggccagagctgcatttcgcccacccgcatt
ctcgtaccccgcgaacgcgaagccgaagccgcggcgttcgtttcg
gcgatgtactcggcaaccccggtcggcgatccgatgcaggaaggt
gcgcacatcggcccggtggtcaacaaggcgcagttcgacaagatc
cgcggcctgatccagtcggcgatcgacgaaggcgcgaagctcgag
accggcggccccgacctcccggccaacgtcaaccgcggctactac
atcaagcccacggtcttctccggcgtcacgcccgacatgcgcatt
gcgcaggaggaaatcttcggcccggtcgcgacgatcatggcgtac
gacagcctcgaggaggccatcgagatcgccaacgacaccgcctat
ggcctgtcggcctgcatcaccggcgatccggcgaaggcggctgaa
gtcgcgcccgagcttcgcgccggcatggtcgcgatcaacaactgg
ggccccaccccgggcgcgccgttcggcggctacaagcagtccggc
aacggccgcgaggggggctctatggcctcaaggacttcatggaaa
tgaaggcgatcagcggcctgcctgcctga
Saro_1104 Protein Sequence
(SEQ ID NO: 10)
MRERLQQYIDGKWVDSEGGKRHEVINPTTEEPCCVITLGTQADVD
KAVAAAQRAFKTFSKTTREERLALLERIVEEYKKRVPDLAAAMAE
EMGAPVSFASTAQVGAGIGAFLGTMAALRNFSFVEDNGAFKVAYE
PIGVVGMITPWNWPLNQIALKVAPALAAGNTMILKPSEECPTNAA
IFTEILDAAGVPPGVFNLIQGDGPGVGTAISSHPGIDMVSFTGST
RAGILVAKAAADTVKRVHQELGGKSPNVVLPDADFAKYLPSTASG
PLVNSGQSCISPTRILVPREREAEAAAFVSAMYSATPVGDPMQEG
AHIGPVVNKAQFDKIRGLIQSAIDEGAKLETGGPDLPANVNRGYY
IKPTVFSGVTPDMRIAQEEIFGPVATIMAYDSLEEAIEIANDTAY
GLSACITGDPAKAAEVAPELRAGMVAINNWGPTPGAPFGGYKQSG
NGREGGLYGLKDFMEMKAISGLPA*
Saro_1197 Coding Sequence
(SEQ ID NO: 11)
atgactgccccgaccgccgccgacctttccgccgacatcgcacgc
gtcttcgcactccagcaggcgcacatgtgggaggccaaggcctcc
accgcggccgagcgcaaggaaaagctcgcgcgcctcaaggccgcc
gtcgaagcccacgccgacgacatcgtcgccgccgtcctcgaagac
acgcgcaagccggttggcgaaatccgcgtgaccgaagtcctcaac
gtcaccgccaacatccagcgcaacatcgacaatctcgatgaatgg
atgaagccggtcgaggtcgccacctcgctcaatcccgccgaccgc
gcgcagatcatccacgaagcgcgcggcgtctgcctgatccttggc
ccctggaacttccccctcggcctcgcgctcggtccggtcgccgct
gccatcgccgcaggcaacacctgcatcgtgaagctcaccgacctc
tgccccgccaccgcaagggtggcctcggtgatcgtcagggaagcg
ttcgacgaaaaggatgtggctctgttcgaaggcgacgtctcggtc
gccaccgcgctcctcgatctgccgttcaaccacgtcttcttcacc
ggctcgccccgcgtcggcaagatcgtgatggccgctgccgcaaag
cacctcaccagcgtcacgctcgaacttgggggaagtcgcccgtca
tcgtcgacgatagcgccgacatcgatcaggtcgccgcccagctcg
ccgcggccaagcagttcaacgggggcaggcctgcatcagcccgga
ctacgtcttcgtgaaggaagacaagaaggccgcgctggtcgaagg
cttccgggccaacgtgcagaagaacctctatgacgatgccggcaa
cctgaagaaggacagcatcgcccaggtggtcaacaaggcgaactt
cgaccgcgtgaaggccatgttcgacgatgccgtcgccaagggcgc
gaccgtcgccgccggcggaacgttcgaagccgatgacctcaccat
ccatccgaccatgctgaccggcgtcaccccgcagatgaccatcct
ccaggacgaaatcttcgcccccgtcatcccggtgatgacctacga
cacgctcgaccaggcgatcggctacatcgaagcccgcgacaagcc
gctcgcactctatgtctacagcaaggacgaagcgaacgtcgaaaa
ggtcctcgcccgcacctcgtcgggcggtgtcacggtgaatggcgt
gttctcgcactacctggaaaacaacctgccgttcggcggcgtcaa
caccagcggcatgggcagctaccacggcgtgttcggcttcaagtg
cttcagccacgaacgggctgtctaccgccaccagcagtaa
Saro_1197 Protein Sequence
(SEQ ID NO: 12)
MTAPTAADLSADIARVFALQQAHMWEAKASTAAERKEKLARLKAA
VEAHADDIVAAVLEDTRKPVGEIRVTEVLNVTANIQRNIDNLDEW
MKPVEVATSLNPADRAQIIHEARGVCLILGPWNFPLGLALGPVAA
AIAAGNTCIVKLTDLCPATARVASVIVREAFDEKDVALFEGDVSV
ATALLDLPFNHVFFTGSPRVGKIVMAAAAKHLTSVTLELGGKSPV
IVDDSADIDQVAAQLAAAKQFNGGQACISPDYVFVKEDKKAALVE
GFRANVQKNLYDDAGNLKKDSIAQVVNKANFDRVKAMEDDAVAKG
ATVAAGGTFEADDLTIHPTMLTGVTPQMTILQDEIFAPVIPVMTY
DTLDQAIGYIEARDKPLALYVYSKDEANVEKVLARTSSGGVTVNG
VFSHYLENNLPFGGVNTSGMGSYHGVFGFKCFSHERAVYRHQQ*
Saro_2869 Coding Sequence
(SEQ ID NO: 13)
atgaacgacatgaccaccatctcgcgcacgcagcgcgaatactcg
gaggccgccaaggccttcctcgcgcgcaagccgcagttgttcatc
aacaacgagtgggtcgacagcagccacgacgccgtgatcgaggtg
gaagacccctcgaacggcaggatcgtcggtcatgtcgtcgatgcc
tcggacaaggacgtcgaccgggcggttgccgcggcgcgcgccgcg
ttcgacgatggccgctggtccaacctgccgccaatggtccgcgat
cgcaccatgaatcgcctggccgacctgcttgaagccaacgccgat
ctctttgccgagctcgaagcgatcgacaacggcaagcccaagggc
atggccggcgccgtcgacatccccggcgcgatcagccagctccgc
ttcatggccggctgggccagcaaggtcgcgggcgagacgacgcag
ccctacacgatgcccaacggcaccgtgttcagctacaccgtcaag
gaacccgtcggcgtctgcgcgcagatcgtgccgtggaacttcccg
ctgctgatggcctcgctcaagatcgccccggcgctggcggctggc
tgcaccctggtgctgaagcccgccgaacagacctcgcttaccgcg
ctcaagcttgccgatctcgtggtcgaggccggcttccctgcgggc
gtgatcaacatcatcaccggcaacggccacaccgccggtgaccgc
atggtcaagcatcccgacgtcgacaaggtcgccttcaccggctcg
accgagatcggcaagctgatcaatcgcaacgccaccaccacgctc
aagcgggtcacgctcgaactggggggaagagccccgtcgtggtca
tgcccgacgtcgacgtggcgcagaccgcgcctggcgttgccggcg
cgatcttcttcaacgcgggccaggtctgcgttgccggttcgcgtc
tctatgcgcaccgttcggtgttcgattccgtgctcgaaggcatga
cccagaccgcgccgttctgggcgccgcgcccctcgctggatcccg
aagcccacatgggcccgttggtcagcaaggagcagcacgaccgcg
tgatgggctacatcgaggcgggcaagcgcgatggcgccagcgtcg
tcatgggcggcgattgccccagcgccgatggcgggtactacgtca
acccgacgatccttgcagacgtgaacccgcagatgtcggtcgtgc
gcgaggaaatcttcggccccgtcgtcgtcgcccagcgcttcgacg
atctcgatgaagtggcgaagatggccaacgacacctgcttcggcc
tcggcgcgggcgtgtggacgcgcgatgtcgcggtgatgcacaagc
ttgcctcgaagatcaaatcgggcaccgtgtggggcaactgccacg
ccctgatcgataccgcgctgccctttggcggctacaaggaatcgg
gcctgggccgcgaacaggggcgcgccggcatcgacgcctacctcg
agaccaagaccgtcatcatccagatgtaa
Saro_2869 Protein Sequence
(SEQ ID NO: 14)
MNDMTTISRTQREYSEAAKAFLARKPQLFINNEWVDSSHDAVIEV
EDPSNGRIVGHVVDASDKDVDRAVAAARAAFDDGRWSNLPPMVRD
RTMNRLADLLEANADLFAELEAIDNGKPKGMAGAVDIPGAISQLR
FMAGWASKVAGETTQPYTMPNGTVFSYTVKEPVGVCAQIVPWNFP
LLMASLKIAPALAAGCTLVLKPAEQTSLTALKLADLVVEAGFPAG
VINIITGNGHTAGDRMVKHPDVDKVAFTGSTEIGKLINRNATTTL
KRVTLELGGKSPVVVMPDVDVAQTAPGVAGAIFFNAGQVCVAGSR
LYAHRSVFDSVLEGMTQTAPFWAPRPSLDPEAHMGPLVSKEQHDR
VMGYIEAGKRDGASVVMGGDCPSADGGYYVNPTILADVNPQMSVV
REEIFGPVVVAQRFDDLDEVAKMANDTCFGLGAGVWTRDVAVMHK
LASKIKSGTVWGNCHALIDTALPEGGYKESGLGREQGRAGIDAYL
ETKTVIIQM*
PcfL (Saro_0796) Coding Sequence
(SEQ ID NO: 15)
Gtgtccgatagcaatcagattgccgcgctcgaaagccgcctgaac
gacctcgaaaggcgcctgacggtgcgcgaggacgagctggacgta
cgcaagctccagcatctctacggctacctgatcgacaagtgcatg
tataacgagaccgtggacctgttcaccgaagatggcgaagtgcgc
ttcttcggcggcgtctggaagggcaaggagggcatccgccgtctc
tacgtcgaacgtttccagaagcgcttcacctacggcaacaacggc
ccgatcgacggcttcctgctcgatcacccccagcttcaggacatc
atccacgtgcaggatgacggggtcaccgctctcggccgcgcgcgg
tcgatgatgcaggccggtcgccacaaggattacgagggcgatgcc
ccgcacctcaaggcgcgccagtggtgggaaggcggcatctacgag
aacacctacaagaaggggacggcgtgtggcggatgcacatcctca
actacatgccgatctggcacgccgatttcgaaagcggctgggcca
acaccccgcacgaatacgtgccgttccccaaggtcacctatcccg
aagacccgaccggaccggacgaactgatcgccgaccactggctct
ggccgacccacaagctgaaccccttccacatgaagcacccggtga
cgggcgaggaaatggtcgcgcagcgctggcagggcgacatcgacc
gcgagaacgcgcggaaataa
PcfL (Saro_0796) Protein Sequence
(SEQ ID NO: 16)
VSDSNQIAALESRLNDLERRLTVREDELDVRKLQHLYGYLIDKCM
YNETVDLFTEDGEVRFFGGVWKGKEGIRRLYVERFQKRFTYGNNG
PIDGFLLDHPQLQDIIHVQDDGVTALGRARSMMQAGRHKDYEGDA
PHLKARQWWEGGIYENTYKKVDGVWRMHILNYMPIWHADFESGWA
NTPHEYVPFPKVTYPEDPTGPDELIADHWLWPTHKLNPFHMKHPV
TGEEMVAQRWQGDIDRENARK*
LsdD (Saro_0802) Coding Sequence
(SEQ ID NO: 17)
atggcccaatttccgaacacccccagcttcacgggattcaacacg
ccgtcgcggatcgaggcggatatcgccgatctggcccacgaaggc
acgattccgcaagggttaaacggcgcattctaccgcgtccagccc
gacccgcagtttcctccccgcctcgacgacgacatcgccttcaac
ggcgacggcatgatcacccgcttccacatccacgacggccaggtc
gacttccgccagcgctgggcgaagaccgacaagtggaagctggag
aacgccgccggaaaggccctgttcggcgcctaccgcaacccgctg
accgacgacgaggcggtcaagggcgagatccgttcgaccgccaac
accaacgccttcgtgttcggcggcaagctgtgggcgatgaaggag
gacagtcccgccctcgtcatggacccggcgacgatggaaaccttc
gggttcgagaagttcggcggcaagatgaccggccagacctttacc
gcccaccccaaggtcgatccgaagaccggcaacatggtcgccatc
ggctatgccgcaagcgggctgtgcaccgacgatgtgacctacatg
gaagtgagcccggagggcgagcttgtccgcgaagtgtggttcaag
gtgccgtactactgcatgatgcacgacttcggcatcaccgaggat
tacctcgtgctgcacatcgtgccttccatcggaagctgggaaagg
ctggaacagggcaagccgcacttcggcttcgacacgaccatgccg
gtgcacctcggcatcatcccgcgccgcgacggcgtgcgccaggaa
gacatccgctggttcacgcgggacaactgctttgccagccatgtc
ctgaacgcctggcaagaggggaccaagatccacttcgtgacctgc
gaggcgaagaacaacatgttcccgttcttccccgacgtccacggc
gcgcccttcaacggcatggaggccatgagccatccgaccgactgg
gtggtcgacatggccagcaacggcgaggactttgccgggatcgtg
aagctttccgacacagccgccgagttcccgcgcatcgacgaccgc
tttaccggccagaagacccgccatggctggttcctcgaaatggac
atgaagcgcccggtggaattgcgcggcggcagcgccggcggcctg
ctgatgaactgcctgttccacaaggacttcgaaacgggtcgcgag
cagcactggtggtgcggcccggtgtcgagccttcaggagccgtgc
ttcgtgccgcgcgccaaggatgcccccgaaggcgacggctggatc
gtgcaggtttgcaaccggctggaagagcagcgcagcgacttgctg
atcttcgacgcgctcgacatcgagaaaggcccggtggccacggtc
aacatccccatccgcctgcgcttcggccttcacggcaactgggcg
aatgccgacgaaatcggccttgccgagaaggtcctggccgcatga
LsdD (Saro_0802) Protein Sequence
(SEQ ID NO: 18)
MAQFPNTPSFTGFNTPSRIEADIADLAHEGTIPQGLNGAFYRVQP
DPQFPPRLDDDIAFNGDGMITRFHIHDGQVDFRQRWAKTDKWKLE
NAAGKALFGAYRNPLTDDEAVKGEIRSTANTNAFVFGGKLWAMKE
DSPALVMDPATMETFGFEKFGGKMTGQTFTAHPKVDPKTGNMVAI
GYAASGLCTDDVTYMEVSPEGELVREVWFKVPYYCMMHDFGITED
YLVLHIVPSIGSWERLEQGKPHFGFDTTMPVHLGIIPRRDGVRQE
DIRWFTRDNCFASHVLNAWQEGTKIHFVTCEAKNNMFPFFPDVHG
APFNGMEAMSHPTDWVVDMASNGEDFAGIVKLSDTAAEFPRIDDR
FTGQKTRHGWFLEMDMKRPVELRGGSAGGLLMNCLFHKDFETGRE
QHWWCGPVSSLQEPCFVPRAKDAPEGDGWIVQVCNRLEEQRSDLL
IFDALDIEKGPVATVNIPIRLRFGLHGNWANADEIGLAEKVLAA*
LigW (Saro_0799) Coding Sequence
(SEQ ID NO: 19)
atgacacaagaccttaagaccggcggcgagcagggctacctgcgc
atcgccaccgaggaagccttcgccacgcgcgagatcatcgacgtc
tacctgcgcatgatccgcgatggcactgccgacaagggcatggtc
tcgctctggggcttctacgcccagtccccctcagagcgcgccacc
cagatcctcgaacgcctgctcgatcttggcgagcgccgcatcgcc
gacatggacgcgaccggcatcgacaaggctatcctcgcgctgacc
tcgcccggcgtccagccgctgcacgaccttgacgaggccaggacg
ctcgccacccgcgccaacgacacgcttgccgacgcgtgccaaaag
tacccagaccgcttcatcggcatgggcaccgtcgccccgcaggac
ccggaatggtccgcgcgcgagatccatcgtggtgccagggaactg
ggcttcaagggcatccagatcaacagccacacgcaagggcgctac
ctcgacgaggagttcttcgacccgatcttccgcgccctcgttgaa
gtcgaccagccgctctacatccaccctgccacttcgcccgattcc
atgatcgacccgatgctcgaagcgggcctcgacggcgccatcttc
ggcttcggcgtggagacgggcatgcacctgctgcgcctcatcacc
atcggcatcttcgacaagtatcccagccttcagatcatggtcggc
cacatgggcgaggcgctgccctactggctctaccgcctggactac
atgcaccaggccggtgtccgctcgcagcgctacgaacgcatgaag
cccctgaagaagaccatcgagggctacctcaagtccaacgtcctc
gtcaccaattcgggcgtcgcgtgggaacctgcgatcaagttctgc
cagcaggtcatgggcgaggaccgcgttatgtacgcgatggactac
ccctaccagtacgttgccgacgaggtgcgcgcgatggacgccatg
gacatgagtgcgcaaacgaagaagaagttcttccagaccaacgcg
gagaagtggttcaagctttga
LigW (Saro_0799) Protein Sequence
(SEQ ID NO: 20)
MTQDLKTGGEQGYLRIATEEAFATREIIDVYLRMIRDGTADKGMV
SLWGFYAQSPSERATQILERLLDLGERRIADMDATGIDKAILALT
SPGVQPLHDLDEARTLATRANDTLADACQKYPDRFIGMGTVAPQD
PEWSAREIHRGARELGFKGIQINSHTQGRYLDEEFFDPIFRALVE
VDQPLYIHPATSPDSMIDPMLEAGLDGAIFGFGVETGMHLLRLIT
IGIFDKYPSLQIMVGHMGEALPYWLYRLDYMHQAGVRSQRYERMK
PLKKTIEGYLKSNVLVTNSGVAWEPAIKFCQQVMGEDRVMYAMDY
PYQYVADEVRAMDAMDMSAQTKKKFFQTNAEKWFKL*

### EXEMPLARY VERSIONS OF THE INVENTION

1. A recombinant microorganism comprising any one or more, any two or more, any three or more, any four or more, or each of:
one or more recombinant alcohol dehydrogenase genes encoding:
   FdhA of Novosphingobium aromaticivorans (SEQ ID NO:2) or a homolog thereof;
   Saro_0995 of Novosphingobium aromaticivorans (SEQ ID NO:4) or a homolog thereof; and/or
   Saro_3899 of Novosphingobium aromaticivorans (SEQ ID NO:6) or a homolog thereof;
one or more recombinant aldehyde dehydrogenase genes encoding:
   FerD of Novosphingobium aromaticivorans (SEQ ID NO:8) or a homolog thereof;
   Saro_1104 of Novosphingobium aromaticivorans (SEQ ID NO:10) or a homolog thereof;
   Saro_1197 of Novosphingobium aromaticivorans (SEQ ID NO:12) or a homolog thereof; and/or
   Saro_2869 of Novosphingobium aromaticivorans (SEQ ID NO:14) or a homolog thereof;
a recombinant γ-formaldehyde lyase gene encoding PcfL of Novosphingobium aromaticivorans (SEQ ID NO:16) or a homolog thereof;
a recombinant lignostilbene dioxygenase gene encoding LsdD of Novosphingobium aromaticivorans (SEQ ID NO:18) or a homolog thereof; and
a recombinant aromatic acid decarboxylase gene encoding LigW of Novosphingobium aromaticivorans (SEQ ID NO:20) or a homolog thereof.

2. The recombinant microorganism of version 1, comprising any two or more, any three or more, any four or more, or each of:
the one or more recombinant alcohol dehydrogenase genes;
the one or more recombinant aldehyde dehydrogenase genes;
the recombinant γ-formaldehyde lyase gene;
the recombinant lignostilbene dioxygenase gene; and
the recombinant aromatic acid decarboxylase gene.

3. The recombinant microorganism of version 1, comprising any three or more, any four or more, or each of:
the one or more recombinant alcohol dehydrogenase genes;
the one or more recombinant aldehyde dehydrogenase genes;
the recombinant γ-formaldehyde lyase gene;
the recombinant lignostilbene dioxygenase gene; and
the recombinant aromatic acid decarboxylase gene.

4. The recombinant microorganism of version 1, comprising any four or more or each of:
the one or more recombinant alcohol dehydrogenase genes;
the one or more recombinant aldehyde dehydrogenase genes;
the recombinant γ-formaldehyde lyase gene;
the recombinant lignostilbene dioxygenase gene; and
the recombinant aromatic acid decarboxylase gene.

5. The recombinant microorganism of version 1, comprising each of:
the one or more recombinant alcohol dehydrogenase genes;
the one or more recombinant aldehyde dehydrogenase genes;
the recombinant γ-formaldehyde lyase gene;
the recombinant lignostilbene dioxygenase gene; and
the recombinant aromatic acid decarboxylase gene.

6. The recombinant microorganism of any prior version, comprising the one or more recombinant alcohol dehydrogenase genes.

7. The recombinant microorganism of any prior version, wherein, when present, the one or more recombinant alcohol dehydrogenase genes encode:
FdhA of Novosphingobium aromaticivorans (SEQ ID NO:2), a protein comprising a sequence at least 80%, at least 85%, at least 90%, at least 95%, or at least 99% identical to SEQ ID NO:2, an ortholog of FdhA of Novosphingobium aromaticivorans, or a recombinant variant of the ortholog of FdhA of Novosphingobium aromaticivorans;
Saro_0995 of Novosphingobium aromaticivorans (SEQ ID NO:4), a protein comprising a sequence at least 80%, at least 85%, at least 90%, at least 95%, or at least 99% identical to SEQ ID NO:4, an ortholog of Saro_0995 of Novosphingobium aromaticivorans, or a recombinant variant of the ortholog of Saro_0995 of Novosphingobium aromaticivorans; and/or
Saro_3899 of Novosphingobium aromaticivorans (SEQ ID NO:6), a protein comprising a sequence at least 80%, at least 85%, at least 90%, at least 95%, or at least 99% identical to SEQ ID NO:6, an ortholog of Saro_3899 of Novosphingobium aromaticivorans, or a recombinant variant of the ortholog of Saro_3899 of Novosphingobium aromaticivorans.

8. The recombinant microorganism of any prior version comprising the one or more recombinant aldehyde dehydrogenase genes.

9. The recombinant microorganism of any prior version, wherein, when present, the one or more recombinant aldehyde dehydrogenase genes encode:
FerD of Novosphingobium aromaticivorans (SEQ ID NO:8), a protein comprising a sequence at least 80%, at least 85%, at least 90%, at least 95%, or at least 99% identical to SEQ ID NO:8, an ortholog of FerD of Novosphingobium aromaticivorans, or a recombinant variant of the ortholog of FerD of Novosphingobium aromaticivorans;
Saro_1104 of Novosphingobium aromaticivorans (SEQ ID NO:10), a protein comprising a sequence at least 80%, at least 85%, at least 90%, at least 95%, or at least 99% identical to SEQ ID NO:10, an ortholog of Saro_1104 of Novosphingobium aromaticivorans, or a recombinant variant of the ortholog of Saro_1104 of Novosphingobium aromaticivorans;
Saro_1197 of Novosphingobium aromaticivorans (SEQ ID NO:12), a protein comprising a sequence at least 80%, at least 85%, at least 90%, at least 95%, or at least 99% identical to SEQ ID NO:12, an ortholog of Saro_1197 of Novosphingobium aromaticivorans, or a recombinant variant of the ortholog of Saro_1197 of Novosphingobium aromaticivorans; and/or
Saro_2869 of Novosphingobium aromaticivorans (SEQ ID NO:14), a protein comprising a sequence at least 80%, at least 85%, at least 90%, at least 95%, or at least 99% identical to SEQ ID NO:14, an ortholog of Saro_2869 of Novosphingobium aromaticivorans, or a recombinant variant of the ortholog of Saro_2869 of Novosphingobium aromaticivorans.

10. The recombinant microorganism of any prior version, comprising the recombinant 7-formaldehyde lyase gene.

11. The recombinant microorganism of any prior version, wherein, when present, the recombinant γ-formaldehyde lyase gene encodes PcfL of Novosphingobium aromaticivorans (SEQ ID NO:16), a protein comprising a sequence at least 80%, at least 85%, at least 90%, at least 95%, or at least 99% identical to SEQ ID NO:16, an ortholog of PcfL of Novosphingobium aromaticivorans, a recombinant variant of the ortholog of PcfL of Novosphingobium aromaticivorans.

12. The recombinant microorganism of any prior version, comprising the recombinant lignostilbene dioxygenase gene.

13. The recombinant microorganism of any prior version, wherein, when present, the recombinant lignostilbene dioxygenase gene encodes LsdD of Novosphingobium aromaticivorans (SEQ ID NO:18), a protein comprising a sequence at least 80% identical, at least 85% identical, at least 90% identical, at least 95% identical, or at least 99% identical to SEQ ID NO:18, an ortholog of LsdD of Novosphingobium aromaticivorans, or a recombinant variant of the ortholog of LsdD of Novosphingobium aromaticivorans.

14. The recombinant microorganism of any prior version, comprising the recombinant aromatic acid decarboxylase gene.
a recombinant aromatic acid decarboxylase gene encoding LigW of Novosphingobium aromaticivorans (SEQ ID NO:20) or a homolog thereof.

15. The recombinant microorganism of any prior version, wherein, when present, the recombinant aromatic acid decarboxylase gene encodes LigW of Novosphingobium aromaticivorans (SEQ ID NO:20), a protein comprising a sequence at least 80% identical, at least 85% identical, at least 90% identical, at least 95% identical, or at least 99% identical to SEQ ID NO:20, an ortholog of LigW of Novosphingobium aromaticivorans, or a recombinant variant of the ortholog of LigW of Novosphingobium aromaticivorans.

16. The recombinant microorganism of any prior version, wherein the recombinant microorganism is a bacterium.

17. The recombinant microorganism of any prior version, wherein the recombinant microorganism is an Alphaproteobacterium.

18. The recombinant microorganism of any prior version, wherein the recombinant microorganism is from an order selected from the group consisting of Sphingomonadales, Actinomyces, Gammaproteobacteria, Betaproteobacteria, and Bacilli.

19. A method of catabolizing a lignin aromatic, the method comprising culturing the recombinant microorganism of any prior version in a medium comprising the lignin aromatic to thereby catabolize the lignin aromatic.

20. The method of version 19, wherein the lignin aromatic comprises a β-5 linked lignin aromatic.

21. The method of any one of versions 19-20, wherein the lignin aromatic comprises one or more of dehydrodiconiferyl alcohol (DC-A), dehydrodiconiferyl aldehyde (DC-L), dehydrodiconiferyl carboxylic acid (DC-C), dehydrodiconiferyl stilbene carboxylic acid (DC-S-C), 5-formyl ferulate (5-FF), 5-carboxyferulate (5-CF), and 4-hydroxyphenyl and syringyl analogs thereof.

What is claimed is:

1. A recombinant microorganism comprising any one or more of:
one or more recombinant alcohol dehydrogenase genes encoding:
FdhA of Novosphingobium aromaticivorans (SEQ ID NO:2) or a homolog thereof;
Saro_0995 of Novosphingobium aromaticivorans (SEQ ID NO:4) or a homolog thereof; and/or
Saro_3899 of Novosphingobium aromaticivorans (SEQ ID NO:6) or a homolog thereof;
one or more recombinant aldehyde dehydrogenase genes encoding:
FerD of Novosphingobium aromaticivorans (SEQ ID NO:8) or a homolog thereof;
Saro_1104 of Novosphingobium aromaticivorans (SEQ ID NO:10) or a homolog thereof;
Saro_1197 of Novosphingobium aromaticivorans (SEQ ID NO:12) or a homolog thereof; and/or
Saro_2869 of Novosphingobium aromaticivorans (SEQ ID NO:14) or a homolog thereof;
a recombinant γ-formaldehyde lyase gene encoding PcfL of Novosphingobium aromaticivorans (SEQ ID NO:16) or a homolog thereof;
a recombinant lignostilbene dioxygenase gene encoding LsdD of Novosphingobium aromaticivorans (SEQ ID NO:18) or a homolog thereof; and
a recombinant aromatic acid decarboxylase gene encoding LigW of Novosphingobium aromaticivorans (SEQ ID NO:20) or a homolog thereof.

2. The recombinant microorganism of claim 1, comprising any two or more of:
the one or more recombinant alcohol dehydrogenase genes;
the one or more recombinant aldehyde dehydrogenase genes;
the recombinant γ-formaldehyde lyase gene;
the recombinant lignostilbene dioxygenase gene; and
the recombinant aromatic acid decarboxylase gene.

3. The recombinant microorganism of claim 1, comprising any three or more of:
the one or more recombinant alcohol dehydrogenase genes;
the one or more recombinant aldehyde dehydrogenase genes;
the recombinant γ-formaldehyde lyase gene;
the recombinant lignostilbene dioxygenase gene; and
the recombinant aromatic acid decarboxylase gene.

4. The recombinant microorganism of claim 1, comprising any four or more of:
the one or more recombinant alcohol dehydrogenase genes;
the one or more recombinant aldehyde dehydrogenase genes;
the recombinant γ-formaldehyde lyase gene;
the recombinant lignostilbene dioxygenase gene; and
the recombinant aromatic acid decarboxylase gene.

5. The recombinant microorganism of claim 1, comprising each of:
the one or more recombinant alcohol dehydrogenase genes;
the one or more recombinant aldehyde dehydrogenase genes;
the recombinant γ-formaldehyde lyase gene;
the recombinant lignostilbene dioxygenase gene; and
the recombinant aromatic acid decarboxylase gene.

6. The recombinant microorganism of claim 1, comprising the one or more recombinant alcohol dehydrogenase genes.

7. The recombinant microorganism of claim 6, wherein the one or more recombinant alcohol dehydrogenase genes encode:
FdhA of Novosphingobium aromaticivorans (SEQ ID NO:2), a protein comprising a sequence at least 95% identical to SEQ ID NO:2, an ortholog of FdhA of Novosphingobium aromaticivorans, or a recombinant variant of the ortholog of FdhA of Novosphingobium aromaticivorans;
Saro_0995 of Novosphingobium aromaticivorans (SEQ ID NO:4), a protein comprising a sequence at least 95% identical to SEQ ID NO:4, an ortholog of Saro_0995 of Novosphingobium aromaticivorans, or a recombinant variant of the ortholog of Saro_0995 of Novosphingobium aromaticivorans; and/or
Saro_3899 of Novosphingobium aromaticivorans (SEQ ID NO:6), a protein comprising a sequence at least 95% identical to SEQ ID NO:6, an ortholog of Saro_3899 of Novosphingobium aromaticivorans, or a recombinant variant of the ortholog of Saro_3899 of Novosphingobium aromaticivorans.

8. The recombinant microorganism of claim 1 comprising the one or more recombinant aldehyde dehydrogenase genes.

9. The recombinant microorganism of claim 8, wherein, when present, the one or more recombinant aldehyde dehydrogenase genes encode:
FerD of Novosphingobium aromaticivorans (SEQ ID NO:8), a protein comprising a sequence at least 95% identical to SEQ ID NO:8, an ortholog of FerD of Novosphingobium aromaticivorans, or a recombinant variant of the ortholog of FerD of Novosphingobium aromaticivorans;
Saro_1104 of Novosphingobium aromaticivorans (SEQ ID NO:10), a protein comprising a sequence at least 95% identical to SEQ ID NO:10, an ortholog of Saro_1104 of Novosphingobium aromaticivorans, or a recombinant variant of the ortholog of Saro_1104 of Novosphingobium aromaticivorans;
Saro_1197 of Novosphingobium aromaticivorans (SEQ ID NO:12), a protein comprising a sequence at least 95% identical to SEQ ID NO:12, an ortholog of Saro_1197 of Novosphingobium aromaticivorans, or a recombinant variant of the ortholog of Saro_1197 of Novosphingobium aromaticivorans; and/or
Saro_2869 of Novosphingobium aromaticivorans (SEQ ID NO:14), a protein comprising a sequence at least 95% identical to SEQ ID NO:14, an ortholog of Saro_2869 of Novosphingobium aromaticivorans, or a recombinant variant of the ortholog of Saro_2869 of Novosphingobium aromaticivorans.

10. The recombinant microorganism of claim 1, comprising the recombinant T-formaldehyde lyase gene.

11. The recombinant microorganism of claim 10, wherein, when present, the recombinant T-formaldehyde lyase gene encodes PcfL of Novosphingobium aromaticivorans (SEQ ID NO:16), a protein comprising a sequence at least 95% identical to SEQ ID NO:16, an ortholog of PcfL of Novosphingobium aromaticivorans, a recombinant variant of the ortholog of PcfL of Novosphingobium aromaticivorans.

12. The recombinant microorganism of claim 1, comprising the recombinant lignostilbene dioxygenase gene.

13. The recombinant microorganism of claim 12, wherein, when present, the recombinant lignostilbene dioxygenase gene encodes LsdD of Novosphingobium aromaticivorans (SEQ ID NO:18), a protein comprising a sequence at least 95% identical to SEQ ID NO:18, an ortholog of LsdD of Novosphingobium aromaticivorans, or a recombinant variant of the ortholog of LsdD of Novosphingobium aromaticivorans.

14. The recombinant microorganism of claim 1, comprising the recombinant aromatic acid decarboxylase gene.

15. The recombinant microorganism of claim 14, wherein, when present, the recombinant aromatic acid decarboxylase gene encodes LigW of Novosphingobium aromaticivorans (SEQ ID NO:20), a protein comprising a sequence at least 95% identical to SEQ ID NO:20, an ortholog of LigW of Novosphingobium aromaticivorans, or a recombinant variant of the ortholog of LigW of Novosphingobium aromaticivorans.

16. The recombinant microorganism of claim 1, wherein the recombinant microorganism is a bacterium.

17. The recombinant microorganism of claim 1, wherein the recombinant microorganism is an Alphaproteobacterium.

18. The recombinant microorganism of claim 1, wherein the recombinant microorganism is from an order selected from the group consisting of Sphingomonadales, Actinomyces, Gammaproteobacteria, Betaproteobacteria, and Bacilli.

19. A method of catabolizing a lignin aromatic, the method comprising culturing the recombinant microorganism of claim 1 in a medium comprising the lignin aromatic to thereby catabolize the lignin aromatic.

20. The method of claim 19, wherein the lignin aromatic comprises a β-5 linked lignin aromatic.
